(19) United States
(12) Patent Application Publication
(10) Pub. No.: US 2024/0218457 A1
(43) Pub. Date: Jul. 4, 2024

(54) METHOD FOR DIAGNOSING ACTIVE TUBERCULOSIS AND PROGRESSION TO ACTIVE TUBERCULOSIS

(71) Applicant: The Board of Trustees of the Leland Stanford Junior University, Stanford, CA (US)

(72) Inventors: Aditya Manohar RAO, Stanford, CA (US); Purvesh KHATRI, Menlo Park, CA (US); Madeleine SCOTT, Stanford, CA (US)

(21) Appl. No.: 18/288,765

(22) PCT Filed: Apr. 29, 2022
(86) PCT No.: PCT/US2022/027063

§ 371 Date: Oct. 27, 2023

Related U.S. Application Data
(60) Provisional application No. 63/183,432, filed on May 3, 2021.

(51) Int. Cl. C12Q 1/689; C12Q 1/6883

(52) U.S. Cl. CPC C12Q 1/689; C12Q 1/6883; C12Q 2600/118

(57) ABSTRACT

Methods for diagnosing active tuberculosis and progression to active tuberculosis are disclosed. In some embodiments, the method may comprise (a) obtaining a sample of RNA from a subject, e.g., a subject having latent tuberculosis and/or symptoms of active tuberculosis; and (b) measuring the amount of RNA transcripts encoded by PLAAT4, CYB561, GBP5, SMAD7, LAX1, CDKN1C, CA5B, EPHA4 and CD83 in the sample, to produce gene expression data. This data can be used for the diagnosis and to make treatment decisions.

### CROSS-REFERENCING

This application claims the benefit of U.S. provisional application Ser. No. 63/183,432, filed on May 3, 2021, which application is incorporated by reference for all purposes.

### STATEMENT REGARDING FEDERALLY SPONSORED RESEARCH OR DEVELOPMENT

This invention was made with Government support under contract W81XWH-18-1-0253 awarded by the Department of Defense. The Government has certain rights in the invention.

### BACKGROUND

Tuberculosis (TB) is a worldwide public health issue, with 9 million new infections and 1.5 million deaths in 2013 (Global Tuberculosis Programme, World Health Organization. Global tuberculosis report. Geneva, Switzerland: World Health Organisation; 2012:volumes). Despite advances in diagnosis and treatment, there is still a large burden of disease. TB is difficult to accurately diagnose; traditional methods such as tuberculin skin testing and interferon gamma release assays (IGRAs) are unable to distinguish between latent TB (LTB) and active TB (ATB), and have lower sensitivity in HIV-positive patients. Although the Xpert MTB/RIF assay has significantly improved diagnostic power, it suffers from reduced accuracy in HIV-positive patients, and is not useful for monitoring treatment response (Steingart et al. (2014) Cochrane Database Syst. Rev. 1:CD009593; Friedrich et al. (2013) Lancet Respir. Med 1:462-470). Further, it relies on induced sputum, which can be difficult to obtain from adults after symptomatic improvement or from pediatric patients at any time. Current methods could thus potentially be complemented by an accurate, HIV-invariant blood-based diagnostic and treatment-response test.

Several studies have investigated the host response to tuberculosis infection using microarray-based whole genome expression profiles in peripheral blood. However, the results from these studies have not translated into clinical practice so far, due largely to poor generalizability. For instance, different gene signatures, with minimal overlap, have been proposed for distinguishing ATB from other diseases (OD) or LTB (REF Nature and PloS Medicine) and in children and adults (Anderson et al. (2014) N. Engl. J. Med. 370:1712-1723; Kaforou et al. (2014) J. Infect 69 Suppl. 1:S28-31). Many of these studies have now been deposited in publically accessible databases such as the NTH Gene Expression Omnibus (GEO), allowing their further analysis and re-use.

There remains a need for sensitive and specific diagnostic tests for tuberculosis that can distinguish between latent and active disease and better methods of monitoring responses to treatment.

### SUMMARY

Provided herein is a method for a sample, the method comprising: (a) obtaining a sample of RNA from a subject, e.g., a subject having latent tuberculosis and/or symptoms of active tuberculosis; and (b) measuring the amount of RNA transcripts encoded of at least two of (e.g., 3, 4, 5, 6, 7, 8 or all of) PLAAT4, CYB561, GBP5, SMAD7, LAX1, CDKN1C, CA5B, EPHA4 and CD83 in the sample, to produce gene expression data. The sample can be RNA isolated from whole blood, white blood cells, neutrophils, peripheral blood mononuclear cells (PBMCs), or buffy coat, for example.

In some embodiment, the method may comprise (c) generating a report indicating whether the subject has active tuberculosis or is progressing to active tuberculosis based on the gene expression data, wherein: (i) increased PLAAT4, CYB561, and GBP5 expression indicates that the subject has active tuberculosis or is progressing to active tuberculosis; and (ii) increased SMAD7, LAX1, CDKN1C, CA5B, EPHA4, and CD83 indicates that the subject does not have active tuberculosis and is not progressing to active tuberculosis. In some embodiments, the method may comprise calculating a tuberculosis score based on the levels of expression of the RNA transcripts in the subject, wherein a higher tuberculosis score for the subject indicates that the subject is more likely to have active tuberculosis or progression to active tuberculosis.

If the subject is identified as having active tuberculosis or progression to active tuberculosis based on the gene expression data, then the method may comprise administering antibiotics, one or more of isoniazid, rifampin, pyrazinamide, and ethambutol, to the patient.

Also provided is a method for treating a subject. In some embodiments, this method may comprise: (a) identifying a patient as having active tuberculosis or progression to active tuberculosis based on the amount of RNA transcripts encoded by at least two of (e.g., 3, 4, 5, 6, 7, 8 or all of) PLAAT4, CYB561, GBP5, SMAD7, LAX1, CDKN1C, CA5B, EPHA4 and CD83 in a sample from the subject; and (b) treating the subject with antibiotics, e.g., one or more of isoniazid, rifampin, pyrazinamide, and ethambutol. In some embodiments, the identifying may be done by reviewing a report indicating whether the subject has active tuberculosis or is progressing to active tuberculosis. In these embodiments, the report may comprise a tuberculosis score, wherein a higher tuberculosis score for the subject indicates that the subject is more likely to have active tuberculosis or progression to active tuberculosis.

Also provided is a kit for diagnosing active tuberculosis or progression to active tuberculosis. In some embodiments, the kit may comprise reagents for measuring the amount of RNA transcripts encoded by at least two of (e.g., 3, 4, 5, 6, 7, 8 or all of) PLAAT4, CYB561, GBP5, SMAD7, LAX1, CDKN1C, CA5B, EPHA4 and CD83.

### BRIEF DESCRIPTION OF THE FIGURES

FIG. 1 shows a MANATEE Framework Diagram. Schematic of the novel multi-cohort analysis workflow that was used for training and validation of the ATB vs. Other signature. Countries that contributed <3 samples were not included in country tabulations. Numbers for the Independent Validation datasets were calculated using any datasets that were used in either the pooled Independent Validation data, the individual Independent Validation cohorts, or both.

FIGS. 2A-2B shows performance of the 9-gene signature in independent retrospective validation data. (A) Receiver operating characteristics (ROC) curves for diagnosing active tuberculosis in pooled independent validation data for ATB vs. Healthy, LTBI, and other diseases (OD). (B) Performance accuracy of the 9-gene signature for HIV-free or HIV-coinfected patients in the pooled Independent Validation data.

FIGS. 3A-3E shows the 9-gene signature correlates with treatment response in pooled retrospective validation datasets such that it reduces with longer time of treatment. (A) Beeswarm, violin, and box plots of the 9-gene signature score in patients with ATB being treated over time and in healthy controls. Each point represents a blood sample. P-values at the top of each beeswarm plot is compared to healthy controls. The horizontal line in the middle of each box indicates the median, while the bottom and top borders of the box represent the first and third quartile, respectively. The whiskers above and below represent the range of non-outlier values. (B) Pearson correlation between local inflammation measured using PET-CT as Total Glycolytic Activity Index (TGAI) and peripheral inflammation represented by 9-gene signature in blood at diagnosis, 4 weeks after treatment and 24 weeks after treatment (end of treatment) in the Catalysis Treatment Response Cohort. (C) Pearson correlation between local inflammation measured using PET-CT at 24 weeks of treatment and peripheral inflammation represented by 9-gene signature in blood at diagnosis demonstrating the 9-gene signature could identify those at higher risk of persistent lung inflammation at the end of treatment at the time of diagnosis in the Catalysis Treatment Response Cohort. (D) 9-gene signature at each time point in those with persistent lung inflammation and those with clear lungs at the end of treatment demonstrating those with persistent lung inflammation have consistently higher 9-gene signature than those with clear lung at each time point. At the end of treatment (24 weeks), 9-gene signature in those with persistent lung inflammation is higher than healthy controls, but not in those with clear lungs. (E) 9-gene signature in blood correlates with bacterial load in lung as measured by time to negativity using MGIT culture.

FIGS. 4A and 4B show the 9-gene signature distinguishes ATB from healthy controls and those with LTBI in two prospective cohorts from Moldova and Brazil with higher accuracy than the 3-gene signature. (A) In the Moldova household contact study, the 9-gene signature distinguished patients with ATB from healthy controls and those with LTBI with an overall AUROC of 0.885. (B) In the Brazil active screening cohort, the 9-gene signature accuracy for distinguishing patients with ATB was consistently higher than that of the 3-gene signature. Accuracy of both the 9-gene and 3-gene signatures correlated with the bacterial load. Solid lines represent 9-gene signature, dotted lines represent the 3-gene signature.

FIGS. 5A-5C show the 9-gene signature predicts progression from latent to active tuberculosis in the ACS cohort and the Moldova cohort. (A) ROC curves for the 3-gene signature (dotted lines) and 9-gene signature (solid lines) distinguishing progressors and non-progressor samples collected at different time points. The 9-gene signature and 3-gene signature had similar accuracy in samples collected between 8-180 days prior to diagnosis of ATB. The 9-gene signature had significantly higher accuracy for predicting progression from LTBI to ATB in samples collected between 181-360 samples than the 3-gene signature. (B) the 9-gene signature identified each progressor in the Moldova household contact study prior to clinical diagnosis. (C) Pairwise comparison of the 9-gene and 3-gene signatures in all validation cohorts showed that the 9-gene signature has consistently higher accuracy than the 3-gene signature.

### DETAILED DESCRIPTION

All publications, patents and patent applications cited herein, whether supra or infra, are hereby incorporated by reference in their entireties.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that, as used in this specification and the appended claims, the singular forms “a”, “an” and “the” include plural referents unless the content clearly dictates otherwise. Thus, for example, reference to “an agonist” includes a mixture of two or more such agonists, and the like.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As noted above, a method of analyzing a sample is provided. In some embodiments the method comprises (a) obtaining a sample of RNA from a subject having latent tuberculosis and/or symptoms of active tuberculosis; and (b) measuring the amount of RNA transcripts encoded by PLAAT4, CYB561, GBP5, SMAD7, LAX1, CDKN1C, CA5B, EPHA4 and CD83 in the sample, to produce gene expression data. The method may be used in a variety of diagnostic and therapeutic methods, as described below.

It is noted that at least some of the listed genes may be referred to by a different name in the literature. For example, the gene PLAAT4 may be referred to as RARRES3 in some publications.

### Diagnostic Methods

As noted above, the method may be used to determine if a subject has active tuberculosis or is progressing to active tuberculosis. In some embodiments, the method may comprise: (a) obtaining a sample of RNA from a subject; (b) measuring the amount of RNA transcripts encoded PLAAT4, CYB561, GBP5, SMAD7, LAX1, CDKN1C, CA5B, EPHA4 and CD83 in the sample, to produce gene expression data and (c) providing a report indicating whether the subject has active tuberculosis or is progressing to active tuberculosis, wherein: (i) increased PLAAT4, CYB561, and GBP5 expression indicates that the subject has active tuberculosis or is progressing to active tuberculosis; and (ii) increased SMAD7, LAX1, CDKN1C, CA5B, EPHA4, and CD83 indicates that the subject does not have active tuberculosis or is not progressing to active tuberculosis.

The subject from which the sample is obtained may have latent tuberculosis and/or symptoms of active tuberculosis. Subjects with latent tuberculosis generally do not feel ill, do not exhibit of tuberculosis, and are not infectious. However, they are nevertheless infected by M. tuberculosis and have a positive reaction to the tuberculin skin test or tuberculosis blood test. Overall, without treatment, about 5 to 10% of people with latent tuberculosis will develop TB disease at some time in their lives. Patients with active tuberculosis, on the hand, exhibit the following symptoms: coughing for three or more weeks, coughing up blood or mucus, chest pain, or pain with breathing or coughing, unintentional weight loss, fatigue, fever, night sweats, chills, and loss of appetite. These subjects are contagious and also have a positive reaction to the tuberculin skin test or tuberculosis blood test (if they are tested). In addition, the sample may be obtained from a subject that has been exposed to a subject with active tuberculosis and/or a subject that is more susceptible to an active infection because they have a weakened immune system or other conditions or disorders, e.g., HIV/AIDS, diabetes, severe kidney disease, some cancers, malnutrition or low body weight, very young or advanced age, or have been treated with drigs that might have an effect on the immune system, such as chemotherapy, drugs (e.g., steroids) to prevent rejection of transplanted organs and some drugs used to treat rheumatoid arthritis, Crohn's disease or psoriasis.

The measuring step can be done using any suitable method. For example, the amount of the RNA transcripts in the sample may be measured by RNA-seq (see, e.g., Morin et al BioTechniques 2008 45: 81-94; Wang et al 2009 Nature Reviews Genetics 10: 57-63), RT-PCR (Freeman et al BioTechniques 1999 26: 112-22, 124-5), or by labeling the RNA or cDNA made from the same and hybridizing the labeled RNA or cDNA to an array. An array may contain spatially-addressable or optically-addressable sequence-specific oligonucleotide probes that specifically hybridize to transcripts being measured, or cDNA made from the same. Spatially-addressable arrays (which are commonly referred to as “microarrays” in the art) are described in, e.g., Sealfon et al (see, e.g., Methods Mol Biol. 2011; 671:3-34). Optically-addressable arrays (which are commonly referred to as “bead arrays” in the art) use beads that internally dyed with fluorophores of differing colors, intensities and/or ratios such that the beads can be distinguished from each other, where the beads are also attached to an oligonucleotide probe. Exemplary bead-based assays are described in Dupont et al (J. Reprod Immunol. 2005 66:175-91) and Khalifian et al (J Invest Dermatol. 2015 135: 1-5). The abundance of transcripts in a sample can also be analyzed by quantitative RT-PCR or isothermal amplification method such as those described in Gao et al (J. Virol Methods. 2018 255: 71-75), Pease et al (Biomed Microdevices (2018) 20: 56) or Nixon et (Biomol. Det. and Quant 2014 2: 4-10), for example. Many other methods for measuring the amount of an RNA transcript in a sample are known in the art.

The sample of RNA obtained from the subject may comprise RNA isolated from whole blood, white blood cells, peripheral blood mononuclear cells (PBMC), neutrophils or buffy coat, for example. Methods for making total RNA, polyA+ RNA, RNA that has been depleted for abundant transcripts, and RNA that has been enriched for the transcripts being measured are well known (see, e.g., Hitchen et al J Biomol Tech. 2013 24: S43-S44). If the method involves making cDNA from the RNA, then the cDNA may be made using an oligo(d)T primer, a random primer or a population of gene-specific primers that hybridize to the transcripts being analyzed.

In measuring the transcript, the absolute amount of each transcript may be determined, or the amount of each transcript relative to one or more control transcript, e.g., actin or the like, may be determined. Whether the amount of a transcript is increased or decreased may be in relation to the amount of the transcript (e.g., the average amount of the transcript) in control samples (e.g., in blood samples collected from a population of at least 100, at least 200, or at least 500 subjects that are known or not known to have an active tuberculosis or that are progressing to active tuberculosis). As such, in any embodiment, whether the amount of a transcript is increased or decreased may be in relation to the amount of the same transcript in a population of subjects that do not have active tuberculosis and that are not progressing to active tuberculosis from late tuberculosis.

In some embodiments, the method may comprise providing a report indicating whether the subject has active tuberculosis or is progressing to active tuberculosis based on the measurements of the amounts of the transcripts. In some embodiments, this step may involve calculating a score based on the amounts of each of the transcripts, where the scores correlates with the phenotype and can be a number such as a probability, likelihood or score out of 10, for example. In these embodiments, the method may comprise inputting the amounts of each of the transcripts into one or more algorithms, executing the algorithms, and receiving a score for each phenotype based on the calculations. In these embodiments, other measurements from the subject may be input into the algorithm. For example, as illustrated below, the score may be be the geometric mean of the expression of genes that are positively correlated with the response variable minus the geometric mean of the expression of the negatively correlated genes.

In some embodiments, the method may involve creating the report e.g., in an electronic form, and forwarding the report to a doctor or other medical professional to help identify a suitable course of action, e.g., to identify a suitable therapy for the subject. The report may be used along with other metrics as a diagnostic to determine whether the subject has active tuberculosis or is progressing to active tuberculosis

In any embodiment, report can be forwarded to a “remote location”, where “remote location,” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like. In certain embodiments, the report may be analyzed by an MD or other qualified medical professional, and a report based on the results of the analysis of the image may be forwarded to the subject from which the sample was obtained.

In computer-related embodiments, a system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining whether the subject has active tuberculosis or is progressing to active tuberculosis using the measurements described above as inputs. The computer processor is coupled to the storage component and configured to execute the instructions stored in the storage component in order to receive patient data and analyze patient data according to one or more algorithms. The display component may display information regarding the diagnosis of the patient.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein.

The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

### Therapeutic Methods

Therapeutic methods are also provided. In some embodiments, these methods may comprise identifying a subject as having active tuberculosis or progression to active tuberculosis using the methods described above, and treating a subject based on whether the subject is indicated as having active tuberculosis or progression to active tuberculosis. In some embodiments, this method may comprise receiving a report indicating whether the subject has active tuberculosis or progression to active tuberculosis, wherein the report is based on the gene expression data obtained by measuring the amount of RNA transcripts encoded by the genes, and treating a subject based on whether the subject accordingly. In some embodiments the method may comprise: (a) identifying a patient as having active tuberculosis or progression to active tuberculosis based on the amount of RNA transcripts encoded by PLAAT4, CYB561, GBP5, SMAD7, LAX1, CDKN1C, CA5B, EPHA4 and CD83 in a sample from the subject; and (b) treating the subject with antibiotics one or more of isoniazid, rifampin, pyrazinamide, and ethambutol. Methods for administering and dosages for administering the antibiotics listed above are known in the art or can be derived from the art. In some embodiments the identifying may done by reviewing a report indicating whether the subject has active tuberculosis or is progressing to active tuberculosis, as described above. As noted above, in some embodiments the report comprises a tuberculosis score, wherein a higher tuberculosis score for the subject indicates that the subject is more likely to have active tuberculosis or progression to active tuberculosis. This score can be used to monitor the subject's response to antibiotics. For example, the method may comprise calculating a tuberculosis score before and after treatment, wherein an increasing tuberculosis score indicates that the tuberculosis infection is worsening and a decreasing tuberculosis score indicates tat the subject is recovering.

### Kits

Also provided by this disclosure are kits for practicing the subject methods, as described above. In some embodiments, the kit may contain reagents for measuring the amount of RNA transcripts encoded by PLAAT4, CYB561, GBP5, SMAD7, LAX1, CDKN1C, CA5B, EPHA4 and CD83. In some embodiments, the kit may comprise, for each RNA transcript, a sequence-specific oligonucleotide that hybridizes to the transcript. In some embodiments, the sequence-specific oligonucleotide may be biotinylated and/or labeled with an optically-detectable moiety. In some embodiments, the kit may comprise, for each RNA transcript, a pair of PCR primers that amplify a sequence from the RNA transcript, or cDNA made from the same. In some embodiments, the kit may comprise an array of oligonucleotide probes, wherein the array comprises, for each RNA transcript, at least one sequence-specific oligonucleotide that hybridizes to the transcript. The oligonucleotide probes may be spatially addressable on the surface of a planar support, or tethered to optically addressable beads, for example.

In embodiments in which a quantitative isothermal amplification method is used, the kit may comprise reagents comprise multiple reaction vessels, each vessel comprising at least one (e.g., 2, 3, 4, 5, or 6) sequence-specific isothermal amplification primers that hybridizes to a single transcript, e.g., a transcript from a single gene selected from PLAAT4, CYB561, GBP5, SMAD7, LAX1, CDKN1C, CA5B, EPHA4 and CD83, or cDNA made from the same. As such, in some embodiments, the kit may contain at least 9 reaction vessels, where each reaction vessels contain one or more primers for detection of an RNA transcript encoded by a single gene. In some embodiments, the kit may contain reagents for measuring the amount of up to a total of 30, 50 or 100 RNA transcripts.

The various components of the kit may be present in separate containers or certain compatible components may be precombined into a single container, as desired.

In addition to the above-mentioned components, the subject kit may further include instructions for using the components of the kit to practice the subject method.

### Additional Embodiments

In any embodiment, the method can be practiced by measuring the amount of RNA transcripts encoded by at least two of the nine listed genes, e.g., by measuring the amount of RNA transcripts encoded by 2, 3, 4, 5, 6, 7 or 8 of PLAAT4, CYB561, GBP5, SMAD7, LAX1, CDKN1C, CA5B, EPHA4 and CD83. The total number of transcripts measured in some embodiments may be up to 50 or 100 RNA transcripts. AUROC for all pairwise combinations of these genes can be found in tables 5, 6, and 7 below. In addition, other genes can be analyzed in addition to the nine listed genes or subset thereof. For example, in any embodiment, the method may further comprise measuring the amount of RNA transcripts of other genes associated with TB, or one or more other genes listed in Tables 5, 6, and 7 below.

In some embodiments, the method may be practiced by measuring the amount of RNA transcripts of a set of any number of genes (e.g., at least 2, at least 3, at least 4, at least 5, at least 6, or at least 7 genes, up to 30 or 50 genes), where the set of genes includes any pair of genes listed in Tables, 5, 6 and 7 as well as optionally other genes (e.g., at least 2, at least 3, at least 4, at least 5, at least 6, or at least 7 other genes) that are independently listed or not listed in Tables, 5, 6 and 7. These embodiments, may be described by the following clauses.

Embodiment 1: The method of analyzing a sample, the method comprising: (a) obtaining a sample of RNA from a subject, e.g., a subject having latent tuberculosis and/or symptoms of active tuberculosis; and (b) measuring the amount of RNA transcripts encoded of at least two (e.g., at least 2, at least 3, at least 4, at least 5, at least 6, at least 7 or at least 8) of PLAAT4, CYB561, GBP5, SMAD7, LAX1, CDKN1C, CA5B, EPHA4 and CD83 in the sample, to produce gene expression data.

Embodiment 2. A method of analyzing a sample, the method comprising: (a) obtaining a sample of RNA from a subject, e.g., a subject having latent tuberculosis and/or symptoms of active tuberculosis; and (b) measuring the amount of RNA transcripts encoded of at least two (e.g., at least 2, at least 3, at least 4, at least 5, at least, at least 6, at least 7 or at least 8) genes listed in Tables 5, 6, and 7 in the sample, to produce gene expression data.

Embodiment 3. The method of any prior embodiment, wherein the sample comprises RNA isolated from whole blood, white blood cells, neutrophils, peripheral blood mononuclear cells (PBMCs), or buffy coat.

Embodiment 4. The method of any prior embodiment, further comprising: (c) based on the gene expression data, generating a report indicating whether the subject has active tuberculosis or is progressing to active tuberculosis, wherein: (i) increased PLAAT4, CYB561, GBP5 expression indicates that the subject has active tuberculosis or is progressing to active tuberculosis; and (ii) decreased SMAD7, LAX1, CDKN1C, CA5B, EPHA4, CD83 indicates that the subject has active tuberculosis or is progressing to active tuberculosis.

Embodiment 5. The method of any prior embodiment, further comprising calculating a tuberculosis score based on the levels of expression of the RNA transcripts in the subject, wherein a higher tuberculosis score for the subject indicates that the subject is more likely to have active tuberculosis or progression to active tuberculosis.

Embodiment 6. The method of any prior embodiment further comprising diagnosing the patent as having active tuberculosis or progression to active tuberculosis based on the gene expression data, wherein: (i) increased PLAAT4, CYB561, GBP5 expression indicates that the subject has active tuberculosis or is progressing to active tuberculosis; and (ii) decreased SMAD7, LAX1, CDKN1C, CA5B, EPHA4, CD83 indicates that the subject has active tuberculosis or is progressing to active tuberculosis.

Embodiment 7. The method of any prior embodiment, further comprising identifying the subject as having active tuberculosis or progression to active tuberculosis based on the gene expression data, and administering antibiotics to the patient.

Embodiment 8. The method of embodiment 7, wherein the antibiotics comprise one or more of isoniazid, rifampin, pyrazinamide, and ethambutol.

Embodiment 9. The method of embodiment any prior embodiment, wherein the measuring step is done by RT-PCR.

Embodiment 10. The method of any of embodiments 1-8, wherein the measuring step is done using a quantitative isothermal amplification method.

Embodiment 11. The method of any of embodiments 1-8, wherein the measuring step is done by sequencing.

Embodiment 12. The method of any of embodiments 1-8, wherein the measuring step is done by labeling the RNA or cDNA made from the same and hybridizing the labeled RNA or cDNA to a support.

Embodiment 13. A method for treating a subject, comprising: (a) identifying a patient as having active tuberculosis or progression to active tuberculosis based on the amount of RNA transcripts encoded by at least two (e.g., at least 2, at least 3, at least 4, at least 5, at least 6, at least 7 or at least 8) of PLAAT4, CYB561, GBP5, SMAD7, LAX1, CDKN1C, CA5B, EPHA4 and CD83 in a sample from the subject; and (b) treating the subject with antibiotics.

Embodiment 14. A method for treating a subject, comprising: (a) identifying a patient as having active tuberculosis or progression to active tuberculosis based on the amount of RNA transcripts encoded by at least two (e.g., at least 2, at least 3, at least 4, at least 5, at least 6, at least 7 or at least 8) genes listed in Tables 5, 6, and 9 in the sample; and (b) treating the subject with antibiotics.

Embodiment 15. The method of embodiment 13 or 14, wherein the antibiotics comprise one or more of isoniazid, rifampin, pyrazinamide, and ethambutol.

Embodiment 16. The method of any of embodiments 13-15, wherein the identifying is done by reviewing a report indicating whether the subject has active tuberculosis or is progressing to active tuberculosis.

Embodiment 17. The method of embodiment 16, wherein the report comprises a tuberculosis score, wherein a higher tuberculosis score for the subject indicates that the subject is more likely to have active tuberculosis or progression to active tuberculosis.

Embodiment 18. The method of any of embodiments 13-17, further comprising monitoring the subject's response to antibiotics.

Embodiment 19. The method of embodiment 18, further comprising calculating a tuberculosis score before and after treatment, wherein an increasing tuberculosis score indicates that the tuberculosis infection is worsening and a decreasing tuberculosis score indicates tat the subject is recovering.

Embodiment 20. A kit for diagnosing active tuberculosis or progression to active tuberculosis, comprising reagents for measuring the amount of RNA transcripts encoded by at least two (e.g., at least 2, at least 3, at least 4, at least 5, at least 6, at least 7 or at least 8) of PLAAT4, CYB561, GBP5, SMAD7, LAX1, CDKN1C, CA5B, EPHA4 and CD83 and at least two (e.g., at least 2, at least 3, at least 4, at least 5, at least 6, at least 7 or at least 8) genes listed in Tables 5, 6, and 9 in the sample.

Embodiment 21. The kit of embodiment 20, wherein the reagents comprise, for each RNA transcript, a sequence-specific oligonucleotide that hybridizes to the transcript.

Embodiment 22. The kit of embodiment 21, wherein sequence-specific oligonucleotide is biotinylated and/or labeled with an optically-detectable moiety.

Embodiment 23. The kit of any of embodiments 20-22, wherein the reagents comprises, for each RNA transcript, at least a pair of PCR primers that amplify a sequence from the RNA transcript, or cDNA made from the same.

Embodiment 24. The kit of embodiment 20, wherein the reagents comprise multiple reaction vessels, each comprising at least one sequence-specific isothermal amplification primer that hybridizes to the transcript, or cDNA made from the same.

### Examples

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

The World Health Organization (WHO) has reported that approximately 10 million people developed active tuberculosis (ATB) in 2020, of which only 5.8 million were diagnosed, and 1.5 million patients die. Globally, TB was estimated to lead to 122 million disability-adjusted-life-years (DALYs) in 2019. Despite high rates of morbidity and mortality, if diagnosed early and accurately, ATB can be treated and cured. However, the current reference-standards for diagnosis of ATB, sputum culture and smear microscopy, suffer from several limitations including low sensitivity, difficulty in obtaining sputum samples (particularly in children and people with HIV), and an inability to predict progression from latent TB infection (LTBI) to ATB. Tuberculin Skin Tests (TST) and Interferon Gamma Release Assays (IGRA), both of which measure the immune response to Mycobacterium tuberculosis (Mtb) cannot distinguish LTBI, which is estimated to affect roughly a quarter of the world's population, from the active disease. More recent advances, such as the sputum based Xpert MTb/RIF assay, can diagnose ATB as well as drug resistance with a fast turnaround time on the order of hours. This is a significant improvement over older culture-based methods that often take weeks to produce results. However, the diagnostic accuracy of the Xpert MTb/RIF assay is drastically reduced for people with HIV, and the test suffers each of the limitations of sputum-based tests.

As part of its strategy to end the global tuberculosis epidemic, the WHO has identified the need for non-sputum-based triage tests, with minimum target product profiles (TPP) of 90% sensitivity and 70% specificity for diagnosis of ATB and 75% sensitivity and 75% specificity for predicting progression from LTBI to ATB. Although several blood-based gene expression signatures have been proposed, most were developed using single-cohort studies, which do not generalize to the more heterogeneous global population.

### Materials and Methods

Systematic Search, Collection, and Curation of Gene Expression Datasets from Public Repositories

A systematic search was preformed in the NIH Gene Expression Omnibus (GEO) and European Bioinformatics Institute (EBI) ArrayExpress for publicly available gene expression profiles of peripheral blood from humans with TB or other diseases. Datasets were excluded if they (i) were nonclinical, e.g. in-vitro studies, (ii) were profiled using tissues other than WB or PBMCs, or (iii) did not provide enough information to identify whether the infection was caused by M. tuberculosis or another pathogen. In total, 95 unique datasets were identified that met the criteria (Table 1, Table 2). These cohorts included transcriptomic profiles of 9,504 blood samples across 49 countries from 1,615 patients with ATB, 2,615 healthy controls, 742 patients with LTBI, and 4,532 patients with other diseases. Healthy controls and patients with either LTBI or other diseases were referred to collectively as “Other” samples.

TABLE 1
Performance of the ATB vs. Other score in Discovery, Hold-out Validation, and Independent Validation data.

| | | | | | PPV at | NPV at | | | |
|---|---|---|---|---|---|---|---|---|---|
| | | AUROC | | | 2% | 2% | | | |
| Analysis | | (95% | Sensitivity | Specificity | prevalence | prevalence | | | |
| stage | Comparison | CI) | (%) | (%) | (%) | (%) | LR+ | LR− | DOR |
| Discovery | ATB vs. All | 0.9 | 90 | 74.4 | 6.69 | 99.7 | 3.51 | 0.134 | 26.2 |
| | | (0.876-0.924) | | | | | | | |
| | ATB vs. Healthy | 0.953 | 90 | 88.1 | 13.4 | 99.8 | 7.58 | 0.113 | 67.1 |
| | | (0.936-0.97) | | | | | | | |
| | ATB vs. LTBI | 0.936 | 90 | 75.2 | 6.91 | 99.7 | 3.64 | 0.132 | 27.5 |
| | | (0.913-0.959) | | | | | | | |
| | ATB vs.OD | 0.855 | 90 | 62.4 | 4.66 | 99.7 | 2.4 | 0.16 | 15 |
| | | (0.826-0.883) | | | | | | | |
| Hold-out | ATB vs. All | 0.89 | 90.3 | 76.8 | 7.37 | 99.7 | 3.9 | 0.127 | 30.8 |
| Validation | | (0.849-0.93) | | | | | | | |
| | ATB vs. Healthy | 0.949 | 90.3 | 90.2 | 15.9 | 99.8 | 9.23 | 0.108 | 85.6 |
| | | (0.92-0.977) | | | | | | | |
| | ATB vs. LTBI | 0.951 | 91.2 | 89.7 | 15.4 | 99.8 | 8.89 | 0.099 | 90.1 |
| | | (0.92-0.983) | | | | | | | |
| | ATB vs.OD | 0.832 | 90.3 | 63.9 | 4.85 | 99.7 | 2.5 | 0.152 | 16.4 |
| | | (0.783-0.88) | | | | | | | |
| Independent | ATB vs. All | 0.923 | 90.1 | 81 | 8.83 | 99.8 | 4.74 | 0.122 | 39 |
| Validation | | (0.905-0.94) | | | | | | | |
| (pooled) | ATB vs. Healthy | 0.959 | 90.1 | 90.6 | 16.4 | 99.8 | 9.58 | 0.109 | 88 |
| | | (0.946-0.972) | | | | | | | |
| | ATB vs. LTBI | 0.926 | 90.1 | 81.1 | 8.85 | 99.8 | 4.76 | 0.122 | 39.1 |
| | | (0.906-0.946) | | | | | | | |
| | ATB vs.OD | 0.901 | 90.1 | 75.4 | 6.95 | 99.7 | 3.66 | 0.131 | 28 |
| | | (0.881-0.92) | | | | | | | |
| Independent | ATB vs. All | 0.9 | 90.4 | 75.2 | 6.91 | 99.7 | 3.57 | 0.14 | 25.4 |
| Validation | | (0.791-1) | | | | | | | |
| (individual) | ATB vs. Healthy | 0.95 | 90.6 | 86.1 | 11.8 | 99.8 | 7.05 | 0.115 | 61.5 |
| | | (0.861-1) | | | | | | | |
| | ATB vs. LTBI | 0.914 | 91.7 | 80.9 | 8.91 | 99.8 | 4.54 | 0.11 | 41.2 |
| | | (0.818-1) | | | | | | | |
| | ATB vs.OD | 0.859 | 90.9 | 60 | 4.44 | 99.7 | 2.32 | 0.151 | 15.3 |
| | | (0.752-0.966) | | | | | | | |

Sensitivity and specificity were chosen to maximize the sum of sensitivity and specificity while ensuring that the sensitivity remained above 90%. For individual independent validation, summary AUROCs were calculated using a summary ROC curve. PPV, NPV, LR+, LR−, and diagnostics odds ratio (DOR) statistics were calculated using the R package mada. ATB, active tuberculosis; LTBI, latent tuberculosis infection; OD, other diseases; AUROC, area under the receiver operating curve; CI, confidence interval; PPV, positive predictive value; NPV, negative predictive value, positive likelihood ratio, LR+; negative likelihood ratio, LR-

TABLE 2
Performance of the ATB vs. Other score for distinguishing
TB progressors from non-progressors in the ACS cohort

| | | | | PPV at | NPV at | | | |
|---|---|---|---|---|---|---|---|---|
| Days prior | AUROC | | | 2% | 2% | Positive | Negative | |
| to sputum | (95% | Sensitivity | Specificity | prevalence | prevalence | likelihood | likelihood | Diagnostic |
| conversion | CI) | (%) | (%) | (%) | (%) | ratio | ratio | odds ratio |
| 8-180 | 0.931 | 78.6 | 91.9 | 16.5 | 99.5 | 9.69 | 0.233 | 41.6 |
| | (0.835-1) | | | | | | | |
| 181-360 | 0.836 | 76 | 83.3 | 8.51 | 99.4 | 4.56 | 0.288 | 15.8 |
| | (0.726-0.945) | | | | | | | |
| 361-540 | 0.619 | 80 | 44.4 | 2.85 | 99.1 | 1.44 | 0.45 | 3.2 |
| | (0.449- | | | | | | | |
| 541-720 | 0.49 | 75 | 24 | 1.97 | 97.9 | 0.987 | 1.04 | 0.947 |
| | (0.258-0.722) | | | | | | | |

Sensitivity and specificity were chosen to maximize the sum of sensitivity and specificity while ensuring that the sensitivity remained above 75%. ATB, active tuberculosis; TB, tuberculosis; ACS, Adolescent Cohort Study; AUROC, area under the receiver operating curve; CI, confidence interval; PPV, positive predictive value; NPV, negative predictive value.

When available, normalized microarray data available from GEO was directly used. When normalized data was not provided by authors, the raw microarray data was normalized using conventional methods, such as GC robust multiarray average (gcRMA) or RMA to normalize Affymetrix microarrays with or without mismatch probes respectively and normal-exponential background correction followed by quantile normalization for Illumina, Agilent, GE, and other commercial arrays. Custom microarrays were not normalized and used data as provided by the authors was used. After log 2-transformation, a fixed-effect model was used to summarize multiple probes mapping to a gene within each study. Within each study, cohorts assayed with different microarray types were treated as independent.

### Multicohort Analyses to Identify the 9-Gene Signature

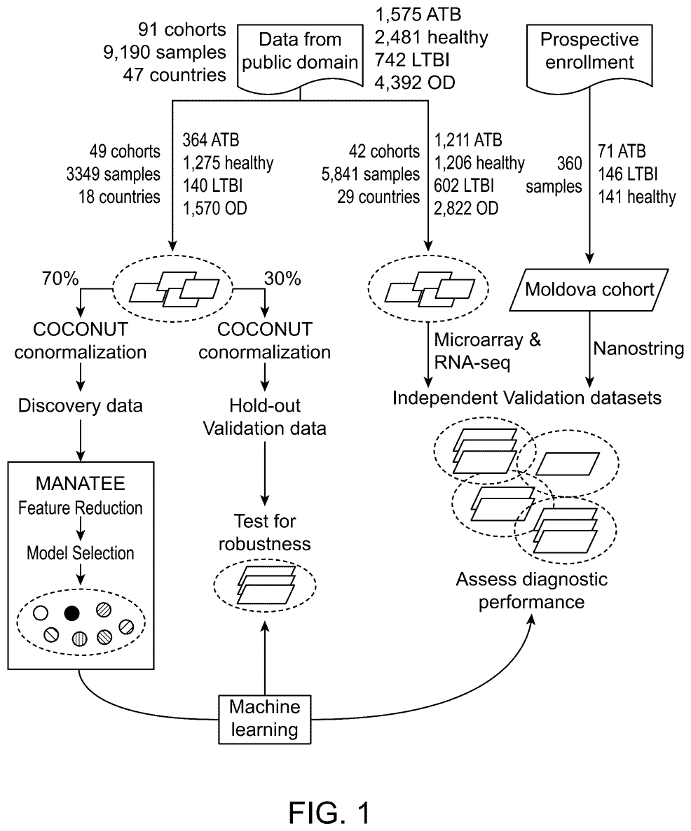

Multicohort ANalysis with AggregaTed gEne Expression (MANATEE) is a multicohort analysis framework for (1) integrating gene expression datasets with COCONUT, (2) identifying a list of differentially expressed genes (DEGs) that distinguish cases from controls, (3) applying feature selection methods on the list of DEGs to identify a parsimonious gene-signature that is optimized for diagnostic accuracy, and (4) to validate the discovered signature in independent data (FIG. 1).

Of the 95 datasets, 49 datasets was used to identify the signature (training cohorts) and the remaining 46 datasets as independent validation cohorts. The samples were further split in the training cohort into discovery (70%) and hold-out validation (30%). The discovery and hold-out validation data was conormalized independently using COCONUT, a previously described method to remove batch effects between the datasets.

To ensure that the differential expression analysis in the discovery was not biased towards diseases with more samples, the “Other” samples were split into nine mutually exclusive, equally weighted groups: (1) healthy, (2) LTBI, (3) bacterial respiratory infection, (4) viral respiratory infection, (5) respiratory infection with an unknown, non-mycobacterial pathogen, (6) sarcoidosis, (7) COPD, (8) idiopathic pulmonary fibrosis (IPF), and (9) lung cancer. For each pairwise comparison between ATB and each of these nine categories, four differential expression statistics were calculated for each gene: 1) the SAM score (from the Significance Analysis of Microarrays), 2) the Benjamini-Hochberg FDR corrected P value from a t-test, 3) the effect size (ES), estimated as Hedges' adjusted g to account for small sample bias, and 4) the fold change (FC). The 9 values of the SAM score, ES, and FC were summarized for each gene by computing a weighted mean across pairwise comparisons, and the 9 FDR values were summarized for each gene using a weighted Z-test.

A leave-one-study-out (LOSO) analysis was preformed to ensure that the results of the differential expression analysis were not driven by a small number of datasets. For the LOSO analysis, each study that accounted for at least 5% of the samples in discovery were iteratively removed and re-calculated the pairwise differential expression statistics for the remaining data. Each iteration is referred to as a LOSO fold. Statistical thresholds for the SAM score, FDR, ES, and/or FC, were set and a gene was defined as being differentially expressed if it exceeded these thresholds, both in the entire Discovery data as well as in every LOSO fold.

From the full list of differentially expressed genes, the 200 genes with the highest absolute summary SAM scores were selected and with summary FDR values less than 0.01. A greedy forward search on these 200 genes was used to identify a reduced set of genes that can diagnose ATB. The objective function of the greedy forward search was a weighted mean of AUROCs for classifying ATB from each of the 9 groups, to ensure that the reduced gene set would not be biased towards diseases with more samples.

### Defining Cases and Controls

### Calculation of a 9-Gene ATB Score

A 9-gene ATB score of a sample, si, was defined as the difference between geometric means of over-expressed genes and that of under-expressed in ATB patients compared with healthy controls and patients with LTBI or other diseases. (Eq. 1). It was empirically determined that including GBP5 twice in the formula increased this model's diagnostic performance.

$${s}_{i} = {( \prod ( GBP 5 , GBP 5 , RARRES 3 , CYB 561 ) )}^{\frac{1}{4}} - {( \prod ( SMAD 7 , LAX 1 , CDKN 1 C , CA 5 B , EPHA 4 , CD 83 ) )}^{\frac{1}{6}}$$

### Summary ROC Curves

Summary ROC curves represent a weighted average of multiple independent ROC curves. Briefly, linear interpolation was used to approximate the true positive rate (TPR) values for each curve for a fixed range of false positive rate (FPR) values. Weights were assigned to each ROC curve based on the number of samples used to create the curve and calculated a summary ROC curve by taking the weighted mean of the corresponding TPR values. In addition, the weighted standard deviation was calculated for each TPR, which is represented by a grey area on the plot. Rhe trapezoid rule was used to calculate the area under the summary ROC curve, and the pooled standard error of the individual curves to calculate the 95% confidence interval.

### Moldova Cohort

To evaluate the diagnostic performance of the 9-gene signature in the Moldova cohort, (1) the progressor samples (7 patients, 14 samples) from the controls was removed and (2) samples from ATB patients that were measured more than 7 days post-TB treatment was removed.

### Results

### Data Collection, Curation, and Preprocessing

The public repositories were searched for blood transcriptome profiles from patients with Mtb infection or a respiratory disease (Methods). 95 datasets composed of 9,190 samples from patients across 47 countries were identified (FIG. 1). Overall, these datasets included a broad spectrum of biological, clinical, and technical heterogeneity represented by blood samples profiled from children and adults using either microarray or RNA sequencing.

Identification of a 9-Gene Signature for Distinguishing ATB from Healthy Controls, LTBI and Other Diseases

MANATEE was used (Multicohort ANalysis of AggregaTed gEne Expression), a multicohort analysis framework for differential expression and machine learning analyses across independent heterogeneous gene expression datasets to identify a gene signature for diagnosing ATB (FIG. 1). 49 datasets comprised of 3,615 samples were used (1,393 HC, 140 LTBI, 404 ATB, and 1,678 OD) for identifying differentially expressed genes and training a classification model (Data not shown). The 3,615 samples were randomly divided into 70% (2,621 samples) for discovery (994 HC, 101 LTBI, 291 ATB, and 1,235 OD) and the remaining 30% (994 samples) as hold-out validation samples (399 HC, 39 LTBI, 113 ATB, and 443 OD). Importantly, the remaining 46 datasets comprised of 5,892 samples were used as independent validation (1,222 HC, 521 LTBI, 1295 ATB, and 2,854 OD) (Data not shown).

After COCONUT co-normalization of the discovery samples, MANATEE identified three upregulated (PLAAT4, CYB561, GBP5) and six downregulated (SMAD7, LAX1, CDKN1C, CA5B, EPHA4, CD83) genes in patients with ATB compared to healthy controls, LTBI, and other diseases (Methods). A 9-gene TB score was defined for each sample as the difference in geometric mean of the up- and down-regulated genes (Methods) as geometric mean-based classification model has been shown to be generalizable to other cohorts in TB. In the discovery samples, the 9-gene TB score distinguished ATB from healthy controls (AUROC=0.953, 95% CI: 0.936-0.97), LTBI (AUROC=0.936, 95% CI: 0.913-0.959), and other diseases (AUROC=0.855, 95% CI: 0.826-0.883) (Table 1), with similar AUROCs in the hold-out samples that were from the same datasets as the discovery samples (Table 1). Overall, in both discovery and hold-out validation samples, the 9-gene TB score had >70% specificity and >90% sensitivity for identifying patients with ATB.

### The 9-Gene Signature Meets WHO TPPs for Diagnosing Active TB in Independent Retrospective Validation Datasets Irrespective of Age, Sex, Race, and HIV-Coinfection.

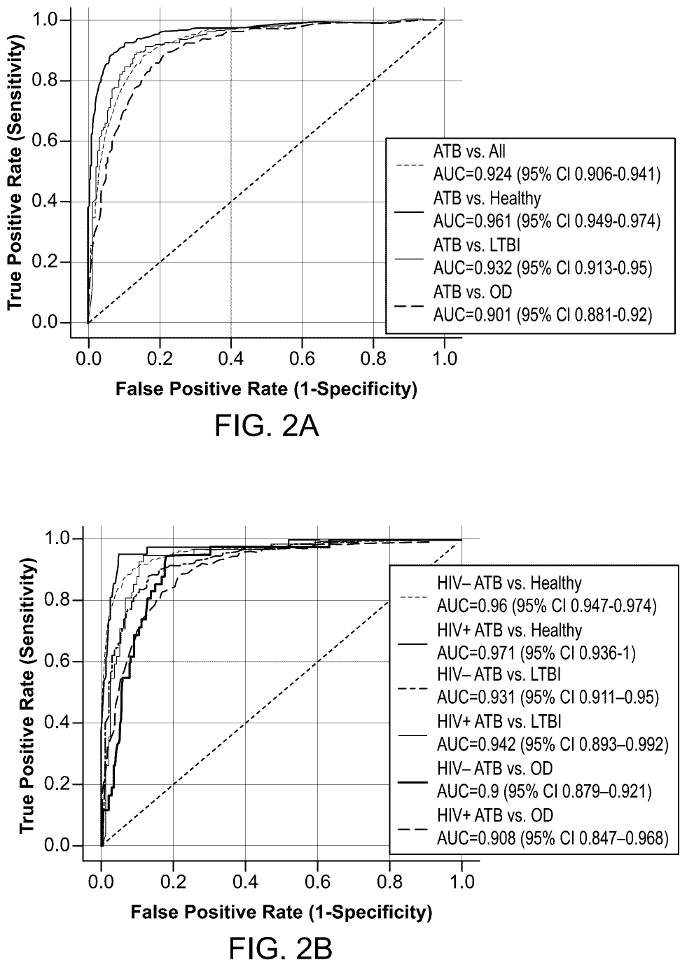

Next, the differential expression of the nine genes and the 9-gene TB score was validated in 5,892 samples from 46 retrospective datasets that were not used in either discovery or the hold-out validation, referred to as independent validation datasets (Data not shown). Each of the 9 genes were significantly differentially expressed in the validation datasets (Data not shown). Of these, 32 datasets (3,836 samples) included healthy controls and could be co-normalized using COCONUT. In COCONUT co-normalized 3,836 samples (1,196 HC, 190 LTBI, 436 ATB, and 2,014 OD), the 9-gene TB score distinguished ATB from all other groups with an overall AUROC of 0.92 (FIG. 2A, Table 1). This discriminatory power was not driven by a single group of samples as the 9-gene TB score distinguished ATB from HC, LTBI, and OD with AUROC of 0.96 (95% CI: 0.949-0.974), 0.93 (95% CI: 0.913-0.95), and 0.9 (95% CI: 0.881-0.92), respectively (FIG. 2A, Table 3). At 90% sensitivity, the score had a specificity of 81% for distinguishing ATB from all other groups. Importantly, when comparing ATB with HC, LTBI, and OD individually, the 9-gene TB score had 91%, 81%, and 75% specificity, respectively, at 90% sensitivity. The high specificities and sensitivities for the 9-gene TB score were not driven by a small number of datasets due to COCONUT conormalization. The 9-gene TB score also distinguished ATB from other groups with high accuracy when applied to each dataset without COCONUT conormalization (Table 1).

TABLE 3
Cohorts used in the TB treatment analysis.

| Accession | Author | Platform | Tissue | Location | Demographic |
|---|---|---|---|---|---|
| E-MTAB-4257 | Noursadeghi | GPL13497 | WB | UK, India, | HIV-negative |
| | | | | Somalia, | adults with |
| | | | | Pakistan, | smear- and |
| | | | | Nigeria, Nepal, | culture-positive |
| | | | | Afganistan, | TB |
| | | | | Vietnam, | |
| | | | | Tanzania, Sri | |
| | | | | Lanka, South | |
| | | | | Africa, etc. | |
| GSE122485 | Sambarey | GPL15520 | WB | India | Adults with TB |
| GSE31348 | Cliff | GPL570 | WB | South Africa | Adults with TB |
| GSE36238 | Cliff | GPL570 | WB | South Africa | Adults with TB |
| GSE40553 | Bloom | GPL10558 | WB | South Africa | TB patients |
| (SA) | | | | | undergoing |
| | | | | | therapy |
| GSE40553 | Bloom | GPL10558 | WB | UK | TB patients |
| (UK) | | | | | undergoing |
| | | | | | therapy |
| GSE54992 | Cai | GPL570 | PBMC | China | Adults with TB |
| GSE56153 | Ottenhoff | GPL6883 | WB | Indonesia | Adults with TB |
| GSE62147 | Tientcheu | GPL6480 | WB | | HIV-negative |
| | | | | | TB patients |
| GSE84076 | de Araujo | GPL16791 | WB | Brazil | Adults with TB |

| | # of | # of post- | # of | Post- |
|---|---|---|---|---|
| | ATB | treatment | cured | treatment |
| Accession | samples | samples | samples | timepoints |
| E-MTAB-4257 | 66 | 31 | 31 | 2-4 years |
| GSE122485 | 4 | 7 | 4 | 6 months, 1 year |
| GSE31348 | 27 | 108 | 27 | 1 week, 2 weeks, |
| | | | | 4 weeks, 6 months |
| GSE36238 | 9 | 9 | 9 | 6 months |
| GSE40553 | 29 | 103 | 29 | 2 weeks, 2 months, |
| (SA) | | | | 6 months, 1 year |
| GSE40553 | 8 | 26 | 0 | 2 weeks, 2 months, |
| (UK) | | | | 4 months, 6 months |
| GSE54992 | 9 | 18 | 0 | 3 months, 6 months |
| GSE56153 | 18 | 35 | 0 | 2 months, 6 months |
| GSE62147 | 26 | 26 | 0 | 6 months |
| GSE84076 | 6 | 2 | 0 | 6 months |

TB, tuberculosis; WB, whole blood; PBMC, peripheral blood mononuclear cells; ATB, active tuberculosis; SA, South Africa; UK, United Kingdom; HIV, human immunodeficiency virus.

Further, in a subset of datasets that provided sample-level demographic information, the 9-gene TB score met the WHO TPP irrespective of the age, sex, and race of the patients, demonstrating its generalizability to the global patient population (Data not shown). HIV coinfection increases the risk of progression from LTBI to ATB by 20-fold compared to individuals without HIV coinfection. The 9-gene TB score maintained high accuracy for distinguishing ATB from other groups even in HIV-positive patients and met the WHO TPP (FIG. 2B). Finally, in a recent dataset that included a small number of patients with multi-drug resistant (MDR) ATB, GSE157657 [28], the 9-gene TB score maintained its accuracy and correctly identified each patient with MDR ATB (Data not shown).

Taken together, the 9-gene TB score met the WHO TPP of 90% sensitivity and 70% specificity for ATB diagnosis in independent retrospective validation datasets, despite the biological, clinical, and technical heterogeneity across tens of datasets. Further, it was not confounded by age, sex, race, and HIV coinfection, demonstrating its generalizability to the global patient population.

The 9-Gene TB Score Significantly Correlates with Anti-TB Treatment Response

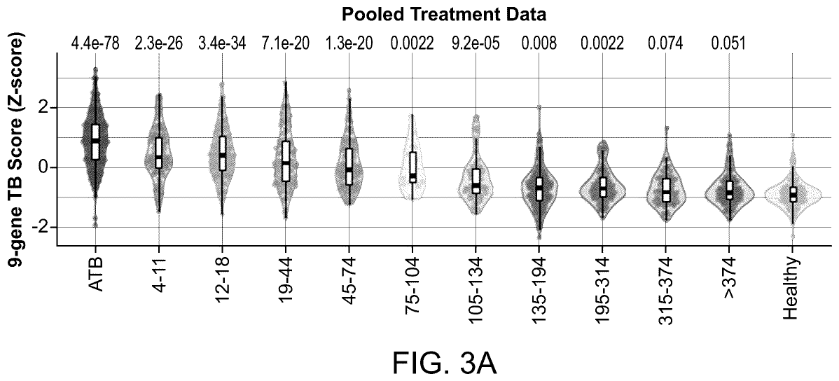

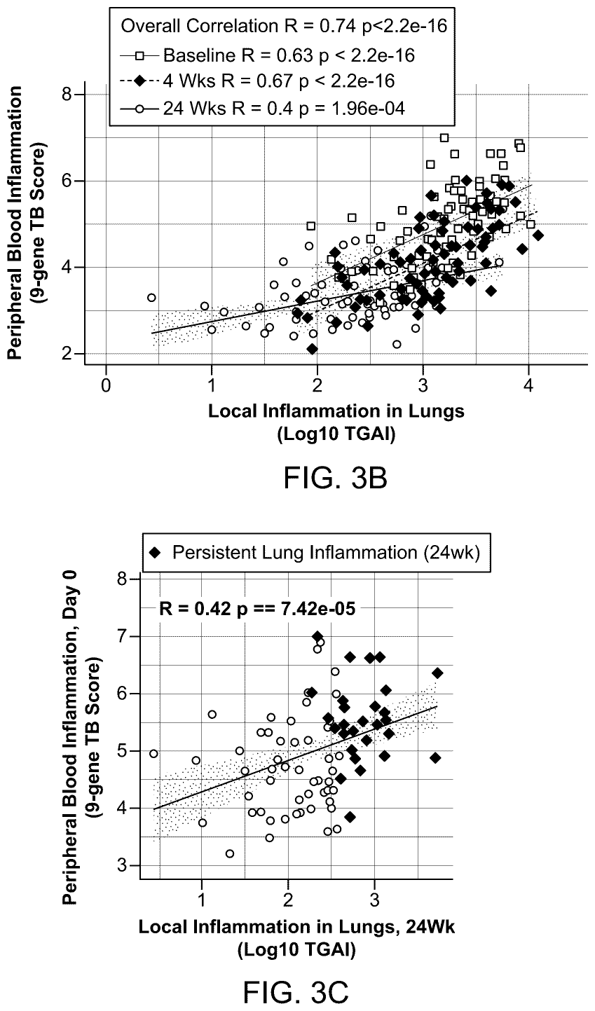

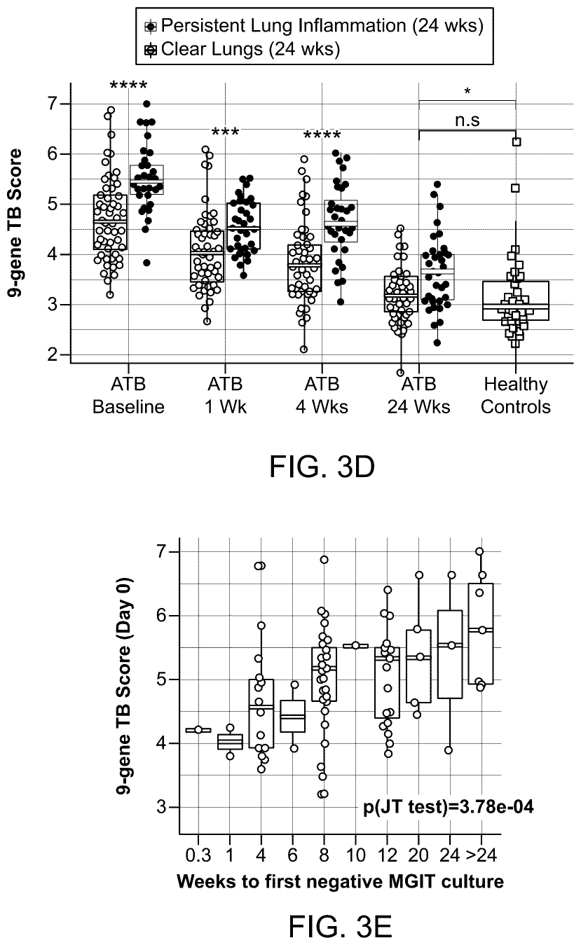

According to the WHO, one of the requirements for the non-sputum-based triage test is that a successful test should be negative in individuals who have completed TB treatment and who are considered cured. Therefore, whether the 9-gene TB score correlated with treatment response and whether it was negative in those who were cured following TB treatment was investigated. 11 independent datasets were identified, comprising of 948 samples from patients with ATB across 6 countries, who had undergone treatment for ATB (Data not shown). Across these II datasets, the 9-gene TB score was inversely correlated with the length of treatment (FIG. 3A). Notably, after approximately 5 months of treatment, the 9-gene TB score in patients with ATB was statistically the same as the healthy controls (FIG. 3A). Specifically, in 5 datasets comprised of 100 patients with ATB, who completed the treatment and remained disease-free during the follow up, the 9-gene TB score patients with ATB from cured patients in all 5 cohorts and with a summary AUROC of 0.976 (95% CI: 0.92-1), suggesting that the 9-gene TB score returned to similar levels as healthy controls upon successful treatment (Data not shown).

A recent longitudinal study, Catalysis Treatment Response Cohort (CTRC), of patients with ATB using positron emission tomography and computerized tomography (PET-CT) imaging found that a substantial proportions of patients with ATB, irrespective of durable cure or recurrent TB, had non-resolving and intensifying lesions consistent with active disease up to 1 year follow up after a 6-month treatment. CTRC also profiled peripheral blood samples from these patients at four time points, which provides a unique opportunity to investigate whether the 9-gene TB score remains

The Catalysis Treatment Response Cohort (CTRC) is a previously described well-studied cohort, which longitudinally profiled HIV-negative ATB patients who were treated for 6 months and had blood samples collected, for transcriptomic and other analysis, at diagnosis (baseline) and after 1 week, 4 weeks and 24 weeks of treatment. Lung pathology in treated patients was measured by the Total Glycolytic Activity Index (TGAI) and was obtained from PET-CT imaging at the same set of time points as the transcriptomic data. Previous studies have reported that the presence of Mtb messenger RNA with non-resolving and intensifying lesions on PET-CT images indicated that many apparently cured patients may have subclinical ATB. Indeed, of the patients with ATB in the CTRC who had received 6 months of treatment, a substantial proportion, termed persistors, showed lung pathology consistent with ATB at the end of treatment (EOT), while the remaining patients showed cleared lung inflammation (Thompson et al., Tuberculosis (Edinb), 2017) Here, the ability of the 9-gene TB score to correlate with the treatment response in the CTRC was investigated and predict persistent lung inflammation prior to the end of treatment.

First, the 9-gene TB score was shown that (1) at any time point was significantly correlated with the TGAI values at the corresponding time point (overall correlation p<2.2e-16, R=0.74, FIG. 3B) and (2) at baseline significantly correlated with TGAI values at EOT (p=7.4e-05, R=0.42, FIG. 3C), suggesting the potential use of the signature as a proxy for radiological lung imaging. Second, at any given time point, the distribution of the 9-gene TB score in patients with radiographically clear lungs at the EOT was significantly lower than that in persistors, suggesting an ability to predict persistent lung inflammation prior to the end of treatment (FIG. 3D). Notably, at EOT the 9-gene score for patients with clear lungs returned to the same level as healthy controls but was significantly higher in persistors (p=0.01; FIG. 3D), showing that the signature can indicate when patients are cured. Finally, the 9-gene TB score at baseline was also significantly correlated with the number of weeks needed for ATB patients to obtain their first negative MGIT culture (p=3.78e-04; FIG. 3E), suggesting that the signature can predict the duration of treatment required for an ATB patient.

Overall, the analyses found that the 9-gene TB score in whole blood correlated with treatment response and lung pathology measured using PET-CT, identified those likely to have persistent lung inflammation despite treatment, and returned to the same level as healthy controls in those with radiographically clear lung at the EOT.

Validation of the 9-Gene TB Score in Two Prospective Cohorts from Moldova and Brazil

The 9-gene signature was profiled in two prospective cohorts using Nanostring: (1) a household contact study in Moldova (Moldova cohort) and (2) and an active screening study in Brazil (Brazil cohort). To serve as a benchmark, a previously described 3-gene signature was profiled in both cohorts. The 3-gene signature is the most validated signature to date. It has been repeatedly shown to be the most accurate signature for diagnosis of ATB and has been translated into a point-of-care test that has been further validated in independent retrospective and prospective cohorts.

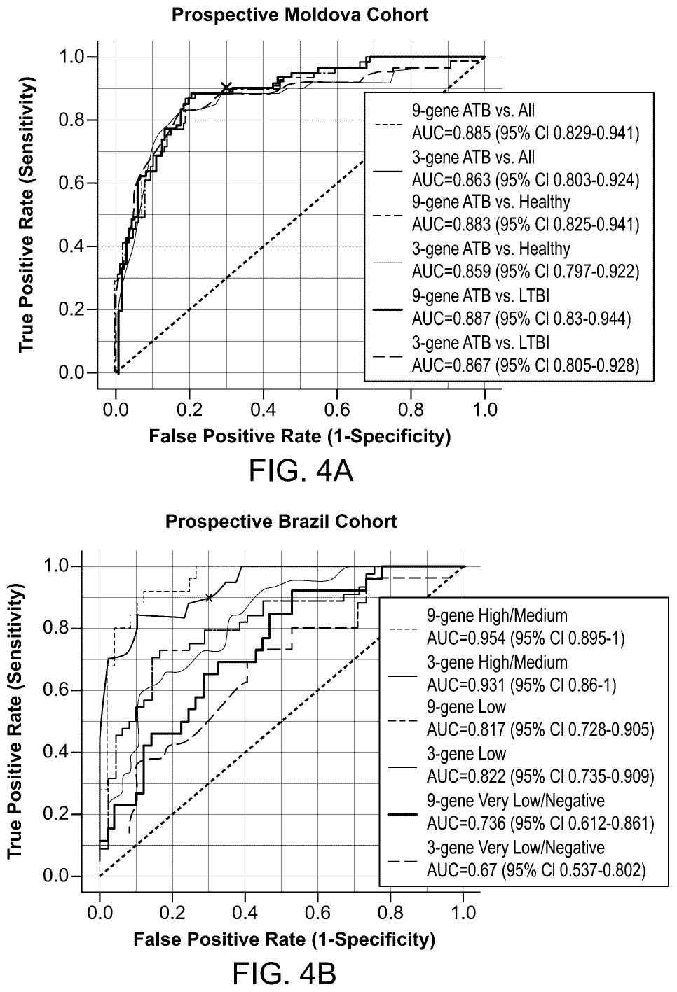

In the Moldova cohort, 360 samples were collected (71 ATB, 146 LTBI, 141 healthy controls and 2 unknown samples) from prospectively enrolled subjects. 5 subjects (2 unknown and 3 clinically diagnosed, culture-negative ATB) were removed from further analysis. When comparing index ATB cases with healthy controls and those with LTBI that did not progress to ATB in the next 12 months, the 9-gene and 3-gene TB scores had AUROC of 0.885 and 0.863, respectively (FIG. 4A). The 9-gene TB score had 69% specificity at 90% sensitivity, and distinguished ATB from healthy controls and LTBI with equal accuracy.

In the Brazil cohort, subjects were enrolled from two prisons in Brazil as described before (Methods). The 9-gene and 3-gene TB scores had AUROC of 0.831 and 0.809, respectively. At 70% specificity, the 9-gene and 3-gene TB scores had 81% and 70% sensitivity, respectively. The 9-gene and 3-gene TB scores were correlated with sputum semi-quantitative Xpert MTB/RIF G4 values. However, the 9-gene TB scores consistently were more accurate than the 3-gene TB scores for each group in the Brazil cohort. At 70% specificity, the 9-gene TB scores had 100%, 80%, and 68% sensitivity for high/medium, low, and very low/negative group, respectively, whereas the 3-gene TB scores had 90%, 72%, and 47% specificity (FIG. 4B).

The 9-Gene TB Score Predicts Progression from LTBI to ATB 1 Year Prior to Sputum Conversion

Several host transcriptome signatures for predicting TB disease progression have been identified that meet minimum WHO TPP of 75% specificity at 75% sensitivity, with the highest performance noted within 3-6 months of TB disease diagnosis. Whether the 9-gene TB scores also predicted progression to ATB and earlier than 6 months was investigated. The Adolescent Cohort Study (ACS) profiled transcriptome of blood samples from 143 adolescents with LTBI from South Africa that were followed for 2 years, of which 34 adolescents progressed to ATB (progressors), diagnosed using sputum smear microscopy. The remaining 109 adolescents (non-progressors) remained latently infected. Each of the subject was sampled every 6 months.

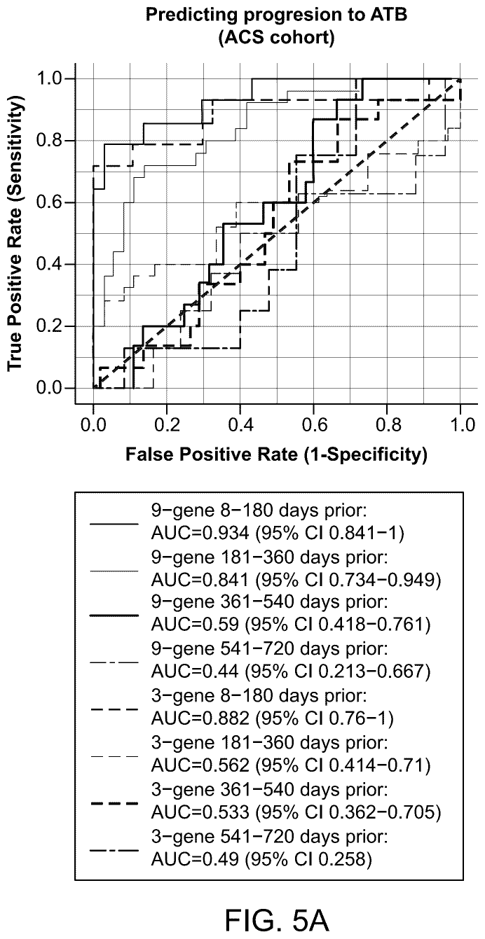

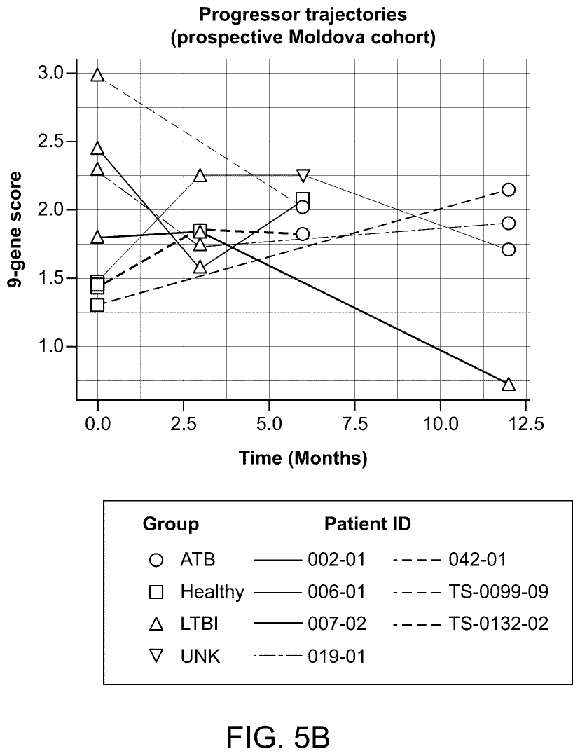

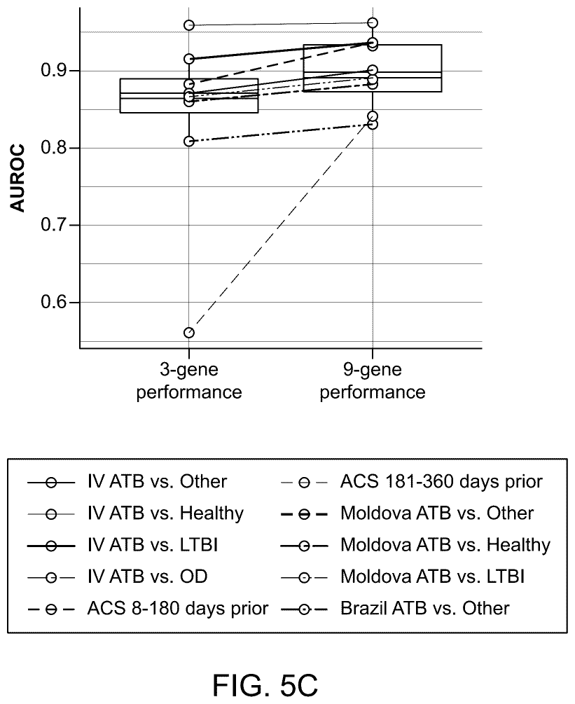

The 9-gene TB scores were significantly higher in progressors than non-progressors 180-360 days prior to sputum conversion, and further increased in progressors 8-180 days prior to sputum conversion (Data not shown). Local regression with LOESS (locally estimated scatterplot smoothing), which modeled the change in the 9-gene TB scores over time, found that 9-gene TB scores in progressors were significantly higher than non-progressors approximately 11 months priors to sputum conversion (Data not shown), the 95% confidence intervals of the regression lines were non-overlapping up to 11.2 months prior to positive sputum microscopy, suggesting that the score could separate the two groups approximately a year before sputum conversion. Indeed, the 9-gene TB score predicted progression to ATB more than 6 months prior with 0.83 AUROC, (FIG. 5A, Table 4), meeting the WHO TPP for predicting progression to ATB up to a year prior to sputum conversion.

To reflect real-world prevalence, the positive predictive value (PPV) of the score at 2% prevalence [CITE] was measured for each time interval (Table 4). For up to a year prior to sputum conversion, the 9-gene score maintained a PPV of over 8.5%, exceeding the performance requested by FIND of 5.8% PPV at 2% prevalence.

TABLE 4
Performance of the ATB vs. Other score in prospective data from Brazil

| | | | | PPV at 2% | NPV at 2% | Positive | Negative | Diagnostic |
|---|---|---|---|---|---|---|---|---|
| | AUROC | Sensitivity | Specificity | prevalence | prevalence | likelihood | likelihood | odds |
| Category | (95% CI) | (%) | (%) | (%) | (%) | ratio | ratio | ratio |
| Overall | 0.826 | 92.6 | 40.8 | 3.1 | 99.6 | 1.57 | 0.181 | 8.67 |
| performance | (0.76-0.892) | | | | | | | |
| Semi-quantitative | | | | | | | | |
| bacterial load | | | | | | | | |
| Medium/High | 0.958 | 92 | 91.8 | 18.7 | 99.8 | 11.3 | 0.087 | 129 |
| | (0.901-1) | | | | | | | |
| Low | 0.808 | 90.9 | 32.7 | 2.68 | 99.4 | 1.35 | 0.278 | 4.85 |
| | (0.718-0.898) | | | | | | | |
| Negative/ | 0.729 | 92.3 | 40.8 | 3.08 | 99.6 | 1.56 | 0.188 | 8.28 |
| Very Low | (0.603-0.855) | | | | | | | |
| Cycle threshold | | | | | | | | |
| <28 | 0.858 | 90.1 | 57.1 | 4.12 | 99.6 | 2.1 | 0.173 | 12.2 |
| | (0.792-0.923) | | | | | | | |
| ≥28 | 0.742 | 92 | 40.8 | 3.07 | 99.6 | 1.55 | 0.196 | 7.93 |
| | (0.616-0.868) | | | | | | | |

Sensitivity and specificity were chosen to maximize the sum of sensitivity and specificity while ensuring that the sensitivity remained above 90%. ATB, active tuberculosis; AUROC, area under the receiver operating curve; CI, confidence interval; PPV, positive predictive value; NPV, negative predictive value.

In the Moldova cohort, 7 when the optimal cutoff value for a minimum sensitivity of 90% was applied to the 7 progressor patients, the 9-gene signature was able to diagnose 4 of the progressors at baseline (FIG. 5B).

Taken together, the results demonstrate the capability of the 9-gene signature for predicting progression from LTBI to ATB, significantly earlier than existing diagnostics. The performance of the 3 gene signature relative to the 9 gene signature is shown in FIG. 5C.

### REFERENCES

[1] “Global tuberculosis report 2021.” (accessed Oct. 27, 2021).
[2] N. A. Menzies et al., “Lifetime burden of disease due to incident tuberculosis: a global reappraisal including post-tuberculosis sequelae,” The Lancet Global Health, vol. 9, no. 12, pp. e1679-e1687, December 2021, doi: 10.1016/S2214-109X(21)00367-3.
[3] Geneva: World Health Organization, “High-priority target product profiles for new tuberculosis diagnostics: report of a consensus meeting,” 2014.
[4] P. Pahal and S. Sharma, “PPD Skin Test,” in StatPearls, Treasure Island (FL): StatPearls Publishing, 2021. Accessed: Nov. 24, 2021. [Online].
[5] B. Saavedra et al., “Diagnostic performance of the Abbott RealTime MTB assay for tuberculosis diagnosis in people living with HIV,” Sci Rep, vol. 11, no. 1, p. 19271, September 2021, doi: 10.1038/s41598-021-96922-3.
[6] World Health Organization, “The End TB Strategy,” 2014.
[7] H. Warsinske, R. Vashisht, and P. Khatri, “Host-response-based gene signatures for tuberculosis diagnosis: A systematic comparison of 16 signatures,” PLOS Medicine, vol. 16, no. 4, p. e1002786, April 2019, doi: 10.1371/journal.pmed.1002786.
[8] S. Suliman et al., “Four-Gene Pan-African Blood Signature Predicts Progression to Tuberculosis,” Am J Respir Crit Care Med, vol. 197, no. 9, pp. 1198-1208, May 2018, doi: 10.1164/rccm.201711-23400C.
[9] T. E. Sweeney, L. Braviak, C. M. Tato, and P. Khatri, “Genome-wide expression for diagnosis of pulmonary tuberculosis: a multicohort analysis,” Lancet Respir Med, vol. 4, no. 3, pp. 213-224, March 2016, doi: 10.1016/S2213-2600(16)00048-5.
[10] J. S. Sutherland et al., “Diagnostic accuracy of the Cepheid 3-gene host response fingerstick blood test in a prospective, multi-site study: interim results,” Clin Infect Dis, p. ciab839, September 2021, doi: 10.1093/cid/ciab839.
[11] R. K. Gupta et al., “Concise whole blood transcriptional signatures for incipient tuberculosis: a systematic review and patient-level pooled meta-analysis,” The Lancet Respiratory Medicine, vol. 8, no. 4, pp. 395-406, April 2020, doi: 10.1016/S2213-2600(19)30282-6.
[12] M. A. Behr, P. H. Edelstein, and L. Ramakrishnan, “Revisiting the timetable of tuberculosis,” BMJ, vol. 362, p. k2738, August 2018, doi: 10.1136/bmj.k2738.
[13] A. Pawlowski, M. Jansson, M. Skold, M. E. Rottenberg, and G. Kullenius, “Tuberculosis and HIV Co-Infection,” PLoS Pathog, vol. 8, no. 2, p. e1002464, February 2012, doi: 10.1371/journal.ppat.1002464.
[14] C. T. Turner et al., “Blood transcriptional biomarkers for active pulmonary tuberculosis in a high-burden setting: a prospective, observational, diagnostic accuracy study,” Lancet Respir Med, vol. 8, no. 4, pp. 407-419, April 2020, doi: 10.1016/52213-2600(19)30469-2.
[15] E. Södersten et al., “Diagnostic accuracy study of a novel blood-based assay for identification of TB in people living with HIV,” Journal of Clinical Microbiology, vol. 59, no. 3, December 2020, doi: 10.1128/jcm.01643-20.
[16] F. M. F. Moreira et al., “Blood-based host biomarker diagnostics in active case finding for pulmonary tuberculosis: A diagnostic case-control study,” EClinicalMedicine, vol. 33, p. 100776, March 2021, doi: 10.1016/j.eclinm.2021.100776.

While the preferred embodiments of the invention have been illustrated and described, it will be appreciated that various changes can be made therein without departing from the spirit and scope of the invention.

TABLE 5

| Gene 1 | Gene 2 | AUROC |
|---|---|---|
| GBP5 | ELF1 | 0.925 |
| GBP5 | ACP5 | 0.923 |
| GBP5 | CAPN2 | 0.923 |
| GBP5 | GNG11 | 0.922 |
| GBP5 | KLF6 | 0.921 |
| GBP5 | NOLC1 | 0.921 |
| GBP5 | YEATS2 | 0.92 |
| GBP5 | PFDN5 | 0.92 |
| GBP5 | STK16 | 0.918 |
| GBP5 | MCM6 | 0.918 |
| GBP5 | DNAJC4 | 0.917 |
| GBP5 | COX6B1 | 0.917 |
| GBP5 | XPO1 | 0.917 |
| GBP5 | TNFAIP3 | 0.916 |
| GBP5 | ARHGEF3 | 0.916 |
| GBP5 | RBM28 | 0.916 |
| GBP5 | SPEN | 0.916 |
| GBP5 | TOPBP1 | 0.916 |
| GBP5 | LIMK1 | 0.916 |
| GBP5 | ARID1A | 0.916 |
| GBP5 | SMARCC1 | 0.916 |
| GBP5 | NDRG1 | 0.916 |
| GBP5 | GSTO1 | 0.915 |
| GBP5 | TIPARP | 0.915 |
| GBP5 | OSBP | 0.915 |
| GBP5 | SECTM1 | 0.915 |
| GBP5 | SLCO4C1 | 0.915 |
| GBP5 | CTCF | 0.915 |
| GBP5 | STAG1 | 0.915 |
| GBP5 | SLC38A1 | 0.915 |
| GBP5 | NENF | 0.915 |
| GBP5 | CCS | 0.915 |
| GBP5 | LIG1 | 0.914 |
| GBP5 | MCM2 | 0.914 |
| GBP5 | TP53BP2 | 0.914 |
| GBP5 | TMED3 | 0.914 |
| GBP5 | TRAPPC3 | 0.914 |
| GBP5 | SYNCRIP | 0.913 |
| GBP5 | PHF3 | 0.913 |
| GBP5 | HIGD2A | 0.913 |
| GBP5 | KNTC1 | 0.913 |
| GBP5 | MSH6 | 0.913 |
| GBP5 | SYNJ2 | 0.913 |
| GBP5 | MSH2 | 0.913 |
| GBP5 | POLB | 0.913 |
| GBP5 | PUM1 | 0.913 |
| GBP5 | DIAPH1 | 0.913 |
| GBP5 | RGS10 | 0.913 |
| GBP5 | EIF1 | 0.913 |
| GBP5 | BAHD1 | 0.913 |
| GBP5 | HMGCR | 0.913 |
| GBP5 | SLTM | 0.913 |
| NPC2 | P2RY14 | 0.913 |
| GBP5 | NCOA1 | 0.912 |
| GBP5 | C1QA | 0.912 |
| GBP5 | AKAP1 | 0.912 |
| GBP5 | NRG1 | 0.912 |
| GBP5 | GIMAP6 | 0.912 |
| GBP5 | RUNX2 | 0.912 |
| GBP5 | PRDM4 | 0.912 |
| GBP5 | NDUFA1 | 0.912 |
| GBP5 | TOB2 | 0.912 |
| GBP5 | PLAGL2 | 0.912 |
| GBP5 | ZBTB16 | 0.912 |
| POLB | NDRG1 | 0.912 |
| GBP5 | TIMELESS | 0.912 |
| GBP5 | RFC1 | 0.912 |
| P2RY14 | PPP3CA | 0.912 |
| GBP5 | NFATC3 | 0.912 |
| GBP5 | GFI1B | 0.912 |
| GBP5 | FYCO1 | 0.912 |
| GBP5 | ETS1 | 0.912 |
| GBP5 | NUP210 | 0.912 |
| GBP5 | SMARCD3 | 0.912 |
| GBP5 | SEC63 | 0.911 |
| GBP5 | LAPTM4A | 0.911 |
| GBP5 | ZNHIT1 | 0.911 |
| P2RY14 | C1QA | 0.911 |
| GBP5 | TSR1 | 0.911 |
| GBP5 | GPBP1L1 | 0.911 |
| GBP5 | MAP3K4 | 0.911 |
| GBP5 | DHX9 | 0.911 |
| GBP5 | TULP4 | 0.911 |
| GBP5 | BARD1 | 0.911 |
| GBP5 | SATB1 | 0.911 |
| GSTO1 | P2RY14 | 0.911 |
| GBP5 | CEP250 | 0.911 |
| GBP5 | SRPK2 | 0.911 |
| GBP5 | RPS14 | 0.911 |
| GBP5 | MAP4 | 0.911 |
| GBP5 | TMEM59 | 0.911 |
| GBP5 | FOLR2 | 0.911 |
| GBP5 | NCL | 0.911 |
| GBP5 | NIPBL | 0.911 |
| GBP5 | NCOR1 | 0.91 |
| GBP5 | OSBPL9 | 0.91 |
| GBP5 | ANKS1A | 0.91 |
| GBP5 | RRM1 | 0.91 |
| GBP5 | MTMR6 | 0.91 |
| POLB | IL15RA | 0.91 |
| GBP5 | TAF2 | 0.91 |
| GBP5 | TSPAN14 | 0.91 |
| GBP5 | SLC16A6 | 0.91 |
| GBP5 | BCR | 0.91 |
| GBP5 | PLCG1 | 0.91 |
| GBP5 | OXSR1 | 0.91 |
| GBP5 | CAMK4 | 0.91 |
| GBP5 | PFAS | 0.91 |
| P2RY14 | SECTM1 | 0.91 |
| POLB | P2RY14 | 0.91 |
| GBP5 | TMSB10 | 0.91 |
| GBP5 | ZFR | 0.91 |
| GBP5 | RNGTT | 0.91 |
| GBP5 | GLG1 | 0.91 |
| GBP5 | MAML1 | 0.91 |
| GBP5 | DGKD | 0.91 |
| GBP5 | ATF7IP | 0.91 |
| GBP5 | USP8 | 0.909 |
| GBP5 | POLD4 | 0.909 |
| GBP5 | POGZ | 0.909 |
| GBP5 | NPC2 | 0.909 |
| GBP5 | TMEM39B | 0.909 |
| GBP5 | MCM3 | 0.909 |
| GBP5 | DDX27 | 0.909 |
| GBP5 | NFE2L1 | 0.909 |
| GBP5 | MAX | 0.909 |
| GBP5 | GYPC | 0.909 |
| GBP5 | PPBP | 0.909 |
| GBP5 | PPP3CA | 0.909 |
| GBP5 | FAM3A | 0.909 |
| GBP5 | NPAT | 0.909 |
| GBP5 | ZNF189 | 0.909 |
| GBP5 | ALMS1 | 0.909 |
| GBP5 | AP4E1 | 0.909 |
| GBP5 | AP1G1 | 0.909 |
| SECTM1 | NDRG1 | 0.909 |
| GBP5 | KLF3 | 0.909 |
| GBP5 | MX2 | 0.909 |
| GBP5 | POLG | 0.909 |
| GBP5 | UPF2 | 0.909 |
| GBP5 | PRKCQ | 0.909 |
| POLB | SECTM1 | 0.909 |
| GBP5 | STK38 | 0.909 |
| GBP5 | PAXIP1 | 0.909 |
| GBP5 | ITPR3 | 0.909 |
| GBP5 | USP1 | 0.908 |
| GBP5 | ACBD3 | 0.908 |
| GBP5 | SS18 | 0.908 |
| GBP5 | ADNP | 0.908 |
| GBP5 | RTN2 | 0.908 |
| GBP5 | ESD | 0.908 |
| GBP5 | UBE2L3 | 0.908 |
| GBP5 | ERBB2 | 0.908 |
| GBP5 | ENSA | 0.908 |
| GBP5 | MSN | 0.908 |
| GBP5 | ATP2B4 | 0.908 |
| GBP5 | ILF3 | 0.908 |
| GBP5 | RFWD3 | 0.908 |
| GBP5 | ACAD8 | 0.908 |
| GBP5 | SMAD3 | 0.908 |
| GBP5 | SON | 0.908 |
| GBP5 | FBXW2 | 0.908 |
| GBP5 | SAR1A | 0.908 |
| GBP5 | TM9SF4 | 0.908 |
| GBP5 | RAB11B | 0.908 |
| GBP5 | NDUFB6 | 0.908 |
| GBP5 | POLR3B | 0.908 |
| GBP5 | MAK | 0.908 |
| P2RY14 | STAG1 | 0.908 |
| GBP5 | PRDM2 | 0.908 |
| GBP5 | HERC1 | 0.908 |
| GBP5 | POU2F1 | 0.908 |
| GBP5 | ZC3H7A | 0.908 |
| GBP5 | ZZEF1 | 0.908 |
| GBP5 | PTP4A1 | 0.908 |
| GBP5 | PFKP | 0.908 |
| GBP5 | AKR1A1 | 0.908 |
| GBP5 | GTPBP4 | 0.908 |
| GBP5 | REV3L | 0.908 |
| P2RY14 | SATB1 | 0.908 |
| GBP5 | TFAM | 0.908 |
| GBP5 | TLE4 | 0.908 |
| GBP5 | ANP32B | 0.908 |
| GBP5 | USP48 | 0.908 |
| GBP5 | RBBP4 | 0.907 |
| GBP5 | TPP2 | 0.907 |
| GBP5 | PPP2R5B | 0.907 |
| GBP5 | PSMB1 | 0.907 |
| GBP5 | PPRC1 | 0.907 |
| GBP5 | CBLB | 0.907 |
| COX681 | P2RY14 | 0.907 |
| P2RY14 | LIMK1 | 0.907 |
| GBP5 | OSBPL11 | 0.907 |
| GBP5 | WDR13 | 0.907 |
| P2RY14 | SLCO4C1 | 0.907 |
| GBP5 | RPAP1 | 0.907 |
| GBP5 | ATP2A2 | 0.907 |
| P2RY14 | ELF1 | 0.907 |
| GBP5 | PSMD5 | 0.907 |
| GBP5 | MYC | 0.907 |
| GBP5 | OXCT1 | 0.907 |
| GBP5 | SLC39A8 | 0.907 |
| GBP5 | MYCBP2 | 0.907 |
| GBP5 | ST6GAL1 | 0.907 |
| GBP5 | PIK3R1 | 0.907 |
| GBP5 | PSME1 | 0.907 |
| P2RY14 | RBM28 | 0.907 |
| GBP5 | MTRR | 0.907 |
| GBP5 | NCBP2 | 0.907 |
| GBP5 | GUK1 | 0.907 |
| GBP5 | DUSP22 | 0.907 |
| GBP5 | TRAF1 | 0.907 |
| GBP5 | LSG1 | 0.907 |
| GBP5 | ASXL2 | 0.907 |
| GBP5 | DENND4A | 0.907 |
| GBP5 | AUTS2 | 0.907 |
| GBP5 | ARFGEF1 | 0.907 |
| GBP5 | TRAF3 | 0.907 |
| GBP5 | CENPJ | 0.907 |
| GBP5 | ABR | 0.907 |
| GBP5 | ZNF318 | 0.907 |
| GBP5 | ZNF365 | 0.907 |
| GBP5 | NUP155 | 0.907 |
| GBP5 | REEP4 | 0.907 |
| GBP5 | PIK3C2B | 0.906 |
| GBP5 | TOB1 | 0.906 |
| GBP5 | KPNA6 | 0.906 |
| GBP5 | LRRC8D | 0.906 |
| GBP5 | SLA | 0.906 |
| GBP5 | VPS4B | 0.906 |
| GBP5 | DENND2D | 0.906 |
| GBP5 | SAP18 | 0.906 |
| GBP5 | GOLGA3 | 0.906 |
| GBP5 | LRRFIP1 | 0.906 |
| GBP5 | EPHA4 | 0.906 |
| GBP5 | DAD1 | 0.906 |
| GBP5 | UBP1 | 0.906 |
| GBP5 | BCOR | 0.906 |
| GBP5 | VPS13D | 0.906 |
| GBP5 | SDC1 | 0.906 |
| GBP5 | SIDT1 | 0.906 |
| GBP5 | CNOT2 | 0.906 |
| PSME1 | P2RY14 | 0.906 |
| GBP5 | CHAF1B | 0.906 |
| GBP5 | CAND1 | 0.906 |
| GBP5 | ZNF236 | 0.906 |
| GBP5 | DBR1 | 0.906 |
| GBP5 | KLF11 | 0.906 |
| GBP5 | GEMIN4 | 0.906 |
| GBP5 | MAT2A | 0.906 |
| GBP5 | PRKAR2A | 0.906 |
| GBP5 | ERCC3 | 0.906 |
| GBP5 | LRIG1 | 0.906 |
| GBP5 | NOC3L | 0.906 |
| GBP5 | USP47 | 0.906 |
| GBP5 | TXK | 0.906 |
| GBP5 | STT3A | 0.906 |
| P2RY14 | NOLC1 | 0.906 |
| GBP5 | UBAP2 | 0.906 |
| ACP5 | P2RY14 | 0.906 |
| GBP5 | SUCLG1 | 0.906 |
| GBP5 | TMEM87A | 0.906 |
| GBP5 | NR1D2 | 0.906 |
| GBP5 | IL21R | 0.906 |
| GBP5 | ADAM17 | 0.906 |
| GBP5 | PPP4R1 | 0.906 |
| GBP5 | PTGER2 | 0.906 |
| GBP5 | NFX1 | 0.906 |
| GBP5 | IL15RA | 0.906 |
| GBP5 | SNAPC3 | 0.905 |
| GBP5 | RRAGC | 0.905 |
| GBP5 | KDELR1 | 0.905 |
| P2RY14 | SRPK2 | 0.905 |
| GBP5 | ANKRD27 | 0.905 |
| GBP5 | CCND2 | 0.905 |
| GBP5 | TMEM123 | 0.905 |
| GBP5 | YTHDC1 | 0.905 |
| GBP5 | PLAA | 0.905 |
| GBP5 | P2RY14 | 0.905 |
| GBP5 | RAPGEF1 | 0.905 |
| GBP5 | TOR1AIP1 | 0.905 |
| GBP5 | BAX | 0.905 |
| GBP5 | ANK3 | 0.905 |
| GBP5 | DDIT4 | 0.905 |
| GBP5 | PDIA4 | 0.905 |
| GBP5 | DCTN4 | 0.905 |
| GBP5 | ABL1 | 0.905 |
| GBP5 | NFKB1 | 0.905 |
| GBP5 | MTSS1 | 0.905 |
| GBP5 | TNFRSF14 | 0.905 |
| GBP5 | MYD88 | 0.905 |
| GBP5 | MYO9A | 0.905 |
| GBP5 | CPVL | 0.905 |
| GBP5 | TCF12 | 0.905 |
| GBP5 | POLR1D | 0.905 |
| GBP5 | PDHA1 | 0.905 |
| GBP5 | FBXO21 | 0.905 |
| GBP5 | MTR | 0.905 |
| GBP5 | SFXN1 | 0.905 |
| GBP5 | NUMA1 | 0.905 |
| GBP5 | OGDH | 0.905 |
| GBP5 | CTNNB1 | 0.905 |
| GBP5 | ABCA2 | 0.905 |
| GBP5 | TRIB2 | 0.905 |
| GBP5 | ING4 | 0.905 |
| GBP5 | GTF2E1 | 0.905 |
| GBP5 | TXN2 | 0.905 |
| GBP5 | SMARCC2 | 0.905 |
| GBP5 | ZNF76 | 0.905 |
| GBP5 | DPM2 | 0.905 |
| GBP5 | CNOT8 | 0.905 |
| GBP5 | DNAJA3 | 0.905 |
| GBP5 | AQR | 0.905 |
| P2RY14 | RPS6KA3 | 0.905 |
| GBP5 | XPC | 0.905 |
| GBP5 | PPAT | 0.905 |
| GBP5 | RACGAP1 | 0.905 |
| GBP5 | SLC39A6 | 0.905 |
| GBP5 | NT5C2 | 0.905 |
| P2RY14 | VAMP5 | 0.905 |
| GBP5 | RNF111 | 0.905 |
| GBP5 | RRN3 | 0.905 |
| GBP5 | FOXJ2 | 0.905 |
| GBP5 | CALU | 0.905 |
| POLB | KLF6 | 0.905 |
| GBP5 | VDAC3 | 0.905 |
| GBP5 | MAPK6 | 0.905 |
| GBP5 | MFAP1 | 0.904 |
| GBP5 | MAPK13 | 0.904 |
| P2RY14 | ADD3 | 0.904 |
| GBP5 | KHDRBS1 | 0.904 |
| GBP5 | EHD4 | 0.904 |
| P2RY14 | SLC38A1 | 0.904 |
| GBP5 | MBTPS1 | 0.904 |
| GBP5 | CANX | 0.904 |
| GBP5 | KLF13 | 0.904 |
| GBP5 | DPF2 | 0.904 |
| GBP5 | CORO1B | 0.904 |
| GBP5 | TCF7 | 0.904 |
| GBP5 | MALT1 | 0.904 |
| GBP5 | MAN2A1 | 0.904 |
| P2RY14 | ZNF189 | 0.904 |
| GBP5 | POLR1B | 0.904 |
| P2RY14 | KLF6 | 0.904 |
| GBP5 | ITGA7 | 0.904 |
| GBP5 | ATP8B2 | 0.904 |
| GBP5 | HIC2 | 0.904 |
| GBP5 | GOLPH3 | 0.904 |
| GBP5 | RNF38 | 0.904 |
| GBP1 | ELF1 | 0.904 |
| GBP5 | GMEB1 | 0.904 |
| P2RY14 | HMGCR | 0.904 |
| GBP5 | GPD1L | 0.904 |
| GBP5 | GTF3C3 | 0.904 |
| GBP5 | PPP2R5A | 0.904 |
| GBP5 | NFATC1 | 0.904 |
| GBP5 | MECP2 | 0.904 |
| POLB | LIMK1 | 0.904 |
| POLB | AGPAT3 | 0.904 |
| GBP5 | MKRN2 | 0.904 |
| POLB | SMARCC1 | 0.904 |
| GBP5 | SIRT5 | 0.904 |
| GBP5 | ADD3 | 0.904 |
| GBP5 | GEMIN7 | 0.904 |
| GBP5 | CLASP1 | 0.904 |
| GBP5 | HYOU1 | 0.904 |
| GBP5 | BCL2L11 | 0.904 |
| GBP5 | COMMD4 | 0.904 |
| GBP5 | APH1A | 0.904 |
| GBP5 | CDC23 | 0.904 |
| GBP5 | STAG2 | 0.904 |
| GBP5 | PIGK | 0.904 |
| P2RY14 | RNGTT | 0.904 |
| GBP5 | TM7SF2 | 0.904 |
| GBP5 | ETS2 | 0.904 |
| GBP5 | GFOD1 | 0.904 |
| GBP5 | RAP2C | 0.904 |
| GBP5 | SAP130 | 0.904 |
| P2RY14 | RUNX2 | 0.904 |
| GBP5 | ELAC2 | 0.904 |
| GBP5 | GNE | 0.904 |
| GBP5 | CUL4A | 0.904 |
| GBP5 | RAB14 | 0.904 |
| GBP5 | BBS7 | 0.904 |
| GBP5 | SLC6A6 | 0.904 |
| GBP5 | POLD3 | 0.904 |
| GBP5 | PCBD1 | 0.904 |
| GBP5 | CHMP6 | 0.904 |
| GBP5 | SNRK | 0.904 |
| GBP5 | TACC1 | 0.903 |
| GBP5 | DDX46 | 0.903 |
| GBP5 | ATP6V1E1 | 0.903 |
| P2RY14 | REV3L | 0.903 |
| P2RY14 | ETS1 | 0.903 |
| APOL2 | NDRG1 | 0.903 |
| P2RY14 | APOL2 | 0.903 |
| GBP5 | HERC2 | 0.903 |
| P2RY14 | OSBPL11 | 0.903 |
| P2RY14 | TAF2 | 0.903 |
| GBP5 | DLEC1 | 0.903 |
| GBP5 | SRRM1 | 0.903 |
| GBP5 | ITPR1 | 0.903 |
| POLB | POLR3B | 0.903 |
| GBP5 | ARIH2 | 0.903 |
| GBP5 | RER1 | 0.903 |
| P2RY14 | MSH2 | 0.903 |
| GBP5 | UBE4A | 0.903 |
| GBP5 | EML4 | 0.903 |
| GBP5 | UBE21 | 0.903 |
| GBP5 | PER2 | 0.903 |
| GBP5 | EXOSC10 | 0.903 |
| GBP5 | NR3C2 | 0.903 |
| GBP5 | TMEM43 | 0.903 |
| GBP5 | TTF1 | 0.903 |
| GBP5 | PTPN11 | 0.903 |
| GBP5 | CCDC47 | 0.903 |
| GBP5 | AFF4 | 0.903 |
| GBP5 | CYB5R3 | 0.903 |
| P2RY14 | ADNP | 0.903 |
| GBP5 | SLC30A5 | 0.903 |
| GBP5 | RPS6KA3 | 0.903 |
| P2RY14 | KLF3 | 0.903 |
| GBP5 | VCP | 0.903 |
| GBP5 | APBA2 | 0.903 |
| GBP5 | S100A9 | 0.903 |
| GBP5 | POLR2C | 0.903 |
| GBP5 | UROS | 0.903 |
| GBP5 | TRIM68 | 0.903 |
| SECTM1 | PXN | 0.903 |
| GBP5 | EHBP1 | 0.903 |
| GBP5 | SIRT6 | 0.903 |
| GBP5 | ST8SIA4 | 0.903 |
| P2RY14 | TOPBP1 | 0.903 |
| GBP5 | DYNC1L12 | 0.903 |
| GBP5 | SNIP1 | 0.903 |
| P2RY14 | AKAP1 | 0.903 |
| GBP5 | MAPKAPK5 | 0.903 |
| GBP5 | MYH9 | 0.903 |
| GBP5 | PARP6 | 0.903 |
| GBP5 | RBL2 | 0.903 |
| UBE2L6 | P2RY14 | 0.903 |
| P2RY14 | SLTM | 0.903 |
| GBP5 | CDK5RAP1 | 0.903 |
| GBP5 | PSMD11 | 0.903 |
| GBP5 | CD74 | 0.903 |
| GBP5 | PPP2RSE | 0.903 |
| GBP5 | MDM2 | 0.903 |
| GBP5 | ZCCHC14 | 0.903 |
| GBP5 | ANKRD11 | 0.903 |
| GBP5 | PAPOLA | 0.903 |
| GBP5 | PPM1D | 0.903 |
| GBP5 | CD40 | 0.903 |
| GBP5 | HTRA2 | 0.903 |
| GBP5 | PDIA6 | 0.903 |
| P2RY14 | STK16 | 0.903 |
| GBP5 | CSE1L | 0.903 |
| GBP5 | HK2 | 0.903 |
| P2RY14 | APOL1 | 0.903 |
| GBP5 | SSRP1 | 0.903 |
| SMARCD3 | P2RY14 | 0.903 |
| P2RY14 | TRAPPC3 | 0.903 |
| GBP5 | PON2 | 0.903 |
| GBP5 | WBP11 | 0.903 |
| GBP5 | AGPAT3 | 0.903 |
| GBP5 | PARP16 | 0.903 |
| GBP5 | TNPO1 | 0.902 |
| GBP5 | RRS1 | 0.902 |
| GBP5 | HMG20A | 0.902 |
| GBP5 | WRN | 0.902 |
| GBP5 | APOL1 | 0.902 |
| GBP5 | SEC23B | 0.902 |
| GBP5 | TSC1 | 0.902 |
| GBP5 | ODF2 | 0.902 |
| GBP5 | GRAMD1C | 0.902 |
| GBP5 | FNBP1 | 0.902 |
| GBP5 | RUNX3 | 0.902 |
| GBP5 | MAPK1 | 0.902 |
| GBP5 | DNPEP | 0.902 |
| GBP5 | ALDOC | 0.902 |
| GBP5 | THUMPD1 | 0.902 |
| GBP5 | CALCOCO2 | 0.902 |
| P2RY14 | DENND4A | 0.902 |
| GBP5 | TGFBRAP1 | 0.902 |
| GBP5 | PARVB | 0.902 |
| GBP5 | CACNA2D2 | 0.902 |
| GBP5 | PRPF8 | 0.902 |
| P2RY14 | SMARCC1 | 0.902 |
| P2RY14 | DNAJC4 | 0.902 |
| GBP5 | ARCN1 | 0.902 |
| GBP5 | UROD | 0.902 |
| GBP5 | TMCC1 | 0.902 |
| GBP5 | AACS | 0.902 |
| P2RY14 | MAK | 0.902 |
| GBP5 | ITCH | 0.902 |
| GBP5 | NET1 | 0.902 |
| GBP5 | ABCC10 | 0.902 |
| GBP5 | CYB5R1 | 0.902 |
| GBP5 | MAF | 0.902 |
| GBP5 | RRBP1 | 0.902 |
| GBP5 | ATG3 | 0.902 |
| GBP5 | DLG4 | 0.902 |
| GBP5 | MTCH2 | 0.902 |
| GBP5 | CDYL | 0.902 |
| GBP5 | APOL2 | 0.902 |
| GBP5 | ZHX2 | 0.902 |
| GBP5 | PXN | 0.902 |
| GBP5 | EIF4G1 | 0.902 |
| GBP5 | POLH | 0.902 |
| GBP5 | LAS1L | 0.902 |
| GBP5 | DDX23 | 0.902 |
| P2RY14 | XPO1 | 0.902 |
| GBP5 | VAMP5 | 0.902 |
| P2RY14 | OSBP | 0.902 |
| GBP5 | DHX15 | 0.902 |
| GBP5 | TERF2 | 0.902 |
| GBP5 | NUP50 | 0.902 |
| GBP5 | LAX1 | 0.902 |
| GBP5 | ADI1 | 0.902 |
| P2RY14 | ZFR | 0.902 |
| GBP5 | NDUFB2 | 0.902 |
| GBP5 | SDHB | 0.902 |
| GBP5 | ADAM15 | 0.902 |
| GBP5 | E2F3 | 0.902 |
| AKR1A1 | P2RY14 | 0.902 |
| GBP5 | COMMD9 | 0.902 |
| GBP5 | PCSK6 | 0.902 |
| P2RY14 | SEC63 | 0.902 |
| P2RY14 | TMED3 | 0.902 |
| GBP5 | TNK2 | 0.902 |
| POLB | STAG1 | 0.902 |
| P2RY14 | ACBD3 | 0.902 |
| P2RY14 | HIGD2A | 0.902 |
| GBP5 | DLAT | 0.902 |
| GBP5 | LSM1 | 0.902 |
| GBP5 | SSBP1 | 0.902 |
| GBP5 | ZCCHC8 | 0.902 |
| GBP5 | PDPK1 | 0.902 |
| POLB | OSBP | 0.902 |
| GBP5 | DCUN1D2 | 0.902 |
| GBP5 | RFC5 | 0.902 |
| POLB | CTCF | 0.902 |
| GBP5 | CAMSAP1 | 0.902 |
| GBP5 | ELF4 | 0.902 |
| GBP5 | NUP107 | 0.901 |
| GBP5 | SERINC3 | 0.901 |
| POLB | C1QA | 0.901 |
| GBP5 | SESN1 | 0.901 |
| GBP5 | PSMB6 | 0.901 |
| GBP5 | SMU1 | 0.901 |
| GBP5 | ZNF337 | 0.901 |
| GBP5 | OGFOD1 | 0.901 |
| P2RY14 | ZNF318 | 0.901 |
| GBP5 | GALNT1 | 0.901 |
| GBP5 | SKP2 | 0.901 |
| GBP5 | SERTAD3 | 0.901 |
| GBP5 | PLCD1 | 0.901 |
| GBP5 | RBBP7 | 0.901 |
| GBP5 | MGST3 | 0.901 |
| GBP5 | AP3B1 | 0.901 |
| GBP5 | ATP9B | 0.901 |
| GBP5 | UBIAD1 | 0.901 |
| GBP5 | ZMPSTE24 | 0.901 |
| GBP5 | CAMK2G | 0.901 |
| P2RY14 | RNF38 | 0.901 |
| P2RY14 | GPBP1L1 | 0.901 |
| GBP5 | FANCE | 0.901 |
| GBP5 | VPS11 | 0.901 |
| GBP5 | VILL | 0.901 |
| GBP5 | ARHGEF7 | 0.901 |
| GBP5 | ELP3 | 0.901 |
| GBP5 | VPS39 | 0.901 |
| GBP5 | DENND4C | 0.901 |
| GBP5 | ECHS1 | 0.901 |
| GBP5 | RECQL | 0.901 |
| GBP5 | PEX1 | 0.901 |
| P2RY14 | USP1 | 0.901 |
| GBP5 | RAB11FIP2 | 0.901 |
| GBP5 | TBX21 | 0.901 |
| GBP5 | PRKCH | 0.901 |
| GBP5 | EIF4ENIF1 | 0.901 |
| GBP5 | DHX38 | 0.901 |
| GBP5 | SLC25A12 | 0.901 |
| GBP5 | PIK3R5 | 0.901 |
| GBP5 | CTDSPL | 0.901 |
| P2RY14 | MSH6 | 0.901 |
| GBP5 | PPP2R5D | 0.901 |
| GBP5 | EP300 | 0.901 |
| GBP5 | PUS7 | 0.901 |
| GBP5 | DOCK2 | 0.901 |
| P2RY14 | PHF3 | 0.901 |
| GBP5 | PHKG2 | 0.901 |
| GBP5 | JARID2 | 0.901 |
| GBP5 | EP400 | 0.901 |
| GBP5 | ATG12 | 0.901 |
| GBP5 | TBC1D22A | 0.901 |
| P2RY14 | PUM1 | 0.901 |
| P2RY14 | MTMR6 | 0.901 |
| GBP5 | CSNK1G3 | 0.901 |
| POLB | RUNX2 | 0.901 |
| GBP5 | PAM | 0.901 |
| GBP5 | TMEM115 | 0.901 |
| P2RY14 | CAMK4 | 0.901 |
| GBP5 | BRD3 | 0.901 |
| GBP5 | DUSP3 | 0.901 |
| GBP5 | CETN2 | 0.9 |
| POLB | DHX9 | 0.9 |
| GBP5 | PTGER4 | 0.9 |
| GBP5 | MGAT4A | 0.9 |
| GBP5 | PPP2R5C | 0.9 |
| GBP5 | UPB1 | 0.9 |
| GBP5 | GANAB | 0.9 |
| P2RY14 | IL15RA | 0.9 |
| GBP5 | TRAP1 | 0.9 |
| GBP5 | CORO2A | 0.9 |
| GBP5 | NMT1 | 0.9 |
| GBP5 | MAGED1 | 0.9 |
| GBP5 | SLC35A3 | 0.9 |
| GBP5 | DENND1A | 0.9 |
| GBP5 | PIK3CB | 0.9 |
| GBP5 | REL | 0.9 |
| ZNHIT1 | P2RY14 | 0.9 |
| GBP5 | PIGB | 0.9 |
| GBP5 | HSPAS | 0.9 |
| GBP5 | MTHFD1 | 0.9 |
| GBP5 | KRIT1 | 0.9 |
| GBP5 | RFC2 | 0.9 |
| GBP5 | CDK6 | 0.9 |
| GBP5 | SIRT1 | 0.9 |
| GBP5 | PARP2 | 0.9 |
| GBP5 | JAM3 | 0.9 |
| GBP5 | MRPS12 | 0.9 |
| NDUFA1 | PZRY14 | 0.9 |
| GBP5 | RPA1 | 0.9 |
| GBP5 | SCMH1 | 0.9 |
| GBP5 | SLC25A17 | 0.9 |
| GBP5 | SRD5A1 | 0.9 |
| GBP5 | GALNT2 | 0.9 |
| GBP5 | DNAL4 | 0.9 |
| GBP5 | WDR12 | 0.9 |
| P2RY14 | NENF | 0.9 |
| GBP5 | ETF1 | 0.9 |
| GBP5 | DALRD3 | 0.9 |
| GBP5 | PITRM1 | 0.9 |
| P2RY14 | RNF111 | 0.9 |
| GBP5 | SMARCE1 | 0.9 |
| GBP5 | CTBP2 | 0.9 |
| GBP5 | ZMYM5 | 0.9 |
| GBP5 | BCKDK | 0.9 |
| GBP5 | ACTR8 | 0.9 |
| GBP5 | SERPINB6 | 0.9 |
| GBP5 | INPP1 | 0.9 |
| GBP5 | NOTCH2 | 0.9 |
| GBP5 | ABHD11 | 0.9 |
| GBP5 | BCL2 | 0.9 |
| GBP5 | H6PD | 0.9 |
| GBP5 | CLDND1 | 0.9 |
| GBP5 | IL12RB1 | 0.9 |
| GBP5 | GTF2B | 0.9 |
| GBP5 | SLC43A1 | 0.9 |
| GBP5 | TAF4 | 0.9 |
| GBP5 | PPP1R12A | 0.9 |
| GBP5 | GRWD1 | 0.9 |
| GBP5 | FLNB | 0.9 |
| GBP5 | F13A1 | 0.9 |
| GBP5 | ACTN4 | 0.9 |
| GBP5 | EIF5 | 0.9 |
| GBP5 | PF4V1 | 0.9 |
| GBP5 | UNC119 | 0.9 |
| GBP5 | ATP6V1A | 0.9 |
| GBP5 | INPPSE | 0.9 |
| P2RY14 | GALNT1 | 0.9 |
| GBP5 | TEX10 | 0.9 |
| GBP5 | MIS12 | 0.9 |
| GBP5 | KLHL7 | 0.9 |
| GBP5 | TAF6 | 0.9 |
| P2RY14 | TMSB10 | 0.9 |
| P2RY14 | TOB2 | 0.9 |
| GBP5 | CDCA4 | 0.9 |
| GBP5 | PINK1 | 0.9 |
| GBP5 | AP3M2 | 0.9 |
| P2RY14 | SPEN | 0.9 |
| GBP5 | CHMP7 | 0.9 |
| GBP5 | CD244 | 0.9 |
| GBP5 | STIM1 | 0.9 |
| GBP5 | STAT6 | 0.9 |
| GBP5 | KIF3B | 0.9 |
| GBP5 | VPS33A | 0.9 |
| GBP5 | SEH1L | 0.9 |
| GBP5 | MDM1 | 0.9 |
| GBP5 | CYB561 | 0.899 |
| GBP5 | ZAP70 | 0.899 |
| GBP5 | EXOSC9 | 0.899 |
| GBP5 | DHCR7 | 0.899 |
| GBP5 | SNW1 | 0.899 |
| GBP5 | RHOC | 0.899 |
| GBP5 | PHF20 | 0.899 |
| GBP5 | YWHAQ | 0.899 |
| GBP5 | DNAJC1 | 0.899 |
| GBP5 | BTBD1 | 0.899 |
| GBP5 | SNX16 | 0.899 |
| GBP5 | ACO1 | 0.899 |
| GBP5 | MDK | 0.899 |
| GBP5 | PDK3 | 0.899 |
| GBP5 | DNM1L | 0.899 |
| GBP5 | DHX29 | 0.899 |
| P2RY14 | ARFGEF1 | 0.899 |
| GBP5 | MAPRE2 | 0.899 |
| SECTM1 | RIN3 | 0.899 |
| P2RY14 | HERC1 | 0.899 |
| GBP5 | ECHDC2 | 0.899 |
| GBP5 | YIPF3 | 0.899 |
| GBP5 | PPP2CA | 0.899 |
| P2RY14 | DGKD | 0.899 |
| GBP5 | SLBP | 0.899 |
| GBP5 | MRPL49 | 0.899 |
| P2RY14 | TCF12 | 0.899 |
| GBP5 | TTLL12 | 0.899 |
| GBP5 | FTSJ3 | 0.899 |
| P2RY14 | BAX | 0.899 |
| GBP5 | DDB2 | 0.899 |
| GBP5 | ANGEL1 | 0.899 |
| GBP5 | YWHAZ | 0.899 |
| GBP5 | POLDIP2 | 0.899 |
| GBP5 | STK4 | 0.899 |
| P2RY14 | ARID1A | 0.899 |
| POLB | PRDM2 | 0.899 |
| GBP5 | ABLIM1 | 0.899 |
| GBP5 | CACNA1I | 0.899 |
| GBP5 | CASP6 | 0.899 |
| P2RY14 | PLCG1 | 0.899 |
| GBP5 | RAD1 | 0.899 |
| P2RY14 | TIPARP | 0.899 |
| GBP5 | YTHDF1 | 0.899 |
| GBP5 | PCSK7 | 0.899 |
| GBP5 | UBTF | 0.899 |
| GBP5 | BTG1 | 0.899 |
| GBP5 | NKTR | 0.899 |
| GBP5 | ADAT1 | 0.899 |
| GBP5 | RNF170 | 0.899 |
| GBP5 | CRY2 | 0.899 |
| GBP5 | NRGN | 0.899 |
| GBP5 | DDX24 | 0.899 |
| GBP5 | USP4 | 0.899 |
| GBP5 | TMEM97 | 0.899 |
| GBP5 | TRIM37 | 0.899 |
| GBP5 | ZXDC | 0.899 |
| GBP5 | ARMCX3 | 0.899 |
| GBP5 | SYNGR1 | 0.899 |
| P2RY14 | DCTN4 | 0.899 |
| GBP5 | CHMP2A | 0.899 |
| GBP5 | PPM1B | 0.899 |
| GBP5 | EXOC7 | 0.899 |
| GBP5 | RBM22 | 0.899 |
| GBP5 | ABCF2 | 0.899 |
| GBP5 | RNMT | 0.899 |
| GBP5 | TSEN2 | 0.899 |
| GBP5 | DPH2 | 0.899 |
| GBP5 | SETD3 | 0.899 |
| P2RY14 | PSMB1 | 0.899 |
| GBP5 | FZD2 | 0.899 |
| GBP5 | IRF3 | 0.899 |
| GBP5 | ASB1 | 0.899 |
| P2RY14 | NPAT | 0.899 |
| GBP5 | CCNK | 0.899 |
| POLB | MAX | 0.899 |
| GBP5 | TXNL1 | 0.899 |
| GBP5 | POLR2D | 0.899 |
| GBP5 | LY86 | 0.899 |
| GBP5 | PRF1 | 0.899 |
| GBP5 | TRIM24 | 0.899 |
| P2RY14 | TEAM | 0.898 |
| GBP5 | CDC34 | 0.898 |
| GBP5 | USP11 | 0.898 |
| GBP5 | POMT1 | 0.898 |
| P2RY14 | UBE4A | 0.898 |
| GBP5 | BSDC1 | 0.898 |
| GBP5 | SACS | 0.898 |
| GBP5 | CLEC10A | 0.898 |
| GBP5 | SLC35D2 | 0.898 |
| GBP5 | RAB32 | 0.898 |
| GBP5 | CA5B | 0.898 |
| P2RY14 | RBL2 | 0.898 |
| GBP5 | ITPKB | 0.898 |
| GBP5 | IL10RA | 0.898 |
| GBP5 | OVGP1 | 0.898 |
| POLB | DGKD | 0.898 |
| GBP5 | BRF2 | 0.898 |
| GBP5 | YIPF5 | 0.898 |
| GBP5 | CPSF1 | 0.898 |
| GBP5 | PRKD2 | 0.898 |
| GBP5 | POP7 | 0.898 |
| GBP5 | EXOSC2 | 0.898 |
| P2RY14 | EIF1 | 0.898 |
| GBP5 | PCYOX1 | 0.898 |
| GBP5 | OSBPL2 | 0.898 |
| GBP5 | R3HDM2 | 0.898 |
| POLB | DDX27 | 0.898 |
| P2RY14 | PSMB6 | 0.898 |
| GBP5 | CYB561D2 | 0.898 |
| P2RY14 | STAG2 | 0.898 |
| GBP5 | ARMC1 | 0.898 |
| P2RY14 | RFC1 | 0.898 |
| GBP5 | IL2RB | 0.898 |
| GBP5 | ANKRD10 | 0.898 |
| GBP5 | PAK1IP1 | 0.898 |
| GBP5 | NUP98 | 0.898 |
| GBP5 | ARMCX5 | 0.898 |
| GBP5 | MGAT2 | 0.898 |
| GBP5 | HIF1A | 0.898 |
| GBP5 | CPNE3 | 0.898 |
| UBE2L6 | NDRG1 | 0.898 |
| GBP5 | HMGCL | 0.898 |
| GBP5 | CDC42 | 0.898 |
| GBP5 | UBE4B | 0.898 |
| GBP5 | HNMT | 0.898 |
| GBP5 | HSPA14 | 0.898 |
| GBP5 | RPL39L | 0.898 |
| GBP5 | AQP3 | 0.898 |
| GBP5 | PREP | 0.898 |
| GBP5 | NUP54 | 0.898 |
| GBP5 | AXIN1 | 0.898 |
| GBP5 | GPR137 | 0.898 |
| GBP5 | CBX1 | 0.898 |
| GBP5 | STARD5 | 0.898 |
| GBP5 | NDUFS4 | 0.898 |
| GBP5 | MDH2 | 0.898 |
| GBP5 | KYNU | 0.898 |
| GBP5 | ATPAF2 | 0.898 |
| GBP5 | RBM15 | 0.898 |
| GBP5 | PPARD | 0.898 |
| GBP5 | RAF1 | 0.898 |
| GBP5 | UBE2G1 | 0.898 |
| GBP5 | PXMP4 | 0.898 |
| GBP5 | SUZ12 | 0.898 |
| GBP5 | METAP1 | 0.898 |
| GBP5 | UPF3A | 0.898 |
| GBP5 | IL16 | 0.898 |
| GBP5 | CDCA8 | 0.898 |
| GBP5 | LAMP2 | 0.898 |
| GNG11 | P2RY14 | 0.898 |
| GBP5 | IARS2 | 0.898 |
| GBP5 | ZNF217 | 0.898 |
| GBP5 | PLEK2 | 0.898 |
| GBP5 | CD33 | 0.898 |
| GBP5 | HSPH1 | 0.898 |
| GBP5 | TUFT1 | 0.898 |
| GBP5 | AASDHPPT | 0.898 |
| GBP5 | STARD8 | 0.898 |
| GBP5 | RIN2 | 0.898 |
| GBP5 | CBX4 | 0.898 |
| GBP5 | USP3 | 0.898 |
| SECTM1 | TSPAN14 | 0.898 |
| GBP5 | SETBP1 | 0.898 |
| GBP5 | ISOC2 | 0.898 |
| GBP5 | ZNF672 | 0.898 |
| POLB | TSR1 | 0.898 |
| GBP5 | ZNF394 | 0.898 |
| GBP5 | COG5 | 0.898 |
| GBP5 | PMM2 | 0.898 |
| GBP5 | ZNF232 | 0.898 |
| GBP5 | COX7A2 | 0.898 |
| GBP5 | UCHL5 | 0.898 |
| GBP5 | GNA11 | 0.898 |
| P2RY14 | CTCF | 0.898 |
| P2RY14 | TP53BP2 | 0.898 |
| GBP5 | NGLY1 | 0.898 |
| GBP5 | MGST2 | 0.898 |
| P2RY14 | SESN1 | 0.898 |
| GBP5 | CRK | 0.898 |
| POLB | ACP5 | 0.898 |
| GBP5 | CASK | 0.898 |
| GBP5 | TAF1C | 0.898 |
| GBP5 | CHD4 | 0.898 |
| GBP5 | SRP54 | 0.898 |
| GBP5 | RASGRP1 | 0.898 |
| GBP5 | MNAT1 | 0.897 |
| GBP5 | IRF4 | 0.897 |
| GBP5 | COASY | 0.897 |
| P2RY14 | EPHA4 | 0.897 |
| P2RY14 | PPP1R12A | 0.897 |
| GBP5 | TTC19 | 0.897 |
| GBP5 | ZNF212 | 0.897 |
| UBE2L6 | POLB | 0.897 |
| GBP5 | TBCD | 0.897 |
| GBP5 | DAPP1 | 0.897 |
| GBP5 | PRKACA | 0.897 |
| GBP5 | INSIG2 | 0.897 |
| GBP5 | FNBP4 | 0.897 |
| P2RY14 | USP8 | 0.897 |
| GBP5 | TUBD1 | 0.897 |
| P2RY14 | SYNJ2 | 0.897 |
| GBP5 | SPG21 | 0.897 |
| POLB | LIG1 | 0.897 |
| GBP5 | ABCE1 | 0.897 |
| GBP5 | IER5 | 0.897 |
| GBP5 | TGOLN2 | 0.897 |
| GBP5 | ITGAL | 0.897 |
| GBP5 | VPS54 | 0.897 |
| GBP5 | TEX261 | 0.897 |
| GBP5 | CHFR | 0.897 |
| GBP5 | ATP6V1H | 0.897 |
| GBP5 | ZNF573 | 0.897 |
| POLB | MAP3K4 | 0.897 |
| P2RY14 | OXSR1 | 0.897 |
| GBP5 | DAAM1 | 0.897 |
| GBP5 | TES | 0.897 |
| GBP5 | KPTN | 0.897 |
| GBP5 | EIF5A2 | 0.897 |
| GBP5 | ATP13A1 | 0.897 |
| P2RY14 | RAB11B | 0.897 |
| GBP5 | CLMN | 0.897 |
| GBP5 | GALNT11 | 0.897 |
| GBP5 | RIOK2 | 0.897 |
| GBP5 | SOCS2 | 0.897 |
| P2RY14 | PFAS | 0.897 |
| GBP5 | PUS1 | 0.897 |
| GBP5 | MRPL19 | 0.897 |
| P2RY14 | TULP4 | 0.897 |
| GBP5 | RASSF1 | 0.897 |
| GBP5 | MYO9B | 0.897 |
| GBP5 | GLUL | 0.897 |
| GBP5 | SMAD7 | 0.897 |
| GBP5 | PHF10 | 0.897 |
| GBP5 | IFT122 | 0.897 |
| GBP5 | GMFG | 0.897 |
| GBP5 | SLC35F2 | 0.897 |
| GBP5 | ZNF330 | 0.897 |
| GBP5 | CDC25B | 0.897 |
| GBP5 | LASP1 | 0.897 |
| GBP5 | ASB8 | 0.897 |
| GBP5 | SMAD4 | 0.897 |
| GBP5 | ECD | 0.897 |
| GBP5 | B4GALT3 | 0.897 |
| GBP5 | BTG3 | 0.897 |
| GBP5 | HSF2 | 0.897 |
| GBP5 | ANXA7 | 0.897 |
| GBP5 | UBE2L6 | 0.897 |
| GBP5 | CASP2 | 0.897 |
| GBP5 | MAP3K14 | 0.897 |
| GBP5 | POP4 | 0.897 |
| GBP5 | CLK4 | 0.897 |
| GBP5 | SPIN1 | 0.897 |
| GBP5 | MBD1 | 0.897 |
| GBP5 | ABHD6 | 0.897 |
| GBP5 | VTI1B | 0.897 |
| GBP5 | LPIN1 | 0.897 |
| GBP5 | NUDT2 | 0.897 |
| POLB | AKAP1 | 0.897 |
| GBP5 | ITGA2B | 0.897 |
| GBP5 | LBR | 0.897 |
| GBP5 | LRP1 | 0.897 |
| GBP5 | YEATS4 | 0.897 |
| POLB | NIPBL | 0.897 |
| P2RY14 | YEATS2 | 0.897 |
| GBP5 | CIDEB | 0.897 |
| GBP5 | SMAP1 | 0.897 |
| GBP5 | RCOR3 | 0.897 |
| POLB | CALCOCO2 | 0.897 |
| GBP5 | GPR132 | 0.897 |
| GBP5 | TIMM44 | 0.897 |
| GBP5 | SDK2 | 0.897 |
| GBP5 | TNFRSF9 | 0.897 |
| GBP5 | LSR | 0.897 |
| GBP5 | SLC25A22 | 0.897 |
| GBP5 | NISCH | 0.897 |
| GBP5 | OSGEPL1 | 0.897 |
| GBP5 | TRIM46 | 0.897 |
| GBP5 | PTPN2 | 0.897 |
| GBP5 | HARS2 | 0.897 |
| GBP5 | SQLE | 0.897 |
| GBP5 | SOAT1 | 0.897 |
| GBP5 | TMED5 | 0.897 |
| GBP5 | TSFM | 0.897 |
| GBP5 | ELAVL1 | 0.897 |
| P2RY14 | PIK3R1 | 0.897 |
| GBP5 | SMARCD1 | 0.897 |
| GBP5 | BRPF1 | 0.896 |
| GBP5 | NAPA | 0.896 |
| GBP5 | RUVBL1 | 0.896 |
| P2RY14 | NIPBL | 0.896 |
| GBP5 | RAB8B | 0.896 |
| GBP5 | SLC2A9 | 0.896 |
| GBP5 | CSTF2 | 0.896 |
| GBP5 | JOSD1 | 0.896 |
| GBP5 | YPEL1 | 0.896 |
| GBP5 | TRAF3IP3 | 0.896 |
| GBP5 | KCTD17 | 0.896 |
| GBP5 | SLAMF1 | 0.896 |
| GBP5 | RAD23B | 0.896 |
| GBP5 | PSMD1 | 0.896 |
| P2RY14 | PRDM4 | 0.896 |
| GBP5 | BECN1 | 0.896 |
| GBP5 | KLF2 | 0.896 |
| GBP5 | CLPX | 0.896 |
| GBP5 | S100A8 | 0.896 |
| P2RY14 | ATP2B4 | 0.896 |
| GBP5 | GGA1 | 0.896 |
| GBP5 | AMMECR1 | 0.896 |
| GBP5 | VAMP3 | 0.896 |
| GBP5 | PSD4 | 0.896 |
| GBP5 | CCDC25 | 0.896 |
| GBP5 | CORO1C | 0.896 |
| P2RY14 | LRIG1 | 0.896 |
| P2RY14 | THUMPD1 | 0.896 |
| GBP5 | ZNF589 | 0.896 |
| GBP5 | CLEC4A | 0.896 |
| GBP5 | TOE1 | 0.896 |
| GBP5 | PKNOX1 | 0.896 |
| P2RY14 | REEP4 | 0.896 |
| GBP5 | PDHX | 0.896 |
| GBP5 | VAC14 | 0.896 |
| GBP5 | RAB11FIP3 | 0.896 |
| GBP5 | MAP2K1 | 0.896 |
| GBP5 | DCP2 | 0.896 |
| GBP5 | STX8 | 0.896 |
| GBP5 | SPAG9 | 0.896 |
| GBP5 | ERCC2 | 0.896 |
| GBP5 | NARF | 0.896 |
| GBP5 | PSMB7 | 0.896 |
| GBP5 | ELMO1 | 0.896 |
| GBP5 | SEPHS2 | 0.896 |
| GBP5 | TBXA2R | 0.896 |
| GBP5 | GNB1 | 0.896 |
| GBP5 | RAB11FIP1 | 0.896 |
| GBP5 | CNGB1 | 0.896 |
| GBP5 | DDX50 | 0.896 |
| GBP5 | GMPR2 | 0.896 |
| GBP5 | CIC | 0.896 |
| P2RY14 | CAPN2 | 0.896 |
| GBP5 | RABSB | 0.896 |
| P2RY14 | NCOA1 | 0.896 |
| GBP5 | ASTE1 | 0.896 |
| GBP5 | CEP68 | 0.896 |
| GBP5 | MS4A6A | 0.896 |
| GBP5 | ACP1 | 0.896 |
| GBP5 | PBXIP1 | 0.896 |
| GBP5 | NMT2 | 0.896 |
| GBP5 | BLCAP | 0.896 |
| GBP5 | ASB6 | 0.896 |
| GBP5 | LHFPL2 | 0.896 |
| GBP5 | CCR4 | 0.896 |
| GBP5 | FZD5 | 0.896 |
| ACP5 | GBP1 | 0.896 |
| GBP5 | PAOX | 0.896 |
| P2RY14 | MTRR | 0.896 |
| GBP5 | CLSTN1 | 0.896 |
| GBP5 | GAMT | 0.896 |
| GBP5 | ITIHS | 0.896 |
| GBP5 | LCK | 0.896 |
| GBP5 | PPP3CC | 0.896 |
| P2RY14 | GPD1L | 0.896 |
| P2RY14 | PML | 0.896 |
| GBP5 | CKAP2 | 0.896 |
| GBP5 | PRMT7 | 0.896 |
| P2RY14 | TPP2 | 0.896 |
| GBP5 | SERPINB8 | 0.896 |
| GBP5 | ZNF274 | 0.896 |
| P2RY14 | VPS4B | 0.896 |
| GBP5 | RNF130 | 0.896 |
| GBP5 | DCAKD | 0.896 |
| GBP5 | TSPAN32 | 0.896 |
| GBP5 | ZBTB33 | 0.896 |
| GBP5 | TMEM106C | 0.896 |
| GBP5 | PCNA | 0.896 |
| GBP5 | SOX4 | 0.896 |
| GBP5 | CD59 | 0.896 |
| GBP5 | THAP1 | 0.896 |
| GBP5 | TM7SF3 | 0.896 |
| GBP5 | MYL9 | 0.896 |
| GBP5 | EXOSC8 | 0.896 |
| GBP5 | TAL1 | 0.896 |
| GBP5 | TLE2 | 0.896 |
| GBP5 | PSMD2 | 0.896 |
| P2RY14 | DDX46 | 0.896 |
| GBP5 | PSMB2 | 0.896 |
| GBP5 | VPS33B | 0.896 |
| GBP5 | PTPN22 | 0.896 |
| GBP5 | EIF2B5 | 0.896 |
| P2RY14 | MAML1 | 0.896 |
| POLB | DNAJC4 | 0.896 |
| GBP5 | PGRMC2 | 0.896 |
| GBP5 | WIPI2 | 0.896 |
| P2RY14 | RRN3 | 0.896 |
| GBP5 | XYLT2 | 0.896 |
| P2RY14 | PTPN11 | 0.896 |
| GBP5 | FLII | 0.896 |
| P2RY14 | NR1D2 | 0.896 |
| GBP5 | MINK1 | 0.896 |
| GBP5 | SPSB3 | 0.896 |
| GBP5 | NKRF | 0.896 |
| GBP5 | BRD9 | 0.896 |
| GBP5 | HBS1L | 0.896 |
| GBP5 | ING1 | 0.896 |
| GBP5 | PICALM | 0.896 |
| P2RY14 | ZC3H7A | 0.896 |
| P2RY14 | SON | 0.896 |
| P2RY14 | BAHD1 | 0.896 |
| GBP5 | PRPF4 | 0.896 |
| GBP5 | PTPN7 | 0.896 |
| GBP5 | MAP4K4 | 0.895 |
| GBP5 | PDE4A | 0.895 |
| GBP5 | BAG3 | 0.895 |
| GBP5 | FKRP | 0.895 |
| P2RY14 | ZNF236 | 0.895 |
| GBP5 | ARHGEF6 | 0.895 |
| GBP5 | SNAPC4 | 0.895 |
| GBP5 | TPM1 | 0.895 |
| GBP5 | MAGEH1 | 0.895 |
| GBP5 | MLX | 0.895 |
| GBP5 | GTDC1 | 0.895 |
| GBP5 | TCTA | 0.895 |
| GBP5 | DTX3 | 0.895 |
| GBP5 | KLF10 | 0.895 |
| GBP5 | MCM5 | 0.895 |
| GBP5 | KLHL3 | 0.895 |
| GBP5 | GTF3C2 | 0.895 |
| P2RY14 | TXK | 0.895 |
| GBP5 | TOP2B | 0.895 |
| GBP5 | NMUR1 | 0.895 |
| GBP5 | SLC1A4 | 0.895 |
| P2RY14 | ACAD8 | 0.895 |
| P2RY14 | PAXIP1 | 0.895 |
| GBP5 | TIMM8B | 0.895 |
| GBP5 | DHRS7B | 0.895 |
| GBP5 | RFXANK | 0.895 |
| GBP5 | CCT5 | 0.895 |
| GBP5 | GSPT2 | 0.895 |
| GBP5 | MED9 | 0.895 |
| GBP5 | FLAD1 | 0.895 |
| GBP5 | PDK4 | 0.895 |
| GBP5 | MRPL9 | 0.895 |
| GBP5 | AMPD3 | 0.895 |
| GBP5 | RELA | 0.895 |
| GBP5 | FAM53C | 0.895 |
| P2RY14 | ALMS1 | 0.895 |
| GBP5 | PDCD6IP | 0.895 |
| GBP5 | PIGH | 0.895 |
| GBP5 | GPA33 | 0.895 |
| GBP5 | LANCL1 | 0.895 |
| GBP5 | SHC1 | 0.895 |
| GBP5 | ZNF614 | 0.895 |
| P2RY14 | AP4E1 | 0.895 |
| KDELR1 | P2RY14 | 0.895 |
| P2RY14 | SYNCRIP | 0.895 |
| GBP5 | LETM1 | 0.895 |
| GBP5 | ZNF266 | 0.895 |
| GBP5 | UBAP2L | 0.895 |
| GBP5 | CCNG2 | 0.895 |
| GBP5 | MAN1C1 | 0.895 |
| GBP5 | PYCARD | 0.895 |
| P2RY14 | GFI1B | 0.895 |
| GBP5 | CHRNB1 | 0.895 |
| P2RY14 | OXCT1 | 0.895 |
| P2RY14 | OSBPL9 | 0.895 |
| GBP5 | MPI | 0.895 |
| GBP5 | TICAM1 | 0.895 |
| GBP5 | KLF9 | 0.895 |
| GBP5 | MRPS27 | 0.895 |
| P2RY14 | SIRT1 | 0.895 |
| GBP5 | ADIPOR2 | 0.895 |
| GBP5 | ADAM19 | 0.895 |
| GBP5 | WDR4 | 0.895 |
| DAD1 | P2RY14 | 0.895 |
| P2RY14 | HK2 | 0.895 |
| GBP5 | CCT6A | 0.895 |
| GBP5 | ING3 | 0.895 |
| GBP5 | CRNKL1 | 0.895 |
| GBP5 | ITGB5 | 0.895 |
| GBP5 | SLC9A1 | 0.895 |
| GBP5 | MSRB2 | 0.895 |
| GBP5 | TGFBR3 | 0.895 |
| P2RY14 | ADAM17 | 0.895 |
| GBP5 | MCM7 | 0.895 |
| GBP5 | POLR2B | 0.895 |
| GBP5 | KATNA1 | 0.895 |
| P2RY14 | TNFRSF14 | 0.895 |
| GBP5 | ETFDH | 0.895 |
| P2RY14 | PLEKHF2 | 0.895 |
| GBP5 | PRNP | 0.895 |
| GBP5 | ZNF235 | 0.895 |
| P2RY14 | AFF4 | 0.895 |
| GBP5 | PRRG4 | 0.895 |
| GBP5 | SLC38A2 | 0.895 |
| GBP5 | EXT1 | 0.895 |
| GBP5 | PLCL2 | 0.895 |
| GBP5 | MUTYH | 0.895 |
| GBP5 | TMC6 | 0.895 |
| P2RY14 | SLC39A6 | 0.895 |
| GBP5 | CXCR3 | 0.895 |
| GBP5 | ZBTB25 | 0.895 |
| GBP5 | ATIC | 0.895 |
| GBP5 | MAZ | 0.895 |
| POLB | CEP250 | 0.895 |
| GBP5 | PRDX4 | 0.895 |
| GBP5 | TSPYL2 | 0.895 |
| P2RY14 | RAB14 | 0.895 |
| GBP5 | MPPE1 | 0.895 |
| GBP5 | TCFL5 | 0.895 |
| GBP5 | STAM | 0.895 |
| GBP5 | ARNTL | 0.895 |
| GBP5 | CSDE1 | 0.895 |
| P2RY14 | EML4 | 0.895 |
| GBP5 | PRPSAP1 | 0.895 |
| POLB | PHF3 | 0.895 |
| GBP5 | GTPBP3 | 0.895 |
| GBP5 | RANBP3 | 0.895 |
| GBP5 | PPP1CC | 0.895 |
| P2RY14 | POGZ | 0.895 |
| POLB | HMGCR | 0.895 |
| GBP5 | MTAP | 0.894 |
| GBP5 | PCK2 | 0.894 |
| GBP5 | TBC1D1 | 0.894 |
| GBP5 | BTN2A2 | 0.894 |
| GBP5 | UBE2D4 | 0.894 |
| GBP5 | IDS | 0.894 |
| GBP5 | RNF122 | 0.894 |
| P2RY14 | SDC1 | 0.894 |
| P2RY14 | CORO1B | 0.894 |
| GBP5 | CACNB3 | 0.894 |
| GBP5 | PRPF3 | 0.894 |
| GBP5 | POLD1 | 0.894 |
| CCS | P2RY14 | 0.894 |
| GBP5 | GORASP2 | 0.894 |
| GBP5 | PLEKHF2 | 0.894 |
| GBP5 | DNAJC7 | 0.894 |
| SECTM1 | CTDP1 | 0.894 |
| P2RY14 | UBE2L3 | 0.894 |
| POLB | USP48 | 0.894 |
| GBP5 | PIP5K1C | 0.894 |
| GBP5 | DRG1 | 0.894 |
| P2RY14 | SNRK | 0.894 |
| GBP5 | DUSP11 | 0.894 |
| GBP5 | INPPL1 | 0.894 |
| GBP5 | UGP2 | 0.894 |
| GBP5 | SH2D2A | 0.894 |
| GBP5 | LRFN3 | 0.894 |
| GBP5 | PPP1R8 | 0.894 |
| GBP5 | EML3 | 0.894 |
| GBP5 | ATP6V0A1 | 0.894 |
| GBP5 | FBXL12 | 0.894 |
| P2RY14 | ASXL2 | 0.894 |
| POLB | PDHA1 | 0.894 |
| P2RY14 | SMAD3 | 0.894 |
| GBP5 | BZW2 | 0.894 |
| GBP5 | HPS1 | 0.894 |
| GBP5 | ARHGAP12 | 0.894 |
| P2RY14 | MTR | 0.894 |
| GBP5 | CTDP1 | 0.894 |
| GBP5 | TBRG4 | 0.894 |
| GBP5 | NCK1 | 0.894 |
| GBP5 | PCBP4 | 0.894 |
| GBP5 | CYP20A1 | 0.894 |
| GBP5 | BCCIP | 0.894 |
| P2RY14 | TXN2 | 0.894 |
| GBP5 | PUS7L | 0.894 |
| P2RY14 | ST6GAL1 | 0.894 |
| GBP5 | SLC4A7 | 0.894 |
| P2RY14 | ANKRD27 | 0.894 |
| GBP5 | COPS7A | 0.894 |
| GBP5 | MORF4L1 | 0.894 |
| P2RY14 | NOC3L | 0.894 |
| GBP5 | PML | 0.894 |
| P2RY14 | SUCLG1 | 0.894 |
| P2RY14 | MAX | 0.894 |
| GBP5 | SLC25A28 | 0.894 |
| GBP5 | BCORL1 | 0.894 |
| GBP5 | DFFA | 0.894 |
| GBP5 | NONO | 0.894 |
| GBP5 | UBE3A | 0.894 |
| GBP5 | CDK10 | 0.894 |
| GBP5 | AGL | 0.894 |
| P2RY14 | COMMD4 | 0.894 |
| GBP5 | GNRH1 | 0.894 |
| P2RY14 | UBP1 | 0.894 |
| GBP5 | PPIG | 0.894 |
| P2RY14 | USP48 | 0.894 |
| P2RY14 | FYCO1 | 0.894 |
| GBP5 | SCCPDH | 0.894 |
| GBP5 | ATP6V1G1 | 0.894 |
| GBP5 | DDX54 | 0.894 |
| PSME1 | POLB | 0.894 |
| GBP5 | AVEN | 0.894 |
| GBP5 | CASP9 | 0.894 |
| P2RY14 | ATP6V1E1 | 0.894 |
| GBP5 | MRPL16 | 0.894 |
| GBP5 | RECQLS | 0.894 |
| P2RY14 | KNTC1 | 0.894 |
| GBP5 | PTGIR | 0.894 |
| FOLR2 | P2RY14 | 0.894 |
| GBP5 | CREB3 | 0.894 |
| GBP5 | ICMT | 0.894 |
| GBP5 | MAGED2 | 0.894 |
| GBP5 | SYT11 | 0.894 |
| P2RY14 | DHX9 | 0.894 |
| GBP5 | RCL1 | 0.894 |
| GBP5 | GMDS | 0.894 |
| GBP5 | AAMP | 0.894 |
| P2RY14 | SS18 | 0.894 |
| GBP5 | NARS2 | 0.894 |
| POLB | GPBP1L1 | 0.894 |
| GBP5 | APEH | 0.894 |
| GBP5 | CDKAL1 | 0.894 |
| P2RY14 | LIG1 | 0.894 |
| GBP5 | TRAM1 | 0.894 |
| P2RY14 | MAP3K4 | 0.894 |
| GBP5 | GFI1 | 0.894 |
| P2RY14 | NFATC3 | 0.894 |
| GBP5 | PITPNC1 | 0.894 |
| GBP5 | KEAP1 | 0.894 |
| GBP1 | MX2 | 0.894 |
| GBP5 | SERINC1 | 0.894 |
| GBP5 | BACH2 | 0.894 |
| GBP5 | NOL11 | 0.894 |
| GBP5 | STMN1 | 0.894 |
| GBP5 | MPHOSPH10 | 0.894 |
| P2RY14 | CAND1 | 0.894 |
| GBP5 | ZCWPW1 | 0.894 |
| GBP5 | TP5313 | 0.893 |
| GBP5 | ALG6 | 0.893 |
| GBP5 | CASP1 | 0.893 |
| P2RY14 | PRDM2 | 0.893 |
| GBP5 | COG2 | 0.893 |
| GBP5 | ATP2A3 | 0.893 |
| GBP5 | SLC7A7 | 0.893 |
| GBP5 | GLMN | 0.893 |
| GBP5 | KIF23 | 0.893 |
| P2RY14 | PIGK | 0.893 |
| GBP5 | SH3TC1 | 0.893 |
| GBP5 | TNFSF8 | 0.893 |
| P2RY14 | APH1A | 0.893 |
| GBP5 | NUDT21 | 0.893 |
| P2RY14 | GRAMD1C | 0.893 |
| GBP5 | SNX11 | 0.893 |
| GBP5 | TAOK2 | 0.893 |
| TMEM59 | P2RY14 | 0.893 |
| P2RY14 | PFDN5 | 0.893 |
| GBP5 | ACVR1 | 0.893 |
| GBP5 | CPT2 | 0.893 |
| GBP5 | PLK3 | 0.893 |
| POLB | DENND1A | 0.893 |
| GBP5 | BNIP1 | 0.893 |
| GBP5 | ACP2 | 0.893 |
| P2RY14 | NCOR1 | 0.893 |
| P2RY14 | CD74 | 0.893 |
| POLB | LHFPL2 | 0.893 |
| GBP5 | MAPRE1 | 0.893 |
| GBP5 | IFRD1 | 0.893 |
| P2RY14 | DUSP3 | 0.893 |
| GBP5 | ASNS | 0.893 |
| GBP5 | SUV39H1 | 0.893 |
| P2RY14 | USP47 | 0.893 |
| GBP5 | VRK3 | 0.893 |
| GBP5 | ZNF426 | 0.893 |
| PCBD1 | P2RY14 | 0.893 |
| P2RY14 | MYO9A | 0.893 |
| GBP5 | DNAJB1 | 0.893 |
| GBP5 | SLCO3A1 | 0.893 |
| POLB | MSH6 | 0.893 |
| GBP5 | HADHA | 0.893 |
| GBP5 | TBC1D13 | 0.893 |
| POLB | PML | 0.893 |
| GBP5 | E2F1 | 0.893 |
| GBP5 | SCRIB | 0.893 |
| GBP5 | CCDC51 | 0.893 |
| GBP5 | SPATA2 | 0.893 |
| GBP5 | PEA15 | 0.893 |
| GBP5 | CTSF | 0.893 |
| GBP5 | PDCL | 0.893 |
| GBP5 | ARHGEF2 | 0.893 |
| GBP5 | VPS35 | 0.893 |
| GBP5 | RAD51C | 0.893 |
| GBP5 | STX10 | 0.893 |
| GBP5 | CLCF1 | 0.893 |
| GBP5 | ITGA5 | 0.893 |
| GBP5 | MBD2 | 0.893 |
| GBP5 | MED8 | 0.893 |
| GBP5 | PGM1 | 0.893 |
| GBP5 | IL1R1 | 0.893 |
| GBP5 | GBE1 | 0.893 |
| GBP5 | DECR1 | 0.893 |
| GBP5 | TSPAN2 | 0.893 |
| GBP5 | RGS12 | 0.893 |
| GBP5 | ODC1 | 0.893 |
| GBP5 | DUSP2 | 0.893 |
| GBP5 | DDX1 | 0.893 |
| GBP5 | GTPBP8 | 0.893 |
| GBP5 | PBX1 | 0.893 |
| GBP5 | IMMT | 0.893 |
| GBP5 | TFRC | 0.893 |
| POLB | NCL | 0.893 |
| P2RY14 | PPM1D | 0.893 |
| GBP5 | FUCA1 | 0.893 |
| GBP5 | ATP7B | 0.893 |
| GBP5 | ZNF7 | 0.893 |
| P2RY14 | MGAT4A | 0.893 |
| GBP5 | ADCY9 | 0.893 |
| GBP5 | CD1A | 0.893 |
| GBP1 | CAPN2 | 0.893 |
| GBP5 | ECE1 | 0.893 |
| GBP5 | CBR1 | 0.893 |
| GBP5 | MLH1 | 0.893 |
| GBP5 | DNAJC17 | 0.893 |
| GBP5 | APOL3 | 0.893 |
| GBP5 | NDUFS1 | 0.893 |
| P2RY14 | SACS | 0.893 |
| GBP5 | CCNB1 | 0.893 |
| P2RY14 | MALT1 | 0.893 |
| P2RY14 | IL12RB1 | 0.893 |
| GBP5 | PKD2 | 0.893 |
| GBP5 | ELL | 0.893 |
| P2RY14 | CCNG2 | 0.893 |
| GBP5 | PRPSAP2 | 0.893 |
| P2RY14 | TMEM39B | 0.893 |
| GBP5 | CTSC | 0.893 |
| GBP5 | TLE3 | 0.893 |
| GBP5 | HCFC2 | 0.893 |
| GBP5 | DNM2 | 0.893 |
| GBP5 | KCNAB2 | 0.893 |
| GBP5 | RNF41 | 0.893 |
| GBP5 | AP1B1 | 0.892 |
| GBP5 | CTSS | 0.892 |
| POLB | CNOT2 | 0.892 |
| GBP5 | GAS7 | 0.892 |
| GBP5 | G6PD | 0.892 |
| GBP5 | TFB2M | 0.892 |
| GBP5 | CDK5RAP3 | 0.892 |
| GBP5 | XPNPEP1 | 0.892 |
| GBP5 | NPEPPS | 0.892 |
| P2RY14 | MKRN2 | 0.892 |
| GBP5 | PRKAG1 | 0.892 |
| POLB | CAMK2G | 0.892 |
| GBP5 | MCFD2 | 0.892 |
| GBP5 | CBLL1 | 0.892 |
| GBP5 | BCAT1 | 0.892 |
| GBP5 | MAPK7 | 0.892 |
| GBP5 | DDB1 | 0.892 |
| GBP5 | SLC22A1 | 0.892 |
| GBP5 | MOAP1 | 0.892 |
| GBP5 | RHEB | 0.892 |
| GBP5 | PSMD7 | 0.892 |
| GBP5 | FADD | 0.892 |
| GBP5 | ZNF562 | 0.892 |
| GBP5 | GOLT1B | 0.892 |
| GBP5 | GMIP | 0.892 |
| GBP5 | ZNF211 | 0.892 |
| GBP5 | KDELR2 | 0.892 |
| GBP5 | POLDIP3 | 0.892 |
| GBP5 | ISOC1 | 0.892 |
| P2RY14 | ILF3 | 0.892 |
| GBP5 | NMD3 | 0.892 |
| GBP5 | CCNE1 | 0.892 |
| POLB | RNGTT | 0.892 |
| GBP5 | WBP4 | 0.892 |
| GBP5 | BFAR | 0.892 |
| GBP5 | DNAJB6 | 0.892 |
| GBP5 | PPP2CB | 0.892 |
| GBP5 | PDK2 | 0.892 |
| GBP5 | GRAP2 | 0.892 |
| GBP5 | KCNK3 | 0.892 |
| GBP5 | MAP2K5 | 0.892 |
| GBP5 | FZD1 | 0.892 |
| GBP5 | DR1 | 0.892 |
| GBP5 | SLC5A6 | 0.892 |
| GBP5 | PPCDC | 0.892 |
| GBP5 | CRLF3 | 0.892 |
| GBP5 | PRKAR1A | 0.892 |
| P2RY14 | AP1G1 | 0.892 |
| GBP5 | ADD1 | 0.892 |
| P2RY14 | ZNF217 | 0.892 |
| GBP5 | NME4 | 0.892 |
| GBP5 | PTS | 0.892 |
| GBP5 | FAH | 0.892 |
| P2RY14 | NCBP2 | 0.892 |
| POLB | OSBPL9 | 0.892 |
| GBP5 | PIGA | 0.892 |
| GBP5 | TNNC2 | 0.892 |
| GBP5 | ITPA | 0.892 |
| GBP5 | IRS2 | 0.892 |
| GBP5 | SLC35C1 | 0.892 |
| P2RY14 | KLF13 | 0.892 |
| GBP5 | USP21 | 0.892 |
| GBP5 | NUP37 | 0.892 |
| P2RY14 | ITCH | 0.892 |
| GBP5 | TSNAX | 0.892 |
| GBP5 | PSMA2 | 0.892 |
| GBP5 | CCT4 | 0.892 |
| GBP5 | MFGE8 | 0.892 |
| GBP5 | DCTN5 | 0.892 |
| GBP5 | NADSYN1 | 0.892 |
| GBP5 | ATP1B2 | 0.892 |
| GBP5 | PEX7 | 0.892 |
| GBP5 | SLC25A4 | 0.892 |
| GBP5 | ACOT7 | 0.892 |
| GBP5 | SLC25A20 | 0.892 |
| GBP5 | SEC61A1 | 0.892 |
| GBP5 | PDCD2 | 0.892 |
| GBP5 | RPGR | 0.892 |
| GBP5 | IL7R | 0.892 |
| POLB | APOL2 | 0.892 |
| GBP5 | STOML1 | 0.892 |
| GBP5 | CD9 | 0.892 |
| GBP5 | AHR | 0.892 |
| POLB | SETD3 | 0.892 |
| GBP5 | ABCC3 | 0.892 |
| GBP5 | TARBP2 | 0.892 |
| GBP5 | BCL11B | 0.892 |
| GBP5 | RAC2 | 0.892 |
| GBP5 | ELMO3 | 0.892 |
| GBP5 | SLC35E3 | 0.892 |
| P2RY14 | ZCCHC14 | 0.892 |
| GBP5 | MEN1 | 0.892 |
| P2RY14 | WDR13 | 0.892 |
| GBP5 | ACTL6A | 0.892 |
| GBP5 | VDR | 0.892 |
| P2RY14 | PAPOLA | 0.892 |
| GBP5 | OGFR | 0.892 |
| P2RY14 | ARHGAP12 | 0.892 |
| GBP5 | DEDD | 0.892 |
| P2RY14 | NDRG1 | 0.892 |
| GBP5 | LRRC47 | 0.892 |
| GBP5 | BLMH | 0.892 |
| GBP5 | PHF1 | 0.892 |
| GBP5 | DOLPP1 | 0.892 |
| GBP5 | PECR | 0.892 |
| GBP5 | TMEM127 | 0.892 |
| GBP5 | WTAP | 0.892 |
| GBP5 | STEAP3 | 0.892 |
| GBP5 | WWC3 | 0.892 |
| GBP5 | YWHAB | 0.892 |
| P2RY14 | MYC | 0.892 |
| GBP5 | MDFIC | 0.892 |
| GBP5 | EFHC1 | 0.892 |
| GBP5 | FAM49B | 0.892 |
| POLB | ILF3 | 0.892 |
| GBP5 | SMUG1 | 0.892 |
| GBP5 | DAAM2 | 0.892 |
| GBP5 | POLRMT | 0.892 |
| POLB | POLG | 0.892 |
| GBP5 | ALDH9A1 | 0.891 |
| GBP5 | SLC3A2 | 0.891 |
| GBP5 | PUM2 | 0.891 |
| GBP5 | SLC16A5 | 0.891 |
| POLB | ARID1A | 0.891 |
| P2RY14 | ATF7IP | 0.891 |
| GBP5 | MNT | 0.891 |
| GBP5 | PWP1 | 0.891 |
| GBP5 | RAB20 | 0.891 |
| GBP5 | BUB3 | 0.891 |
| GBP5 | RP2 | 0.891 |
| GBP5 | MON1B | 0.891 |
| GBP5 | SPATA5L1 | 0.891 |
| GBP5 | NFYC | 0.891 |
| GBP5 | RPUSD2 | 0.891 |
| GBP5 | DGKA | 0.891 |
| GBP5 | APTX | 0.891 |
| GBP5 | TSTA3 | 0.891 |
| P2RY14 | ATP8B2 | 0.891 |
| GBP5 | GOLGA7 | 0.891 |
| GBP5 | QPRT | 0.891 |
| P2RY14 | POLR3B | 0.891 |
| GBP5 | ZNF419 | 0.891 |
| GBP5 | CD96 | 0.891 |
| GBP5 | HMGN4 | 0.891 |
| GBP5 | PTGS1 | 0.891 |
| P2RY14 | TSC1 | 0.891 |
| GBP5 | SLC22A18 | 0.891 |
| POLB | STK38 | 0.891 |
| GBP5 | THOC1 | 0.891 |
| GBP5 | RSAD1 | 0.891 |
| GBP5 | ANKRD12 | 0.891 |
| GBP5 | CAP1 | 0.891 |
| GBP5 | RPL3L | 0.891 |
| GBP5 | SEPHS1 | 0.891 |
| P2RY14 | AGL | 0.891 |
| GBP5 | RPA2 | 0.891 |
| GBP5 | TMBIM4 | 0.891 |
| GBP5 | TAPBPL | 0.891 |
| GBP1 | NOLC1 | 0.891 |
| P2RY14 | PPP2R5B | 0.891 |
| P2RY14 | NUP210 | 0.891 |
| POLB | SEC63 | 0.891 |
| GBP5 | CD2BP2 | 0.891 |
| GBP5 | UBE2A | 0.891 |
| P2RY14 | WRN | 0.891 |
| GBP5 | PRKX | 0.891 |
| GBP5 | TBL2 | 0.891 |
| GBP5 | PSMD6 | 0.891 |
| P2RY14 | CLK4 | 0.891 |
| P2RY14 | RAP2C | 0.891 |
| GBP5 | UFM1 | 0.891 |
| GBP5 | TSC2 | 0.891 |
| GBP5 | HDAC1 | 0.891 |
| P2RY14 | SAP18 | 0.891 |
| GBP5 | ACTR2 | 0.891 |
| GBP5 | PKIG | 0.891 |
| APOL1 | NDRG1 | 0.891 |
| P2RY14 | PSMB2 | 0.891 |
| GBP5 | MAP3K10 | 0.891 |
| GBP5 | MBNL2 | 0.891 |
| GBP5 | ARMC7 | 0.891 |
| P2RY14 | KLF11 | 0.891 |
| P2RY14 | KPTN | 0.891 |
| GBP5 | GOT1 | 0.891 |
| P2RY14 | MTSS1 | 0.891 |
| P2RY14 | VPS13D | 0.891 |
| GBP5 | PHKA2 | 0.891 |
| GBP5 | ECE2 | 0.891 |
| P2RY14 | PIK3C2B | 0.891 |
| GBP5 | GAK | 0.891 |
| POLB | SRPK2 | 0.891 |
| P2RY14 | ANKRD12 | 0.891 |
| GBP5 | MTMR9 | 0.891 |
| GNG11 | GBP1 | 0.891 |
| GBP5 | GNL2 | 0.891 |
| P2RY14 | DNAJA3 | 0.891 |
| GBP5 | AMFR | 0.891 |
| GBP5 | FAF1 | 0.891 |
| GBP5 | ZFYVE21 | 0.891 |
| GBP5 | RIN3 | 0.891 |
| GBP5 | VSIG4 | 0.891 |
| GBP5 | CDC7 | 0.891 |
| GBP5 | STAT5A | 0.891 |
| P2RY14 | TRAF1 | 0.891 |
| GBP5 | DSTN | 0.891 |
| P2RY14 | SDHB | 0.891 |
| GBP5 | ABCC5 | 0.891 |
| P2RY14 | ANK3 | 0.891 |
| GBP5 | CDV3 | 0.891 |
| P2RY14 | NR3C2 | 0.891 |
| P2RY14 | DLEC1 | 0.891 |
| GBP5 | USP16 | 0.891 |
| POLB | VAMP5 | 0.891 |
| P2RY14 | APBA2 | 0.891 |
| GBP5 | PLA2G12A | 0.891 |
| GBP5 | CLK3 | 0.891 |
| GBP5 | PSMD10 | 0.891 |
| GBP5 | TMEM50B | 0.891 |
| P2RY14 | CEP250 | 0.891 |
| GBP5 | GPR137B | 0.891 |
| GBP5 | ABCF3 | 0.891 |
| GBP5 | NDE1 | 0.891 |
| POLB | VILL | 0.891 |
| GBP5 | UBE2E1 | 0.891 |
| GBP5 | EHD3 | 0.891 |
| GBP5 | DAZAP1 | 0.891 |
| GBP5 | AAAS | 0.891 |
| GBP5 | SLC25A13 | 0.891 |
| P2RY14 | GTF2E1 | 0.891 |
| GBP5 | APLP2 | 0.891 |
| GBP5 | GLRX5 | 0.891 |
| P2RY14 | AGPAT3 | 0.891 |
| GBP5 | LETMD1 | 0.891 |
| ACP5 | SECTM1 | 0.891 |
| GBP5 | PNPLA2 | 0.89 |
| POLB | TULP4 | 0.89 |
| P2RY14 | SMAD4 | 0.89 |
| GBP5 | HTATIP2 | 0.89 |
| POLB | TTF1 | 0.89 |
| POLB | TMCC1 | 0.89 |
| POLB | GLG1 | 0.89 |
| P2RY14 | CTNNB1 | 0.89 |
| GBP5 | CPA3 | 0.89 |
| GBP5 | VIPR1 | 0.89 |
| GBP5 | CALCOCO1 | 0.89 |
| P2RY14 | PRKAR2A | 0.89 |
| GBP5 | TRIM8 | 0.89 |
| P2RY14 | SLC39A8 | 0.89 |
| GBP5 | TMEM109 | 0.89 |
| GBP5 | AOC3 | 0.89 |
| GBP5 | TNFAIP8 | 0.89 |
| GBP5 | BRD4 | 0.89 |
| GBP5 | SFXN3 | 0.89 |
| GBP5 | KIFC3 | 0.89 |
| GBP5 | RBM10 | 0.89 |
| GBP5 | ICOS | 0.89 |
| GBP5 | MRPS30 | 0.89 |
| P2RY14 | RBBP4 | 0.89 |
| P2RY14 | NFX1 | 0.89 |
| P2RY14 | CDC23 | 0.89 |
| GBP5 | POLR2H | 0.89 |
| GBP5 | TFG | 0.89 |
| GBP5 | CC2D1A | 0.89 |
| GBP5 | MUS81 | 0.89 |
| GBP5 | RYBP | 0.89 |
| GBP5 | AGTPBP1 | 0.89 |
| GBP5 | HSDL2 | 0.89 |
| GBP5 | CAV2 | 0.89 |
| GBP5 | FXR2 | 0.89 |
| GBP5 | CHST2 | 0.89 |
| GBP5 | ACOX3 | 0.89 |
| GBP5 | TRPC4AP | 0.89 |
| P2RY14 | PEX1 | 0.89 |
| SECTM1 | POR | 0.89 |
| GBP5 | HSD17B4 | 0.89 |
| GBP5 | MAP2K4 | 0.89 |
| P2RY14 | DENND4C | 0.89 |
| GBP5 | BCS1L | 0.89 |
| P2RY14 | COMMD9 | 0.89 |
| GBP5 | BYSL | 0.89 |
| GBP5 | ULK1 | 0.89 |
| P2RY14 | SNAPC3 | 0.89 |
| GBP5 | DERA | 0.89 |
| GBP5 | TBX19 | 0.89 |
| GBP5 | TNIP1 | 0.89 |
| P2RY14 | FBXO21 | 0.89 |
| P2RY14 | ADAM15 | 0.89 |
| GBP5 | SAE1 | 0.89 |
| POLB | GOLGA3 | 0.89 |
| GBP5 | ARHGAP1 | 0.89 |
| GBP5 | LRRC42 | 0.89 |
| POLB | IL16 | 0.89 |
| GBP5 | PHYH | 0.89 |
| GBP5 | METAP2 | 0.89 |
| GBP5 | IVNS1ABP | 0.89 |
| P2RY14 | POLG | 0.89 |
| GBP5 | CIAPIN1 | 0.89 |
| GBP5 | ATP1B3 | 0.89 |
| GBP5 | TPRKB | 0.89 |
| GBP5 | PAFAH2 | 0.89 |
| GBP5 | MOSPD2 | 0.89 |
| GBP5 | FOSL2 | 0.89 |
| P2RY14 | KRIT1 | 0.89 |
| POLB | RBM28 | 0.89 |
| GBP5 | RABGGTB | 0.89 |
| P2RY14 | TOB1 | 0.89 |
| GBP5 | MCCC1 | 0.89 |
| GBP5 | MRPL44 | 0.89 |
| GBP5 | RAB35 | 0.89 |
| SECTM1 | CYB561 | 0.89 |
| GBP5 | COG4 | 0.89 |
| GBP5 | PILRA | 0.89 |
| GBP5 | OGG1 | 0.89 |
| P2RY14 | TSR1 | 0.89 |
| P2RY14 | NUP54 | 0.89 |
| GBP5 | CLN6 | 0.89 |
| GBP5 | NCALD | 0.89 |
| GBP5 | NRBP1 | 0.89 |
| GBP5 | CWF19L1 | 0.89 |
| GBP5 | HEXIM1 | 0.89 |
| UBE2L6 | SECTM1 | 0.89 |
| UBE2L6 | GNG11 | 0.89 |
| P2RY14 | ITPR3 | 0.89 |
| POLB | SDC1 | 0.89 |
| GBP5 | VRK1 | 0.89 |
| P2RY14 | SLC38A2 | 0.89 |
| GBP5 | UBE2N | 0.89 |
| GBP5 | KLHL22 | 0.89 |
| GBP5 | RNF138 | 0.89 |
| P2RY14 | NKTR | 0.89 |
| GBP5 | GALE | 0.89 |
| POLB | GEMIN4 | 0.89 |
| P2RY14 | FBXW2 | 0.89 |
| GBP5 | XRCC5 | 0.89 |
| GBP5 | SHB | 0.89 |
| GBP5 | PPT1 | 0.89 |
| GBP5 | LEF1 | 0.89 |
| POLD4 | P2RY14 | 0.89 |
| GBP5 | VWF | 0.89 |
| GBP5 | PRKCD | 0.89 |
| GBP5 | TIAL1 | 0.89 |
| GBP5 | TM2D3 | 0.89 |
| POLB | RBBP4 | 0.89 |
| P2RY14 | ZMPSTE24 | 0.89 |
| P2RY14 | MIS12 | 0.889 |
| GBP5 | WDR74 | 0.889 |
| GBP5 | BAIAP2 | 0.889 |
| GBP5 | PEX14 | 0.889 |
| GBP1 | XPO1 | 0.889 |
| GBP5 | RBKS | 0.889 |
| GBP5 | ILKAP | 0.889 |
| P2RY14 | UPF2 | 0.889 |
| GBP5 | CYCS | 0.889 |
| GBP5 | GGA2 | 0.889 |
| GBP5 | GPR68 | 0.889 |
| GBP5 | CFDP1 | 0.889 |
| P2RY14 | SAR1A | 0.889 |
| GBP1 | KLF6 | 0.889 |
| P2RY14 | LSG1 | 0.889 |
| GBP5 | PYGM | 0.889 |
| P2RY14 | MSRB2 | 0.889 |
| GBP5 | PSME3 | 0.889 |
| GBP5 | PTK2 | 0.889 |
| P2RY14 | PPAT | 0.889 |
| P2RY14 | CYB561 | 0.889 |
| GBP5 | CASP8 | 0.889 |
| GBP5 | FGR | 0.889 |
| GBP5 | PIGV | 0.889 |
| POLB | RFC1 | 0.889 |
| UBE2L6 | MX2 | 0.889 |
| P2RY14 | ENSA | 0.889 |
| P2RY14 | ATG3 | 0.889 |
| NDUFB6 | P2RY14 | 0.889 |
| GBP5 | MRPS31 | 0.889 |
| P2RY14 | DNPEP | 0.889 |
| GBP5 | ZFPL1 | 0.889 |
| POLB | USP4 | 0.889 |
| GBP5 | SSH3 | 0.889 |
| GBP5 | ACTR1B | 0.889 |
| GBP5 | YIPF1 | 0.889 |
| GBP5 | RBMX2 | 0.889 |
| P2RY14 | SLC30A5 | 0.889 |
| GBP5 | GNB5 | 0.889 |
| P2RY14 | ARIH2 | 0.889 |
| P2RY14 | PTGER4 | 0.889 |
| GBP5 | WDR19 | 0.889 |
| POLB | YEATS2 | 0.889 |
| P2RY14 | SIDT1 | 0.889 |
| GBP5 | TRIT1 | 0.889 |
| GBP5 | P2RY13 | 0.889 |
| P2RY14 | SIRT6 | 0.889 |
| GBP5 | TPD52L2 | 0.889 |
| GBP5 | TUBG1 | 0.889 |
| P2RY14 | POLDIP2 | 0.889 |
| GBP5 | HMGN3 | 0.889 |
| GBP5 | DNAJB9 | 0.889 |
| P2RY14 | PSMD5 | 0.889 |
| GBP5 | LMBR1L | 0.889 |
| GBP5 | PDE6B | 0.889 |
| PSME1 | NDRG1 | 0.889 |
| GBP5 | B4GALT1 | 0.889 |
| POLB | SIRT5 | 0.889 |
| GBP5 | DCK | 0.889 |
| GBP5 | RASGRP2 | 0.889 |
| P2RY14 | ANKS1A | 0.889 |
| GBP5 | MBIP | 0.889 |
| GBP5 | TRAF3IP2 | 0.889 |
| P2RY14 | TAF4 | 0.889 |
| GBP5 | UNC45A | 0.889 |
| GBP5 | EIF2AK1 | 0.889 |
| P2RY14 | CHMP6 | 0.889 |
| GBP5 | PVR | 0.889 |
| P2RY14 | CNOT8 | 0.889 |
| GBP5 | RBM3 | 0.889 |
| GBP5 | PTPRN2 | 0.889 |
| S100A9 | P2RY14 | 0.889 |
| GBP5 | BIRC3 | 0.889 |
| GBP1 | PFDN5 | 0.889 |
| GBP5 | ANAPC5 | 0.889 |
| POLB | PPP3CA | 0.889 |
| GBP5 | TRIB3 | 0.889 |
| P2RY14 | MAT2A | 0.889 |
| POLB | MSH2 | 0.889 |
| P2RY14 | BTBD1 | 0.889 |
| P2RY14 | SLC35A3 | 0.889 |
| P2RY14 | HMG20A | 0.889 |
| GBP5 | RPS6KA1 | 0.889 |
| POLB | TRAPPC3 | 0.889 |
| GBP5 | PLXND1 | 0.889 |
| GBP5 | MTHFR | 0.889 |
| CYB5R3 | P2RY14 | 0.889 |
| GBP5 | HS2ST1 | 0.889 |
| GBP5 | BANK1 | 0.889 |
| GBP5 | LZTR1 | 0.889 |
| P2RY14 | BCOR | 0.889 |
| GBP5 | ST14 | 0.889 |
| GBP5 | ACAD10 | 0.889 |
| C1QA | GBP1 | 0.889 |
| GBP5 | CLDN15 | 0.889 |
| GBP5 | IMP4 | 0.889 |
| P2RY14 | CCDC47 | 0.889 |
| GBP5 | HERPUD1 | 0.889 |
| GBP5 | LY9 | 0.889 |
| GBP5 | SLC35A2 | 0.889 |
| POLB | TERF2 | 0.889 |
| GBP5 | ALOX12 | 0.889 |
| GBP5 | WDR26 | 0.889 |
| GBP5 | DVL3 | 0.889 |
| GBP5 | CNIH4 | 0.889 |
| POLB | CSE1L | 0.889 |
| GBP5 | ICAM2 | 0.889 |
| GBP5 | ASRGL1 | 0.889 |
| GBP5 | PSTPIP2 | 0.889 |
| GBP5 | ITGB3BP | 0.889 |
| GBP5 | LTBR | 0.889 |
| GBP5 | INPP5A | 0.889 |
| GBP5 | GAB2 | 0.889 |
| P2RY14 | ITPR1 | 0.889 |
| GBP5 | PAFAH1B2 | 0.889 |
| GBP5 | SCARB1 | 0.889 |
| GBP5 | PITPNM1 | 0.888 |
| GBP5 | ITGB1BP1 | 0.888 |
| GBP5 | GNPAT | 0.888 |
| GBP5 | MTA2 | 0.888 |
| CHMP2A | P2RY14 | 0.888 |
| GBP5 | HEBP1 | 0.888 |
| GBP5 | GALK2 | 0.888 |
| LAPTM4A | P2RY14 | 0.888 |
| GBP5 | ASCL2 | 0.888 |
| P2RY14 | TMCC1 | 0.888 |
| P2RY14 | EIF5 | 0.888 |
| P2RY14 | RGS10 | 0.888 |
| GBP5 | KIFAP3 | 0.888 |
| P2RY14 | ZNF365 | 0.888 |
| GBP5 | COPS6 | 0.888 |
| GBP5 | METTL1 | 0.888 |
| P2RY14 | ZBTB16 | 0.888 |
| GBP5 | THOP1 | 0.888 |
| GBP5 | PSMD3 | 0.888 |
| GBP5 | FNTA | 0.888 |
| GBP1 | TMEM123 | 0.888 |
| P2RY14 | TRIB2 | 0.888 |
| GBP5 | BCAT2 | 0.888 |
| P2RY14 | BCL2 | 0.888 |
| POLB | SSRP1 | 0.888 |
| GBP5 | CHIC2 | 0.888 |
| GBP5 | IFNAR2 | 0.888 |
| GBP5 | ALAS1 | 0.888 |
| GBP5 | DGCR2 | 0.888 |
| GBP5 | SLC4A2 | 0.888 |
| P2RY14 | NUP50 | 0.888 |
| P2RY14 | STK38 | 0.888 |
| GBP5 | PLEKHA1 | 0.888 |
| GBP5 | SRPRB | 0.888 |
| P2RY14 | CAMK2G | 0.888 |
| GBP5 | AGPAT4 | 0.888 |
| GBP5 | KCNMA1 | 0.888 |
| GBP5 | TM9SF2 | 0.888 |
| GBP5 | SSBP2 | 0.888 |
| GBP5 | RCN3 | 0.888 |
| GBP5 | ZDHHC7 | 0.888 |
| GBP5 | RLF | 0.888 |
| GBP5 | FAM50B | 0.888 |
| P2RY14 | CALCOCO2 | 0.888 |
| GBP5 | RNF146 | 0.888 |
| GBP5 | PRSS23 | 0.888 |
| P2RY14 | OGFR | 0.888 |
| P2RY14 | MDK | 0.888 |
| P2RY14 | CPVL | 0.888 |
| GBP5 | TSPAN31 | 0.888 |
| GBP5 | EIF2B1 | 0.888 |
| GBP5 | ENC1 | 0.888 |
| P2RY14 | SIRT5 | 0.888 |
| GBP5 | TPM3 | 0.888 |
| GBP5 | CLK2 | 0.888 |
| GBP5 | FKBP4 | 0.888 |
| GBP5 | NXF1 | 0.888 |
| GBP5 | SELP | 0.888 |
| GBP5 | PIGT | 0.888 |
| GBP5 | PPM1G | 0.888 |
| P2RY14 | CDYL | 0.888 |
| GBP5 | UBASH3A | 0.888 |
| P2RY14 | CASP1 | 0.888 |
| GBP5 | STAT4 | 0.888 |
| P2RY14 | DLG4 | 0.888 |
| BCKDK | P2RY14 | 0.888 |
| GBP5 | RASSF2 | 0.888 |
| GBP5 | BET1 | 0.888 |
| GBP5 | TCEAL1 | 0.888 |
| P2RY14 | NET1 | 0.888 |
| GBP5 | DHDDS | 0.888 |
| P2RY14 | TRAF3 | 0.888 |
| POLB | USP47 | 0.888 |
| P2RY14 | TMEM43 | 0.888 |
| P2RY14 | CSNK1G3 | 0.888 |
| P2RY14 | KHDRBS1 | 0.888 |
| GBP5 | IGFBP3 | 0.888 |
| P2RY14 | VDAC3 | 0.888 |
| P2RY14 | MDM1 | 0.888 |
| GBP5 | SREBF1 | 0.888 |
| P2RY14 | GEMIN7 | 0.888 |
| P2RY14 | EHBP1 | 0.888 |
| GBP5 | MRFAP1L1 | 0.888 |
| GBP5 | CD83 | 0.888 |
| GBP5 | CLNS1A | 0.888 |
| POLB | GMEB1 | 0.888 |
| GBP5 | ATP1A1 | 0.888 |
| GBP5 | AKAP8L | 0.888 |
| GBP5 | CASP7 | 0.888 |
| GBP5 | RERE | 0.888 |
| P2RY14 | ZZEF1 | 0.888 |
| GBP5 | FYN | 0.888 |
| P2RY14 | MAPK6 | 0.888 |
| P2RY14 | SUZ12 | 0.888 |
| P2RY14 | DYNC1LI2 | 0.888 |
| P2RY14 | PARP16 | 0.888 |
| GBP5 | SLC2A4RG | 0.888 |
| GBP5 | RABIF | 0.888 |
| GBP5 | PPP1R16B | 0.888 |
| P2RY14 | RFWD3 | 0.888 |
| POLB | NFATC1 | 0.888 |
| P2RY14 | MAPK1 | 0.888 |
| GBP5 | CTSK | 0.888 |
| GBP5 | SUOX | 0.888 |
| GBP5 | DHRS3 | 0.888 |
| GBP5 | DUT | 0.888 |
| GBP5 | HOOK2 | 0.888 |
| GBP5 | GALK1 | 0.888 |
| ACP2 | P2RY14 | 0.888 |
| GBP5 | SDCBP | 0.888 |
| P2RY14 | DENND1A | 0.888 |
| GBP5 | RAB27A | 0.888 |
| ECHS1 | P2RY14 | 0.888 |
| GBP5 | LIPT1 | 0.888 |
| GBP5 | GATA1 | 0.888 |
| GBP5 | METTL7A | 0.888 |
| SECTM1 | DGKD | 0.888 |
| POLB | LAS1L | 0.888 |
| GBP5 | NEDD4L | 0.888 |
| P2RY14 | RRM1 | 0.888 |
| P2RY14 | SLC16A6 | 0.887 |
| P2RY14 | RFC2 | 0.887 |
| GBP5 | E4F1 | 0.887 |
| GBP5 | RRAS | 0.887 |
| GBP5 | TRMT1 | 0.887 |
| P2RY14 | TCF7 | 0.887 |
| GBP5 | ATG5 | 0.887 |
| GBP5 | BCL2L2 | 0.887 |
| GBP5 | PXMP2 | 0.887 |
| GBP5 | ACTR5 | 0.887 |
| GBP5 | WIPI1 | 0.887 |
| GBP5 | COQ2 | 0.887 |
| GBP5 | STIP1 | 0.887 |
| GBP5 | PARP3 | 0.887 |
| GBP5 | SEC14L4 | 0.887 |
| GBP5 | SMPD1 | 0.887 |
| GBP5 | TNFSF14 | 0.887 |
| P2RY14 | GOLGA3 | 0.887 |
| GBP5 | GSR | 0.887 |
| GBP5 | PPP1R2 | 0.887 |
| P2RY14 | NUP107 | 0.887 |
| POLB | PPP2R5B | 0.887 |
| GBP5 | RGS14 | 0.887 |
| GBP5 | NUP88 | 0.887 |
| GBP5 | SLC25A37 | 0.887 |
| GBP5 | PIAS4 | 0.887 |
| GBP5 | FCRL2 | 0.887 |
| GBP5 | GPKOW | 0.887 |
| P2RY14 | DCP2 | 0.887 |
| P2RY14 | DIAPH1 | 0.887 |
| GBP5 | CCDC22 | 0.887 |
| IL15RA | SECTM1 | 0.887 |
| GBP5 | NXT1 | 0.887 |
| UBE2L6 | TSPAN14 | 0.887 |
| P2RY14 | CNOT2 | 0.887 |
| P2RY14 | CUL4A | 0.887 |
| GBP5 | SAMHD1 | 0.887 |
| P2RY14 | FZD2 | 0.887 |
| GBP5 | MED4 | 0.887 |
| GBP5 | ZNF324 | 0.887 |
| GBP5 | PREB | 0.887 |
| POLB | SNW1 | 0.887 |
| GBP5 | CACYBP | 0.887 |
| GBP5 | POGK | 0.887 |
| P2RY14 | AASDHPPT | 0.887 |
| GBP5 | POFUT2 | 0.887 |
| GBP5 | RB1CC1 | 0.887 |
| POLB | CAMK4 | 0.887 |
| POLB | POU2F1 | 0.887 |
| GBP5 | CDK4 | 0.887 |
| GBP5 | CPSF4 | 0.887 |
| GBP1 | YEATS2 | 0.887 |
| P2RY14 | ITGA7 | 0.887 |
| P2RY14 | NAPA | 0.887 |
| GSTO1 | POLB | 0.887 |
| GBP5 | YY1AP1 | 0.887 |
| GBP5 | MAPKAP1 | 0.887 |
| GBP5 | USP5 | 0.887 |
| GBP5 | CNTNAP1 | 0.887 |
| P2RY14 | MBTPS1 | 0.887 |
| P2RY14 | GOLPH3 | 0.887 |
| GBP5 | WWOX | 0.887 |
| GBP5 | PEBP1 | 0.887 |
| GBP5 | ST7L | 0.887 |
| GBP5 | ZNF696 | 0.887 |
| P2RY14 | PTP4A1 | 0.887 |
| GBP5 | IDH2 | 0.887 |
| POLB | CDK5RAP1 | 0.887 |
| POLB | SLTM | 0.887 |
| GBP5 | UTP14A | 0.887 |
| GBP5 | NFE2 | 0.887 |
| P2RY14 | SFXN1 | 0.887 |
| GBP5 | RNASEH1 | 0.887 |
| P2RY14 | PHF20 | 0.887 |
| GBP5 | TIMM8A | 0.887 |
| GBP5 | SORT1 | 0.887 |
| P2RY14 | RCOR3 | 0.887 |
| GBP5 | HSD17B12 | 0.887 |
| GBP5 | SLC27A5 | 0.887 |
| GBP5 | NDFIP1 | 0.887 |
| P2RY14 | ZNF672 | 0.887 |
| P2RY14 | RAB11FIP2 | 0.887 |
| GBP5 | CBX7 | 0.887 |
| SECTM1 | TRIM8 | 0.887 |
| GBP5 | TFPI | 0.887 |
| POLB | DAPP1 | 0.887 |
| GBP5 | RAB3IL1 | 0.887 |
| SECTM1 | KLF6 | 0.887 |
| GBP5 | FBLN5 | 0.887 |
| GBP5 | GCM1 | 0.887 |
| P2RY14 | LRRC8D | 0.887 |
| GBP5 | ROM1 | 0.887 |
| POLB | BAHD1 | 0.887 |
| GBP5 | PSEN1 | 0.887 |
| POLB | BCR | 0.887 |
| GBP5 | AFF3 | 0.887 |
| P2RY14 | RECQL | 0.887 |
| GBP5 | AP2A2 | 0.887 |
| GBP5 | ST3GAL2 | 0.887 |
| P2RY14 | PER2 | 0.887 |
| GBP5 | PANK4 | 0.887 |
| GBP5 | ADAM8 | 0.887 |
| GBP5 | ZNF395 | 0.887 |
| GBP5 | SLC2A8 | 0.887 |
| GBP1 | SLCO4C1 | 0.887 |
| POLB | GTPBP4 | 0.887 |
| POLB | PPARD | 0.887 |
| GBP5 | RPN1 | 0.887 |
| GBP5 | XBP1 | 0.887 |
| GBP5 | LRRN3 | 0.887 |
| UBE2L6 | MCM2 | 0.887 |
| P2RY14 | APOL3 | 0.887 |
| P2RY14 | ZNF76 | 0.887 |
| P2RY14 | ZCCHC8 | 0.887 |
| P2RY14 | SEH1L | 0.887 |
| PCK2 | P2RY14 | 0.887 |
| POLB | PBXIP1 | 0.887 |
| GBP5 | CCNG1 | 0.887 |
| GBP5 | ARL2BP | 0.887 |
| P2RY14 | AUTS2 | 0.887 |
| GBP5 | MAP1LC3B | 0.887 |
| P2RY14 | CAMSAP1 | 0.887 |
| GBP5 | CHSY1 | 0.887 |
| GBP5 | POMT2 | 0.886 |
| UBE2L6 | NOLC1 | 0.886 |
| GBP5 | PSMD14 | 0.886 |
| GBP5 | ABHD5 | 0.886 |
| GBP5 | LDLRAP1 | 0.886 |
| UBE2L6 | LIMK1 | 0.886 |
| GBP5 | SCAP | 0.886 |
| P2RY14 | KYNU | 0.886 |
| GBP5 | MYLK | 0.886 |
| P2RY14 | LHFPL2 | 0.886 |
| SECTM1 | GBP1 | 0.886 |
| GBP5 | ALKBH4 | 0.886 |
| P2RY14 | DDX27 | 0.886 |
| P2RY14 | NCL | 0.886 |
| P2RY14 | AQR | 0.886 |
| GBP5 | HMGA1 | 0.886 |
| GBP5 | SLC39A9 | 0.886 |
| GBP5 | CPT1A | 0.886 |
| GBP5 | CASP10 | 0.886 |
| GBP5 | BAG5 | 0.886 |
| GBP5 | EIF4EBP2 | 0.886 |
| POLB | CYB561 | 0.886 |
| GBP5 | STK24 | 0.886 |
| SECTM1 | GLG1 | 0.886 |
| GBP5 | SCRN1 | 0.886 |
| P2RY14 | ARMC1 | 0.886 |
| P2RY14 | TNPO1 | 0.886 |
| GBP5 | KMO | 0.886 |
| GBP5 | CLEC7A | 0.886 |
| GBP5 | TMEM14A | 0.886 |
| GBP5 | FHOD1 | 0.886 |
| GBP5 | ASNSD1 | 0.886 |
| GBP5 | MAPKAPK2 | 0.886 |
| GBP5 | CAMK1D | 0.886 |
| GBP5 | GYS1 | 0.886 |
| GBP5 | IFT57 | 0.886 |
| GBP5 | SH3GLB2 | 0.886 |
| GBP5 | ERGIC2 | 0.886 |
| P2RY14 | GBP1 | 0.886 |
| GBP5 | TXNL4B | 0.886 |
| P2RY14 | SRRM1 | 0.886 |
| P2RY14 | PPM1B | 0.886 |
| P2RY14 | PIK3CB | 0.886 |
| POLB | DLEC1 | 0.886 |
| GBP5 | SNAP29 | 0.886 |
| P2RY14 | LARS2 | 0.886 |
| P2RY14 | POLD3 | 0.886 |
| GBP5 | MARK2 | 0.886 |
| P2RY14 | INSIG2 | 0.886 |
| GBP5 | C1GALT1C1 | 0.886 |
| GBP5 | ELP4 | 0.886 |
| GBP5 | UBL3 | 0.886 |
| GBP5 | SLC25A36 | 0.886 |
| GBP5 | WDR73 | 0.886 |
| GBP5 | NDUFA9 | 0.886 |
| GBP5 | ERAL1 | 0.886 |
| P2RY14 | SERTAD3 | 0.886 |
| GBP5 | ZNF174 | 0.886 |
| GBP5 | AIM2 | 0.886 |
| GBP5 | LCP2 | 0.886 |
| GBP5 | SMNDC1 | 0.886 |
| POLB | SPEN | 0.886 |
| P2RY14 | ANKRD10 | 0.886 |
| GBP5 | MANSC1 | 0.886 |
| GBP1 | MCM6 | 0.886 |
| GBP5 | ACAT2 | 0.886 |
| P2RY14 | FOXJ2 | 0.886 |
| P2RY14 | MDM2 | 0.886 |
| GBP5 | CERK | 0.886 |
| GBP5 | PPIF | 0.886 |
| POLB | IFT122 | 0.886 |
| GBP5 | CUTC | 0.886 |
| GBP5 | IRF2 | 0.886 |
| GBP5 | LIPA | 0.886 |
| P2RY14 | ABLIM1 | 0.886 |
| P2RY14 | WDR12 | 0.886 |
| GBP5 | PTGES2 | 0.886 |
| P2RY14 | MRPS12 | 0.886 |
| GBP5 | DDX28 | 0.886 |
| GBP5 | NMI | 0.886 |
| P2RY14 | RER1 | 0.886 |
| P2RY14 | YTHDC1 | 0.886 |
| GBP5 | KCTD5 | 0.886 |
| GBP5 | ATP6AP1 | 0.886 |
| GBP5 | NAGLU | 0.886 |
| P2RY14 | UBAP2 | 0.886 |
| GBP5 | DNASE2 | 0.886 |
| POLB | ZXDC | 0.886 |
| GBP5 | GMEB2 | 0.886 |
| GBP5 | SYPL1 | 0.886 |
| P2RY14 | UROD | 0.886 |
| P2RY14 | ST8SIA4 | 0.886 |
| GBP5 | FXR1 | 0.886 |
| GBP5 | HPRT1 | 0.886 |
| GBP5 | ZDHHC6 | 0.886 |
| GBP5 | ATG4A | 0.886 |
| GBP5 | GRB2 | 0.886 |
| GBP5 | IRF1 | 0.886 |
| GBP5 | FBN2 | 0.886 |
| GBP5 | TERF2IP | 0.886 |
| GBP5 | TPP1 | 0.886 |
| GBP5 | PIPSK1B | 0.886 |
| P2RY14 | RTN2 | 0.886 |
| COX6B1 | GBP1 | 0.886 |
| GBP5 | TIPRL | 0.886 |
| GBP5 | PRMT2 | 0.886 |
| P2RY14 | CDK6 | 0.886 |
| GBP5 | RBM4B | 0.886 |
| P2RY14 | KLHL3 | 0.886 |
| GBP5 | STX1A | 0.886 |
| P2RY14 | CBLB | 0.886 |
| P2RY14 | MAN2A1 | 0.886 |
| GBP5 | SRF | 0.886 |
| P2RY14 | SPAG9 | 0.886 |
| P2RY14 | PUM2 | 0.886 |
| P2RY14 | RASGRP1 | 0.886 |
| GBP5 | CHKA | 0.886 |
| GBP5 | RNF40 | 0.886 |
| GBP5 | PNRC1 | 0.886 |
| P2RY14 | HIC2 | 0.886 |
| GBP5 | MKKS | 0.886 |
| GBP5 | ASB7 | 0.886 |
| P2RY14 | CENPJ | 0.886 |
| GBP5 | HGS | 0.886 |
| SECTM1 | MSN | 0.886 |
| GBP5 | WSB2 | 0.886 |
| GBP5 | KATNB1 | 0.886 |
| GBP5 | AK2 | 0.886 |
| UBE2L6 | KLF6 | 0.886 |
| GBP1 | TOPBP1 | 0.886 |
| GBP5 | ERF | 0.886 |
| GBP5 | GCDH | 0.886 |
| GBP5 | ZFP36L1 | 0.886 |
| GBP5 | RAB3D | 0.886 |
| TMEM59 | UBE2L6 | 0.886 |
| P2RY14 | DLAT | 0.886 |
| P2RY14 | ESD | 0.885 |
| GBP5 | PPOX | 0.885 |
| P2RY14 | MCM6 | 0.885 |
| GBP5 | RELB | 0.885 |
| GBP5 | IL15 | 0.885 |
| GBP5 | ALDH1A1 | 0.885 |
| GBP5 | DLGAP4 | 0.885 |
| P2RY14 | PCSK6 | 0.885 |
| GBP5 | CEP63 | 0.885 |
| P2RY14 | ACOT7 | 0.885 |
| POLB | TLE4 | 0.885 |
| POLB | ZZEF1 | 0.885 |
| P2RY14 | GUK1 | 0.885 |
| GBP5 | SMOX | 0.885 |
| GBP5 | LRRC41 | 0.885 |
| GBP5 | PTP4A3 | 0.885 |
| MGST3 | P2RY14 | 0.885 |
| GBP5 | POLE3 | 0.885 |
| GBP5 | PES1 | 0.885 |
| P2RY14 | PTGER2 | 0.885 |
| GBP5 | RDH11 | 0.885 |
| GBP1 | LIMK1 | 0.885 |
| GBP5 | PRCP | 0.885 |
| GBP5 | UXS1 | 0.885 |
| P2RY14 | ATP2A2 | 0.885 |
| POLB | DPF2 | 0.885 |
| GBP5 | BAP1 | 0.885 |
| P2RY14 | HTRA2 | 0.885 |
| DAPP1 | KLF6 | 0.885 |
| LSM1 | P2RY14 | 0.885 |
| GBP5 | OSBPL10 | 0.885 |
| GBP5 | SPHK2 | 0.885 |
| SECTM1 | SMARCC1 | 0.885 |
| GBP5 | CDR2L | 0.885 |
| GBP5 | PUS3 | 0.885 |
| GBP5 | SLC24A3 | 0.885 |
| APOL2 | KLF6 | 0.885 |
| IL15RA | NDRG1 | 0.885 |
| GBP5 | CITED2 | 0.885 |
| P2RY14 | GTPBP4 | 0.885 |
| POLB | SLC25A17 | 0.885 |
| P2RY14 | RAPGEF1 | 0.885 |
| P2RY14 | ZNF337 | 0.885 |
| P2RY14 | RPAP1 | 0.885 |
| GBP5 | PHB | 0.885 |
| GBP5 | SLC43A3 | 0.885 |
| GBP5 | CHST7 | 0.885 |
| POLB | PHF20 | 0.885 |
| GBP5 | IPO13 | 0.885 |
| P2RY14 | NFATC1 | 0.885 |
| GBP5 | CAB39L | 0.885 |
| GBP5 | DDIT3 | 0.885 |
| GBP5 | NIT2 | 0.885 |
| P2RY14 | MAP4 | 0.885 |
| P2RY14 | ANKRD11 | 0.885 |
| GBP5 | TRIP6 | 0.885 |
| P2RY14 | TSPAN2 | 0.885 |
| GBP5 | PPP6C | 0.885 |
| P2RY14 | PRKCQ | 0.885 |
| GBP5 | XRCC1 | 0.885 |
| GBP5 | BMP2K | 0.885 |
| P2RY14 | NUP155 | 0.885 |
| P2RY14 | FNBP4 | 0.885 |
| GBP5 | DCTD | 0.885 |
| GBP5 | SCFD1 | 0.885 |
| GBP5 | ITGAX | 0.885 |
| P2RY14 | PHF10 | 0.885 |
| GBP5 | DRG2 | 0.885 |
| UBE2L6 | PLAGL2 | 0.885 |
| GBP5 | OSTF1 | 0.885 |
| P2RY14 | SCMH1 | 0.885 |
| P2RY14 | PON2 | 0.885 |
| GBP5 | CHRNA10 | 0.885 |
| GBP5 | GNPDA1 | 0.885 |
| GBP5 | ENO3 | 0.885 |
| GBP5 | TOMM20 | 0.885 |
| P2RY14 | PARVB | 0.885 |
| P2RY14 | SLC25A12 | 0.885 |
| GBP5 | MRPS35 | 0.885 |
| POLB | GBP1 | 0.885 |
| P2RY14 | ZNF394 | 0.885 |
| UBE2L6 | C1QA | 0.885 |
| P2RY14 | GPR137 | 0.885 |
| GBP5 | ABHD4 | 0.885 |
| P2RY14 | CKAP2 | 0.885 |
| P2RY14 | ING4 | 0.885 |
| GBP5 | MLYCD | 0.885 |
| P2RY14 | EP400 | 0.885 |
| P2RY14 | MYCBP2 | 0.885 |
| POLB | PARP16 | 0.885 |
| POLB | SYNJ2 | 0.885 |
| POLB | XPC | 0.885 |
| P2RY14 | DHX15 | 0.885 |
| GBP5 | RNF8 | 0.885 |
| RFXANK | P2RY14 | 0.885 |
| P2RY14 | DENND2D | 0.885 |
| P2RY14 | PLAA | 0.885 |
| POLB | ELAC2 | 0.885 |
| GBP5 | CDK9 | 0.885 |
| GBP5 | PTTG1IP | 0.885 |
| ACP5 | LHFPL2 | 0.885 |
| GBP5 | BIN2 | 0.885 |
| P2RY14 | BCR | 0.885 |
| CALCOCO2 | SECTM1 | 0.885 |
| GBP5 | ARL1 | 0.885 |
| P2RY14 | POLR1B | 0.885 |
| GBP5 | BACE2 | 0.885 |
| P2RY14 | SKP2 | 0.885 |
| GBP5 | THAP7 | 0.885 |
| GBP5 | LMAN2L | 0.885 |
| GBP5 | LMO4 | 0.885 |
| GBP5 | CD36 | 0.885 |
| P2RY14 | TLE4 | 0.885 |
| GBP5 | CDADC1 | 0.885 |
| POLB | PLCG1 | 0.885 |
| GBP5 | RAPSN | 0.885 |
| GBP5 | ITPKC | 0.885 |
| IL15RA | APOL2 | 0.885 |
| GBP5 | ITGB2 | 0.885 |
| GBP5 | ATP1B1 | 0.885 |
| GBP5 | SCAMP2 | 0.885 |
| POLB | LSG1 | 0.885 |
| GBP5 | CHMP2B | 0.885 |
| GBP5 | AHSA1 | 0.885 |
| GBP5 | RASSF4 | 0.884 |
| GBP5 | EBAG9 | 0.884 |
| POLB | TSC1 | 0.884 |
| GBP5 | ANKRA2 | 0.884 |
| GBP5 | BCL7B | 0.884 |
| GBP5 | PACS1 | 0.884 |
| GBP5 | NDUFAF1 | 0.884 |
| GBP5 | TXNDC9 | 0.884 |
| GBP5 | MAP4K1 | 0.884 |
| GBP5 | CAPN1 | 0.884 |
| GBP5 | ING2 | 0.884 |
| GBP5 | WDR70 | 0.884 |
| GBP5 | CX3CR1 | 0.884 |
| GBP5 | TAF7 | 0.884 |
| P2RY14 | ARHGEF3 | 0.884 |
| GBP5 | VPS37B | 0.884 |
| P2RY14 | WBP11 | 0.884 |
| GBP5 | KCNMB1 | 0.884 |
| GBP5 | ARRB1 | 0.884 |
| P2RY14 | NGLY1 | 0.884 |
| GBP5 | HRAS | 0.884 |
| GBP5 | DYNLT3 | 0.884 |
| GBP5 | PDLIM5 | 0.884 |
| HMGCL | P2RY14 | 0.884 |
| SECTM1 | APOL2 | 0.884 |
| GBP5 | WDR1 | 0.884 |
| GBP5 | ACIN1 | 0.884 |
| P2RY14 | RPGR | 0.884 |
| P2RY14 | ITPKB | 0.884 |
| GBP5 | ZFAND1 | 0.884 |
| POLB | LRIG1 | 0.884 |
| P2RY14 | SERPINB6 | 0.884 |
| P2RY14 | TNFAIP3 | 0.884 |
| POLB | PPP2R5A | 0.884 |
| POLB | ODF2 | 0.884 |
| DAPP1 | ELF1 | 0.884 |
| APOL2 | GLG1 | 0.884 |
| POLB | BTG1 | 0.884 |
| P2RY14 | DBR1 | 0.884 |
| GBP5 | SCYL3 | 0.884 |
| P2RY14 | TACC1 | 0.884 |
| P2RY14 | VPS54 | 0.884 |
| P2RY14 | BBS7 | 0.884 |
| SECTM1 | BCR | 0.884 |
| GBP5 | CYP11A1 | 0.884 |
| P2RY14 | ISOC2 | 0.884 |
| GBP5 | THBD | 0.884 |
| SECTM1 | DPF2 | 0.884 |
| P2RY14 | SLC4A7 | 0.884 |
| GBP5 | LEPROTL1 | 0.884 |
| GBP5 | OPN3 | 0.884 |
| SECTM1 | TTLL12 | 0.884 |
| GBP5 | ABT1 | 0.884 |
| GBP5 | TM2D1 | 0.884 |
| GBP5 | VPS53 | 0.884 |
| POLB | RRS1 | 0.884 |
| P2RY14 | POLH | 0.884 |
| GBP5 | UBE2Q1 | 0.884 |
| P2RY14 | ATPAF2 | 0.884 |
| GBP5 | PKIA | 0.884 |
| NPC2 | NDRG1 | 0.884 |
| SECTM1 | ABR | 0.884 |
| GBP1 | ZFR | 0.884 |
| GBP5 | RNF7 | 0.884 |
| GBP5 | NT5E | 0.884 |
| P2RY14 | BACH2 | 0.884 |
| GBP5 | ARSB | 0.884 |
| GBP5 | SERPINF1 | 0.884 |
| POLB | APOL1 | 0.884 |
| GBP5 | GSS | 0.884 |
| P2RY14 | ARMCX5 | 0.884 |
| P2RY14 | PPP4R1 | 0.884 |
| GBP5 | OPRL1 | 0.884 |
| GBP5 | GPR27 | 0.884 |
| PSME1 | SECTM1 | 0.884 |
| P2RY14 | TSPAN14 | 0.884 |
| P2RY14 | GTF3C3 | 0.884 |
| POLB | FANCE | 0.884 |
| GBP5 | INVS | 0.884 |
| GBP5 | CXCR4 | 0.884 |
| GBP5 | UBE2G2 | 0.884 |
| GBP5 | STXBP3 | 0.884 |
| GBP5 | TSPAN3 | 0.884 |
| GBP5 | ZNF200 | 0.884 |
| GBP5 | SMYD3 | 0.884 |
| POLB | DCUN1D2 | 0.884 |
| GBP5 | U2AF1 | 0.884 |
| GBP5 | COMMD10 | 0.884 |
| P2RY14 | KPNA6 | 0.884 |
| RHOC | P2RY14 | 0.884 |
| P2RY14 | SLC25A22 | 0.884 |
| GBP5 | STAM2 | 0.884 |
| IRF1 | P2RY14 | 0.884 |
| PML | NDRG1 | 0.884 |
| GBP5 | TMCO1 | 0.884 |
| P2RY14 | MTCH2 | 0.884 |
| GBP5 | IER2 | 0.884 |
| GBP5 | EBP | 0.884 |
| P2RY14 | TIMELESS | 0.884 |
| SECTM1 | TMEM127 | 0.884 |
| GBP5 | STK25 | 0.884 |
| P2RY14 | MAPKAPK5 | 0.884 |
| P2RY14 | FLNB | 0.884 |
| GBP5 | TOR3A | 0.884 |
| P2RY14 | TMEM115 | 0.884 |
| GBP5 | PSMD9 | 0.884 |
| GBP5 | BCL2L13 | 0.884 |
| UBE2L6 | ELF1 | 0.884 |
| P2RY14 | TEX10 | 0.884 |
| P2RY14 | POLR1D | 0.884 |
| GBP5 | KLHL2 | 0.884 |
| P2RY14 | CEP68 | 0.884 |
| GBP5 | PRCC | 0.884 |
| UBE2L6 | CAPN2 | 0.884 |
| P2RY14 | ZMYM5 | 0.884 |
| GBP5 | CSRP1 | 0.884 |
| GBP5 | NUP214 | 0.884 |
| NDUFA1 | GBP1 | 0.884 |
| GBP5 | FUT7 | 0.884 |
| GBP5 | RFX5 | 0.884 |
| P2RY14 | SMARCE1 | 0.884 |
| P2RY14 | ATP6V1A | 0.884 |
| GBP5 | RABEPK | 0.884 |
| P2RY14 | ABCE1 | 0.884 |
| GBP5 | ABHD10 | 0.884 |
| POP4 | P2RY14 | 0.884 |
| GBP5 | SNX4 | 0.884 |
| P2RY14 | GEMIN4 | 0.884 |
| GBP5 | PLA2G6 | 0.884 |
| P2RY14 | SNX16 | 0.884 |
| P2RY14 | TSNAX | 0.884 |
| GBP5 | POR | 0.884 |
| POLB | SMARCE1 | 0.884 |
| GBP5 | LZTFL1 | 0.884 |
| GBP5 | NUCB2 | 0.884 |
| P2RY14 | HERC2 | 0.884 |
| POLB | YTHDC1 | 0.884 |
| P2RY14 | LRRFIP1 | 0.884 |
| P2RY14 | CANX | 0.884 |
| GBP5 | PSMC2 | 0.884 |
| GBP5 | RNF121 | 0.884 |
| P2RY14 | ATP9B | 0.884 |
| GBP5 | CYBB | 0.884 |
| P2RY14 | GIMAP6 | 0.884 |
| P2RY14 | BARD1 | 0.883 |
| GBP5 | THADA | 0.883 |
| GBP5 | IGFBP2 | 0.883 |
| GBP5 | MRPS18A | 0.883 |
| P2RY14 | DCUN1D2 | 0.883 |
| GBP5 | MAN2C1 | 0.883 |
| GBP5 | COQ6 | 0.883 |
| GBP5 | PTDSS1 | 0.883 |
| GBP5 | IL1B | 0.883 |
| P2RY14 | MDH2 | 0.883 |
| POLB | GNG11 | 0.883 |
| POLB | ATG3 | 0.883 |
| GBP5 | ANXA11 | 0.883 |
| GBP5 | LDHA | 0.883 |
| POLB | ABCF2 | 0.883 |
| GBP5 | HEXB | 0.883 |
| GBP5 | KCNN4 | 0.883 |
| GBP5 | HCCS | 0.883 |
| GBP5 | RNASEH2A | 0.883 |
| P2RY14 | FAM3A | 0.883 |
| GBP1 | NENF | 0.883 |
| P2RY14 | CASB | 0.883 |
| SECTM1 | OGDH | 0.883 |
| P2RY14 | ANP32B | 0.883 |
| GBP5 | PLOD1 | 0.883 |
| GBP5 | SGSH | 0.883 |
| PSME1 | CEP250 | 0.883 |
| P2RY14 | XPC | 0.883 |
| GBP5 | GADD45B | 0.883 |
| LIMK1 | NDRG1 | 0.883 |
| UBE2L6 | CTCF | 0.883 |
| GBP5 | RAD9A | 0.883 |
| GBP5 | SYK | 0.883 |
| GNG11 | C1QA | 0.883 |
| GBP5 | RAB33A | 0.883 |
| GBP5 | IFNGR2 | 0.883 |
| GBP5 | MITF | 0.883 |
| GBP5 | ARL6IP5 | 0.883 |
| GBP5 | MYO1F | 0.883 |
| GBP5 | VPS41 | 0.883 |
| POLB | MCM3 | 0.883 |
| GBP5 | FAS | 0.883 |
| SECTM1 | VCP | 0.883 |
| P2RY14 | PPP2R5E | 0.883 |
| GBP5 | SSR3 | 0.883 |
| GBP5 | ST3GAL4 | 0.883 |
| SECTM1 | ARMC7 | 0.883 |
| P2RY14 | PCYOX1 | 0.883 |
| GBP5 | KLHDC2 | 0.883 |
| GBP5 | ASH2L | 0.883 |
| ACP5 | CASP1 | 0.883 |
| GBP5 | RPS6KC1 | 0.883 |
| P2RY14 | AMPD3 | 0.883 |
| P2RY14 | CASK | 0.883 |
| GBP5 | DERL2 | 0.883 |
| GBP5 | ZFAND3 | 0.883 |
| GBP5 | ASF1A | 0.883 |
| GBP5 | LRRFIP2 | 0.883 |
| GBP5 | TRPV2 | 0.883 |
| IRF3 | P2RY14 | 0.883 |
| POLB | HIC2 | 0.883 |
| GBP1 | MYD88 | 0.883 |
| P2RY14 | PDPK1 | 0.883 |
| GBP5 | RFX2 | 0.883 |
| GBP5 | ZNF701 | 0.883 |
| GBP5 | CANT1 | 0.883 |
| GBP5 | BLVRA | 0.883 |
| GBP5 | EPHB6 | 0.883 |
| P2RY14 | MECP2 | 0.883 |
| P2RY14 | POLR2C | 0.883 |
| GBP5 | ATXN10 | 0.883 |
| GBP5 | RTN3 | 0.883 |
| GBP5 | BIN1 | 0.883 |
| P2RY14 | STAM | 0.883 |
| P2RY14 | SLC25A17 | 0.883 |
| GBP5 | P2RY10 | 0.883 |
| GBP5 | LUC7L | 0.883 |
| POLB | MYC | 0.883 |
| GBP5 | RUFY3 | 0.883 |
| GBP5 | DCPS | 0.883 |
| GBP5 | CRADD | 0.883 |
| GBP1 | RBM28 | 0.883 |
| GBP5 | HIP1 | 0.883 |
| SECTM1 | CEP250 | 0.883 |
| POLB | SESN1 | 0.883 |
| GBP5 | ST3GAL6 | 0.883 |
| GBP5 | TOP3A | 0.883 |
| GBP5 | PLOD3 | 0.883 |
| GBP5 | CLCN6 | 0.883 |
| GBP5 | TMEM38B | 0.883 |
| P2RY14 | ABL1 | 0.883 |
| GBP5 | UBE2W | 0.883 |
| GBP5 | ZMYM3 | 0.883 |
| GBP1 | CTCF | 0.883 |
| GBP5 | SAMSN1 | 0.883 |
| GBP5 | SERGEF | 0.883 |
| GBP5 | CNOT4 | 0.883 |
| GBP5 | CD300A | 0.883 |
| GBP5 | FMNL1 | 0.883 |
| P2RY14 | ZBTB25 | 0.883 |
| POLB | SMU1 | 0.883 |
| GBP5 | APEX2 | 0.883 |
| P2RY14 | STK4 | 0.883 |
| GBP5 | HDAC6 | 0.883 |
| P2RY14 | KLHL7 | 0.883 |
| P2RY14 | UBE2G1 | 0.883 |
| GBP5 | SETMAR | 0.883 |
| P2RY14 | ANGEL1 | 0.883 |
| P2RY14 | ARHGEF7 | 0.883 |
| GBP5 | TCEAL4 | 0.883 |
| UBE2L6 | DIAPH1 | 0.883 |
| P2RY14 | DHX29 | 0.883 |
| GBP5 | ATP13A2 | 0.883 |
| GBP5 | NCDN | 0.883 |
| GBP5 | PLAUR | 0.883 |
| GBP5 | GLO1 | 0.883 |
| GBP5 | CCNC | 0.883 |
| P2RY14 | TOP2B | 0.883 |
| GBP5 | ECHDC1 | 0.883 |
| POLB | NCOA1 | 0.883 |
| P2RY14 | NKRF | 0.883 |
| GBP5 | GTF3C5 | 0.883 |
| POLB | TOB2 | 0.883 |
| POLB | CRY2 | 0.883 |
| P2RY14 | AACS | 0.883 |
| P2RY14 | COG5 | 0.883 |
| GBP5 | RBM23 | 0.883 |
| GBP5 | SLC6A12 | 0.883 |
| GBP5 | FKBP3 | 0.883 |
| SECTM1 | ATP6AP1 | 0.883 |
| GBP5 | DERL1 | 0.883 |
| POLB | MFAP1 | 0.883 |
| P2RY14 | GTF2B | 0.883 |
| NRGN | P2RY14 | 0.882 |
| P2RY14 | RLF | 0.882 |
| SECTM1 | DIAPH1 | 0.882 |
| P2RY14 | CCND2 | 0.882 |
| GBP5 | DUSP12 | 0.882 |
| GBP5 | PIGF | 0.882 |
| GBP5 | PSEN2 | 0.882 |
| GBP5 | CCT2 | 0.882 |
| GBP5 | DNASE1L1 | 0.882 |
| P2RY14 | THAP1 | 0.882 |
| GBP1 | SLTM | 0.882 |
| APOL2 | SMARCC1 | 0.882 |
| APOL2 | CEP250 | 0.882 |
| P2RY14 | TOR1AIP1 | 0.882 |
| GBP5 | ICA1 | 0.882 |
| P2RY14 | SAP130 | 0.882 |
| GBP5 | BRD8 | 0.882 |
| SECTM1 | IMPDH1 | 0.882 |
| GBP5 | PAK4 | 0.882 |
| GBP5 | DYNLT1 | 0.882 |
| GBP5 | FBX09 | 0.882 |
| POLB | ERCC3 | 0.882 |
| GBP5 | ZNF32 | 0.882 |
| P2RY14 | RNF170 | 0.882 |
| GBP5 | SUMO3 | 0.882 |
| GBP5 | CDC42EP4 | 0.882 |
| P2RY14 | SMAP1 | 0.882 |
| GBP5 | CABPS | 0.882 |
| GBP5 | SNN | 0.882 |
| GBP5 | ZNF160 | 0.882 |
| GBP5 | CXXC1 | 0.882 |
| GBP5 | AVIL | 0.882 |
| POLB | PPP2R5D | 0.882 |
| C1QA | DAPP1 | 0.882 |
| DAPP1 | VAMP5 | 0.882 |
| P2RY14 | E2F3 | 0.882 |
| GBP5 | PSMA4 | 0.882 |
| SECTM1 | MAX | 0.882 |
| GBP5 | CBR3 | 0.882 |
| P2RY14 | HSF2 | 0.882 |
| GBP5 | MVD | 0.882 |
| GYPC | P2RY14 | 0.882 |
| P2RY14 | PRNP | 0.882 |
| GBP5 | NDUFA8 | 0.882 |
| GBP5 | ZFP36 | 0.882 |
| GBP5 | ARPCSL | 0.882 |
| POLB | OXSR1 | 0.882 |
| GBP5 | P2RX5 | 0.882 |
| GBP5 | ENY2 | 0.882 |
| P2RY14 | TAPBPL | 0.882 |
| GBP5 | TRIM28 | 0.882 |
| GBP5 | TESK1 | 0.882 |
| GBP5 | ALOX5 | 0.882 |
| GBP1 | RPS14 | 0.882 |
| P2RY14 | PPP2R5A | 0.882 |
| P2RY14 | METAP1 | 0.882 |
| POLB | ZCCHC14 | 0.882 |
| PRDX4 | P2RY14 | 0.882 |
| GBP5 | TNFRSF21 | 0.882 |
| P2RY14 | UBE3A | 0.882 |
| GBP5 | MFN1 | 0.882 |
| P2RY14 | PDK3 | 0.882 |
| GBP5 | TYMS | 0.882 |
| P2RY14 | DNAJC1 | 0.882 |
| P2RY14 | SPIN1 | 0.882 |
| P2RY14 | ACTR8 | 0.882 |
| SECTM1 | SLC16A5 | 0.882 |
| POLB | GRWD1 | 0.882 |
| P2RY14 | POLR2B | 0.882 |
| P2RY14 | TSEN2 | 0.882 |
| GBP5 | SAP30BP | 0.882 |
| AIM2 | P2RY14 | 0.882 |
| P2RY14 | BCL2L11 | 0.882 |
| GBP1 | PLAGL2 | 0.882 |
| GBP1 | SLC38A1 | 0.882 |
| GBP1 | SATB1 | 0.882 |
| P2RY14 | SOAT1 | 0.882 |
| P2RY14 | TRIM24 | 0.882 |
| GBP5 | SIT1 | 0.882 |
| SECTM1 | PLK3 | 0.882 |
| P2RY14 | OSGEPL1 | 0.882 |
| GNG11 | SECTM1 | 0.882 |
| P2RY14 | NDUFB2 | 0.882 |
| GBP5 | MRPS7 | 0.882 |
| POLB | PLAA | 0.882 |
| CALCOCO2 | NDRG1 | 0.882 |
| P2RY14 | ZNF235 | 0.882 |
| GBP1 | MTMR6 | 0.882 |
| P2RY14 | TMEM87A | 0.882 |
| P2RY14 | PYCARD | 0.882 |
| P2RY14 | CHMP7 | 0.882 |
| PHKG2 | P2RY14 | 0.882 |
| P2RY14 | RRS1 | 0.882 |
| GBP5 | NECAP2 | 0.882 |
| GBP5 | RNF113A | 0.882 |
| P2RY14 | CPNE3 | 0.882 |
| P2RY14 | HIF1A | 0.882 |
| P2RY14 | TNFAIP8 | 0.882 |
| GBP5 | NAT10 | 0.882 |
| GBP5 | GNAZ | 0.882 |
| ACP5 | DAPP1 | 0.882 |
| POLB | FBXW2 | 0.882 |
| P2RY14 | PPBP | 0.882 |
| GBP5 | PEPD | 0.882 |
| PSME1 | VILL | 0.882 |
| GBP5 | GOSR2 | 0.882 |
| P2RY14 | DAAM1 | 0.882 |
| GBP5 | HS1BP3 | 0.882 |
| GBP5 | ARF4 | 0.882 |
| P2RY14 | MBNL2 | 0.882 |
| DAPP1 | STAG1 | 0.882 |
| P2RY14 | POP7 | 0.882 |
| GBP5 | RNF25 | 0.882 |
| GBP5 | WDR61 | 0.882 |
| GBP5 | NRIP3 | 0.882 |
| P2RY14 | KLF9 | 0.882 |
| POLB | NCBP2 | 0.882 |
| POLB | DUSP3 | 0.882 |
| GBP5 | SORL1 | 0.882 |
| P2RY14 | PUS7 | 0.882 |
| STK16 | GBP1 | 0.882 |
| P2RY14 | CD2BP2 | 0.882 |
| P2RY14 | JARID2 | 0.882 |
| GBP5 | PSMA1 | 0.882 |
| P2RY14 | SMARCC2 | 0.882 |
| GBP5 | GBP1 | 0.882 |
| GBP5 | GDPD5 | 0.882 |
| P2RY14 | ARMCX3 | 0.882 |
| UBE2L6 | MAP4 | 0.882 |
| POLB | PUS1 | 0.882 |
| GBP5 | SEC61A2 | 0.882 |
| GBP5 | SETD4 | 0.882 |
| GBP5 | SMARCD2 | 0.882 |
| GBP5 | ARPC2 | 0.882 |
| GBP5 | GDI2 | 0.882 |
| PSMB7 | P2RY14 | 0.882 |
| P2RY14 | TRIM37 | 0.882 |
| GBP5 | ACTR6 | 0.881 |
| GBP5 | LMO2 | 0.881 |
| POLB | PRMT7 | 0.881 |
| P2RY14 | STOML1 | 0.881 |
| GBP1 | SLC16A6 | 0.881 |
| GBP5 | NAGPA | 0.881 |
| GBP5 | E2F5 | 0.881 |
| GBP5 | ME2 | 0.881 |
| P2RY14 | RAD1 | 0.881 |
| GBP5 | MPL | 0.881 |
| P2RY14 | CLPX | 0.881 |
| UBE2L6 | EHD4 | 0.881 |
| GBP5 | SEC14L1 | 0.881 |
| IL15RA | CALCOCO2 | 0.881 |
| P2RY14 | TUBD1 | 0.881 |
| GBP5 | SIGLEC7 | 0.881 |
| P2RY14 | ARHGEF6 | 0.881 |
| P2RY14 | ABHD11 | 0.881 |
| GBP5 | HK1 | 0.881 |
| P2RY14 | FNBP1 | 0.881 |
| GBP5 | BIN3 | 0.881 |
| POLB | XPO1 | 0.881 |
| GBP5 | REXO2 | 0.881 |
| POLB | PRDM4 | 0.881 |
| POLB | ITGA7 | 0.881 |
| P2RY14 | GFOD1 | 0.881 |
| P2RY14 | VAC14 | 0.881 |
| SECTM1 | LIMK1 | 0.881 |
| POLB | TRAF1 | 0.881 |
| GBP5 | GUSB | 0.881 |
| GBP5 | PAF1 | 0.881 |
| GBP5 | ANGPT1 | 0.881 |
| P2RY14 | STX10 | 0.881 |
| GBP5 | POLR3K | 0.881 |
| P2RY14 | CSE1L | 0.881 |
| GBP5 | GSN | 0.881 |
| UBE2L6 | CYB561 | 0.881 |
| GBP5 | CSAD | 0.881 |
| GBP5 | TNFRSF1B | 0.881 |
| P2RY14 | DNM1L | 0.881 |
| P2RY14 | POMT1 | 0.881 |
| POLB | ACAD8 | 0.881 |
| GBP5 | CCT7 | 0.881 |
| UBEZL6 | BCR | 0.881 |
| GBP5 | CBX3 | 0.881 |
| POLB | POGZ | 0.881 |
| ATG3 | NDRG1 | 0.881 |
| POLB | NOLC1 | 0.881 |
| GBP5 | RHOA | 0.881 |
| POLB | AACS | 0.881 |
| GBP5 | ARFGAP1 | 0.881 |
| GBP5 | BTN3A1 | 0.881 |
| GBP5 | PEMT | 0.881 |
| SECTM1 | DAPP1 | 0.881 |
| P2RY14 | SLC6A6 | 0.881 |
| GBP1 | MCM2 | 0.881 |
| P2RY14 | NRG1 | 0.881 |
| APOL2 | VILL | 0.881 |
| P2RY14 | GLG1 | 0.881 |
| P2RY14 | NUMA1 | 0.881 |
| DRG1 | P2RY14 | 0.881 |
| POLB | ALMS1 | 0.881 |
| GBP5 | CSTF3 | 0.881 |
| P2RY14 | ALG6 | 0.881 |
| GBP5 | PTPN6 | 0.881 |
| GBP5 | SQSTM1 | 0.881 |
| GBP5 | TKTL1 | 0.881 |
| P2RY14 | SERINC3 | 0.881 |
| P2RY14 | OVGP1 | 0.881 |
| GBP5 | DAZAP2 | 0.881 |
| P2RY14 | IL21R | 0.881 |
| GBP5 | COX15 | 0.881 |
| GBP5 | TYK2 | 0.881 |
| GBP1 | ETS1 | 0.881 |
| GBP5 | PHKB | 0.881 |
| P2RY14 | COPS7A | 0.881 |
| GBP5 | ID2 | 0.881 |
| GBP5 | CD320 | 0.881 |
| P2RY14 | ASCL2 | 0.881 |
| GBP5 | LAMA5 | 0.881 |
| SECTM1 | USP4 | 0.881 |
| UBE2L6 | SLC3A2 | 0.881 |
| P2RY14 | UBE4B | 0.881 |
| GBP5 | MAP3K8 | 0.881 |
| GBP5 | WDR25 | 0.881 |
| GBP5 | MAT2B | 0.881 |
| POLB | RPAP1 | 0.881 |
| GBP1 | RIN2 | 0.881 |
| GBP5 | LAMP1 | 0.881 |
| P2RY14 | CLASP1 | 0.881 |
| GBP5 | TBC1D15 | 0.881 |
| GBP5 | SF1 | 0.881 |
| P2RY14 | ASB1 | 0.881 |
| POLB | TFAM | 0.881 |
| P2RY14 | RFC5 | 0.881 |
| GBP5 | EPM2A | 0.881 |
| GBP5 | CHCHD7 | 0.881 |
| P2RY14 | TRIM68 | 0.881 |
| GBP5 | PDCD10 | 0.881 |
| POLB | TRAF3IP3 | 0.881 |
| GBP5 | PPP1R15A | 0.881 |
| P2RY14 | TMED5 | 0.881 |
| GBP1 | TNFAIP3 | 0.881 |
| GBP5 | SLC37A4 | 0.881 |
| UBE2L6 | ACP5 | 0.881 |
| GBP5 | TBCC | 0.881 |
| P2RY14 | LANCL1 | 0.881 |
| SECTM1 | OSBPL9 | 0.881 |
| GBP5 | EIF2B2 | 0.881 |
| GBP5 | PGK1 | 0.881 |
| GBP5 | EAF2 | 0.881 |
| GBP5 | PMPCA | 0.881 |
| P2RY14 | LRP1 | 0.881 |
| P2RY14 | GBE1 | 0.881 |
| P2RY14 | PPP1CC | 0.881 |
| MYL9 | P2RY14 | 0.881 |
| GBP5 | PBX3 | 0.881 |
| GBP5 | PLEKHB2 | 0.881 |
| GBP5 | IFNGR1 | 0.881 |
| GBP5 | KCNMB4 | 0.881 |
| TMEM59 | POLB | 0.881 |
| POLB | PFAS | 0.881 |
| P2RY14 | LAX1 | 0.881 |
| GBP5 | PRDX3 | 0.881 |
| GBP5 | MKLN1 | 0.881 |
| GBP5 | PTPN18 | 0.881 |
| P2RY14 | YIPF5 | 0.881 |
| P2RY14 | AP3M2 | 0.881 |
| GNG11 | APOL2 | 0.881 |
| P2RY14 | ECHDC2 | 0.881 |
| GBP1 | NDRG1 | 0.881 |
| GBP5 | RALBP1 | 0.881 |
| GBP5 | ATRN | 0.881 |
| POLB | RBM22 | 0.881 |
| P2RY14 | POU2F1 | 0.881 |
| GBP5 | DEF6 | 0.881 |
| P2RY14 | ZNF614 | 0.881 |
| POLB | ZBTB16 | 0.881 |
| GBP5 | PRKAB1 | 0.881 |
| POLB | DNAJA3 | 0.881 |
| GBP5 | STX7 | 0.881 |
| GBP5 | CCNH | 0.881 |
| GBP5 | SCPEP1 | 0.881 |
| GBP5 | TAX1BP1 | 0.881 |
| P2RY14 | DPM2 | 0.881 |
| SECTM1 | ARID1A | 0.881 |
| P2RY14 | RNMT | 0.881 |
| POLB | PCSK6 | 0.881 |
| POLB | TSPAN14 | 0.881 |
| GBP5 | PSMA5 | 0.881 |
| GBP5 | COPB2 | 0.881 |
| GBP5 | RNF13 | 0.88 |
| GBP5 | NAT1 | 0.88 |
| GBP5 | CRYM | 0.88 |
| UBE2L6 | LIG1 | 0.88 |
| GBP5 | CTNNBIP1 | 0.88 |
| GBP1 | PHF3 | 0.88 |
| P2RY14 | ERBB2 | 0.88 |
| GBP5 | ME1 | 0.88 |
| P2RY14 | PPRC1 | 0.88 |
| P2RY14 | SDK2 | 0.88 |
| P2RY14 | MAPK13 | 0.88 |
| GBP5 | GGCX | 0.88 |
| POLB | PPRC1 | 0.88 |
| GBP5 | GTPBP2 | 0.88 |
| GBP5 | EHD1 | 0.88 |
| GBP5 | HMGB2 | 0.88 |
| P2RY14 | CD40 | 0.88 |
| GBP5 | FBXO22 | 0.88 |
| GBP5 | RCE1 | 0.88 |
| GBP5 | RIPK2 | 0.88 |
| POLB | FYCO1 | 0.88 |
| GBP5 | HDDC2 | 0.88 |
| GBP5 | PCCB | 0.88 |
| GBP5 | SH3GL1 | 0.88 |
| GBP5 | DAPK2 | 0.88 |
| GBP5 | COPS7B | 0.88 |
| GBP5 | BCL11A | 0.88 |
| SMARCD3 | GBP1 | 0.88 |
| C1QA | NDRG1 | 0.88 |
| GBP1 | SYNJ2 | 0.88 |
| NPC2 | GBP1 | 0.88 |
| GBP5 | PSMB5 | 0.88 |
| P2RY14 | RPA1 | 0.88 |
| GBP5 | SNX5 | 0.88 |
| COX7A2 | P2RY14 | 0.88 |
| STX8 | P2RY14 | 0.88 |
| GNG11 | APOL1 | 0.88 |
| GBP5 | ARL3 | 0.88 |
| P2RY14 | CYB561D2 | 0.88 |
| GBP5 | DCTN6 | 0.88 |
| POLB | CDCA4 | 0.88 |
| P2RY14 | VRK3 | 0.88 |
| P2RY14 | CREB3 | 0.88 |
| P2RY14 | ASTE1 | 0.88 |
| GBP5 | AOAH | 0.88 |
| PF4V1 | C1QA | 0.88 |
| P2RY14 | CBR1 | 0.88 |
| GBP5 | LITAF | 0.88 |
| SECTM1 | RNF122 | 0.88 |
| POLB | PLCD1 | 0.88 |
| GBP5 | SLC35E1 | 0.88 |
| GBP5 | EEF1E1 | 0.88 |
| POLB | RANBP3 | 0.88 |
| GBP5 | SLC11A2 | 0.88 |
| P2RY14 | EIF4ENIF1 | 0.88 |
| P2RY14 | IRS2 | 0.88 |
| P2RY14 | LBR | 0.88 |
| GBP1 | TP53BP2 | 0.88 |
| GBP5 | ATP6V1C1 | 0.88 |
| GBP5 | FAM111A | 0.88 |
| POLB | MRPL9 | 0.88 |
| P2RY14 | GOLT1B | 0.88 |
| SECTM1 | ATG3 | 0.88 |
| GBP5 | TNFAIP2 | 0.88 |
| GBP5 | MRPL39 | 0.88 |
| GBP5 | NUP43 | 0.88 |
| P2RY14 | MCM3 | 0.88 |
| POLB | SMAD3 | 0.88 |
| GBP5 | PLA2G4C | 0.88 |
| GBP5 | SOCS3 | 0.88 |
| GBP5 | ITGB7 | 0.88 |
| GBP5 | TACC3 | 0.88 |
| P2RY14 | YTHDF1 | 0.88 |
| GBP5 | ZNF576 | 0.88 |
| POLB | SMARCD3 | 0.88 |
| SECTM1 | PGM1 | 0.88 |
| GBP5 | MOCS2 | 0.88 |
| P2RY14 | SNIP1 | 0.88 |
| DAPP1 | LIMK1 | 0.88 |
| P2RY14 | HSPA14 | 0.88 |
| POLB | SS18 | 0.88 |
| GBP5 | CDKN1B | 0.88 |
| POLB | VPS11 | 0.88 |
| POLB | ABR | 0.88 |
| GBP5 | BTG2 | 0.88 |
| SECTM1 | IFT122 | 0.88 |
| POLB | POMT1 | 0.88 |
| GBP5 | NELL2 | 0.88 |
| P2RY14 | TBC1D22A | 0.88 |
| GBP1 | HMGCR | 0.88 |
| P2RY14 | XPNPEP1 | 0.88 |
| POLB | NUP210 | 0.88 |
| P2RY14 | NT5C2 | 0.88 |
| GBP1 | GPBP1L1 | 0.88 |
| GBP5 | AK1 | 0.88 |
| P2RY14 | CALU | 0.88 |
| C1QA | PDE4B | 0.88 |
| LSM1 | SECTM1 | 0.88 |
| CCS | GBP1 | 0.88 |
| GBP5 | FKBP11 | 0.88 |
| GBP5 | ITPK1 | 0.88 |
| LHFPL2 | KLF6 | 0.88 |
| LHFPL2 | SLCO4C1 | 0.88 |
| GBP5 | GLT8D1 | 0.88 |
| SECTM1 | VAMP5 | 0.88 |
| GBP5 | TMEM9B | 0.88 |
| GBP5 | ZBTB32 | 0.88 |
| POLB | SEH1L | 0.88 |
| GBP5 | PPIE | 0.88 |
| SECTM1 | DDX27 | 0.88 |
| GBP5 | MRPS2 | 0.88 |
| P2RY14 | CASP2 | 0.88 |
| GBP5 | MRPS28 | 0.88 |
| GBP5 | OAT | 0.88 |
| P2RY14 | RAB20 | 0.88 |
| GBP5 | ARFIP1 | 0.88 |
| GBP5 | ZNF343 | 0.88 |
| POLB | TP53BP2 | 0.88 |
| GBP5 | KLHL26 | 0.88 |
| GBP5 | PSTPIP1 | 0.88 |
| P2RY14 | ERCC3 | 0.88 |
| P2RY14 | NUDT21 | 0.88 |
| GBP5 | PRPF18 | 0.88 |
| GBP1 | MSH2 | 0.88 |
| GBP1 | RUNX2 | 0.88 |
| GBP5 | TCP11L1 | 0.88 |
| UBE2L6 | RBM28 | 0.88 |
| GBP1 | SMARCC1 | 0.88 |
| GBP5 | ARHGDIA | 0.88 |
| GBP5 | ZNF593 | 0.88 |
| SECTM1 | NFATC1 | 0.88 |
| APOL2 | DGKD | 0.88 |
| GBP5 | NAT9 | 0.88 |
| TCTA | P2RY14 | 0.88 |
| P2RY14 | E2F1 | 0.88 |
| GBP5 | ICOSLG | 0.88 |
| GBP5 | PHGDH | 0.88 |
| GSTO1 | GBP1 | 0.879 |
| POLB | DIAPH1 | 0.879 |
| GBP5 | TRADD | 0.879 |
| GBP5 | TCF3 | 0.879 |
| GBP5 | RNF34 | 0.879 |
| POLB | OGDH | 0.879 |
| GBP5 | DISC1 | 0.879 |
| POLB | MUTYH | 0.879 |
| P2RY14 | ODF2 | 0.879 |
| GBP5 | POLR2K | 0.879 |
| GBP1 | HIGD2A | 0.879 |
| P2RY14 | MED8 | 0.879 |
| GBP5 | FAM20B | 0.879 |
| GBP5 | MAFF | 0.879 |
| P2RY14 | NMT2 | 0.879 |
| APOL2 | PBXIP1 | 0.879 |
| GBP5 | CCDC28B | 0.879 |
| GBP5 | MRPL13 | 0.879 |
| POLB | PSMD11 | 0.879 |
| P2RY14 | PF4V1 | 0.879 |
| GBP5 | STX16 | 0.879 |
| LHFPL2 | SECTM1 | 0.879 |
| POLB | IL12RB1 | 0.879 |
| P2RY14 | YEATS4 | 0.879 |
| POLB | PSMD5 | 0.879 |
| GBP1 | STAG1 | 0.879 |
| GBP5 | CAMKK2 | 0.879 |
| GBP5 | MAP3K6 | 0.879 |
| GBP5 | ZNF22 | 0.879 |
| P2RY14 | CLDND1 | 0.879 |
| P2RY14 | DALRD3 | 0.879 |
| GBP5 | CDC42EP2 | 0.879 |
| P2RY14 | DNASE2 | 0.879 |
| GBP5 | ITGAE | 0.879 |
| GBP5 | HAX1 | 0.879 |
| P2RY14 | DDX50 | 0.879 |
| CD33 | P2RY14 | 0.879 |
| GBP5 | HRH2 | 0.879 |
| GBP5 | PIK3C3 | 0.879 |
| GBP5 | SLC35B1 | 0.879 |
| GBP1 | SPEN | 0.879 |
| GBP5 | FH | 0.879 |
| GBP1 | PTP4A1 | 0.879 |
| GBP5 | TUBGCP2 | 0.879 |
| GBP5 | PAQR4 | 0.879 |
| GBP5 | PDE6D | 0.879 |
| POLB | NFATC3 | 0.879 |
| GBP5 | MAPK14 | 0.879 |
| GBP5 | APBB1IP | 0.879 |
| GBP1 | OSBPL11 | 0.879 |
| P2RY14 | ITGB5 | 0.879 |
| NPC2 | POLB | 0.879 |
| GBP5 | LAPTM5 | 0.879 |
| GBP5 | PYGB | 0.879 |
| GBP5 | ALG8 | 0.879 |
| GBP1 | ARHGEF3 | 0.879 |
| GBP5 | SLC35A1 | 0.879 |
| GBP5 | SH2D1A | 0.879 |
| GBP5 | UNC50 | 0.879 |
| GBP5 | CRELD1 | 0.879 |
| P2RY14 | ZHX2 | 0.879 |
| DAPP1 | SRPK2 | 0.879 |
| P2RY14 | DR1 | 0.879 |
| P2RY14 | PLAGL2 | 0.879 |
| C1QA | APOL2 | 0.879 |
| GBP5 | AMD1 | 0.879 |
| PRRG4 | C1QA | 0.879 |
| FOLR2 | GBP1 | 0.879 |
| GBP1 | ARID1A | 0.879 |
| GBP5 | ARAF | 0.879 |
| P2RY14 | PILRA | 0.879 |
| P2RY14 | RAB5B | 0.879 |
| APOL2 | ARID1A | 0.879 |
| GBP5 | TFE3 | 0.879 |
| GBP5 | TJP2 | 0.879 |
| LHFPL2 | LIMK1 | 0.879 |
| GBP5 | COQ10B | 0.879 |
| GBP5 | SCNN1D | 0.879 |
| SECTM1 | PDHA1 | 0.879 |
| GBP5 | RABGGTA | 0.879 |
| GBP5 | SHMT2 | 0.879 |
| P2RY14 | DHX38 | 0.879 |
| GBP5 | ATN1 | 0.879 |
| P2RY14 | ABCC10 | 0.879 |
| COASY | P2RY14 | 0.879 |
| SECTM1 | APLP2 | 0.879 |
| GBP5 | PTPN9 | 0.879 |
| P2RY14 | BTG1 | 0.879 |
| GBP5 | COL9A2 | 0.879 |
| GBP5 | OSBPL8 | 0.879 |
| APOL2 | POLG | 0.879 |
| GBP5 | DUS1L | 0.879 |
| GBP1 | OSBPL9 | 0.879 |
| P2RY14 | ZXDC | 0.879 |
| P2RY14 | DCK | 0.879 |
| POLB | AXIN1 | 0.879 |
| PSME1 | IL15RA | 0.879 |
| GBP5 | STX12 | 0.879 |
| POLB | RNF122 | 0.879 |
| VAMP5 | SLC3A2 | 0.879 |
| GBP5 | PANK2 | 0.879 |
| P2RY14 | MCM2 | 0.879 |
| P2RY14 | FANCE | 0.879 |
| P2RY14 | STT3A | 0.879 |
| P2RY14 | NMD3 | 0.879 |
| GBP1 | BARD1 | 0.879 |
| GBP1 | DNAJC4 | 0.879 |
| GBP5 | PCBP1 | 0.879 |
| GBP5 | ARMC6 | 0.879 |
| GBP5 | CSK | 0.879 |
| P2RY14 | DUSP22 | 0.879 |
| GBP5 | RUVBL2 | 0.879 |
| UBE2L6 | ATG3 | 0.879 |
| POLB | GANAB | 0.879 |
| P2RY14 | SERINC1 | 0.879 |
| GBP5 | BBS4 | 0.879 |
| GBP5 | ASL | 0.879 |
| GBP5 | LPXN | 0.879 |
| UBE2L6 | SMARCC1 | 0.879 |
| GBP5 | RTN1 | 0.879 |
| UBE2L6 | CALCOCO2 | 0.879 |
| SECTM1 | POU2F1 | 0.879 |
| GYPC | GBP1 | 0.879 |
| GBP5 | KHSRP | 0.879 |
| GBP5 | PALLD | 0.879 |
| POLB | ADNP | 0.879 |
| P2RY14 | NOL11 | 0.879 |
| GBP1 | SYNCRIP | 0.879 |
| GBP5 | EPAS1 | 0.879 |
| POLB | UBE2L3 | 0.879 |
| UBE2L6 | APOL2 | 0.879 |
| POLB | PRRG4 | 0.879 |
| GBP5 | UQCRC2 | 0.879 |
| P2RY14 | SSBP1 | 0.879 |
| SECTM1 | ZBTB16 | 0.879 |
| GBP5 | PPP1R12B | 0.879 |
| P2RY14 | TTC19 | 0.879 |
| GBP5 | ABCD1 | 0.879 |
| GBP5 | PRKCZ | 0.879 |
| GBP5 | SLC23A2 | 0.879 |
| GBP1 | OSBP | 0.879 |
| POLB | MAP4 | 0.879 |
| GBP5 | UBE2B | 0.879 |
| GBP5 | STX6 | 0.879 |
| GBP5 | FBXO3 | 0.879 |
| GBP5 | PPFIA1 | 0.878 |
| GBP5 | HEMK1 | 0.878 |
| TMED3 | GBP1 | 0.878 |
| P2RY14 | GNE | 0.878 |
| POLB | GFI1B | 0.878 |
| POLB | DNAJC1 | 0.878 |
| P2RY14 | AQP3 | 0.878 |
| SECTM1 | FLII | 0.878 |
| GBP5 | LIMK2 | 0.878 |
| GBP5 | CLCN7 | 0.878 |
| GBP5 | TRMT12 | 0.878 |
| P2RY14 | PRRG4 | 0.878 |
| P2RY14 | IL7R | 0.878 |
| GBP5 | MRPS14 | 0.878 |
| P2RY14 | EIF5A2 | 0.878 |
| GBP5 | RUSC1 | 0.878 |
| P2RY14 | LPIN1 | 0.878 |
| P2RY14 | TGOLN2 | 0.878 |
| UBE216 | YEATS2 | 0.878 |
| UBE2L6 | IL15RA | 0.878 |
| APOL1 | GBP1 | 0.878 |
| P2RY14 | PDHA1 | 0.878 |
| GBP1 | TRAPPC3 | 0.878 |
| POLB | MED9 | 0.878 |
| P2RY14 | ECE2 | 0.878 |
| POLB | SAR1A | 0.878 |
| GBP5 | VPS72 | 0.878 |
| ACP5 | APOL2 | 0.878 |
| GBP5 | SLC41A3 | 0.878 |
| GNG11 | VAMP5 | 0.878 |
| GBP5 | DCLRE1C | 0.878 |
| GBP5 | RREB1 | 0.878 |
| GBP5 | ZNF226 | 0.878 |
| P2RY14 | HS2ST1 | 0.878 |
| GBP5 | GIT2 | 0.878 |
| P2RY14 | ING3 | 0.878 |
| UBE2L6 | PFDN5 | 0.878 |
| SECTM1 | RGS14 | 0.878 |
| POLB | UPF2 | 0.878 |
| GBP5 | CASP3 | 0.878 |
| UBE2L6 | GLG1 | 0.878 |
| GBP5 | MGLL | 0.878 |
| GBP5 | GPS1 | 0.878 |
| GBP5 | GOLPH3L | 0.878 |
| POLB | MKRN2 | 0.878 |
| GBP5 | RPGRIP1 | 0.878 |
| GBP5 | ABHD3 | 0.878 |
| P2RY14 | AHR | 0.878 |
| VAMP5 | NDRG1 | 0.878 |
| GBP5 | ZBTB20 | 0.878 |
| POLB | CAPN2 | 0.878 |
| GBP5 | PCGF1 | 0.878 |
| GBP5 | CKS2 | 0.878 |
| POLB | ERBB2 | 0.878 |
| LHFPL2 | APOL2 | 0.878 |
| PSME1 | BCR | 0.878 |
| GBP5 | LYPLA1 | 0.878 |
| P2RY14 | GTDC1 | 0.878 |
| GBP5 | RFX3 | 0.878 |
| CYB5R1 | P2RY14 | 0.878 |
| POLB | PXMP4 | 0.878 |
| POLB | GPD1L | 0.878 |
| P2RY14 | CRY2 | 0.878 |
| GBP5 | BTN2A1 | 0.878 |
| P2RY14 | EXOSC8 | 0.878 |
| DYNLT1 | P2RY14 | 0.878 |
| GBP5 | PRMT1 | 0.878 |
| GBP5 | LGALS8 | 0.878 |
| P2RY14 | PGRMC2 | 0.878 |
| P2RY14 | SYNGR1 | 0.878 |
| POLB | TRAP1 | 0.878 |
| GBP5 | CNDP2 | 0.878 |
| P2RY14 | CRLF3 | 0.878 |
| P2RY14 | RRAS | 0.878 |
| P2RY14 | CASP6 | 0.878 |
| POLB | CD40 | 0.878 |
| P2RY14 | IDS | 0.878 |
| P2RY14 | PTPN22 | 0.878 |
| P2RY14 | RB1CC1 | 0.878 |
| TMSB10 | GBP1 | 0.878 |
| GBP5 | GGA3 | 0.878 |
| GBP5 | TNFRSF10B | 0.878 |
| POLB | MRPL49 | 0.878 |
| UNC119 | P2RY14 | 0.878 |
| GBP5 | NPM3 | 0.878 |
| GBP5 | ACOT9 | 0.878 |
| P2RY14 | AMMECR1 | 0.878 |
| GBP5 | THAP11 | 0.878 |
| P2RY14 | MAF | 0.878 |
| GBP5 | MTCH1 | 0.878 |
| SECTM1 | MLX | 0.878 |
| GBP5 | SENP3 | 0.878 |
| P2RY14 | CACNA1I | 0.878 |
| UBE2L6 | DAPP1 | 0.878 |
| GBP5 | ACBD4 | 0.878 |
| DNAJC7 | P2RY14 | 0.878 |
| GBP1 | MAK | 0.878 |
| IL15RA | LIMK1 | 0.878 |
| GBP5 | GLS | 0.878 |
| GBP5 | RRAGD | 0.878 |
| GBP5 | MUC1 | 0.878 |
| GBP5 | CCR7 | 0.878 |
| P2RY14 | NUDT2 | 0.878 |
| POLB | ITPKB | 0.878 |
| GBP5 | COL18A1 | 0.878 |
| P2RY14 | UFM1 | 0.878 |
| GBP5 | TNIP2 | 0.878 |
| GBP5 | HIF1AN | 0.878 |
| GBP5 | ZNF557 | 0.878 |
| GBP5 | CEACAM21 | 0.878 |
| GBP1 | RNGTT | 0.878 |
| P2RY14 | BLVRA | 0.878 |
| GBP5 | VPS4A | 0.878 |
| P2RY14 | JAM3 | 0.878 |
| UBE2L6 | MCM3 | 0.878 |
| IL15RA | MAX | 0.878 |
| GBP5 | TM6SF1 | 0.878 |
| GBP5 | SLC22A17 | 0.878 |
| GBP1 | GIMAP6 | 0.878 |
| GBP5 | VAV1 | 0.878 |
| GBP1 | USP8 | 0.878 |
| UBE2L6 | APOL1 | 0.878 |
| GBP5 | ME3 | 0.878 |
| PSME1 | LHFPL2 | 0.878 |
| GBP5 | MMD | 0.878 |
| POLB | PARP2 | 0.878 |
| POLB | CASP7 | 0.878 |
| POLB | VCP | 0.877 |
| P2RY14 | PARP2 | 0.877 |
| GBP1 | PUM1 | 0.877 |
| SECTM1 | POLD3 | 0.877 |
| POLB | FTSJ3 | 0.877 |
| GBP5 | MYOM1 | 0.877 |
| UBE2L6 | CEP250 | 0.877 |
| GBP1 | VAMP5 | 0.877 |
| POLB | PTPN2 | 0.877 |
| GBP5 | ZNF586 | 0.877 |
| GBP5 | ADPGK | 0.877 |
| GBP5 | MRPL15 | 0.877 |
| POLB | SON | 0.877 |
| GBP5 | GPR65 | 0.877 |
| P2RY14 | CBX1 | 0.877 |
| GBP1 | NPAT | 0.877 |
| GBP5 | PMM1 | 0.877 |
| POLB | CHMP7 | 0.877 |
| GBP5 | ENTPD5 | 0.877 |
| POLB | TIMM44 | 0.877 |
| GBP5 | TRIAP1 | 0.877 |
| P2RY14 | INPP5E | 0.877 |
| GBP5 | ASPSCR1 | 0.877 |
| P2RY14 | MRPL44 | 0.877 |
| GBP5 | TBC1D22B | 0.877 |
| POLB | BRF2 | 0.877 |
| GBP5 | PIGO | 0.877 |
| GBP5 | SIRPB1 | 0.877 |
| COX6B1 | POLB | 0.877 |
| FAH | P2RY14 | 0.877 |
| P2RY14 | VILL | 0.877 |
| SECTM1 | CAMK2G | 0.877 |
| P2RY14 | SOX4 | 0.877 |
| P2RY14 | MPPE1 | 0.877 |
| GBP5 | ARPCS | 0.877 |
| GBP5 | PRSS21 | 0.877 |
| GBP1 | PPP3CA | 0.877 |
| P2RY14 | MAN1C1 | 0.877 |
| POLB | ING1 | 0.877 |
| GBP5 | COG7 | 0.877 |
| P2RY14 | HSPH1 | 0.877 |
| LHFPL2 | HMGCR | 0.877 |
| SECTM1 | C1QA | 0.877 |
| GBP5 | MRPL22 | 0.877 |
| GBP5 | TBL1X | 0.877 |
| UBE2L6 | MCM6 | 0.877 |
| GBP1 | SRPK2 | 0.877 |
| P2RY14 | PRPF8 | 0.877 |
| P2RY14 | ATG12 | 0.877 |
| GBP5 | ZNF646 | 0.877 |
| SECTM1 | OSBP | 0.877 |
| SECTM1 | IL16 | 0.877 |
| PSMA5 | P2RY14 | 0.877 |
| GBP5 | SAV1 | 0.877 |
| SECTM1 | VILL | 0.877 |
| P2RY14 | RACGAP1 | 0.877 |
| GBP1 | TOB2 | 0.877 |
| GBP5 | ABCG1 | 0.877 |
| GBP5 | PDE4B | 0.877 |
| GBP1 | CAMK4 | 0.877 |
| GBP5 | SLC37A1 | 0.877 |
| P2RY14 | YWHAZ | 0.877 |
| P2RY14 | ZBTB33 | 0.877 |
| P2RY14 | POLDIP3 | 0.877 |
| GBP5 | MTIF2 | 0.877 |
| P2RY14 | SOCS2 | 0.877 |
| UBE2L6 | TLE4 | 0.877 |
| STAT5A | P2RY14 | 0.877 |
| GBP1 | SEC63 | 0.877 |
| P2RY14 | COPS6 | 0.877 |
| P2RY14 | GPA33 | 0.877 |
| GBP5 | ANXA5 | 0.877 |
| GBP1 | DIAPH1 | 0.877 |
| SECTM1 | GALNT2 | 0.877 |
| P2RY14 | RBM15 | 0.877 |
| LTBR | P2RY14 | 0.877 |
| POLB | TEX10 | 0.877 |
| TMEM59 | SECTM1 | 0.877 |
| SECTM1 | NCOA1 | 0.877 |
| P2RY14 | RIOK2 | 0.877 |
| GBP5 | VIM | 0.877 |
| GBP1 | EIF1 | 0.877 |
| GBP5 | STX11 | 0.877 |
| KCTD17 | P2RY14 | 0.877 |
| UBE2L6 | ATP13A1 | 0.877 |
| PSME1 | PPP2R5D | 0.877 |
| APOL2 | DDX27 | 0.877 |
| GBP5 | ZYX | 0.877 |
| P2RY14 | CASP9 | 0.877 |
| GBP5 | BTK | 0.877 |
| C1QA | ZBTB16 | 0.877 |
| POLB | AP3M2 | 0.877 |
| PSME1 | DAPP1 | 0.877 |
| P2RY14 | MDFIC | 0.877 |
| GBP5 | PARD6A | 0.877 |
| P2RY14 | STARD5 | 0.877 |
| GBP5 | SMAD2 | 0.877 |
| GH1B | GBP1 | 0.877 |
| GBP5 | LRRK1 | 0.877 |
| UBE2L6 | MSN | 0.877 |
| P2RY14 | TMEM50B | 0.877 |
| CALCOCO2 | ARID1A | 0.877 |
| RAB32 | P2RY14 | 0.877 |
| UBEZL6 | NDUFB6 | 0.877 |
| GBP1 | ACBD3 | 0.877 |
| POLB | RASGRP2 | 0.877 |
| UBE2L6 | TNFRSF14 | 0.877 |
| P2RY14 | MAGEH1 | 0.877 |
| P2RY14 | FLAD1 | 0.877 |
| P2RY14 | GNA11 | 0.877 |
| POLB | MAZ | 0.877 |
| P2RY14 | MS4A6A | 0.877 |
| P2RY14 | SYPL1 | 0.877 |
| P2RY14 | BTN2A2 | 0.877 |
| P2RY14 | MAGED1 | 0.877 |
| P2RY14 | HSDL2 | 0.877 |
| GBP5 | ABCC2 | 0.877 |
| GBP5 | CCNI | 0.877 |
| SECTM1 | RPS6KA1 | 0.877 |
| POLB | SATB1 | 0.877 |
| SECTM1 | POLR3B | 0.877 |
| P2RY14 | CLEC10A | 0.877 |
| POLB | TCF7 | 0.877 |
| POLB | CNOT8 | 0.877 |
| P2RY14 | UCHL5 | 0.877 |
| AIM2 | SLCO4C1 | 0.877 |
| UROS | P2RY14 | 0.877 |
| GBP1 | TOB1 | 0.877 |
| POLB | TTLL12 | 0.877 |
| P2RY14 | CDK5RAP1 | 0.877 |
| P2RY14 | ATP6V1H | 0.877 |
| GBP5 | PIK3CD | 0.877 |
| GBP5 | SMPD2 | 0.877 |
| GBP5 | CDK7 | 0.877 |
| GBP1 | TLE4 | 0.877 |
| GBP5 | TNFRSF8 | 0.877 |
| POLB | ZNF232 | 0.877 |
| GBP5 | DHRS7 | 0.877 |
| P2RY14 | PKD2 | 0.877 |
| P2RY14 | GMDS | 0.877 |
| P2RY14 | ITGA2B | 0.877 |
| GBP5 | MX11 | 0.877 |
| GBP5 | C1QB | 0.877 |
| NPC2 | UBE2L6 | 0.877 |
| P2RY14 | PDHX | 0.877 |
| GBP5 | DNAJB12 | 0.877 |
| PPBP | GBP1 | 0.877 |
| GBP5 | FANCL | 0.877 |
| POLB | SNIP1 | 0.876 |
| GBP5 | UQCRC1 | 0.876 |
| POLB | PLK3 | 0.876 |
| GBP5 | ATF6 | 0.876 |
| P2RY14 | TCFL5 | 0.876 |
| P2RY14 | TRAM1 | 0.876 |
| GBP5 | LAT2 | 0.876 |
| GBP5 | KCNH2 | 0.876 |
| GBP5 | CDS2 | 0.876 |
| GBP5 | TNFAIP1 | 0.876 |
| P2RY14 | IL16 | 0.876 |
| GBP5 | CAPZB | 0.876 |
| POLB | ANKRD11 | 0.876 |
| GBP1 | TSPAN14 | 0.876 |
| GBP5 | GOLGA2 | 0.876 |
| P2RY14 | CTBP2 | 0.876 |
| GBP5 | RNASET2 | 0.876 |
| P2RY14 | GADD45B | 0.876 |
| PTGIR | P2RY14 | 0.876 |
| GBP1 | TIPARP | 0.876 |
| UBE2L6 | PPP2R5D | 0.876 |
| GBP5 | SLC12A9 | 0.876 |
| GBP5 | ARHGAP24 | 0.876 |
| P2RY14 | DHRS7B | 0.876 |
| P2RY14 | UPF3A | 0.876 |
| GBP5 | SDF2 | 0.876 |
| POLB | SCMH1 | 0.876 |
| POLB | NMT1 | 0.876 |
| P2RY14 | CETN2 | 0.876 |
| GBP5 | FBXL5 | 0.876 |
| P2RY14 | GANAB | 0.876 |
| POLB | AP1G1 | 0.876 |
| UBE2L6 | ATF7IP | 0.876 |
| POLB | DPH2 | 0.876 |
| GBP5 | NCSTN | 0.876 |
| GBP1 | DHX9 | 0.876 |
| GBP5 | SNRPC | 0.876 |
| GBP5 | PMVK | 0.876 |
| P2RY14 | NUP98 | 0.876 |
| GBP5 | SPOP | 0.876 |
| GBP1 | MYC | 0.876 |
| POLB | DENND2D | 0.876 |
| GBP5 | SLC39A1 | 0.876 |
| POLB | UBE4B | 0.876 |
| GBP5 | MRPS11 | 0.876 |
| POLB | ZNF212 | 0.876 |
| GBP5 | BRWD1 | 0.876 |
| POLB | AQR | 0.876 |
| P2RY14 | ELAVL1 | 0.876 |
| P2RY14 | ZNF395 | 0.876 |
| POLB | MSN | 0.876 |
| GBP5 | ALAD | 0.876 |
| GBP5 | TBC1D2B | 0.876 |
| CASP7 | SECTM1 | 0.876 |
| GBP5 | DBP | 0.876 |
| POLB | VPS13D | 0.876 |
| POLB | YTHDF1 | 0.876 |
| P2RY14 | NOTCH2 | 0.876 |
| GBP5 | SSB | 0.876 |
| SLC7A7 | P2RY14 | 0.876 |
| GBP1 | PFAS | 0.876 |
| GBP1 | DGKD | 0.876 |
| GBP5 | CLTCL1 | 0.876 |
| POLB | JARID2 | 0.876 |
| P2RY14 | AGTPBP1 | 0.876 |
| APOL2 | LIG1 | 0.876 |
| GBP5 | CSNK1G2 | 0.876 |
| GBP5 | NSFL1C | 0.876 |
| P2RY14 | TGFBRAP1 | 0.876 |
| GBP5 | ADCK2 | 0.876 |
| P2RY14 | RPS14 | 0.876 |
| GBP1 | MAML1 | 0.876 |
| GBP5 | MFSD1 | 0.876 |
| GBP5 | F8 | 0.876 |
| APOL1 | SECTM1 | 0.876 |
| GBP5 | LSM4 | 0.876 |
| SEPHS2 | P2RY14 | 0.876 |
| POLB | STT3A | 0.876 |
| GBP5 | B4GALT7 | 0.876 |
| P2RY14 | S100A8 | 0.876 |
| UBE2L6 | DDX27 | 0.876 |
| P2RY14 | SRD5A1 | 0.876 |
| GBP5 | CYP1B1 | 0.876 |
| GBP5 | EXOSC5 | 0.876 |
| GBP5 | PRKAG2 | 0.876 |
| P2RY14 | VAMP3 | 0.876 |
| GBP5 | MAP4K2 | 0.876 |
| POLB | LSR | 0.876 |
| P2RY14 | GMPR2 | 0.876 |
| GBP5 | ALG5 | 0.876 |
| P2RY14 | R3HDM2 | 0.876 |
| SECTM1 | MAP4K4 | 0.876 |
| UBE2L6 | GEMIN4 | 0.876 |
| P2RY14 | TRAP1 | 0.876 |
| VAMP5 | MCM6 | 0.876 |
| GBP5 | MFSD5 | 0.876 |
| GBP5 | STRN | 0.876 |
| DAPP1 | PPP3CA | 0.876 |
| GBP1 | GUK1 | 0.876 |
| POLB | BCCIP | 0.876 |
| P2RY14 | TM9SF4 | 0.876 |
| APOL1 | LHFPL2 | 0.876 |
| GBP5 | EIF2B4 | 0.876 |
| SLC22A18 | P2RY14 | 0.876 |
| P2RY14 | TMEM97 | 0.876 |
| P2RY14 | OGFOD1 | 0.876 |
| PF4V1 | VAMP5 | 0.876 |
| GBP5 | TPST2 | 0.876 |
| SECTM1 | ATP2A3 | 0.876 |
| GBP1 | KNTC1 | 0.876 |
| GBP1 | NCOA1 | 0.876 |
| APEH | P2RY14 | 0.876 |
| AKR1A1 | POLB | 0.876 |
| APOL2 | BCR | 0.876 |
| GBP1 | OXSR1 | 0.876 |
| POLB | MECP2 | 0.876 |
| GBP1 | SLA | 0.876 |
| GBP5 | SLC25A16 | 0.876 |
| P2RY14 | YPEL1 | 0.876 |
| SECTM1 | CTCF | 0.876 |
| POLB | VPS4B | 0.876 |
| GBP5 | UBE2Z | 0.876 |
| P2RY14 | PSTPIP2 | 0.876 |
| P2RY14 | EP300 | 0.876 |
| GBP1 | LIG1 | 0.876 |
| GBP5 | VAT1 | 0.876 |
| POLB | PARP6 | 0.876 |
| GBP5 | RNH1 | 0.876 |
| P2RY14 | PIGH | 0.876 |
| GBP5 | SNRPA | 0.876 |
| GBP5 | STAU2 | 0.876 |
| P2RY14 | YWHAQ | 0.876 |
| GBP5 | CAV1 | 0.876 |
| GBP5 | ST3GAL5 | 0.876 |
| DNAL4 | P2RY14 | 0.876 |
| P2RY14 | MFGE8 | 0.876 |
| PDE4B | VAMP5 | 0.876 |
| GBP5 | LRRC20 | 0.876 |
| GBP1 | NFATC3 | 0.876 |
| GBP5 | MTRF1 | 0.876 |
| GBP5 | ALAS2 | 0.876 |
| GBP5 | HSPE1 | 0.876 |
| P2RY14 | PPARD | 0.876 |
| GBP5 | TAZ | 0.876 |
| ATP1B2 | P2RY14 | 0.876 |
| GBP1 | MSH6 | 0.876 |
| P2RY14 | CCR4 | 0.876 |
| GBP5 | DYRK4 | 0.876 |
| GBP5 | NPDC1 | 0.876 |
| GBP5 | DPH5 | 0.876 |
| GBP5 | STARD7 | 0.876 |
| POLB | REEP4 | 0.876 |
| GBP5 | NAP1L1 | 0.876 |
| POLB | TRIM68 | 0.876 |
| LHFPL2 | CYB561 | 0.876 |
| GBP5 | CD46 | 0.876 |
| GBP5 | ARMC8 | 0.876 |
| P2RY14 | CTDSPL | 0.876 |
| POLB | ZC3H7A | 0.876 |
| GBP5 | RAB1A | 0.876 |
| POLB | ELMO1 | 0.876 |
| SECTM1 | AGPAT3 | 0.876 |
| P2RY14 | SPG21 | 0.876 |
| P2RY14 | SLC35D2 | 0.876 |
| P2RY14 | SLC25A28 | 0.876 |
| P2RY14 | ELAC2 | 0.876 |
| P2RY14 | UBIAD1 | 0.876 |
| GBP5 | SNRPD1 | 0.876 |
| P2RY14 | NCK1 | 0.876 |
| SECTM1 | ANXA11 | 0.876 |
| POLB | MCM7 | 0.876 |
| GBP1 | GOLPH3 | 0.876 |
| GBP5 | LILRB2 | 0.876 |
| P2RY14 | ABHD6 | 0.876 |
| P2RY14 | LAS1L | 0.876 |
| GBP1 | RRM1 | 0.876 |
| GBP5 | THBS3 | 0.876 |
| P2RY14 | ZNF274 | 0.876 |
| CALCOCO2 | SMARCC1 | 0.876 |
| P2RY14 | BCL11B | 0.876 |
| P2RY14 | KIF3B | 0.876 |
| SECTM1 | RFX5 | 0.876 |
| POLB | NUMA1 | 0.876 |
| P2RY14 | TTF1 | 0.876 |
| GBP5 | LILRA1 | 0.876 |
| GBP5 | RNASEL | 0.876 |
| UBE2L6 | SSRP1 | 0.876 |
| GBP5 | TSG101 | 0.876 |
| GBP5 | YIPF4 | 0.876 |
| GBP5 | PPM1A | 0.876 |
| GBP5 | HSPBAP1 | 0.875 |
| GBP5 | BIRC2 | 0.875 |
| APOL1 | DDX27 | 0.875 |
| GBP5 | UBR2 | 0.875 |
| P2RY14 | TERF2 | 0.875 |
| POLB | NCOR1 | 0.875 |
| P2RY14 | PPP2R5D | 0.875 |
| GBP5 | CTNNBL1 | 0.875 |
| GBP5 | DOCK5 | 0.875 |
| POLB | DBR1 | 0.875 |
| P2RY14 | SLC2A9 | 0.875 |
| SECTM1 | FADD | 0.875 |
| GBP5 | TRPM6 | 0.875 |
| P2RY14 | RBBP7 | 0.875 |
| SECTM1 | SSH3 | 0.875 |
| GBP5 | CYBRD1 | 0.875 |
| POLB | PKNOX1 | 0.875 |
| GBP5 | ANAPC13 | 0.875 |
| GBP5 | TREML2 | 0.875 |
| P2RY14 | RNF138 | 0.875 |
| POLB | AIM2 | 0.875 |
| GBP5 | MRPL48 | 0.875 |
| GBP5 | SAMM50 | 0.875 |
| P2RY14 | CD300A | 0.875 |
| GBP5 | CGRRF1 | 0.875 |
| SECTM1 | GMEB1 | 0.875 |
| GBP5 | FDFT1 | 0.875 |
| GBP5 | NOL10 | 0.875 |
| GBP1 | TAF2 | 0.875 |
| POLB | ENSA | 0.875 |
| UBE2L6 | NCL | 0.875 |
| GBP5 | UBE2H | 0.875 |
| GBP1 | NUP210 | 0.875 |
| POLB | STK16 | 0.875 |
| P2RY14 | WBP4 | 0.875 |
| P2RY14 | PPP2CA | 0.875 |
| P2RY14 | ZNF419 | 0.875 |
| P2RY14 | MCFD2 | 0.875 |
| P2RY14 | ABCC3 | 0.875 |
| GMFG | P2RY14 | 0.875 |
| UBE216 | MYD88 | 0.875 |
| GBP5 | ASGR1 | 0.875 |
| P2RY14 | RAB11FIP1 | 0.875 |
| GBP5 | TOLLIP | 0.875 |
| P2RY14 | PICALM | 0.875 |
| GBP5 | DDX49 | 0.875 |
| P2RY14 | ABR | 0.875 |
| SECTM1 | RUNX2 | 0.875 |
| GBP1 | ATF7IP | 0.875 |
| SECTM1 | SSRP1 | 0.875 |
| GBP5 | BTN3A2 | 0.875 |
| GBP5 | TNFRSF1A | 0.875 |
| P2RY14 | TLE2 | 0.875 |
| PZRY14 | YIPF3 | 0.875 |
| POLB | HARS2 | 0.875 |
| GBP5 | RBBP6 | 0.875 |
| GBP5 | TRAT1 | 0.875 |
| GBP5 | EFHD2 | 0.875 |
| GBP5 | FCN1 | 0.875 |
| GBP5 | DIABLO | 0.875 |
| UBE2L6 | TRAPPC3 | 0.875 |
| GBP5 | YARS2 | 0.875 |
| PSME1 | GLG1 | 0.875 |
| POLB | MLX | 0.875 |
| P2RY14 | BSDC1 | 0.875 |
| GBP5 | PDXK | 0.875 |
| P2RY14 | MSN | 0.875 |
| POLB | POLD3 | 0.875 |
| NMI | P2RY14 | 0.875 |
| GBP5 | PRKCSH | 0.875 |
| P2RY14 | SMU1 | 0.875 |
| SECTM1 | GPR132 | 0.875 |
| P2RY14 | DDIT4 | 0.875 |
| P2RY14 | PRPSAP2 | 0.875 |
| POLB | MRPS27 | 0.875 |
| GBP5 | THYN1 | 0.875 |
| GBP1 | NCOR1 | 0.875 |
| GBP5 | PACSIN2 | 0.875 |
| GBP5 | EMP1 | 0.875 |
| GBP1 | TSR1 | 0.875 |
| GBP1 | USP1 | 0.875 |
| C1QB | P2RY14 | 0.875 |
| G6PD | P2RY14 | 0.875 |
| GBP5 | PFKM | 0.875 |
| P2RY14 | CCNG1 | 0.875 |
| DUSP3 | KLF6 | 0.875 |
| POLB | DUSP22 | 0.875 |
| GBP5 | FASTK | 0.875 |
| VAMP5 | VILL | 0.875 |
| PRRG4 | VAMP5 | 0.875 |
| P2RY14 | MRPL16 | 0.875 |
| GBP5 | IL2RG | 0.875 |
| GBP5 | PSMC5 | 0.875 |
| UBE216 | SYNJ2 | 0.875 |
| P2RY14 | ZNF266 | 0.875 |
| SECTM1 | AKAP1 | 0.875 |
| POLB | KPTN | 0.875 |
| GBP5 | CCDC59 | 0.875 |
| GBP5 | CCDC28A | 0.875 |
| P2RY14 | GALNT11 | 0.875 |
| P2RY14 | NFE2L1 | 0.875 |
| GBP5 | PHC2 | 0.875 |
| UBE2L6 | NFATC3 | 0.875 |
| GBP5 | PPM1F | 0.875 |
| TMEM59 | LIMK1 | 0.875 |
| P2RY14 | UBL3 | 0.875 |
| P2RY14 | AP381 | 0.875 |
| GBP5 | PSMD4 | 0.875 |
| GBP5 | METTLS | 0.875 |
| GBP5 | SNAPC5 | 0.875 |
| GBP5 | BMP6 | 0.875 |
| PSME1 | CTCF | 0.875 |
| SECTM1 | ALAS1 | 0.875 |
| AIM2 | CD74 | 0.875 |
| GBP1 | ADNP | 0.875 |
| GBP5 | VBP1 | 0.875 |
| SECTM1 | DHX9 | 0.875 |
| CASP1 | KLF6 | 0.875 |
| P2RY14 | SEC23B | 0.875 |
| P2RY14 | LSR | 0.875 |
| APOL1 | DAPP1 | 0.875 |
| P2RY14 | DNAJC17 | 0.875 |
| SECTM1 | PSMD11 | 0.875 |
| UBE2L6 | TSR1 | 0.875 |
| UBE2L6 | TTLL12 | 0.875 |
| GBP5 | PLEKHF1 | 0.875 |
| GBP5 | ZDHHC3 | 0.875 |
| P2RY14 | PPP1R8 | 0.875 |
| DECR1 | P2RY14 | 0.875 |
| P2RY14 | PKNOX1 | 0.875 |
| IL12RB1 | SECTM1 | 0.875 |
| SECTM1 | ARHGAP1 | 0.875 |
| POLB | PAXIP1 | 0.875 |
| GBP5 | CASP5 | 0.875 |
| GBP1 | PLCG1 | 0.875 |
| P2RY14 | MAGED2 | 0.875 |
| GBP5 | ZNF207 | 0.875 |
| GBP5 | NFKBIB | 0.875 |
| GBP5 | ACADS | 0.875 |
| P2RY14 | HBS1L | 0.875 |
| P2RY14 | PPCDC | 0.875 |
| GBP1 | MTRR | 0.875 |
| UBE2L6 | OGDH | 0.875 |
| POLB | DGKA | 0.875 |
| POLB | STAG2 | 0.875 |
| GBP5 | TCEA2 | 0.875 |
| IL15RA | CEP250 | 0.875 |
| GBP1 | KLF3 | 0.875 |
| P2RY14 | GNRH1 | 0.875 |
| GBP5 | TSC22D4 | 0.875 |
| GBP5 | NAALADL1 | 0.875 |
| P2RY14 | PIGA | 0.875 |
| P2RY14 | TM7SF2 | 0.875 |
| GBP5 | DVL2 | 0.875 |
| GBP5 | GEMIN6 | 0.875 |
| UBE2L6 | TIMELESS | 0.875 |
| SLC43A1 | P2RY14 | 0.875 |
| UBE2L6 | NCOR1 | 0.875 |
| PSME1 | ABCF2 | 0.875 |
| GBP5 | EHMT1 | 0.875 |
| UBE2L6 | PFAS | 0.875 |
| POLB | POLR1B | 0.875 |
| GBP1 | ANKS1A | 0.875 |
| SECTM1 | TSR1 | 0.875 |
| P2RY14 | EXOSC10 | 0.875 |
| LIMK1 | MAX | 0.875 |
| GBP5 | VAPA | 0.875 |
| P2RY14 | MTAP | 0.875 |
| P2RY14 | REL | 0.875 |
| P2RY14 | PRKACA | 0.875 |
| LAPTM4A | UBE216 | 0.875 |
| POLB | EPHA4 | 0.875 |
| GBP1 | RGS10 | 0.875 |
| P2RY14 | PBXIP1 | 0.875 |
| GBP5 | NTHL1 | 0.875 |
| P2RY14 | MGAT2 | 0.875 |
| GBP5 | MRPL35 | 0.875 |
| GBP5 | KLRF1 | 0.875 |
| GBP5 | MLF2 | 0.875 |
| P2RY14 | FZD5 | 0.875 |
| P2RY14 | HYOU1 | 0.875 |
| NDUFB6 | GBP1 | 0.875 |
| LAPTM4A | POLB | 0.875 |
| GBP5 | GALNS | 0.875 |
| UBE2L6 | RRBP1 | 0.874 |
| GBP5 | CD22 | 0.874 |
| ACP5 | PSTPIP2 | 0.874 |
| GBP1 | ADD3 | 0.874 |
| GBP5 | MAN181 | 0.874 |
| GBP5 | C1RL | 0.874 |
| P2RY14 | ETS2 | 0.874 |
| GBP5 | MAPKAPK3 | 0.874 |
| POLB | SRRM1 | 0.874 |
| P2RY14 | NFKB1 | 0.874 |
| GBP5 | RANBP10 | 0.874 |
| GBP5 | GCA | 0.874 |
| GBP5 | GCHFR | 0.874 |
| POLB | PER2 | 0.874 |
| P2RY14 | TBXA2R | 0.874 |
| GBP1 | SLC39A8 | 0.874 |
| DAPP1 | SLTM | 0.874 |
| GBP1 | MCM3 | 0.874 |
| CYBER3 | POLB | 0.874 |
| P2RY14 | TFRC | 0.874 |
| P2RY14 | CCDC25 | 0.874 |
| GBP5 | UCHL3 | 0.874 |
| APOL2 | NCL | 0.874 |
| UBE2L6 | NUMA1 | 0.874 |
| SECTM1 | VPS11 | 0.874 |
| P2RY14 | ZNF426 | 0.874 |
| POLB | MCM6 | 0.874 |
| P2RY14 | DCAKD | 0.874 |
| SECTM1 | NFE2L1 | 0.874 |
| GBP1 | LRRC8D | 0.874 |
| P2RY14 | SSRP1 | 0.874 |
| GBP5 | HPS6 | 0.874 |
| P2RY14 | WDR26 | 0.874 |
| UBE216 | UBAP2 | 0.874 |
| POLB | NUP50 | 0.874 |
| P2RY14 | PIP5K1C | 0.874 |
| P2RY14 | ITGB3BP | 0.874 |
| UBE2L6 | VAMP5 | 0.874 |
| P2RY14 | HCFC2 | 0.874 |
| SLC6A12 | P2RY14 | 0.874 |
| P2RY14 | PLCD1 | 0.874 |
| GBP5 | PLTP | 0.874 |
| P2RY14 | PLK3 | 0.874 |
| POLB | SYNCRIP | 0.874 |
| GBP5 | RFC3 | 0.874 |
| GBP5 | S100PBP | 0.874 |
| GBP5 | SP110 | 0.874 |
| POLB | TSEN2 | 0.874 |
| UBE216 | SMARCD3 | 0.874 |
| GBP1 | RAP2C | 0.874 |
| SECTM1 | PPP2R5D | 0.874 |
| GBP5 | APPBP2 | 0.874 |
| UBE2L6 | OSBP | 0.874 |
| GBP1 | FYCO1 | 0.874 |
| GBP5 | ACAT1 | 0.874 |
| LIMK1 | VAMP5 | 0.874 |
| P2RY14 | GOLGA7 | 0.874 |
| GBP5 | CSTF1 | 0.874 |
| GBP5 | GLRX2 | 0.874 |
| P2RY14 | NARS2 | 0.874 |
| P2RY14 | TEX261 | 0.874 |
| GBP5 | CLIC4 | 0.874 |
| SECTM1 | LAS1L | 0.874 |
| P2RY14 | PIGB | 0.874 |
| UBE2L6 | TERF2 | 0.874 |
| P2RY14 | GTPBP3 | 0.874 |
| ALDOC | P2RY14 | 0.874 |
| POLB | TMEM43 | 0.874 |
| TNFRSF14 | LHFPL2 | 0.874 |
| POLB | MYO9A | 0.874 |
| APOL2 | SSRP1 | 0.874 |
| UBE216 | ARID1A | 0.874 |
| GBP5 | CUTA | 0.874 |
| GBP5 | TTC12 | 0.874 |
| GBP5 | CYP4F12 | 0.874 |
| P2RY14 | DAPP1 | 0.874 |
| GBP5 | NUP93 | 0.874 |
| P2RY14 | FAM53C | 0.874 |
| GBP5 | OSBPL1A | 0.874 |
| P2RY14 | GMEB1 | 0.874 |
| GBP5 | KLHL12 | 0.874 |
| GBP5 | HDC | 0.874 |
| POLB | ARIH2 | 0.874 |
| GBP5 | RCN2 | 0.874 |
| UBE2L6 | MAX | 0.874 |
| POLB | PIK3C2B | 0.874 |
| GBP5 | ADRM1 | 0.874 |
| GBP5 | RNF24 | 0.874 |
| P2RY14 | ABCA2 | 0.874 |
| GBP5 | APH1B | 0.874 |
| P2RY14 | CACNA2D2 | 0.874 |
| TNFRSF14 | GBP1 | 0.874 |
| GBP5 | ZNF195 | 0.874 |
| P2RY14 | GSPT2 | 0.874 |
| GBP5 | IGHMBP2 | 0.874 |
| GBP5 | CIRBP | 0.874 |
| P2RY14 | PSMD11 | 0.874 |
| UBE2L6 | ACO1 | 0.874 |
| GBP5 | PPP2R1A | 0.874 |
| POLB | APBA2 | 0.874 |
| GBP5 | NOL6 | 0.874 |
| UBE2L6 | PSMB1 | 0.874 |
| GBP1 | UPF2 | 0.874 |
| P2RY14 | CBX4 | 0.874 |
| P2RY14 | EXOSC2 | 0.874 |
| GBP1 | NRG1 | 0.874 |
| GBP5 | CD300C | 0.874 |
| GBP5 | COPS4 | 0.874 |
| P2RY14 | IRF4 | 0.874 |
| ZNHIT1 | GBP1 | 0.874 |
| GSTO1 | UBE2L6 | 0.874 |
| UBE216 | TOB2 | 0.874 |
| GBP5 | CD53 | 0.874 |
| GBP5 | MSH3 | 0.874 |
| P2RY14 | SLBP | 0.874 |
| VAMP5 | CEP250 | 0.874 |
| VAMP5 | NCL | 0.874 |
| P2RY14 | PRKCH | 0.874 |
| GBP5 | BPHL | 0.874 |
| POLB | PSTPIP2 | 0.874 |
| P2RY14 | KLF10 | 0.874 |
| POLB | CASP6 | 0.874 |
| GBP5 | CBFA2T2 | 0.874 |
| P2RY14 | AFF3 | 0.874 |
| POLB | MBTPS1 | 0.874 |
| GBP5 | MRPS18C | 0.874 |
| GBP5 | ARSD | 0.874 |
| GBP5 | FBXW11 | 0.874 |
| GBP5 | SCP2 | 0.874 |
| SECTM1 | STAG1 | 0.874 |
| P2RY14 | CORO2A | 0.874 |
| P2RY14 | ZNF573 | 0.874 |
| POLB | PUM1 | 0.874 |
| SECTM1 | SLCO3A1 | 0.874 |
| POLB | NET1 | 0.874 |
| POLB | FOXJ2 | 0.874 |
| POLB | MAGED1 | 0.874 |
| POLB | PSMB2 | 0.874 |
| GBP5 | GPI | 0.874 |
| SECTM1 | NMT1 | 0.874 |
| GBP1 | AKAP1 | 0.874 |
| P2RY14 | SETD3 | 0.874 |
| POLB | NFE2L1 | 0.874 |
| POLB | TSFM | 0.874 |
| GBP5 | YIPF6 | 0.874 |
| AAMP | P2RY14 | 0.874 |
| P2RY14 | LEF1 | 0.874 |
| SECTM1 | TNK2 | 0.874 |
| P2RY14 | GTF3C2 | 0.874 |
| GBP5 | NAPG | 0.874 |
| POLB | TIMELESS | 0.874 |
| GBP5 | HSPA4 | 0.874 |
| GBP5 | GPR18 | 0.874 |
| P2RY14 | ANXA7 | 0.874 |
| P2RY14 | NPEPPS | 0.874 |
| P2RY14 | PUS7L | 0.874 |
| GBP1 | PPP2R5B | 0.874 |
| P2RY14 | IL1R1 | 0.874 |
| POLB | PON2 | 0.874 |
| P2RY14 | ICAM2 | 0.874 |
| UBE2L6 | DGKD | 0.874 |
| P2RY14 | OGDH | 0.874 |
| LHFPL2 | C1QA | 0.874 |
| GBP1 | SNAPC3 | 0.874 |
| CALCOCO2 | VAMP5 | 0.874 |
| GBP5 | TRPM2 | 0.874 |
| GBP5 | CASZ1 | 0.874 |
| SECTM1 | NCL | 0.874 |
| POLB | VPS35 | 0.874 |
| ACP5 | VAMP5 | 0.874 |
| PZRY14 | MOAP1 | 0.874 |
| GBP1 | DCTN4 | 0.874 |
| GBP1 | TOR1AIP1 | 0.874 |
| P2RY14 | SLC22A1 | 0.874 |
| GBP1 | NCL | 0.874 |
| GBP5 | SNX13 | 0.874 |
| GBP5 | SP2 | 0.874 |
| MAX | APOL2 | 0.874 |
| P2RY14 | VIPR1 | 0.873 |
| POLB | MORF4L1 | 0.873 |
| GBP5 | GP1BA | 0.873 |
| UBE2L6 | TNK2 | 0.873 |
| GBP5 | ATP6AP2 | 0.873 |
| GBP5 | CBFA2T3 | 0.873 |
| GBP5 | IL4R | 0.873 |
| POLB | ZNF318 | 0.873 |
| GBP5 | EIF4E | 0.873 |
| GBP5 | EXOSC7 | 0.873 |
| AIM2 | KLF6 | 0.873 |
| GBP5 | MTMR2 | 0.873 |
| SECTM1 | MAZ | 0.873 |
| P2RY14 | ZNF589 | 0.873 |
| GBP5 | ALDH6A1 | 0.873 |
| GBP5 | HCLS1 | 0.873 |
| GBP5 | MTHFD2 | 0.873 |
| NPC2 | LHFPL2 | 0.873 |
| GBP5 | SNRPB2 | 0.873 |
| PZRY14 | PPP2R5C | 0.873 |
| P2RY14 | TAF1C | 0.873 |
| VAMP5 | LIG1 | 0.873 |
| UBE2L6 | MYC | 0.873 |
| GBP5 | GUCA1B | 0.873 |
| DUSP3 | NDRG1 | 0.873 |
| P2RY14 | ACVR1 | 0.873 |
| P2RY14 | RECQL5 | 0.873 |
| GBP5 | MR1 | 0.873 |
| P2RY14 | PIK3R5 | 0.873 |
| GBP1 | BAHD1 | 0.873 |
| P2RY14 | EIF4G1 | 0.873 |
| PSME1 | GBP1 | 0.873 |
| GBP5 | RPP40 | 0.873 |
| GBP1 | TIMELESS | 0.873 |
| POLB | ZNF76 | 0.873 |
| GBP5 | MKRN1 | 0.873 |
| GBP1 | TPP2 | 0.873 |
| GBP5 | MYNN | 0.873 |
| POLB | CENPJ | 0.873 |
| SECTM1 | GEMIN4 | 0.873 |
| GBP1 | ZBTB16 | 0.873 |
| GBP5 | GTF2H1 | 0.873 |
| APOL2 | CTCF | 0.873 |
| ACP5 | AIM2 | 0.873 |
| LIMK1 | DGKD | 0.873 |
| POLB | MAPK13 | 0.873 |
| POLB | ZNF189 | 0.873 |
| APOL2 | ILF3 | 0.873 |
| CALCOCO2 | OSBP | 0.873 |
| GBP1 | CYB561 | 0.873 |
| P2RY14 | PXMP4 | 0.873 |
| GBP1 | PPP4R1 | 0.873 |
| POLB | BZW2 | 0.873 |
| P2RY14 | UBTF | 0.873 |
| GBP5 | VASH1 | 0.873 |
| GBP1 | APOL2 | 0.873 |
| GBP1 | NIPBL | 0.873 |
| SECTM1 | TERF2 | 0.873 |
| GBP5 | ITGAM | 0.873 |
| IL15RA | ATG3 | 0.873 |
| TMEM59 | GBP1 | 0.873 |
| SECTM1 | ELAC2 | 0.873 |
| POLB | PPAT | 0.873 |
| SECTM1 | STAT6 | 0.873 |
| P2RY14 | BRPF1 | 0.873 |
| SECTM1 | GANAB | 0.873 |
| UBE2L6 | GRWD1 | 0.873 |
| UBE2L6 | LHFPL2 | 0.873 |
| GBP5 | PRMT5 | 0.873 |
| C1QA | TSPAN14 | 0.873 |
| GBP1 | PRKAR2A | 0.873 |
| APOL2 | TTLL12 | 0.873 |
| MGST2 | P2RY14 | 0.873 |
| GBP5 | DUSP13 | 0.873 |
| SECTM1 | TMCC1 | 0.873 |
| GBP1 | HK2 | 0.873 |
| GBP5 | RXRA | 0.873 |
| P2RY14 | ETF1 | 0.873 |
| SECTM1 | ITGA5 | 0.873 |
| P2RY14 | PARP6 | 0.873 |
| P2RY14 | CAMK1D | 0.873 |
| GBP1 | ZNF189 | 0.873 |
| SECTM1 | PBXIP1 | 0.873 |
| P2RY14 | RAD23B | 0.873 |
| GBP5 | MEFV | 0.873 |
| POLB | ZHX2 | 0.873 |
| GBP5 | LCP1 | 0.873 |
| GBP1 | FBXW2 | 0.873 |
| P2RY14 | CDKSRAP3 | 0.873 |
| P2RY14 | CDC42 | 0.873 |
| GBP1 | MTSS1 | 0.873 |
| GBP5 | TANK | 0.873 |
| GBP5 | STYXL1 | 0.873 |
| GBP5 | ZFYVE16 | 0.873 |
| SECTM1 | LIG1 | 0.873 |
| P2RY14 | CDC34 | 0.873 |
| GBP5 | SLC27A3 | 0.873 |
| P2RY14 | MNAT1 | 0.873 |
| P2RY14 | PDK4 | 0.873 |
| GBP1 | PPP2R5A | 0.873 |
| GBP1 | TCF7 | 0.873 |
| P2RY14 | NRBP1 | 0.873 |
| GBP5 | DNMT3A | 0.873 |
| P2RY14 | USP4 | 0.873 |
| P2RY14 | CD96 | 0.873 |
| POLB | VIPR1 | 0.873 |
| POLB | BRD9 | 0.873 |
| SECTM1 | CIDEB | 0.873 |
| POLB | LRRC8D | 0.873 |
| P2RY14 | PRKAR1A | 0.873 |
| SECTM1 | PRDM2 | 0.873 |
| GBP5 | DBN1 | 0.873 |
| PZRY14 | PREP | 0.873 |
| GBP5 | DCTN2 | 0.873 |
| GBP1 | PRDM4 | 0.873 |
| POLB | BTN2A2 | 0.873 |
| GBP5 | PCDH9 | 0.873 |
| GBP5 | GALC | 0.873 |
| GBP1 | RFC1 | 0.873 |
| UBE2L6 | DHX9 | 0.873 |
| POLB | ZNF589 | 0.873 |
| GBP5 | JUNB | 0.873 |
| P2RY14 | NDUFA9 | 0.873 |
| P2RY14 | ASNS | 0.873 |
| P2RY14 | ARNTL | 0.873 |
| POLB | ITIHS | 0.873 |
| GBP5 | CEBPB | 0.873 |
| UBE2L6 | NUP210 | 0.873 |
| GBP1 | PIK3R1 | 0.873 |
| POLB | RER1 | 0.873 |
| GBP5 | GATAD2A | 0.873 |
| GBP5 | OSM | 0.873 |
| POLB | ABL1 | 0.873 |
| GBP1 | HERC1 | 0.873 |
| APOL1 | MAX | 0.873 |
| POLB | R3HDM2 | 0.873 |
| GBP5 | MARCO | 0.873 |
| GBP1 | SLC39A6 | 0.873 |
| POLB | TBRG4 | 0.873 |
| TMEM59 | APOL2 | 0.873 |
| POLB | EXOSC10 | 0.873 |
| LHFPL2 | HK2 | 0.873 |
| SECTM1 | ABCF2 | 0.873 |
| GBP5 | CUL1 | 0.873 |
| C1QA | SLC3A2 | 0.873 |
| P2RY14 | RNF130 | 0.873 |
| GBP5 | CYB5R4 | 0.873 |
| P2RY14 | CBLL1 | 0.873 |
| GBP1 | EHD4 | 0.873 |
| P2RY14 | MAP2K4 | 0.873 |
| GBP5 | MBD4 | 0.873 |
| P2RY14 | HTATIP2 | 0.873 |
| GBP5 | ATP6V1B2 | 0.873 |
| P2RY14 | ASB8 | 0.873 |
| PZRY14 | MBD1 | 0.873 |
| VAMP5 | CAPN2 | 0.873 |
| SECTM1 | PML | 0.873 |
| GBP1 | LRRFIP1 | 0.873 |
| PSME1 | PBXIP1 | 0.873 |
| POLB | VPS33B | 0.873 |
| UBE2L6 | ILF3 | 0.873 |
| P2RY14 | UBE2 | 0.873 |
| GBP1 | DDX27 | 0.873 |
| P2RY14 | CCT6A | 0.873 |
| POLB | CA5B | 0.873 |
| P2RY14 | CDCA4 | 0.873 |
| UBE2L6 | RPAP1 | 0.873 |
| P2RY14 | H6PD | 0.873 |
| GBP5 | MCRS1 | 0.873 |
| GBP5 | MPDU1 | 0.873 |
| GBP1 | TULP4 | 0.873 |
| GBP5 | MYBPH | 0.873 |
| GBP5 | RPS6KA4 | 0.873 |
| GBP5 | PTCD2 | 0.873 |
| P2RY14 | TES | 0.873 |
| P2RY14 | PDCD6IP | 0.873 |
| P2RY14 | PITPNC1 | 0.873 |
| PZRY14 | IFT122 | 0.873 |
| GBP5 | WDR77 | 0.873 |
| GBP1 | PTGER2 | 0.873 |
| AIM2 | UPB1 | 0.873 |
| GBP5 | ADK | 0.873 |
| P2RY14 | NFYC | 0.873 |
| P2RY14 | ATP6V1G1 | 0.873 |
| POLB | MAT2A | 0.873 |
| P2RY14 | ELP3 | 0.873 |
| GBP5 | ZNF384 | 0.873 |
| GBP5 | GYPA | 0.873 |
| SECTM1 | BTN2A2 | 0.873 |
| P2RY14 | SPATA2 | 0.873 |
| P2RY14 | UPB1 | 0.873 |
| GBP5 | IL23A | 0.873 |
| POLB | DDX54 | 0.873 |
| GBP5 | MLLT10 | 0.873 |
| GBP5 | MATK | 0.873 |
| UBE216 | RIN3 | 0.873 |
| GBP5 | FNBP1L | 0.873 |
| POLB | DDX23 | 0.873 |
| TMEM59 | IL15RA | 0.872 |
| UBE2L6 | RUNX2 | 0.872 |
| POLB | SLC38A1 | 0.872 |
| UBE2L3 | GBP1 | 0.872 |
| GBP5 | CREBL2 | 0.872 |
| P2RY14 | RBM22 | 0.872 |
| GBP5 | ACADVL | 0.872 |
| GBP5 | DFFB | 0.872 |
| P2RY14 | GAK | 0.872 |
| CALCOCO2 | C1QA | 0.872 |
| GBP5 | PCCA | 0.872 |
| UBE2L6 | TP53BP2 | 0.872 |
| GBP5 | GATAD1 | 0.872 |
| DPM2 | GBP1 | 0.872 |
| P2RY14 | UGP2 | 0.872 |
| ACP5 | DUSP3 | 0.872 |
| P2RY14 | HNMT | 0.872 |
| PSME1 | NCL | 0.872 |
| UBE2L6 | CPVL | 0.872 |
| POLD4 | GBP1 | 0.872 |
| GBP5 | MYB | 0.872 |
| P2RY14 | ABCC5 | 0.872 |
| PSME1 | SSRP1 | 0.872 |
| GBP5 | EXT2 | 0.872 |
| GBP1 | AP4E1 | 0.872 |
| ACP5 | ATG3 | 0.872 |
| POLB | EIF1 | 0.872 |
| SECTM1 | RANBP3 | 0.872 |
| UBE2L6 | VPS11 | 0.872 |
| GBP5 | DPM1 | 0.872 |
| LHFPL2 | NDRG1 | 0.872 |
| POLB | CTNNB1 | 0.872 |
| P2RY14 | RRAGC | 0.872 |
| GBP5 | TRAF6 | 0.872 |
| SECTM1 | DHCR7 | 0.872 |
| P2RY14 | MRPS27 | 0.872 |
| GBP1 | ST6GAL1 | 0.872 |
| P2RY14 | NADSYN1 | 0.872 |
| P2RY14 | STAT1 | 0.872 |
| POLB | FLNB | 0.872 |
| GBP1 | ITGA7 | 0.872 |
| GBP5 | PIN1 | 0.872 |
| SECTM1 | SNX11 | 0.872 |
| SECTM1 | POLG | 0.872 |
| P2RY14 | BAG3 | 0.872 |
| GBP1 | PSMB1 | 0.872 |
| GBP5 | ARNT | 0.872 |
| GBP5 | NFYB | 0.872 |
| GBP5 | RAB36 | 0.872 |
| P2RY14 | PCSK7 | 0.872 |
| GBP5 | TFB1M | 0.872 |
| GBP5 | CTDSP2 | 0.872 |
| P2RY14 | CHAF1B | 0.872 |
| P2RY14 | BET1 | 0.872 |
| LHFPL2 | ELF1 | 0.872 |
| POLB | RBM4B | 0.872 |
| P2RY14 | FCRL2 | 0.872 |
| P2RY14 | TP5313 | 0.872 |
| UBE2L6 | VILL | 0.872 |
| P2RY14 | ACTL6A | 0.872 |
| P2RY14 | TMEM123 | 0.872 |
| GBP5 | ARHGEF11 | 0.872 |
| P2RY14 | BANK1 | 0.872 |
| P2RY14 | ZFAND1 | 0.872 |
| P2RY14 | CD59 | 0.872 |
| UBE2L6 | AKAP1 | 0.872 |
| POLB | DDX46 | 0.872 |
| SECTM1 | FTSJ3 | 0.872 |
| POLB | EXOSC2 | 0.872 |
| GBP5 | TRIM62 | 0.872 |
| GBP1 | MAPK6 | 0.872 |
| P2RY14 | MFAP1 | 0.872 |
| GBP1 | MAP3K4 | 0.872 |
| UBE2L6 | BAHD1 | 0.872 |
| P2RY14 | ZNF212 | 0.872 |
| P2RY14 | CASP7 | 0.872 |
| P2RY14 | GLMN | 0.872 |
| P2RY14 | ADAM19 | 0.872 |
| POLB | MPI | 0.872 |
| PSME1 | LIG1 | 0.872 |
| PSME1 | POLR3B | 0.872 |
| GBP5 | HHEX | 0.872 |
| P2RY14 | HSPA5 | 0.872 |
| AIM2 | C1QA | 0.872 |
| GBP5 | AHCY | 0.872 |
| VAMP5 | MCM2 | 0.872 |
| GNG11 | LIMK1 | 0.872 |
| P2RY14 | THOC1 | 0.872 |
| POLB | ANK3 | 0.872 |
| GBP5 | TUSC2 | 0.872 |
| GBP5 | STUB1 | 0.872 |
| GBP1 | CAND1 | 0.872 |
| ACP5 | C1QA | 0.872 |
| POLB | DNAJB1 | 0.872 |
| GBP1 | CNOT8 | 0.872 |
| GBP1 | ILF3 | 0.872 |
| P2RY14 | MORF4L1 | 0.872 |
| POLB | ANXA7 | 0.872 |
| UBE2L6 | PLCG1 | 0.872 |
| P2RY14 | PPP3CC | 0.872 |
| GBP5 | MRPL3 | 0.872 |
| GBP5 | WDTC1 | 0.872 |
| GBP5 | LMNA | 0.872 |
| UBE216 | BTN2A2 | 0.872 |
| UBE216 | NFATC1 | 0.872 |
| APOL1 | ZBTB16 | 0.872 |
| C1QA | ATG3 | 0.872 |
| UBE2L6 | VCP | 0.872 |
| P2RY14 | FNTA | 0.872 |
| GBP5 | ZNF24 | 0.872 |
| P2RY14 | MAP3K14 | 0.872 |
| P2RY14 | ATP1B3 | 0.872 |
| P2RY14 | CYCS | 0.872 |
| GBP5 | PAPSS1 | 0.872 |
| P2RY14 | PEA15 | 0.872 |
| POLB | HERC1 | 0.872 |
| GBP1 | FAM3A | 0.872 |
| POLB | RUVBL1 | 0.872 |
| POLB | ATP9B | 0.872 |
| LHFPL2 | STAG1 | 0.872 |
| GBP1 | PRKCQ | 0.872 |
| P2RY14 | USP16 | 0.872 |
| NDUFB6 | SECTM1 | 0.872 |
| VAMP5 | GLG1 | 0.872 |
| GBP5 | P2RY6 | 0.872 |
| P2RY14 | MAP2K1 | 0.872 |
| POLB | ABLIM1 | 0.872 |
| APOL2 | DPF2 | 0.872 |
| P2RY14 | YIPF1 | 0.872 |
| SECTM1 | MYO9B | 0.872 |
| P2RY14 | CCT5 | 0.872 |
| GBP1 | ITPR3 | 0.872 |
| SECTM1 | XRCC1 | 0.872 |
| POLB | BCS1L | 0.872 |
| GBP5 | TLR6 | 0.872 |
| POLB | TCF12 | 0.872 |
| GBP5 | SLC25A11 | 0.872 |
| SECTM1 | TOB2 | 0.872 |
| GBP5 | TESK2 | 0.872 |
| P2RY14 | DGKA | 0.872 |
| SECTM1 | TLE4 | 0.872 |
| P2RY14 | SQLE | 0.872 |
| GBP5 | RNF11 | 0.872 |
| GBP1 | ANKRD27 | 0.872 |
| GBP1 | SS18 | 0.872 |
| GBP1 | NR1D2 | 0.872 |
| POLB | FXR2 | 0.872 |
| GALK1 | P2RY14 | 0.872 |
| GBP5 | PCMT1 | 0.872 |
| SECTM1 | SLC3A2 | 0.872 |
| P2RY14 | VPS11 | 0.872 |
| POLB | ETF1 | 0.872 |
| GBP5 | ITM2A | 0.872 |
| ATG3 | OSBPL9 | 0.872 |
| POLB | SAP130 | 0.872 |
| POLB | GTF3C3 | 0.872 |
| POLB | FBXL12 | 0.872 |
| P2RY14 | PDCD2 | 0.872 |
| VAMP5 | UQCRC1 | 0.872 |
| P2RY14 | PITRM1 | 0.872 |
| UBE216 | RNF122 | 0.872 |
| PZRY14 | BRD3 | 0.872 |
| GBP5 | IMPDH1 | 0.872 |
| GBP1 | SON | 0.872 |
| GBP1 | VPS4B | 0.872 |
| P2RY14 | MYH9 | 0.872 |
| GSTO1 | SECTM1 | 0.872 |
| P2RY14 | PDIA4 | 0.872 |
| P2RY14 | BZW2 | 0.872 |
| P2RY14 | PPIG | 0.872 |
| GBP5 | LAG3 | 0.872 |
| GBP1 | MAP4 | 0.872 |
| GBP1 | USP48 | 0.872 |
| GBP5 | PHF20L1 | 0.872 |
| SECTM1 | ACP1 | 0.872 |
| P2RY14 | BTN3A1 | 0.872 |
| TARBP2 | P2RY14 | 0.872 |
| POLB | ACTR8 | 0.872 |
| P2RY14 | PAOX | 0.872 |
| GBP5 | NUDCD3 | 0.872 |
| IL15RA | GBP1 | 0.872 |
| SDC1 | GBP1 | 0.872 |
| SECTM1 | SPEN | 0.872 |
| P2RY14 | ETFDH | 0.872 |
| GBP5 | MRPL24 | 0.872 |
| GBP5 | SH3BGRL | 0.872 |
| UBE216 | PLCD1 | 0.872 |
| GBP1 | TM9SF4 | 0.871 |
| C1QA | GLG1 | 0.871 |
| P2RY14 | TRIM46 | 0.871 |
| GBP5 | YPELS | 0.871 |
| P2RY14 | CNIH4 | 0.871 |
| GBP1 | PSMD5 | 0.871 |
| APOL2 | POLR3B | 0.871 |
| P2RY14 | FBXL12 | 0.871 |
| GBP5 | TRIM38 | 0.871 |
| POLB | CHAF1B | 0.871 |
| GBP5 | RSRC1 | 0.871 |
| POLB | MAGEH1 | 0.871 |
| POLB | AP4E1 | 0.871 |
| P2RY14 | INPP1 | 0.871 |
| SECTM1 | ELMO1 | 0.871 |
| P2RY14 | LRRN3 | 0.871 |
| DAPP1 | OSBPL11 | 0.871 |
| GBP1 | CEP250 | 0.871 |
| SECTM1 | MAP4 | 0.871 |
| LY86 | P2RY14 | 0.871 |
| CALCOCO | CTCF | 0.871 |
| 2 | | |
| POLB | HMG20A | 0.871 |
| P2RY14 | ITIHS | 0.871 |
| GBP5 | PFKFB3 | 0.871 |
| GBP1 | ZC3H7A | 0.871 |
| POLB | ARMC7 | 0.871 |
| GBP5 | SPATA20 | 0.871 |
| GBP5 | IL13RA1 | 0.871 |
| P2RY14 | MTMR9 | 0.871 |
| GBP1 | ATP2B4 | 0.871 |
| UBE2L6 | PPRC1 | 0.871 |
| PRKAG1 | P2RY14 | 0.871 |
| UBE2L6 | CHAF1B | 0.871 |
| APOL1 | LAS1L | 0.871 |
| C1QA | CEP250 | 0.871 |
| P2RY14 | ACTR2 | 0.871 |
| GBP5 | PAFAH1B3 | 0.871 |
| GBP5 | VAMP4 | 0.871 |
| APOL2 | PPP2R5D | 0.871 |
| GBP5 | SF3B1 | 0.871 |
| POLB | STAT6 | 0.871 |
| P2RY14 | AOC3 | 0.871 |
| P2RY14 | B4GALT3 | 0.871 |
| GBP5 | PSMC4 | 0.871 |
| P2RY14 | DPH2 | 0.871 |
| P2RY14 | HADHA | 0.871 |
| GBP5 | VAV2 | 0.871 |
| P2RY14 | DUSP11 | 0.871 |
| GBP5 | HBP1 | 0.871 |
| GBP5 | AAK1 | 0.871 |
| CLK3 | P2RY14 | 0.871 |
| POLB | GPR132 | 0.871 |
| PZRY14 | ATP6V0A1 | 0.871 |
| GBP1 | TRIB2 | 0.871 |
| SECTM1 | SDC1 | 0.871 |
| VAMP5 | APOL2 | 0.871 |
| GBP1 | TXK | 0.871 |
| GBP5 | CUEDC2 | 0.871 |
| GBP1 | BCR | 0.871 |
| GBP5 | BID | 0.871 |
| P2RY14 | SLAMF1 | 0.871 |
| GBP5 | ARRB2 | 0.871 |
| GBP1 | ADAM17 | 0.871 |
| GBP1 | STAG2 | 0.871 |
| POLB | TACC1 | 0.871 |
| GBP5 | GSTZ1 | 0.871 |
| P2RY14 | HARS2 | 0.871 |
| P2RY14 | DDX23 | 0.871 |
| GBP1 | MSN | 0.871 |
| UBE2L6 | SYNCRIP | 0.871 |
| P2RY14 | MAPRE2 | 0.871 |
| NPC2 | DAPP1 | 0.871 |
| POLB | TBCD | 0.871 |
| P2RY14 | ABCF2 | 0.871 |
| VAMP5 | LAS1L | 0.871 |
| P2RY14 | OSBPL8 | 0.871 |
| GBP5 | WDFY3 | 0.871 |
| P2RY14 | BRF2 | 0.871 |
| GBP1 | RBL2 | 0.871 |
| PLEK2 | GBP1 | 0.871 |
| GBP5 | EMG1 | 0.871 |
| GBP5 | IGF2BP3 | 0.871 |
| VAMP5 | PDHA1 | 0.871 |
| SECTM1 | VDAC3 | 0.871 |
| GBP5 | GPX7 | 0.871 |
| P2RY14 | MPHOSPH10 | 0.871 |
| UBE216 | PDHA1 | 0.871 |
| GBP5 | MRPS15 | 0.871 |
| POLB | XPNPEP1 | 0.871 |
| UBE216 | ABL1 | 0.871 |
| GBP1 | YTHDC1 | 0.871 |
| GBP1 | NUP155 | 0.871 |
| GBP5 | HAT1 | 0.871 |
| GBP1 | MALT1 | 0.871 |
| GBP5 | LSM5 | 0.871 |
| POLB | GPA33 | 0.871 |
| GBP5 | NFKBIE | 0.871 |
| UBE2L6 | DNAJC1 | 0.871 |
| GBP5 | SLC6A16 | 0.871 |
| GBP5 | TNFSF4 | 0.871 |
| UBE2L6 | SDC1 | 0.871 |
| P2RY14 | PPP1R2 | 0.871 |
| POLB | CASP1 | 0.871 |
| GAMT | P2RY14 | 0.871 |
| GBP5 | ZBTB17 | 0.871 |
| P2RY14 | TFB2M | 0.871 |
| UBE216 | NFE2L1 | 0.871 |
| GBP5 | NUBP2 | 0.871 |
| DAPP1 | GPBP1L1 | 0.871 |
| PSME1 | C1QA | 0.871 |
| P2RY14 | GGA1 | 0.871 |
| CPVL | GBP1 | 0.871 |
| GBP1 | SFXN1 | 0.871 |
| UBE2L6 | DUSP3 | 0.871 |
| GBP5 | PEX13 | 0.871 |
| UBE2L6 | ZBTB16 | 0.871 |
| GBP1 | GTPBP4 | 0.871 |
| POLB | YWHAZ | 0.871 |
| POLB | NFX1 | 0.871 |
| P2RY14 | RUNX3 | 0.871 |
| GBP5 | WAC | 0.871 |
| GBP5 | DHX8 | 0.871 |
| IRF1 | NDRG1 | 0.871 |
| UBE2L6 | EIF4G1 | 0.871 |
| LIMK1 | POLG | 0.871 |
| GBP5 | SIGLEC9 | 0.871 |
| POLB | TMEM39B | 0.871 |
| GBP1 | TFAM | 0.871 |
| GBP1 | NFE2L1 | 0.871 |
| GBP5 | SLC14A1 | 0.871 |
| GBP5 | TRIM21 | 0.871 |
| P2RY14 | CSDE1 | 0.871 |
| SECTM1 | TRAPPC3 | 0.871 |
| P2RY14 | AXIN1 | 0.871 |
| P2RY14 | PLEKHA1 | 0.871 |
| IL15RA | DAPP1 | 0.871 |
| PZRY14 | PKIA | 0.871 |
| P2RY14 | SMAD7 | 0.871 |
| P2RY14 | ARCN1 | 0.871 |
| POLB | IL15 | 0.871 |
| GBP1 | ARFGEF1 | 0.871 |
| GBP1 | POLR3B | 0.871 |
| UBE2L6 | ELAC2 | 0.871 |
| GBP5 | SNTA1 | 0.871 |
| GBP5 | DCTN3 | 0.871 |
| SECTM1 | MAK | 0.871 |
| GBP5 | CTNNA1 | 0.871 |
| GBP1 | GTF2E1 | 0.871 |
| GBP1 | MAT2A | 0.871 |
| SECTM1 | UBE4B | 0.871 |
| LHFPL2 | VAMP5 | 0.871 |
| P2RY14 | RABGGTB | 0.871 |
| GBP5 | AFF1 | 0.871 |
| GBP5 | GSK3B | 0.871 |
| GBP1 | DBR1 | 0.871 |
| P2RY14 | MUTYH | 0.871 |
| LHFPL2 | CAPN2 | 0.871 |
| GBP5 | KRT23 | 0.871 |
| GBP5 | STAT1 | 0.871 |
| GBP5 | ACAA1 | 0.871 |
| P2RY14 | ING1 | 0.871 |
| SECTM1 | TICAM1 | 0.871 |
| GBP5 | COMMD8 | 0.871 |
| GBP5 | NIT1 | 0.871 |
| APOL2 | LAS1L | 0.871 |
| P2RY14 | ACO1 | 0.871 |
| POLB | DNPEP | 0.871 |
| APOL2 | CTDP1 | 0.871 |
| GBP5 | CGGBP1 | 0.871 |
| POLB | RTN2 | 0.871 |
| PCBD1 | GBP1 | 0.871 |
| POLB | NUP155 | 0.871 |
| GBP5 | SOCS1 | 0.871 |
| PSME1 | GEMIN4 | 0.871 |
| GBP5 | IRAK1 | 0.871 |
| PZRY14 | LAMP2 | 0.871 |
| POLB | YIPF5 | 0.871 |
| PSME1 | ILF3 | 0.871 |
| VAMP5 | ATG3 | 0.871 |
| GBP5 | PLEK | 0.871 |
| GALE | P2RY14 | 0.871 |
| POLB | SAP30BP | 0.871 |
| GBP1 | ACAD8 | 0.871 |
| P2RY14 | COS | 0.871 |
| PZRY14 | TNFRSF9 | 0.871 |
| PZRY14 | PAK1IP1 | 0.871 |
| VAMP5 | SSRP1 | 0.871 |
| PML | BCR | 0.871 |
| GBP5 | PPP1R7 | 0.871 |
| GBP1 | RBBP4 | 0.871 |
| POLB | CPSF1 | 0.871 |
| POLB | ACOT7 | 0.871 |
| UBE2L6 | CHD4 | 0.87 |
| GBP5 | ATP2C1 | 0.87 |
| UBE2L6 | GMEB1 | 0.87 |
| GBP5 | DET1 | 0.87 |
| UBE216 | POLG | 0.87 |
| GBP5 | TSPAN13 | 0.87 |
| POLB | ITCH | 0.87 |
| P2RY14 | CLCF1 | 0.87 |
| GBP5 | TLR1 | 0.87 |
| P2RY14 | TSPAN3 | 0.87 |
| GBP5 | TLR8 | 0.87 |
| GBP5 | MKNK2 | 0.87 |
| POLB | KATNA1 | 0.87 |
| GBP1 | CCND2 | 0.87 |
| UBE2L6 | PRRG4 | 0.87 |
| GBP1 | STK38 | 0.87 |
| GBP5 | BCL2L1 | 0.87 |
| GBP5 | MRPL17 | 0.87 |
| APOL2 | OSBP | 0.87 |
| GBP1 | CBLB | 0.87 |
| GBP1 | AP1G1 | 0.87 |
| P2RY14 | SLC35F2 | 0.87 |
| SECTM1 | SAP30BP | 0.87 |
| GBP1 | NT5C2 | 0.87 |
| GBP5 | BANF1 | 0.87 |
| GBP5 | PABPN1 | 0.87 |
| GBP1 | MYCBP2 | 0.87 |
| SECTM1 | AP1B1 | 0.87 |
| POLB | RAB11B | 0.87 |
| SECTM1 | BAHD1 | 0.87 |
| AKR1A1 | GBP1 | 0.87 |
| NDUFB6 | POLB | 0.87 |
| APOL2 | MSN | 0.87 |
| P2RY14 | NARF | 0.87 |
| GBP1 | EPHA4 | 0.87 |
| P2RY14 | ADI1 | 0.87 |
| PF4V1 | GBP1 | 0.87 |
| P2RY14 | NISCH | 0.87 |
| GBP5 | DHTKD1 | 0.87 |
| POLB | RABEPK | 0.87 |
| GBP1 | RRN3 | 0.87 |
| GBP5 | HMBOX1 | 0.87 |
| P2RY14 | SMUG1 | 0.87 |
| P2RY14 | TSPYL2 | 0.87 |
| GBP5 | CKAP4 | 0.87 |
| COX6B1 | DAPP1 | 0.87 |
| GBP1 | ZNF318 | 0.87 |
| POLB | NR3C2 | 0.87 |
| P2RY14 | DOLPP1 | 0.87 |
| GBP1 | PRDM2 | 0.87 |
| LIMK1 | SSRP1 | 0.87 |
| POLB | NONO | 0.87 |
| P2RY14 | ADAT1 | 0.87 |
| GBP5 | MTX2 | 0.87 |
| GBP5 | TRIM23 | 0.87 |
| VAMP5 | TSPAN14 | 0.87 |
| POLB | CRNKL1 | 0.87 |
| GBP5 | IKBKB | 0.87 |
| POLB | BAIAP2 | 0.87 |
| SECTM1 | RPAP1 | 0.87 |
| SECTM1 | BCL2L11 | 0.87 |
| UBE2L6 | TCF7 | 0.87 |
| P2RY14 | RSAD1 | 0.87 |
| GBP1 | DENND2D | 0.87 |
| GBP5 | CNN2 | 0.87 |
| UBE216 | DHCR7 | 0.87 |
| GBP1 | XPC | 0.87 |
| P2RY14 | TAOK2 | 0.87 |
| P2RY14 | RAB11FIP3 | 0.87 |
| POLB | NXT1 | 0.87 |
| POLB | KLF2 | 0.87 |
| GBP5 | PLAGL1 | 0.87 |
| P2RY14 | ZNF211 | 0.87 |
| GBP5 | CRHR1 | 0.87 |
| P2RY14 | KIFC3 | 0.87 |
| F13A1 | P2RY14 | 0.87 |
| P2RY14 | FBLN5 | 0.87 |
| GBP5 | BIRC5 | 0.87 |
| GBP5 | DYNC1LI1 | 0.87 |
| UBE2L6 | NCOA1 | 0.87 |
| SECTM1 | OXSR1 | 0.87 |
| GBP1 | NUMA1 | 0.87 |
| UBE2L6 | RRS1 | 0.87 |
| GBP5 | TBP | 0.87 |
| LHFPL2 | OXSR1 | 0.87 |
| GBP1 | MBTPS1 | 0.87 |
| P2RY14 | COG4 | 0.87 |
| POLB | ZCCHC8 | 0.87 |
| UBE2L6 | TM9SF4 | 0.87 |
| LSM1 | POLB | 0.87 |
| ACP5 | APOL1 | 0.87 |
| P2RY14 | EML3 | 0.87 |
| PZRY14 | PNRC1 | 0.87 |
| POLB | MALT1 | 0.87 |
| AIM2 | SECTM1 | 0.87 |
| GBP5 | CAST | 0.87 |
| DAPP1 | HMGCR | 0.87 |
| P2RY14 | USP11 | 0.87 |
| GBP5 | FBXO2 | 0.87 |
| ALDOC | GBP1 | 0.87 |
| LAPTM4A | GBP1 | 0.87 |
| PSME1 | TERF2 | 0.87 |
| PSTPIP2 | SLCO4C1 | 0.87 |
| POLB | RBM15 | 0.87 |
| GBP1 | TRAF3 | 0.87 |
| POLB | NENF | 0.87 |
| GBP5 | LACTB2 | 0.87 |
| GBP5 | STRN4 | 0.87 |
| IL12RB1 | LHFPL2 | 0.87 |
| P2RY14 | NDFIP1 | 0.87 |
| GBP1 | MKRN2 | 0.87 |
| P2RY14 | GTPBP8 | 0.87 |
| TMEM59 | C1QA | 0.87 |
| P2RY14 | ZFP36L1 | 0.87 |
| P2RY14 | TBC1D1 | 0.87 |
| P2RY14 | STIM1 | 0.87 |
| GBP1 | POLG | 0.87 |
| GBP5 | XAB2 | 0.87 |
| GBP5 | NDUFC1 | 0.87 |
| CASP1 | OSBPL9 | 0.87 |
| UBE2L6 | SERPINB8 | 0.87 |
| UBE216 | ITPKB | 0.87 |
| P2RY14 | TBX19 | 0.87 |
| GBP5 | UNC93B1 | 0.87 |
| SECTM1 | PPARD | 0.87 |
| UBE2L6 | SPEN | 0.87 |
| LHFPL2 | RPS14 | 0.87 |
| P2RY14 | CYP20A1 | 0.87 |
| GBP1 | POGZ | 0.87 |
| APOL2 | MAPK13 | 0.87 |
| GBP5 | DHRS1 | 0.87 |
| P2RY14 | PUS1 | 0.87 |
| DAD1 | GBP1 | 0.87 |
| APOL2 | PPARD | 0.87 |
| POLB | EIF2B5 | 0.87 |
| UBE2L6 | MAP3K4 | 0.87 |
| MAX | VAMP5 | 0.87 |
| GBP5 | SRP19 | 0.87 |
| GBP1 | PAXIP1 | 0.87 |
| GBP5 | AFG3L2 | 0.87 |
| SECTM1 | SETD3 | 0.87 |
| RELB | P2RY14 | 0.87 |
| GBP1 | BCOR | 0.87 |
| PSME1 | DDX27 | 0.87 |
| VAMP5 | MCM5 | 0.87 |
| PRRG4 | SECTM1 | 0.87 |
| P2RY14 | TRAF3IP3 | 0.87 |
| GBP5 | ETFA | 0.87 |
| TMEM39B | GBP1 | 0.87 |
| LHFPL2 | MAK | 0.87 |
| VAMP5 | DDX27 | 0.87 |
| P2RY14 | DDIT3 | 0.87 |
| APOL2 | XPC | 0.87 |
| P2RY14 | ZNF330 | 0.87 |
| GBP5 | BCAP31 | 0.87 |
| GBP5 | SAP30 | 0.87 |
| POLB | KHDRBS1 | 0.87 |
| GBP1 | PTPN11 | 0.87 |
| GBP5 | SETDB1 | 0.87 |
| GBP1 | RFWD3 | 0.87 |
| GBP1 | TCF12 | 0.87 |
| SECTM1 | PKNOX1 | 0.87 |
| P2RY14 | RAB8B | 0.87 |
| GBP5 | NKIRAS2 | 0.87 |
| P2RY14 | NDUFS4 | 0.87 |
| LHFPL2 | CORO1C | 0.87 |
| GBP1 | DENND4A | 0.87 |
| POLB | TTC19 | 0.87 |
| PZRY14 | IFT57 | 0.87 |
| GBP5 | TRPS1 | 0.87 |
| INPPL1 | P2RY14 | 0.87 |
| GBP1 | BAX | 0.87 |
| GBP1 | OXCT1 | 0.87 |
| GBP1 | KHDRBS1 | 0.87 |
| PSME1 | OSBP | 0.87 |
| P2RY14 | CLEC4A | 0.87 |
| P2RY14 | SETBP1 | 0.87 |
| UBE2L6 | STK38 | 0.87 |
| POLB | PRKCQ | 0.87 |
| LHFPL2 | TMSB10 | 0.87 |
| GBP1 | PLAA | 0.87 |
| GBP5 | TCF20 | 0.87 |
| GBP5 | SAR1B | 0.87 |
| GBP5 | CD84 | 0.87 |
| GBP5 | ZNF302 | 0.87 |
| SECTM1 | TPD52L2 | 0.87 |
| UBE216 | RNGTT | 0.87 |
| GBP5 | TIMM9 | 0.87 |
| UBE2L6 | SNX11 | 0.87 |
| POLB | FBXO21 | 0.87 |
| P2RY14 | GRWD1 | 0.87 |
| GBP1 | RRAGC | 0.87 |
| GBP5 | MAST2 | 0.87 |
| PSME1 | UBE216 | 0.87 |
| GBP1 | KLF13 | 0.87 |
| C1QA | FAS | 0.87 |
| SECTM1 | GRWD1 | 0.87 |
| PSTPIP2 | KLF6 | 0.87 |
| GBP1 | UBAP2 | 0.87 |
| GBP1 | ATP2A2 | 0.87 |
| GBP5 | KRT10 | 0.87 |
| C1QA | MAX | 0.87 |
| SECTM1 | CCDC22 | 0.87 |
| POLB | CBX1 | 0.87 |
| P2RY14 | PTGS1 | 0.87 |
| GBP1 | CTNNB1 | 0.87 |
| P2RY14 | TMC6 | 0.87 |
| C1QA | CYB561 | 0.87 |
| PSME1 | POLG | 0.87 |
| P2RY14 | PPP2CB | 0.869 |
| P2RY14 | UNC45A | 0.869 |
| VAMP5 | MCM3 | 0.869 |
| AIM2 | APOL1 | 0.869 |
| UBE216 | MAK | 0.869 |
| C1QA | LIG1 | 0.869 |
| UBE2L6 | ERBB2 | 0.869 |
| P2RY14 | GALNT2 | 0.869 |
| SECTM1 | GNB1 | 0.869 |
| POLB | CDYL | 0.869 |
| P2RY14 | CIC | 0.869 |
| SECTM1 | HMGCR | 0.869 |
| P2RY14 | DOCK2 | 0.869 |
| GBP1 | TXN2 | 0.869 |
| UBE216 | RER1 | 0.869 |
| POLB | RASSF1 | 0.869 |
| PZRY14 | ITPA | 0.869 |
| PZRY14 | TXNL4B | 0.869 |
| GBP5 | IMPAD1 | 0.869 |
| GBP5 | VGLL4 | 0.869 |
| GBP1 | SMAD3 | 0.869 |
| UBE2L6 | FBXW2 | 0.869 |
| GBP5 | FHL2 | 0.869 |
| GBP5 | MLC1 | 0.869 |
| TRIM21 | P2RY14 | 0.869 |
| LHFPL2 | TP53BP2 | 0.869 |
| GBP1 | PPRC1 | 0.869 |
| GNG11 | L15RA | 0.869 |
| UBE2L6 | HYOU1 | 0.869 |
| GBP5 | HPCAL1 | 0.869 |
| APOL2 | TSR1 | 0.869 |
| PZRY14 | WDR19 | 0.869 |
| SECTM1 | STK38 | 0.869 |
| GBP5 | RFK | 0.869 |
| POLB | TOPBP1 | 0.869 |
| P2RY14 | PINK1 | 0.869 |
| UBE2L6 | CAMK4 | 0.869 |
| GBP5 | LRPAP1 | 0.869 |
| GBP1 | STT3A | 0.869 |
| UBE2L6 | RAPGEF1 | 0.869 |
| UBE2L6 | POLR3B | 0.869 |
| GBP5 | LILRB5 | 0.869 |
| GBP5 | YIF1A | 0.869 |
| GBP1 | UBP1 | 0.869 |
| P2RY14 | DTX3 | 0.869 |
| SECTM1 | IL15 | 0.869 |
| PZRY14 | CORO1C | 0.869 |
| ACP5 | IL15RA | 0.869 |
| GBP5 | RSU1 | 0.869 |
| AIM2 | ELF1 | 0.869 |
| UBE2L6 | STAG1 | 0.869 |
| GBP5 | MARCKSL1 | 0.869 |
| POLB | RRM1 | 0.869 |
| POLB | NPAT | 0.869 |
| GBP5 | SNRPB | 0.869 |
| P2RY14 | SLA | 0.869 |
| POLB | TCFL5 | 0.869 |
| P2RY14 | TPM3 | 0.869 |
| GNG11 | STAT1 | 0.869 |
| GBP1 | CANX | 0.869 |
| P2RY14 | PTS | 0.869 |
| SECTM1 | GPBP1L1 | 0.869 |
| GBP1 | PAPOLA | 0.869 |
| SECTM1 | TIAL1 | 0.869 |
| GBP1 | GLG1 | 0.869 |
| GBP1 | ATP8B2 | 0.869 |
| GBP5 | MICAL2 | 0.869 |
| GBP5 | KBTBD4 | 0.869 |
| POLB | TMED3 | 0.869 |
| P2RY14 | JOSD1 | 0.869 |
| SECTM1 | PPP3CA | 0.869 |
| GBP5 | ADIPOR1 | 0.869 |
| PSME1 | SMARCC1 | 0.869 |
| SECTM1 | FXR2 | 0.869 |
| WDR13 | GBP1 | 0.869 |
| GBP1 | PARP16 | 0.869 |
| SECTM1 | ZZEF1 | 0.869 |
| GBP5 | POLR3F | 0.869 |
| GBP5 | GNG5 | 0.869 |
| PRRG4 | LIMK1 | 0.869 |
| REEP4 | GBP1 | 0.869 |
| APOL1 | VAMP5 | 0.869 |
| GBP1 | CALU | 0.869 |
| GBP1 | RAPGEF1 | 0.869 |
| GBP1 | NCBP2 | 0.869 |
| GBP1 | TNPO1 | 0.869 |
| GBP1 | KPNA6 | 0.869 |
| CD74 | GBP1 | 0.869 |
| P2RY14 | TSFM | 0.869 |
| GBP5 | SMYD2 | 0.869 |
| POLB | ELAVL1 | 0.869 |
| GBP1 | RNF111 | 0.869 |
| GBP1 | SAP18 | 0.869 |
| P2RY14 | TAL1 | 0.869 |
| GBP5 | ITM2C | 0.869 |
| UBE2L6 | EIF1 | 0.869 |
| GBP5 | CRYL1 | 0.869 |
| P2RY14 | ALOX12 | 0.869 |
| P2RY14 | GLUL | 0.869 |
| P2RY14 | NMT1 | 0.869 |
| LHFPL2 | ANKS1A | 0.869 |
| GBP5 | CARD8 | 0.869 |
| GBP1 | NOC3L | 0.869 |
| GBP5 | TNFSF10 | 0.869 |
| UBE2L6 | OSBPL9 | 0.869 |
| GBP1 | SEC23B | 0.869 |
| POLB | GTF3C2 | 0.869 |
| VAMP5 | RBM28 | 0.869 |
| GBP5 | SNTB1 | 0.869 |
| P2RY14 | ECD | 0.869 |
| POLB | EHBP1 | 0.869 |
| UBE2L6 | PRDM2 | 0.869 |
| UBE216 | DPF2 | 0.869 |
| POLB | MBD2 | 0.869 |
| GBP5 | OSTM1 | 0.869 |
| GBP1 | KLF11 | 0.869 |
| SECTM1 | TBC1D13 | 0.869 |
| GBP5 | RBM5 | 0.869 |
| GBP1 | GEMIN4 | 0.869 |
| GBP5 | CLPP | 0.869 |
| GBP5 | EZH1 | 0.869 |
| SECTM1 | PTPN2 | 0.869 |
| GBP1 | USP47 | 0.869 |
| P2RY14 | SLC43A3 | 0.869 |
| P2RY14 | ISOC1 | 0.869 |
| GBP5 | LARP1 | 0.869 |
| GBP5 | NEDD4 | 0.869 |
| POLB | BSDC1 | 0.869 |
| GBP5 | AKAP7 | 0.869 |
| UBE216 | FTSJ3 | 0.869 |
| VAMP5 | GRWD1 | 0.869 |
| LHFPL2 | UPB1 | 0.869 |
| GBP1 | PIGK | 0.869 |
| GBP5 | SS18L1 | 0.869 |
| GBP5 | PIGG | 0.869 |
| GBP5 | TTLL5 | 0.869 |
| GBP1 | HIF1A | 0.869 |
| UBE2L6 | GPBP1L1 | 0.869 |
| SECTM1 | ULK1 | 0.869 |
| POLB | RPA1 | 0.869 |
| P2RY14 | PARP3 | 0.869 |
| GBP1 | APBA2 | 0.869 |
| UBE216 | ELMO1 | 0.869 |
| POLB | AQP3 | 0.869 |
| GBP5 | YME1L1 | 0.869 |
| P2RY14 | ZNF7 | 0.869 |
| C1QA | LAS1L | 0.869 |
| P2RY14 | TBCD | 0.869 |
| GBP5 | MFNG | 0.869 |
| P2RY14 | VPS39 | 0.869 |
| GBP5 | SLK | 0.869 |
| PML | CEP250 | 0.869 |
| GBP5 | CCNL1 | 0.869 |
| P2RY14 | EIF2B5 | 0.869 |
| POLB | SERINC3 | 0.869 |
| GBP5 | FBX011 | 0.869 |
| P2RY14 | RP2 | 0.869 |
| GBP5 | CD72 | 0.869 |
| SECTM1 | NUMA1 | 0.869 |
| UBE2L6 | LAS1L | 0.869 |
| POLB | ETS1 | 0.869 |
| POLB | FZD1 | 0.869 |
| LHFPL2 | PSMB1 | 0.869 |
| SECTM1 | TLE3 | 0.869 |
| P2RY14 | IFRD1 | 0.869 |
| GBP5 | NEDD9 | 0.869 |
| GBP5 | G6PC3 | 0.869 |
| PZRY14 | SLC25A13 | 0.869 |
| GBP5 | YWHAH | 0.869 |
| GBP5 | INSM1 | 0.869 |
| POLB | OSBPL2 | 0.869 |
| GBP1 | SARIA | 0.869 |
| GBP5 | PLCB1 | 0.869 |
| GBP1 | DDIT4 | 0.869 |
| POLB | UBAP2 | 0.869 |
| GBP5 | AUP1 | 0.869 |
| P2RY14 | RYBP | 0.869 |
| UBE216 | OGFOD1 | 0.869 |
| P2RY14 | HPS1 | 0.869 |
| POLB | POLR1D | 0.869 |
| NDUFB6 | C1QA | 0.869 |
| UBE216 | PHF3 | 0.869 |
| GBP5 | TBK1 | 0.869 |
| GBP5 | PGD | 0.869 |
| PZRY14 | WIP12 | 0.869 |
| P2RY14 | VCP | 0.869 |
| PSMA4 | P2RY14 | 0.869 |
| GBP1 | RTN2 | 0.869 |
| P2RY14 | DHDDS | 0.869 |
| GBP1 | ALMS1 | 0.869 |
| UBE2L6 | GTPBP4 | 0.869 |
| UBE2L6 | UBIAD1 | 0.869 |
| P2RY14 | EXOSC9 | 0.869 |
| SECTM1 | SMU1 | 0.869 |
| GBP5 | SPTLC1 | 0.869 |
| GBP5 | RRAGA | 0.869 |
| VAMP5 | TTLL12 | 0.869 |
| GBP5 | PMP22 | 0.869 |
| PSME1 | LIMK1 | 0.869 |
| GBP1 | DHX15 | 0.869 |
| C1QA | TTLL12 | 0.869 |
| POLB | CBX4 | 0.869 |
| P2RY14 | DHCR7 | 0.869 |
| POLB | PITRM1 | 0.869 |
| P2RY14 | ZNF232 | 0.869 |
| P2RY14 | TFG | 0.869 |
| POLB | PIGK | 0.869 |
| P2RY14 | ARL2BP | 0.869 |
| GBP5 | PSMD13 | 0.869 |
| ATP7B | P2RY14 | 0.869 |
| GBP1 | DNAJA3 | 0.869 |
| SECTM1 | CIC | 0.869 |
| LHFPL2 | HIGD2A | 0.869 |
| GBP5 | SPTBN4 | 0.869 |
| VAMP5 | NOLC1 | 0.869 |
| GBP1 | ASXL2 | 0.868 |
| GBP5 | PHTF1 | 0.868 |
| GBP5 | SUB1 | 0.868 |
| PSME1 | MAZ | 0.868 |
| POLB | CWF19L1 | 0.868 |
| P2RY14 | KLHL2 | 0.868 |
| GBP5 | DPYSL2 | 0.868 |
| C1QA | NCL | 0.868 |
| SECTM1 | FAS | 0.868 |
| POLB | TMEM87A | 0.868 |
| APOL2 | AKAP1 | 0.868 |
| VAMP5 | DIAPH1 | 0.868 |
| SECTM1 | DNAJC1 | 0.868 |
| GBP5 | GOLGA1 | 0.868 |
| SECTM1 | ITPKC | 0.868 |
| GBP5 | TLK2 | 0.868 |
| POLB | SNX11 | 0.868 |
| GBP1 | MAX | 0.868 |
| GBP5 | CTRL | 0.868 |
| P2RY14 | P2RY10 | 0.868 |
| GBP1 | UBE4A | 0.868 |
| APOL2 | DIAPH1 | 0.868 |
| POLB | MYO9B | 0.868 |
| GBP1 | PIK3C2B | 0.868 |
| P2RY14 | METAP2 | 0.868 |
| P2RY14 | TNFSF8 | 0.868 |
| GBP5 | SDC4 | 0.868 |
| GBP5 | FIBP | 0.868 |
| GBP1 | PDIA4 | 0.868 |
| SECTM1 | SERPINB8 | 0.868 |
| P2RY14 | TTLL12 | 0.868 |
| GBP5 | ETFB | 0.868 |
| PML | DIAPH1 | 0.868 |
| GBP5 | PSMD8 | 0.868 |
| GBP5 | EGLN1 | 0.868 |
| APOL3 | NCL | 0.868 |
| GBP5 | TMEM39A | 0.868 |
| GBP5 | NEK3 | 0.868 |
| P2RY14 | CDV3 | 0.868 |
| IL15RA | TSPAN14 | 0.868 |
| P2RY14 | VPS33A | 0.868 |
| DAPP1 | DGKD | 0.868 |
| GBP5 | RARA | 0.868 |
| IL12RB1 | GBP1 | 0.868 |
| POLB | DDX50 | 0.868 |
| UBE216 | RGS10 | 0.868 |
| P2RY14 | PMM2 | 0.868 |
| GBP5 | KLF4 | 0.868 |
| SECTM1 | MYC | 0.868 |
| GBP1 | GALNT1 | 0.868 |
| PZRY14 | BRD9 | 0.868 |
| GBP5 | MADD | 0.868 |
| GBP1 | SIDT1 | 0.868 |
| SAMHD1 | P2RY14 | 0.868 |
| UBE2L6 | ANKS1A | 0.868 |
| SECTM1 | STT3A | 0.868 |
| P2RY14 | PTPN2 | 0.868 |
| APOL2 | ZBTB16 | 0.868 |
| SECTM1 | EXOC7 | 0.868 |
| GBP1 | SLC30A5 | 0.868 |
| LIMK1 | CEP250 | 0.868 |
| POLB | PRKAR2A | 0.868 |
| TMEM59 | VAMP5 | 0.868 |
| SECTM1 | HS1BP3 | 0.868 |
| GBP1 | MAPK13 | 0.868 |
| GBP5 | B4GALT5 | 0.868 |
| GBP5 | CAMK1 | 0.868 |
| POLB | ASB1 | 0.868 |
| P2RY14 | PLAUR | 0.868 |
| GBP5 | RPA3 | 0.868 |
| GBP1 | BCL2L11 | 0.868 |
| P2RY14 | HEBP1 | 0.868 |
| PSME1 | TTLL12 | 0.868 |
| P2RY14 | PRPF4 | 0.868 |
| P2RY14 | TFPI | 0.868 |
| APOL2 | STK38 | 0.868 |
| POLB | RBBP7 | 0.868 |
| POLB | MAK | 0.868 |
| GBP5 | MRPL2 | 0.868 |
| GBP5 | BAG4 | 0.868 |
| POLB | PHYH | 0.868 |
| P2RY14 | SMNDC1 | 0.868 |
| GBP1 | UPB1 | 0.868 |
| GBP5 | MAN281 | 0.868 |
| P2RY14 | PSMD10 | 0.868 |
| GBP5 | GART | 0.868 |
| GBP5 | LIG3 | 0.868 |
| GBP1 | RAB11B | 0.868 |
| GBP1 | TGFBRAP1 | 0.868 |
| LHFPL2 | PFDN5 | 0.868 |
| P2RY14 | TAF6 | 0.868 |
| TRAPPC3 | APOL2 | 0.868 |
| POLB | FZD5 | 0.868 |
| GBP5 | CDC37 | 0.868 |
| POLB | ARHGEF7 | 0.868 |
| POLB | PRPSAP1 | 0.868 |
| P2RY14 | TRIT1 | 0.868 |
| P2RY14 | PXN | 0.868 |
| POLB | RFWD3 | 0.868 |
| POLB | EIF4ENIF1 | 0.868 |
| SECTM1 | CTBP2 | 0.868 |
| UBE2L6 | KYNU | 0.868 |
| SECTM1 | CLN6 | 0.868 |
| FAS | VAMP5 | 0.868 |
| GBP5 | BCAS4 | 0.868 |
| GBP5 | BICD2 | 0.868 |
| GBP5 | HEBP2 | 0.868 |
| GBP5 | ENO1 | 0.868 |
| POLB | TMSB10 | 0.868 |
| LHFPL2 | ADAM17 | 0.868 |
| GBP5 | LAPTM4B | 0.868 |
| S100A9 | GBP1 | 0.868 |
| GBP1 | SLBP | 0.868 |
| SECTM1 | DNM2 | 0.868 |
| GBP5 | ULK2 | 0.868 |
| P2RY14 | DDX24 | 0.868 |
| UBE216 | GOLGA3 | 0.868 |
| GBP5 | LY96 | 0.868 |
| P2RY14 | RCL1 | 0.868 |
| UBE2L6 | PSMD11 | 0.868 |
| AIM2 | LIMK1 | 0.868 |
| GBP1 | TMEM43 | 0.868 |
| UBE2L6 | ABCF2 | 0.868 |
| LHFPL2 | MCM2 | 0.868 |
| IL15RA | VAMP5 | 0.868 |
| GBP1 | ITPR1 | 0.868 |
| GBP1 | RACGAP1 | 0.868 |
| POLB | KNTC1 | 0.868 |
| P2RY14 | CTSS | 0.868 |
| P2RY14 | WWC3 | 0.868 |
| GBP5 | ALOXSAP | 0.868 |
| C1QA | STAT1 | 0.868 |
| P2RY14 | RNF146 | 0.868 |
| DAPP1 | RUNX2 | 0.868 |
| GBP5 | GZMB | 0.868 |
| SECTM1 | MECP2 | 0.868 |
| P2RY14 | GGA2 | 0.868 |
| P2RY14 | SRP54 | 0.868 |
| P2RY14 | STXBP3 | 0.868 |
| POLB | ST6GAL1 | 0.868 |
| GBP1 | LRIG1 | 0.868 |
| P2RY14 | PRMT7 | 0.868 |
| BLCAP | P2RY14 | 0.868 |
| POLB | TAF6 | 0.868 |
| P2RY14 | APTX | 0.868 |
| GBP1 | ZNF76 | 0.868 |
| P2RY14 | CD244 | 0.868 |
| POLB | PHF1 | 0.868 |
| GBP1 | ZNF236 | 0.868 |
| P2RY14 | DPF2 | 0.868 |
| P2RY14 | CHFR | 0.868 |
| POLB | STK4 | 0.868 |
| GBP5 | GCC1 | 0.868 |
| P2RY14 | PDIA6 | 0.868 |
| POLB | SLC25A22 | 0.868 |
| GBP5 | MDH1 | 0.868 |
| GBP1 | NFKB1 | 0.868 |
| GBP1 | SIRT5 | 0.868 |
| GBP5 | TEX264 | 0.868 |
| GBP5 | TP53BP1 | 0.868 |
| GBP1 | ZNF365 | 0.868 |
| POLB | HSF2 | 0.868 |
| GBP1 | EP300 | 0.868 |
| POLB | CASP2 | 0.868 |
| POLB | NGLY1 | 0.868 |
| P2RY14 | MAP4K4 | 0.868 |
| VAMP5 | ZBTB16 | 0.868 |
| P2RY14 | GPR132 | 0.868 |
| GBP5 | MAP6D1 | 0.868 |
| P2RY14 | NFE2 | 0.868 |
| SECTM1 | RPN1 | 0.868 |
| GBP1 | ERCC3 | 0.868 |
| GBP1 | CDC23 | 0.868 |
| LHFPL2 | PPP3CA | 0.868 |
| SECTM1 | PPP2R5B | 0.868 |
| GBP5 | RAD17 | 0.868 |
| UBE2L6 | PTPN2 | 0.868 |
| GBP5 | PSMC3 | 0.868 |
| CYB561 | VAMP5 | 0.868 |
| GBP1 | REV3L | 0.868 |
| P2RY14 | ELMO1 | 0.868 |
| GBP5 | PFDN1 | 0.868 |
| GBP1 | MTR | 0.868 |
| PML | TSPAN14 | 0.868 |
| SECTM1 | MAT2B | 0.868 |
| UBE216 | EXOSC10 | 0.868 |
| GBP1 | RAB14 | 0.868 |
| POLB | ZNF274 | 0.868 |
| P2RY14 | PFKP | 0.868 |
| POLB | ADAM19 | 0.868 |
| VAMP5 | RIN3 | 0.868 |
| PSME1 | NOLC1 | 0.868 |
| GBP5 | PFKFB4 | 0.868 |
| LIMK1 | PPP2R5D | 0.868 |
| IRF1 | C1QA | 0.868 |
| UBE2L6 | DUSP22 | 0.868 |
| ATG3 | STAG1 | 0.868 |
| APOL1 | C1QA | 0.868 |
| GBP5 | SRRM2 | 0.868 |
| PSME1 | PRMT7 | 0.868 |
| LHFPL2 | DCTN4 | 0.868 |
| GBP5 | SEMA4D | 0.868 |
| P2RY14 | ERCC2 | 0.868 |
| DAPP1 | APOL2 | 0.868 |
| P2RY14 | DDB1 | 0.868 |
| GBP1 | PFKP | 0.868 |
| GBP5 | FKBP14 | 0.868 |
| CASP1 | NDRG1 | 0.868 |
| POLB | SNAPC4 | 0.868 |
| GNG11 | AIM2 | 0.868 |
| P2RY14 | CPSF1 | 0.868 |
| PZRY14 | BUB3 | 0.868 |
| C1QA | VILL | 0.868 |
| C1QA | MCM5 | 0.868 |
| GBP5 | CORO1A | 0.868 |
| GBP1 | ETS2 | 0.868 |
| GBP1 | FOXJ2 | 0.868 |
| SECTM1 | NCOR1 | 0.868 |
| GBP5 | IQCB1 | 0.868 |
| P2RY14 | MBD2 | 0.868 |
| GBP5 | VENTX | 0.868 |
| GBP5 | PRKCE | 0.868 |
| UBE2L6 | STT3A | 0.868 |
| GBP1 | ENSA | 0.868 |
| LHFPL2 | DGKD | 0.868 |
| VAMP5 | CHAF1B | 0.868 |
| GBP5 | SNX27 | 0.868 |
| GBP5 | KCNJ2 | 0.868 |
| POLB | SLC2A4RG | 0.868 |
| VSIG4 | P2RY14 | 0.867 |
| UBE2L6 | CASP1 | 0.867 |
| GBP1 | RPS6KA3 | 0.867 |
| GBP1 | SNRK | 0.867 |
| C1QA | PDHA1 | 0.867 |
| GBP1 | ZMPSTE24 | 0.867 |
| POLB | RPS6KA3 | 0.867 |
| POLB | CDC23 | 0.867 |
| P2RY14 | UBE2N | 0.867 |
| GBP1 | SERINC3 | 0.867 |
| UBE216 | PTP4A1 | 0.867 |
| GBP1 | CHAF1B | 0.867 |
| GBP1 | RAB11FIP2 | 0.867 |
| MDK | GBP1 | 0.867 |
| P2RY14 | LETMD1 | 0.867 |
| UBE2L6 | RRM1 | 0.867 |
| P2RY14 | CASP10 | 0.867 |
| GBP5 | RAD50 | 0.867 |
| POLB | TUBD1 | 0.867 |
| LHFPL2 | RBM28 | 0.867 |
| P2RY14 | RPL39L | 0.867 |
| C1QA | PSTPIP2 | 0.867 |
| P2RY14 | TNIP1 | 0.867 |
| GBP1 | DNPEP | 0.867 |
| PZRY14 | CCNC | 0.867 |
| GBP5 | SOCS5 | 0.867 |
| UBE2L6 | SIRT5 | 0.867 |
| P2RY14 | XYLT2 | 0.867 |
| P2RY14 | POLR2D | 0.867 |
| POLB | OVGP1 | 0.867 |
| P2RY14 | DAAM2 | 0.867 |
| SECTM1 | LSR | 0.867 |
| UBE2L6 | SMAD3 | 0.867 |
| GBP1 | PPM1D | 0.867 |
| CALCOCO2 | LIMK1 | 0.867 |
| GBP5 | ACO2 | 0.867 |
| GBP1 | RPAP1 | 0.867 |
| P2RY14 | FKRP | 0.867 |
| APOL2 | USP4 | 0.867 |
| UBE2L6 | ABR | 0.867 |
| POLB | GRAMD1C | 0.867 |
| APOL2 | TERF2 | 0.867 |
| P2RY14 | LZTFL1 | 0.867 |
| UBE2L6 | PDIA4 | 0.867 |
| GBP5 | DIDO1 | 0.867 |
| GBP5 | ABCC4 | 0.867 |
| SECTM1 | VPS35 | 0.867 |
| GBP1 | MAPKAPK5 | 0.867 |
| GBP5 | MNDA | 0.867 |
| UBE2L6 | RFC1 | 0.867 |
| SECTM1 | PDE4B | 0.867 |
| GBP5 | TIA1 | 0.867 |
| UBE2L6 | TOB1 | 0.867 |
| P2RY14 | KCNMB1 | 0.867 |
| POLB | CTDP1 | 0.867 |
| APOL2 | PDHA1 | 0.867 |
| GBP1 | PIK3CB | 0.867 |
| P2RY14 | TOMM20 | 0.867 |
| GBP1 | TMCC1 | 0.867 |
| GBP1 | PPAT | 0.867 |
| CDC34 | GBP1 | 0.867 |
| POLB | HDAC1 | 0.867 |
| TMED3 | LHFPL2 | 0.867 |
| POLB | CUL4A | 0.867 |
| PZRY14 | MYO9B | 0.867 |
| POLB | ABCF3 | 0.867 |
| GBP5 | TUBG2 | 0.867 |
| SECTM1 | GOLGA3 | 0.867 |
| P2RY14 | NDUFA8 | 0.867 |
| SECTM1 | MSH6 | 0.867 |
| PSME1 | GRWD1 | 0.867 |
| POLB | ATF7IP | 0.867 |
| APOL2 | TSPAN14 | 0.867 |
| P2RY14 | SFXN3 | 0.867 |
| SECTM1 | ABL1 | 0.867 |
| GBP5 | ZW10 | 0.867 |
| GBP5 | HCK | 0.867 |
| POLB | SKP2 | 0.867 |
| UBE2L6 | FYCO1 | 0.867 |
| APOL1 | GLG1 | 0.867 |
| GBP1 | ATP6V1A | 0.867 |
| POLB | PPP2CA | 0.867 |
| GBP1 | LSG1 | 0.867 |
| POLB | EXOC7 | 0.867 |
| ATG3 | KLF6 | 0.867 |
| GBP5 | GALNT3 | 0.867 |
| P2RY14 | PSEN2 | 0.867 |
| P2RY14 | CHST2 | 0.867 |
| LIMK1 | SMARCC1 | 0.867 |
| P2RY14 | BIRC3 | 0.867 |
| SECTM1 | CD40 | 0.867 |
| UBE2L6 | RFWD3 | 0.867 |
| P2RY14 | ZAP70 | 0.867 |
| GBP1 | HMG20A | 0.867 |
| P2RY14 | USP3 | 0.867 |
| GBP5 | CTBS | 0.867 |
| AIM2 | CYB561 | 0.867 |
| DAPP1 | PHF3 | 0.867 |
| GBP1 | NET1 | 0.867 |
| GBP5 | SRI | 0.867 |
| GBP5 | PTOV1 | 0.867 |
| UBE2L6 | MSH6 | 0.867 |
| UBE216 | TRAP1 | 0.867 |
| GBP5 | PLAC8 | 0.867 |
| P2RY14 | PDCL | 0.867 |
| UBE216 | MAZ | 0.867 |
| POLB | SLBP | 0.867 |
| GBP5 | NECAP1 | 0.867 |
| ANP32B | GBP1 | 0.867 |
| P2RY14 | CRK | 0.867 |
| GBP5 | SLC35A5 | 0.867 |
| GBP1 | ESD | 0.867 |
| SECTM1 | CASP1 | 0.867 |
| GBP5 | LIMD2 | 0.867 |
| GBP5 | GNL3L | 0.867 |
| GBP1 | PDIA6 | 0.867 |
| SECTM1 | TAF6 | 0.867 |
| UBE216 | GBP1 | 0.867 |
| GBP5 | DHX35 | 0.867 |
| LHFPL2 | GPBP1L1 | 0.867 |
| GBP5 | VEGFB | 0.867 |
| GBP5 | MMP25 | 0.867 |
| P2RY14 | KATNA1 | 0.867 |
| SECTM1 | SRPK2 | 0.867 |
| SECTM1 | SEC63 | 0.867 |
| C1QA | MCM2 | 0.867 |
| P2RY14 | DGCR2 | 0.867 |
| C1QA | LIMK1 | 0.867 |
| SECTM1 | SNW1 | 0.867 |
| GBP1 | MDM2 | 0.867 |
| GBP1 | WBP11 | 0.867 |
| APOL2 | GOLGA3 | 0.867 |
| GSTO1 | C1QA | 0.867 |
| PZRY14 | MRPL19 | 0.867 |
| SUOX | P2RY14 | 0.867 |
| P2RY14 | PAFAH2 | 0.867 |
| P2RY14 | TM9SF2 | 0.867 |
| UBE2L6 | MCM7 | 0.867 |
| SECTM1 | KLF2 | 0.867 |
| POLB | CTSF | 0.867 |
| UBE2L6 | PRDM4 | 0.867 |
| GBP5 | SPP1 | 0.867 |
| GBP5 | DSC2 | 0.867 |
| GBP5 | MAP3K7 | 0.867 |
| POLB | DENND4A | 0.867 |
| GBP5 | MFN2 | 0.867 |
| LHFPL2 | DIAPH1 | 0.867 |
| AIM2 | MAK | 0.867 |
| SECTM1 | TACC3 | 0.867 |
| P2RY14 | MED4 | 0.867 |
| POLB | CHFR | 0.867 |
| P2RY14 | FTSJ3 | 0.867 |
| P2RY14 | TPP1 | 0.867 |
| GBP5 | HPS5 | 0.867 |
| POLB | XYLT2 | 0.867 |
| GBP5 | CNOT1 | 0.867 |
| P2RY14 | NT5E | 0.867 |
| P2RY14 | SNAPC4 | 0.867 |
| UBE216 | ODF2 | 0.867 |
| POLB | TRIM24 | 0.867 |
| P2RY14 | COG2 | 0.867 |
| POLB | SLC27A5 | 0.867 |
| POLB | PITPNC1 | 0.867 |
| GBP5 | PDSS2 | 0.867 |
| NRGN | GBP1 | 0.867 |
| APOL2 | ABCF2 | 0.867 |
| APOL2 | DHX9 | 0.867 |
| P2RY14 | ERGIC2 | 0.867 |
| DUSP3 | GBP1 | 0.867 |
| P2RY14 | CDK10 | 0.867 |
| SECTM1 | TM9SF4 | 0.867 |
| DNAL4 | GBP1 | 0.867 |
| PSME1 | TSPAN14 | 0.867 |
| GBP5 | EFNA1 | 0.867 |
| C1QA | DDX27 | 0.867 |
| UBE2L6 | MAGED1 | 0.867 |
| TXNL1 | P2RY14 | 0.867 |
| GBP1 | NFX1 | 0.867 |
| C1QA | CAPN2 | 0.867 |
| GBP1 | AQR | 0.867 |
| P2RY14 | LCP2 | 0.867 |
| GBP5 | SPCS3 | 0.867 |
| POLB | ASTE1 | 0.867 |
| P2RY14 | CNGB1 | 0.867 |
| P2RY14 | KCNMA1 | 0.867 |
| UBE216 | PARP6 | 0.867 |
| C1QA | RIN3 | 0.867 |
| POLB | ZNF330 | 0.867 |
| GBP5 | SSR2 | 0.867 |
| UBE2L6 | PSMD5 | 0.867 |
| P2RY14 | NME4 | 0.867 |
| POLB | WDR12 | 0.867 |
| POLB | PRPSAP2 | 0.867 |
| SECTM1 | PFDN5 | 0.867 |
| POLB | RPN1 | 0.867 |
| GBP1 | ABR | 0.867 |
| SECTM1 | CORO2A | 0.867 |
| P2RY14 | TXNDC9 | 0.867 |
| P2RY14 | MPI | 0.867 |
| GBP1 | BRD3 | 0.867 |
| PSME1 | VPS11 | 0.867 |
| P2RY14 | THBD | 0.867 |
| GBP1 | CD40 | 0.867 |
| GBP1 | GOLGA3 | 0.867 |
| IRF2 | P2RY14 | 0.867 |
| C1QA | ST14 | 0.867 |
| KDELR1 | GBP1 | 0.867 |
| GBP1 | COMMD4 | 0.867 |
| LHFPL2 | GBP1 | 0.867 |
| KCNK3 | P2RY14 | 0.867 |
| GBP5 | ARL8B | 0.867 |
| UBE216 | MCM5 | 0.867 |
| GBP5 | LRPPRC | 0.867 |
| GBP1 | CORO1C | 0.867 |
| APOL2 | VCP | 0.867 |
| POLB | FADD | 0.867 |
| P2RY14 | PLCL2 | 0.867 |
| POLB | TPP2 | 0.867 |
| P2RY14 | RPA2 | 0.867 |
| P2RY14 | PDK2 | 0.867 |
| GBP5 | ABHD2 | 0.867 |
| P2RY14 | TIMM8B | 0.867 |
| P2RY14 | ASB6 | 0.867 |
| POLB | MTAP | 0.867 |
| GBP5 | ASCC3 | 0.867 |
| SECTM1 | KCNAB2 | 0.867 |
| UBE216 | RELA | 0.867 |
| PML | MAP4 | 0.867 |
| GBP1 | TMEM87A | 0.867 |
| SECTM1 | PPP2R5A | 0.867 |
| PZRY14 | BCORL1 | 0.867 |
| GBP1 | DYNC1LI2 | 0.867 |
| GBP5 | CAT | 0.867 |
| C1QA | VAMP5 | 0.867 |
| GBP5 | RANBP9 | 0.867 |
| P2RY14 | IL2RB | 0.867 |
| P2RY14 | NCALD | 0.867 |
| POLB | FAS | 0.867 |
| POLB | OXCT1 | 0.867 |
| P2RY14 | PHΥH | 0.867 |
| GBP1 | RNF38 | 0.867 |
| P2RY14 | ASNSD1 | 0.867 |
| GBP5 | PGM3 | 0.867 |
| GBP1 | TMED5 | 0.867 |
| P2RY14 | PRPSAP1 | 0.867 |
| P2RY14 | SLC35E3 | 0.867 |
| GBP5 | MEF2C | 0.867 |
| POLB | CDC42 | 0.867 |
| NPC2 | SECTM1 | 0.867 |
| C1QA | BCR | 0.867 |
| LIMK1 | APOL2 | 0.867 |
| GSTO1 | CYB561 | 0.867 |
| IL15RA | DGKD | 0.867 |
| POLB | ELF1 | 0.867 |
| LSM1 | APOL2 | 0.866 |
| POLB | CCT4 | 0.866 |
| P2RY14 | PNPLA2 | 0.866 |
| POLB | HSD17B4 | 0.866 |
| UBE2L6 | IVNS1ABP | 0.866 |
| GBP5 | PELI2 | 0.866 |
| TMEM59 | LHFPL2 | 0.866 |
| P2RY14 | DERA | 0.866 |
| P2RY14 | STEAP3 | 0.866 |
| APOL2 | MAP3K4 | 0.866 |
| GBP5 | AQP9 | 0.866 |
| APOL2 | MYO9B | 0.866 |
| SECTM1 | RTN3 | 0.866 |
| PZRY14 | MCCC1 | 0.866 |
| GBP5 | CD81 | 0.866 |
| P2RY14 | ACOT9 | 0.866 |
| GBP1 | HYOU1 | 0.866 |
| P2RY14 | GRAP2 | 0.866 |
| GYPC | AIM2 | 0.866 |
| C1QA | SSRP1 | 0.866 |
| ATG3 | APOL2 | 0.866 |
| POLB | MAN1C1 | 0.866 |
| GBP5 | PTAFR | 0.866 |
| SECTM1 | OSBPL2 | 0.866 |
| UBE2L6 | VDAC3 | 0.866 |
| VAMP5 | ATP13A1 | 0.866 |
| P2RY14 | MOSPD2 | 0.866 |
| POLR1D | GBP1 | 0.866 |
| UBE216 | PPBP | 0.866 |
| SECTM1 | PRMT7 | 0.866 |
| POLB | GNE | 0.866 |
| GBP5 | GBF1 | 0.866 |
| NPC2 | AIM2 | 0.866 |
| SECTM1 | PARP6 | 0.866 |
| POLB | ARFGEF1 | 0.866 |
| UBE2L6 | UBTF | 0.866 |
| P2RY14 | TSTA3 | 0.866 |
| GBP1 | PPP1R12A | 0.866 |
| IL15RA | CYB561 | 0.866 |
| GBP5 | LTBP3 | 0.866 |
| GBP5 | VASP | 0.866 |
| P2RY14 | LMBR1L | 0.866 |
| POLB | RFC5 | 0.866 |
| PSME1 | ATG3 | 0.866 |
| GBP5 | ENDOG | 0.866 |
| GBP1 | DDX46 | 0.866 |
| SECTM1 | VPS39 | 0.866 |
| P2RY14 | CNTNAP1 | 0.866 |
| GBP1 | CSNK1G3 | 0.866 |
| GBP1 | OGFOD1 | 0.866 |
| GBP1 | GRAMD1C | 0.866 |
| VAMP5 | TERF2 | 0.866 |
| SECTM1 | MAPK13 | 0.866 |
| GNG11 | PML | 0.866 |
| GBP1 | TRAF1 | 0.866 |
| GBP5 | RAB11A | 0.866 |
| POLB | UBE2G1 | 0.866 |
| GBP5 | PARN | 0.866 |
| IL15RA | LHFPL2 | 0.866 |
| GBP1 | MAPK1 | 0.866 |
| GBP1 | THUMPD1 | 0.866 |
| CTDSPL | GBP1 | 0.866 |
| GBP5 | CRKL | 0.866 |
| GBP5 | PCTP | 0.866 |
| UBE2L6 | SLCO4C1 | 0.866 |
| UBE2L6 | RPS14 | 0.866 |
| GBP5 | ISG20 | 0.866 |
| GBP1 | FBXO21 | 0.866 |
| GBP5 | TMPO | 0.866 |
| GBP5 | MCL1 | 0.866 |
| GBP5 | TMEM62 | 0.866 |
| P2RY14 | ASF1A | 0.866 |
| POLB | PSMD1 | 0.866 |
| POLB | INPPSE | 0.866 |
| GBP1 | SLC6A6 | 0.866 |
| P2RY14 | INPP5A | 0.866 |
| GBP5 | PRR7 | 0.866 |
| GBP1 | VPS13D | 0.866 |
| SECTM1 | SLC9A1 | 0.866 |
| P2RY14 | CDKN1B | 0.866 |
| SECTM1 | BAIAP2 | 0.866 |
| GBP5 | GALNT14 | 0.866 |
| SECTM1 | PSTPIP2 | 0.866 |
| UBE2L6 | OXSR1 | 0.866 |
| GBP5 | PTBP1 | 0.866 |
| UBE2L6 | PITRM1 | 0.866 |
| GBP5 | MAPK3 | 0.866 |
| GYPC | VAMP5 | 0.866 |
| GBP1 | ZNF337 | 0.866 |
| AIM2 | RPS14 | 0.866 |
| P2RY14 | CHD4 | 0.866 |
| POLB | RSAD1 | 0.866 |
| GBP5 | CTSH | 0.866 |
| UBE2L6 | HNMT | 0.866 |
| GBP5 | SLC25A24 | 0.866 |
| UBE2L6 | FOXJ2 | 0.866 |
| PZRY14 | EXOC7 | 0.866 |
| GBP5 | FLT3LG | 0.866 |
| POLB | SMAP1 | 0.866 |
| UBE216 | TUFT1 | 0.866 |
| VAMP5 | TIMM44 | 0.866 |
| PYGM | P2RY14 | 0.866 |
| GBP1 | GTF3C3 | 0.866 |
| SECTM1 | PRKD2 | 0.866 |
| APOL2 | NFATC1 | 0.866 |
| P2RY14 | NONO | 0.866 |
| POLB | HSPA5 | 0.866 |
| P2RY14 | UBE2D4 | 0.866 |
| SECTM1 | PPRC1 | 0.866 |
| GBP1 | CENPJ | 0.866 |
| SECTM1 | YEATS2 | 0.866 |
| P2RY14 | NELL2 | 0.866 |
| SECTM1 | SCMH1 | 0.866 |
| POLB | PRPF3 | 0.866 |
| APOL2 | MCM3 | 0.866 |
| P2RY14 | EHD4 | 0.866 |
| UBE2L6 | ATP6V1G1 | 0.866 |
| CALCOCO2 | GBP1 | 0.866 |
| GBP1 | POLR1B | 0.866 |
| VAMP5 | PPP2R5D | 0.866 |
| PZRY14 | KLHDC2 | 0.866 |
| UROS | GBP1 | 0.866 |
| P2RY14 | MBIP | 0.866 |
| POLB | TNK2 | 0.866 |
| GBP1 | GPD1L | 0.866 |
| GBP5 | DYRK1B | 0.866 |
| POLB | KIF3B | 0.866 |
| IPO13 | P2RY14 | 0.866 |
| GBP5 | RALY | 0.866 |
| GBP1 | SESN1 | 0.866 |
| UBE216 | SMU1 | 0.866 |
| POLB | MDK | 0.866 |
| PF4V1 | APOL1 | 0.866 |
| GBP5 | IDI1 | 0.866 |
| SECTM1 | ADAM19 | 0.866 |
| UBE2L6 | BARD1 | 0.866 |
| GBP1 | DLEC1 | 0.866 |
| PZRY14 | CLMN | 0.866 |
| LIMK1 | GRWD1 | 0.866 |
| GBP1 | EML4 | 0.866 |
| P2RY14 | RERE | 0.866 |
| APOL1 | SSRP1 | 0.866 |
| APOL2 | RBM28 | 0.866 |
| P2RY14 | AMFR | 0.866 |
| POLB | PIGH | 0.866 |
| P2RY14 | VPS35 | 0.866 |
| C1QA | CASP1 | 0.866 |
| GBP1 | ERBB2 | 0.866 |
| GBP5 | TNFAIP6 | 0.866 |
| P2RY14 | STAT6 | 0.866 |
| P2RY14 | TM7SF3 | 0.866 |
| APOL2 | NUMA1 | 0.866 |
| GBP1 | EHBP1 | 0.866 |
| CASP1 | HMGCR | 0.866 |
| GBP1 | ING4 | 0.866 |
| UBE2L6 | MPI | 0.866 |
| GBP5 | THRA | 0.866 |
| IRF1 | KLF6 | 0.866 |
| GBP5 | LSM6 | 0.866 |
| GBP1 | MYH9 | 0.866 |
| SECTM1 | PHF3 | 0.866 |
| P2RY14 | IL15 | 0.866 |
| UBE216 | CIDEB | 0.866 |
| POLB | ATIC | 0.866 |
| PZRY14 | MRPL49 | 0.866 |
| GBP1 | AFF4 | 0.866 |
| POLB | ASB6 | 0.866 |
| P2RY14 | LCK | 0.866 |
| POLB | VPS33A | 0.866 |
| UBE2L6 | PARP16 | 0.866 |
| APOL2 | GEMIN4 | 0.866 |
| ECHS1 | GBP1 | 0.866 |
| GBP1 | MYO9A | 0.866 |
| ROM1 | P2RY14 | 0.866 |
| POLB | PPM1D | 0.866 |
| P2RY14 | FAM49B | 0.866 |
| GBP1 | SLC35A3 | 0.866 |
| P2RY14 | MAP2K5 | 0.866 |
| POLB | VAMP3 | 0.866 |
| CETN2 | GBP1 | 0.866 |
| POLB | ATP2A3 | 0.866 |
| LHFPL2 | NCOR1 | 0.866 |
| POLB | INPP1 | 0.866 |
| APOL1 | NCL | 0.866 |
| P2RY14 | PRPF3 | 0.866 |
| VAMP5 | PLCD1 | 0.866 |
| LHFPL2 | SMARCC1 | 0.866 |
| P2RY14 | HSD17B12 | 0.866 |
| GBP5 | NOSIP | 0.866 |
| UBE216 | PFKP | 0.866 |
| VAMP5 | POLR3B | 0.866 |
| POLB | TAF2 | 0.866 |
| POLB | TAF1C | 0.866 |
| GBP5 | CYC1 | 0.866 |
| LHFPL2 | TSPAN14 | 0.866 |
| P2RY14 | TPK1 | 0.866 |
| VAMP5 | GEMIN4 | 0.866 |
| GBP1 | RRBP1 | 0.866 |
| APOL1 | DPF2 | 0.866 |
| SECTM1 | FBXW2 | 0.866 |
| POLB | SAE1 | 0.866 |
| GBP5 | TBC1D10B | 0.866 |
| POLB | MAPKAPK5 | 0.866 |
| P2RY14 | OSBPL2 | 0.866 |
| C1QA | PML | 0.866 |
| GBP1 | CCDC47 | 0.866 |
| POLB | ATP7B | 0.866 |
| GBP5 | ITM2B | 0.866 |
| SECTM1 | SEC23B | 0.866 |
| DENND1A | GBP1 | 0.866 |
| UBE2L6 | GANAB | 0.866 |
| P2RY14 | SLC25A36 | 0.866 |
| LIMK1 | TTLL12 | 0.866 |
| POLB | APOL3 | 0.866 |
| IL15RA | OSBP | 0.866 |
| POLB | ARMCX5 | 0.866 |
| GBP5 | GCH1 | 0.866 |
| P2RY14 | RUVBL1 | 0.866 |
| GBP5 | HPS4 | 0.866 |
| POLB | KLHL7 | 0.866 |
| LHFPL2 | TLE4 | 0.866 |
| VAMP5 | DGKD | 0.866 |
| POLB | PDIA4 | 0.866 |
| GBP5 | SCML1 | 0.866 |
| GBP1 | VDAC3 | 0.866 |
| APOL2 | FTSJ3 | 0.866 |
| GBP1 | ITGA2B | 0.866 |
| POLB | TRIM8 | 0.866 |
| GBP5 | ELOVL5 | 0.866 |
| GBP1 | PER2 | 0.866 |
| P2RY14 | ATIC | 0.866 |
| GBP1 | SMARCC2 | 0.866 |
| GBP5 | ARIH1 | 0.865 |
| UBE216 | IFT122 | 0.865 |
| LIMK1 | ATG3 | 0.865 |
| UBE2L6 | STIM1 | 0.865 |
| SECTM1 | PSMA2 | 0.865 |
| P2RY14 | LETM1 | 0.865 |
| SELP | P2RY14 | 0.865 |
| P2RY14 | GNB1 | 0.865 |
| GBP5 | RNF167 | 0.865 |
| P2RY14 | CERK | 0.865 |
| UBE216 | PRKAR2A | 0.865 |
| GBP1 | RECQL | 0.865 |
| P2RY14 | DSTN | 0.865 |
| SECTM1 | PLCD1 | 0.865 |
| APOL2 | RASGRP2 | 0.865 |
| P2RY14 | GORASP2 | 0.865 |
| SECTM1 | MAP3K4 | 0.865 |
| LHFPL2 | ZBTB16 | 0.865 |
| P2RY14 | BLMH | 0.865 |
| P2RY14 | ICMT | 0.865 |
| GBP1 | TERF2 | 0.865 |
| POLB | EIF5 | 0.865 |
| POLB | CTBP2 | 0.865 |
| GBP5 | TMOD3 | 0.865 |
| PSME1 | VAMP5 | 0.865 |
| PZRY14 | COQ2 | 0.865 |
| P2RY14 | PSMC2 | 0.865 |
| GBP1 | ZHX2 | 0.865 |
| GBP1 | DLAT | 0.865 |
| POLB | PEX1 | 0.865 |
| P2RY14 | WDR4 | 0.865 |
| P2RY14 | ZNF562 | 0.865 |
| P2RY14 | TMEM106 | 0.865 |
| | C | |
| GBP5 | SMG7 | 0.865 |
| CASP7 | NOLC1 | 0.865 |
| CAPN1 | P2RY14 | 0.865 |
| GBP1 | ZZEF1 | 0.865 |
| POLB | ATP1B2 | 0.865 |
| LSM1 | GBP1 | 0.865 |
| UBE216 | UPB1 | 0.865 |
| ATP6V1G | SECTM1 | 0.865 |
| 1 | | |
| SECTM1 | PPM1G | 0.865 |
| POLB | E2F1 | 0.865 |
| GBP5 | MAPK9 | 0.865 |
| GBP5 | PPIB | 0.865 |
| P2RY14 | NUP37 | 0.865 |
| LMO2 | P2RY14 | 0.865 |
| SECTM1 | CHD4 | 0.865 |
| SECTM1 | RAC2 | 0.865 |
| DNAJC1 | GBP1 | 0.865 |
| GBP1 | CAMK2G | 0.865 |
| APOL2 | BAHD1 | 0.865 |
| GBP1 | TRIM68 | 0.865 |
| ATP6V1E1 | GBP1 | 0.865 |
| UBE2L6 | LSM1 | 0.865 |
| NPC2 | GNG11 | 0.865 |
| GBP5 | STAT3 | 0.865 |
| UBE2L6 | XPC | 0.865 |
| POLB | RBL2 | 0.865 |
| UBE216 | POU2F1 | 0.865 |
| UBE216 | CDCA4 | 0.865 |
| LHFPL2 | RUNX2 | 0.865 |
| PZRY14 | NDUFS1 | 0.865 |
| LHFPL2 | MTMR6 | 0.865 |
| P2RY14 | HGS | 0.865 |
| SECTM1 | NIPBL | 0.865 |
| GBP5 | ELMO2 | 0.865 |
| CD74 | LHFPL2 | 0.865 |
| GBP1 | ATG3 | 0.865 |
| GBP1 | ZCCHC8 | 0.865 |
| P2RY14 | PBX1 | 0.865 |
| P2RY14 | DFFA | 0.865 |
| POLB | INPPSA | 0.865 |
| VAMP5 | AKAP1 | 0.865 |
| GBP1 | WRN | 0.865 |
| DAPP1 | TRAPPC3 | 0.865 |
| UBE2L6 | TOPBP1 | 0.865 |
| POLB | ABCC5 | 0.865 |
| PCBD1 | POLB | 0.865 |
| GBP5 | WAS | 0.865 |
| AIM2 | PFDN5 | 0.865 |
| POLB | EML4 | 0.865 |
| GBP1 | RRS1 | 0.865 |
| C1QA | ZNF200 | 0.865 |
| SECTM1 | ANP32B | 0.865 |
| GBP1 | ARMC1 | 0.865 |
| P2RY14 | ATP2A3 | 0.865 |
| SDC1 | APOL2 | 0.865 |
| P2RY14 | BECN1 | 0.865 |
| SECTM1 | ILF3 | 0.865 |
| P2RY14 | RNF40 | 0.865 |
| GBP1 | DHX29 | 0.865 |
| UBE216 | PPP2R5A | 0.865 |
| SECTM1 | PAF1 | 0.865 |
| APOL2 | PRMT7 | 0.865 |
| UBE2L6 | STK16 | 0.865 |
| POLB | JOSD1 | 0.865 |
| POLB | GTPBP3 | 0.865 |
| GBP1 | TACC1 | 0.865 |
| UBE2L6 | RUVBL1 | 0.865 |
| LHFPL2 | PPP2R5A | 0.865 |
| GBP5 | NDST2 | 0.865 |
| APOL2 | RUNX2 | 0.865 |
| GBP1 | SRRM1 | 0.865 |
| SECTM1 | AACS | 0.865 |
| UBE216 | PTP4A3 | 0.865 |
| P2RY14 | VTI1B | 0.865 |
| P2RY14 | MRFAP1L1 | 0.865 |
| UBE216 | IL12RB1 | 0.865 |
| GBP5 | SLC2A5 | 0.865 |
| VAMP5 | SMARCC1 | 0.865 |
| APOL2 | STAG1 | 0.865 |
| SECTM1 | SNAPC4 | 0.865 |
| UBE2L6 | MRPL49 | 0.865 |
| GBP5 | PIGC | 0.865 |
| GBP1 | PDK4 | 0.865 |
| GBP1 | PARVB | 0.865 |
| GBP5 | MAL | 0.865 |
| SECTM1 | APOL3 | 0.865 |
| P2RY14 | HEXIM1 | 0.865 |
| UBE216 | DDX24 | 0.865 |
| UBE216 | PLAA | 0.865 |
| POLB | RAB20 | 0.865 |
| GBP1 | MAP2K1 | 0.865 |
| APOL2 | PRDM2 | 0.865 |
| POLB | PAK1IP1 | 0.865 |
| POLB | SNAPC3 | 0.865 |
| C1QA | SMARCC1 | 0.865 |
| GBP1 | ZMYM5 | 0.865 |
| GBP1 | ST8SIA4 | 0.865 |
| P2RY14 | RASSF1 | 0.865 |
| P2RY14 | PRKX | 0.865 |
| SECTM1 | MCM5 | 0.865 |
| GBP5 | MRPS33 | 0.865 |
| PRRG4 | KLF6 | 0.865 |
| CD244 | GBP1 | 0.865 |
| GBP5 | CLCN3 | 0.865 |
| GBP5 | VCL | 0.865 |
| UBE2L6 | PRMT7 | 0.865 |
| APOL1 | FAS | 0.865 |
| P2RY14 | MAPRE1 | 0.865 |
| SECTM1 | SIRT5 | 0.865 |
| GBP1 | ABL1 | 0.865 |
| P2RY14 | PDE4A | 0.865 |
| GBP1 | RBBP7 | 0.865 |
| P2RY14 | RAF1 | 0.865 |
| UBE216 | SCMH1 | 0.865 |
| P2RY14 | SEPHS1 | 0.865 |
| C1QA | OSBP | 0.865 |
| RAD9A | P2RY14 | 0.865 |
| P2RY14 | DNAJB6 | 0.865 |
| GBP1 | KYNU | 0.865 |
| VAMP5 | KLF6 | 0.865 |
| SECTM1 | H6PD | 0.865 |
| GBP5 | CD37 | 0.865 |
| C1QA | GRWD1 | 0.865 |
| PSME1 | GNG11 | 0.865 |
| SECTM1 | DUSP22 | 0.865 |
| PML | ATG3 | 0.865 |
| PSME1 | DIAPH1 | 0.865 |
| P2RY14 | MARK2 | 0.865 |
| P2RY14 | SORT1 | 0.865 |
| DAPP1 | ZFR | 0.865 |
| GBP1 | CTBP2 | 0.865 |
| SECTM1 | POLR1D | 0.865 |
| P2RY14 | ACTR1B | 0.865 |
| GBP5 | ADAM10 | 0.865 |
| GBP5 | ANGEL2 | 0.865 |
| SECTM1 | DEDD | 0.865 |
| UBE2L6 | NFX1 | 0.865 |
| P2RY14 | OSBPL10 | 0.865 |
| GBP1 | NDUFB2 | 0.865 |
| POLB | VAC14 | 0.865 |
| SECTM1 | UPB1 | 0.865 |
| POLB | RGS14 | 0.865 |
| GBP5 | RFXAP | 0.865 |
| GBP1 | E2F3 | 0.865 |
| LHFPL2 | NCOA1 | 0.865 |
| LHFPL2 | FBXW2 | 0.865 |
| APOLZ | GRWD1 | 0.865 |
| GBP5 | IDH3G | 0.865 |
| P2RY14 | SMARCD1 | 0.865 |
| P2RY14 | CRNKL1 | 0.865 |
| P2RY14 | SP110 | 0.865 |
| UBE2L6 | IL21R | 0.865 |
| GBP5 | NUDT9 | 0.865 |
| SECTM1 | TP53BP2 | 0.865 |
| SECTM1 | PLAA | 0.865 |
| POLB | TNFRSF14 | 0.865 |
| GBP1 | GNE | 0.865 |
| PZRY14 | BNIP1 | 0.865 |
| GBP5 | ARHGAP17 | 0.865 |
| GBP1 | ARMCX3 | 0.865 |
| GBP5 | CMAS | 0.865 |
| P2RY14 | MLX | 0.865 |
| POLB | ZNF7 | 0.865 |
| PSME1 | MAP3K4 | 0.865 |
| SECTM1 | SYNJ2 | 0.865 |
| UBE2L6 | PRPF8 | 0.865 |
| SIRT6 | GBP1 | 0.865 |
| FCN1 | P2RY14 | 0.865 |
| UBE216 | ATP2A3 | 0.865 |
| MGST3 | GBP1 | 0.865 |
| SECTM1 | PARP16 | 0.865 |
| GBP5 | OCRL | 0.865 |
| POLB | SEC23B | 0.865 |
| PSME1 | PLCD1 | 0.865 |
| GBP5 | CD3G | 0.865 |
| LHFPL2 | PIK3CB | 0.865 |
| P2RY14 | TUFT1 | 0.865 |
| P2RY14 | MINK1 | 0.865 |
| POLB | COG2 | 0.865 |
| LHFPL2 | NENF | 0.865 |
| P2RY14 | SMPD1 | 0.865 |
| POLB | DUSP11 | 0.865 |
| LIMK1 | IL15 | 0.865 |
| UBE216 | FLNB | 0.865 |
| GBP5 | OXA1L | 0.865 |
| GBP1 | MAN2A1 | 0.865 |
| SECTM1 | USP48 | 0.865 |
| SECTM1 | RBM28 | 0.865 |
| PSME1 | IFT122 | 0.865 |
| SECTM1 | FOXJ2 | 0.865 |
| PF4V1 | SECTM1 | 0.865 |
| LIMK1 | PML | 0.865 |
| C1QA | DUSP3 | 0.865 |
| GBP1 | ANK3 | 0.865 |
| GBP1 | CUL4A | 0.865 |
| GBP5 | SLAMF7 | 0.865 |
| UBE216 | PF4V1 | 0.865 |
| GBP1 | PARP6 | 0.865 |
| PZRY14 | RBM3 | 0.865 |
| JAM3 | GBP1 | 0.865 |
| ACP2 | POLB | 0.865 |
| GBP1 | MSRB2 | 0.865 |
| P2RY14 | PSMA2 | 0.865 |
| POLB | RNF41 | 0.865 |
| P2RY14 | TPM1 | 0.865 |
| LHFPL2 | SYNJ2 | 0.865 |
| PSME1 | CALCOCO2 | 0.865 |
| POLB | CAV2 | 0.865 |
| APOL2 | DDX23 | 0.865 |
| GBP1 | EXOSC10 | 0.865 |
| PRRG4 | APOL1 | 0.865 |
| LHFPL2 | KLF3 | 0.865 |
| VAMP5 | BCR | 0.865 |
| ATG3 | HMGCR | 0.864 |
| APH1A | GBP1 | 0.864 |
| GBP5 | CDC40 | 0.864 |
| P2RY14 | TPRKB | 0.864 |
| UBE2L6 | ARMC7 | 0.864 |
| GBP5 | LONRF3 | 0.864 |
| SECTM1 | DDX23 | 0.864 |
| SECTM1 | HIC2 | 0.864 |
| P2RY14 | MEN1 | 0.864 |
| P2RY14 | PDE6B | 0.864 |
| VAMP5 | PPM1G | 0.864 |
| GBP1 | CSE1L | 0.864 |
| UBE2L6 | FAS | 0.864 |
| PSMB6 | GBP1 | 0.864 |
| GBP5 | TFCP2 | 0.864 |
| PML | SMARCC1 | 0.864 |
| P2RY14 | CASP8 | 0.864 |
| P2RY14 | PUS3 | 0.864 |
| IL15RA | APOL1 | 0.864 |
| P2RY14 | TAF7 | 0.864 |
| P2RY14 | TBX21 | 0.864 |
| GBP5 | MAP2K6 | 0.864 |
| GBP5 | STOM | 0.864 |
| GBP1 | CPNE3 | 0.864 |
| P2RY14 | PLEK2 | 0.864 |
| GBP1 | TES | 0.864 |
| PKIG | P2RY14 | 0.864 |
| POLB | TMEM97 | 0.864 |
| UBE2L6 | SERINC3 | 0.864 |
| POLB | PIK3R1 | 0.864 |
| AIM2 | MCM2 | 0.864 |
| P2RY14 | ALDH9A1 | 0.864 |
| POLB | AAAS | 0.864 |
| POLB | NUP107 | 0.864 |
| SECTM1 | TSC2 | 0.864 |
| SECTM1 | PLXND1 | 0.864 |
| UBE216 | CORO2A | 0.864 |
| GBP5 | SERPINB1 | 0.864 |
| P2RY14 | TRPV2 | 0.864 |
| SECTM1 | NFKB1 | 0.864 |
| SECTM1 | VPS33B | 0.864 |
| GBP5 | AUH | 0.864 |
| P2RY14 | CHRNB1 | 0.864 |
| VAMP5 | RRBP1 | 0.864 |
| SECTM1 | TIMM44 | 0.864 |
| APOL1 | SMARCC1 | 0.864 |
| DAPP1 | TOPBP1 | 0.864 |
| NDUFB6 | IL15RA | 0.864 |
| UBE2L6 | DENND1A | 0.864 |
| UBE2L6 | TULP4 | 0.864 |
| GBP1 | ARHGEF7 | 0.864 |
| P2RY14 | MTHFR | 0.864 |
| P2RY14 | XRCC5 | 0.864 |
| GBP5 | CEPT1 | 0.864 |
| GBP1 | MDM1 | 0.864 |
| MYL9 | GBP1 | 0.864 |
| SECTM1 | DNAJC4 | 0.864 |
| POLB | RAF1 | 0.864 |
| GBP5 | MAP4K5 | 0.864 |
| GBP1 | ABLIM1 | 0.864 |
| GBP5 | KLHL18 | 0.864 |
| APOL1 | PBXIP1 | 0.864 |
| LIMK1 | GLG1 | 0.864 |
| IL15RA | PML | 0.864 |
| UBE216 | DBR1 | 0.864 |
| PSME1 | ELAC2 | 0.864 |
| P2RY14 | ACTR5 | 0.864 |
| GBP5 | CDC5L | 0.864 |
| SECTM1 | PIK3R5 | 0.864 |
| P2RY14 | DNAJB1 | 0.864 |
| SECTM1 | RUFY3 | 0.864 |
| P2RY14 | RPUSD2 | 0.864 |
| GBP1 | EIF4G1 | 0.864 |
| GBP1 | AQP3 | 0.864 |
| PSME1 | APOL2 | 0.864 |
| P2RY14 | IL1B | 0.864 |
| GBP5 | MRPL34 | 0.864 |
| PZRY14 | CDR2L | 0.864 |
| UBE2L6 | RBBP4 | 0.864 |
| GBP1 | RER1 | 0.864 |
| POLB | TMC6 | 0.864 |
| UBE2L6 | CD40 | 0.864 |
| POLB | VDAC3 | 0.864 |
| P2RY14 | CFDP1 | 0.864 |
| P2RY14 | NUCB2 | 0.864 |
| GBP5 | MCM3AP | 0.864 |
| P2RY14 | ULK1 | 0.864 |
| GBP1 | TSC1 | 0.864 |
| GBP1 | NR3C2 | 0.864 |
| SECTM1 | RNF25 | 0.864 |
| GBP1 | AUTS2 | 0.864 |
| UBE216 | PBXIP1 | 0.864 |
| UBE2L6 | BCL2L11 | 0.864 |
| VAMP5 | FTSJ3 | 0.864 |
| LHFPL2 | TRAPPC3 | 0.864 |
| GBP1 | TTF1 | 0.864 |
| GBP1 | UROD | 0.864 |
| POLB | TSPYL2 | 0.864 |
| UBE2L6 | PPP2R5B | 0.864 |
| GBP5 | MEA1 | 0.864 |
| FZD2 | GBP1 | 0.864 |
| POLB | XRCC1 | 0.864 |
| UBE216 | TIMM44 | 0.864 |
| PZRY14 | SERPINB8 | 0.864 |
| UBE216 | PRKCQ | 0.864 |
| POLB | TAF4 | 0.864 |
| GBP1 | CHMP7 | 0.864 |
| CHMP6 | GBP1 | 0.864 |
| PML | ARID1A | 0.864 |
| LHFPL2 | YEATS2 | 0.864 |
| APOL2 | RIN3 | 0.864 |
| C1QA | TSR1 | 0.864 |
| POLB | ST8SIA4 | 0.864 |
| GBP1 | PRPF8 | 0.864 |
| LIMK1 | CASP1 | 0.864 |
| P2RY14 | KLF2 | 0.864 |
| P2RY14 | MCM7 | 0.864 |
| IRF1 | POLB | 0.864 |
| GBP1 | TBC1D22A | 0.864 |
| P2RY14 | VPS33B | 0.864 |
| LHFPL2 | SPEN | 0.864 |
| GBP1 | SSBP1 | 0.864 |
| POLB | PIK3R5 | 0.864 |
| P2RY14 | DDX1 | 0.864 |
| GBP1 | CNOT2 | 0.864 |
| APOL2 | NIPBL | 0.864 |
| P2RY14 | BTG3 | 0.864 |
| GBP1 | IL21R | 0.864 |
| TM7SF2 | GBP1 | 0.864 |
| GBP5 | DOK2 | 0.864 |
| C1QA | RPAP1 | 0.864 |
| P2RY14 | EHD3 | 0.864 |
| C1QA | ABCF2 | 0.864 |
| P2RY14 | ALDH1A1 | 0.864 |
| P2RY14 | OPRL1 | 0.864 |
| C1QA | POLR3B | 0.864 |
| C1QA | DGKD | 0.864 |
| GBP5 | WDR37 | 0.864 |
| VAMP5 | OSBP | 0.864 |
| P2RY14 | CTSF | 0.864 |
| LHFPL2 | CTCF | 0.864 |
| GBP1 | NKTR | 0.864 |
| UBE2L6 | AQP3 | 0.864 |
| VAMP5 | PLAGL2 | 0.864 |
| POLB | AOC3 | 0.864 |
| PZRY14 | SH3GLB2 | 0.864 |
| GBP5 | RASA1 | 0.864 |
| IL15RA | C1QA | 0.864 |
| GBP1 | WDR12 | 0.864 |
| GBP1 | PTGER4 | 0.864 |
| SECTM1 | CHAF1B | 0.864 |
| GBP1 | POU2F1 | 0.864 |
| UBE2L6 | SLA | 0.864 |
| POLB | BCAT2 | 0.864 |
| GBP1 | SDHB | 0.864 |
| GBP5 | CTDSP1 | 0.864 |
| SECTM1 | CPSF1 | 0.864 |
| GBP5 | TUFM | 0.864 |
| P2RY14 | LEPROTL1 | 0.864 |
| VAMP5 | TSR1 | 0.864 |
| UBE2L6 | DNAJC4 | 0.864 |
| UBE216 | CTNNB1 | 0.864 |
| VAMP5 | RRS1 | 0.864 |
| GBP1 | CORO1B | 0.864 |
| UBE2L6 | PCSK6 | 0.864 |
| GBP5 | KRAS | 0.864 |
| UBE2L6 | APBA2 | 0.864 |
| IRF3 | GBP1 | 0.864 |
| AIM2 | VAMP5 | 0.864 |
| UBE2L6 | AGPAT3 | 0.864 |
| GBP5 | PEX6 | 0.864 |
| GBP1 | SAP130 | 0.864 |
| UBE216 | TMEM97 | 0.864 |
| P2RY14 | PECR | 0.864 |
| GBP1 | LAMP2 | 0.864 |
| SECTM1 | NOLC1 | 0.864 |
| LIMK1 | PPARD | 0.864 |
| P2RY14 | DNAJB9 | 0.864 |
| GBP1 | SSRP1 | 0.864 |
| GBP5 | MEF2D | 0.864 |
| P2RY14 | ARL6IPS | 0.864 |
| GBP5 | FXYD5 | 0.864 |
| P2RY14 | SCCPDH | 0.864 |
| UBE216 | LSG1 | 0.864 |
| SECTM1 | ZXDC | 0.864 |
| SECTM1 | RAB35 | 0.864 |
| LIMK1 | OSBP | 0.864 |
| TMEM59 | PSME1 | 0.864 |
| P2RY14 | ADIPOR2 | 0.864 |
| APOL2 | PARP6 | 0.864 |
| POLB | SLC25A12 | 0.864 |
| NPC2 | DDX27 | 0.864 |
| GBP1 | PPP2R5E | 0.864 |
| GBP1 | VPS11 | 0.864 |
| GBP1 | PEX1 | 0.864 |
| POLB | MAP4K4 | 0.864 |
| PSME1 | MCM5 | 0.864 |
| P2RY14 | ITGA5 | 0.864 |
| GBP1 | GALNT2 | 0.864 |
| GBP1 | TRAP1 | 0.864 |
| GBP1 | ZCCHC14 | 0.864 |
| GBP1 | CORO2A | 0.864 |
| GBP5 | JUP | 0.864 |
| GBP5 | STAB1 | 0.864 |
| POLB | IL21R | 0.864 |
| APOL2 | ODF2 | 0.864 |
| P2RY14 | ARRB1 | 0.864 |
| GBP1 | RAB8B | 0.864 |
| GBP1 | BTG1 | 0.864 |
| P2RY14 | RGS12 | 0.864 |
| POLB | ECHDC2 | 0.864 |
| SECTM1 | R3HDM2 | 0.864 |
| SECTM1 | PSMD2 | 0.864 |
| P2RY14 | ELMO3 | 0.864 |
| P2RY14 | ITGB1BP1 | 0.864 |
| SECTM1 | SLC6A6 | 0.864 |
| UBE2L6 | SRD5A1 | 0.864 |
| UBE2L6 | KPNA6 | 0.864 |
| POLB | FKRP | 0.864 |
| POLB | TRAF3 | 0.864 |
| GBP5 | APEX1 | 0.864 |
| P2RY14 | MAPKAP1 | 0.864 |
| UBE2L6 | CCNK | 0.864 |
| SECTM1 | DLEC1 | 0.864 |
| UBE2L6 | TRIM8 | 0.864 |
| SECTM1 | XPNPEP1 | 0.864 |
| PZRY14 | OPN3 | 0.864 |
| PZRY14 | IL10RA | 0.864 |
| SECTM1 | RNGTT | 0.864 |
| GBP5 | DNAJC10 | 0.864 |
| P2RY14 | TIMM44 | 0.864 |
| GBP1 | CDYL | 0.864 |
| C1QA | NOLC1 | 0.864 |
| AGPAT3 | GBP1 | 0.864 |
| GBP1 | MECP2 | 0.864 |
| GBP5 | AP2B1 | 0.864 |
| GBP5 | VPS13B | 0.864 |
| POLB | SLC2A9 | 0.864 |
| NPC2 | C1QA | 0.864 |
| C1QA | MCM6 | 0.864 |
| GBP1 | PDHA1 | 0.864 |
| GBP1 | SUZ12 | 0.864 |
| UBE2L6 | NET1 | 0.864 |
| UBE2L6 | SEC63 | 0.864 |
| GBP5 | RBM6 | 0.864 |
| GBP5 | TNS3 | 0.864 |
| P2RY14 | SNX4 | 0.864 |
| SECTM1 | NFATC3 | 0.864 |
| P2RY14 | CASP5 | 0.864 |
| APOL1 | ATG3 | 0.864 |
| GBP1 | PDPK1 | 0.864 |
| SECTM1 | SAP130 | 0.864 |
| P2RY14 | DYNLT3 | 0.864 |
| POLB | MAPK1 | 0.864 |
| VAMP5 | MSN | 0.864 |
| GBP1 | GFOD1 | 0.864 |
| UBE2L6 | CASP7 | 0.864 |
| P2RY14 | CACNB3 | 0.864 |
| P2RY14 | ERAL1 | 0.864 |
| UBE2L6 | CTDP1 | 0.864 |
| SECTM1 | CC2D1A | 0.864 |
| UBE2L6 | JOSD1 | 0.864 |
| UBE216 | VPS33A | 0.864 |
| LHFPL2 | OSBPL9 | 0.864 |
| GBP5 | CUL4B | 0.864 |
| GBP1 | ABCE1 | 0.864 |
| POLB | POLR2H | 0.864 |
| SECTM1 | RELA | 0.864 |
| PSTPIP2 | PPP2R5A | 0.864 |
| POLB | ELP3 | 0.864 |
| GBP5 | CD2 | 0.864 |
| UBE2L6 | PTPN7 | 0.864 |
| GBP5 | ARID4B | 0.864 |
| APOL2 | PPRC1 | 0.864 |
| GBP5 | COX7B | 0.864 |
| POLB | PGM1 | 0.864 |
| UBE2L6 | MECP2 | 0.864 |
| UBE2L6 | DNM2 | 0.863 |
| APOL1 | MYC | 0.863 |
| GBP1 | NFATC1 | 0.863 |
| GBP1 | UBTF | 0.863 |
| C1QA | CPSF1 | 0.863 |
| P2RY14 | CAV2 | 0.863 |
| GBP5 | PPP3CB | 0.863 |
| IVNS1ABP | VAMP5 | 0.863 |
| GBP5 | SEC24D | 0.863 |
| P2RY14 | RASSF4 | 0.863 |
| P2RY14 | UBAPZL | 0.863 |
| VAMP5 | PPP2R5B | 0.863 |
| UBE2L6 | SAP130 | 0.863 |
| GBP1 | ARCN1 | 0.863 |
| GBP1 | SNIP1 | 0.863 |
| SECTM1 | AXIN1 | 0.863 |
| P2RY14 | UBEZA | 0.863 |
| POLB | ANAPC5 | 0.863 |
| GBP1 | CLASP1 | 0.863 |
| P2RY14 | RNF41 | 0.863 |
| GSTO1 | NDRG1 | 0.863 |
| GBP5 | UPF3B | 0.863 |
| POLB | SMARCD1 | 0.863 |
| C1QA | VCP | 0.863 |
| GBP5 | IGFBP7 | 0.863 |
| AOAH | P2RY14 | 0.863 |
| POLB | VTI1B | 0.863 |
| SECTM1 | XPC | 0.863 |
| UBE216 | HIC2 | 0.863 |
| POLB | ZNF419 | 0.863 |
| UBE216 | TBCD | 0.863 |
| GBP5 | AKAP13 | 0.863 |
| POLB | CIAPIN1 | 0.863 |
| POLB | CASK | 0.863 |
| POLB | ATP6V1H | 0.863 |
| AIM2 | NDRG1 | 0.863 |
| GBP5 | BRCA1 | 0.863 |
| P2RY14 | RANBP3 | 0.863 |
| GBP1 | HERC2 | 0.863 |
| UBE2L6 | NRG1 | 0.863 |
| UBE2L6 | TICAM1 | 0.863 |
| SECTM1 | VPS37B | 0.863 |
| GBP1 | COG5 | 0.863 |
| GBP5 | INPP4A | 0.863 |
| GBP1 | ARIH2 | 0.863 |
| VAMP5 | EHD4 | 0.863 |
| POLB | MAP3K14 | 0.863 |
| VAMP5 | ILF3 | 0.863 |
| POLB | BLMH | 0.863 |
| VAMP5 | ELAC2 | 0.863 |
| P2RY14 | PTK2 | 0.863 |
| TMEM59 | PML | 0.863 |
| SECTM1 | ARHGEF2 | 0.863 |
| GBP1 | HTRA2 | 0.863 |
| GSTO1 | DAPP1 | 0.863 |
| AIM2 | L12RB1 | 0.863 |
| PSME1 | AIM2 | 0.863 |
| SDC1 | VAMP5 | 0.863 |
| UBE216 | NUP155 | 0.863 |
| GBP1 | SOCS2 | 0.863 |
| VAMP5 | TRAP1 | 0.863 |
| AIM2 | APOL2 | 0.863 |
| GBP5 | FAM8A1 | 0.863 |
| GBP5 | COPS3 | 0.863 |
| POLB | NAPA | 0.863 |
| SECTM1 | TRAP1 | 0.863 |
| POLB | UBTF | 0.863 |
| POLB | SDHB | 0.863 |
| VWF | P2RY14 | 0.863 |
| UBE2L6 | USP11 | 0.863 |
| GBP1 | PIK3R5 | 0.863 |
| VAMP5 | DHCR7 | 0.863 |
| GBP1 | MFAP1 | 0.863 |
| VAMP5 | MCM7 | 0.863 |
| UBE2L6 | TMCC1 | 0.863 |
| GBP1 | MAPRE2 | 0.863 |
| GBP1 | SPIN1 | 0.863 |
| LIMK1 | DDX27 | 0.863 |
| SECTM1 | DUSP3 | 0.863 |
| VAMP5 | PMVK | 0.863 |
| POLB | DHRS3 | 0.863 |
| P2RY14 | ZFAND3 | 0.863 |
| C1QA | RBM28 | 0.863 |
| GBP1 | OGDH | 0.863 |
| UBE2L6 | NMT1 | 0.863 |
| POLB | SMARCC2 | 0.863 |
| GBP5 | ALPP | 0.863 |
| UBE216 | ATP8B2 | 0.863 |
| PZRY14 | STMN1 | 0.863 |
| P2RY14 | HSD17B4 | 0.863 |
| SECTM1 | CAPN2 | 0.863 |
| PSME1 | MAX | 0.863 |
| P2RY14 | TSPAN32 | 0.863 |
| POLB | SRPRB | 0.863 |
| GBP5 | PPP1CA | 0.863 |
| GBP5 | GNB2 | 0.863 |
| GBP5 | FLOT2 | 0.863 |
| UBE2L6 | INPP1 | 0.863 |
| POLB | KLF3 | 0.863 |
| VAMP5 | ARMC7 | 0.863 |
| POLB | PF4V1 | 0.863 |
| GBP1 | PITRM1 | 0.863 |
| LHFPL2 | ARID1A | 0.863 |
| SECTM1 | HSPA5 | 0.863 |
| VAMP5 | IMP4 | 0.863 |
| P2RY14 | PAM | 0.863 |
| F13A1 | C1QA | 0.863 |
| P2RY14 | RASSF2 | 0.863 |
| GBP1 | MRPS12 | 0.863 |
| LIMK1 | BCR | 0.863 |
| GBP1 | CDK5RAP1 | 0.863 |
| POLB | MCM5 | 0.863 |
| SECTM1 | PFAS | 0.863 |
| GBP5 | RALGPS2 | 0.863 |
| UBE216 | AP181 | 0.863 |
| ATG3 | OXSR1 | 0.863 |
| UBE216 | ITGA5 | 0.863 |
| VAMP5 | PDIA4 | 0.863 |
| SECTM1 | RASGRP2 | 0.863 |
| POLB | PSMD7 | 0.863 |
| P2RY14 | BCAT1 | 0.863 |
| GBP1 | PPP2R5D | 0.863 |
| VAMP5 | GCHFR | 0.863 |
| ABHD11 | GBP1 | 0.863 |
| VAMP5 | OGDH | 0.863 |
| P2RY14 | RNF8 | 0.863 |
| P2RY14 | SNW1 | 0.863 |
| C1QA | UQCRC1 | 0.863 |
| TMSB10 | DAPP1 | 0.863 |
| GBP1 | INPP1 | 0.863 |
| GBP1 | ABCA2 | 0.863 |
| SECTM1 | PLAGL2 | 0.863 |
| POLB | TOB1 | 0.863 |
| GBP1 | ANKRD11 | 0.863 |
| UBE2L6 | AACS | 0.863 |
| P2RY14 | B4GALT1 | 0.863 |
| GBP1 | DCUN1D2 | 0.863 |
| GBP5 | PLAU | 0.863 |
| GBP5 | DCXR | 0.863 |
| P2RY14 | SLC5A6 | 0.863 |
| PSME1 | DHX9 | 0.863 |
| SECTM1 | PLCG1 | 0.863 |
| GBP1 | PPP2CA | 0.863 |
| SECTM1 | STAT1 | 0.863 |
| SECTM1 | ODF2 | 0.863 |
| POLB | SLC30A5 | 0.863 |
| GBP5 | SMARCA4 | 0.863 |
| GBP5 | ANXA1 | 0.863 |
| AIM2 | TNFRSF14 | 0.863 |
| LHFPL2 | PPP4R1 | 0.863 |
| P2RY14 | PPP6C | 0.863 |
| APOL2 | YEATS2 | 0.863 |
| CLEC10A | GBP1 | 0.863 |
| GBP1 | PAK1IP1 | 0.863 |
| GBP1 | POLD3 | 0.863 |
| UBE216 | ETS1 | 0.863 |
| GBP5 | PTDSS2 | 0.863 |
| SECTM1 | ANKRD11 | 0.863 |
| C1QA | GEMIN4 | 0.863 |
| GBP1 | BTBD1 | 0.863 |
| ACP5 | FAS | 0.863 |
| POLB | PPM1G | 0.863 |
| GBP1 | DAAM1 | 0.863 |
| POLB | EP400 | 0.863 |
| LHFPL2 | OSBP | 0.863 |
| GBP1 | ZNF217 | 0.863 |
| GNG11 | LHFPL2 | 0.863 |
| POLB | CASP10 | 0.863 |
| POLB | DLG4 | 0.863 |
| P2RY14 | CDC7 | 0.863 |
| POLB | RPA2 | 0.863 |
| POLB | RAPGEF1 | 0.863 |
| GBP1 | UPF3A | 0.863 |
| VAMP5 | CIDEB | 0.863 |
| GBP5 | ACTN1 | 0.863 |
| UBE2L6 | F13A1 | 0.863 |
| GBP1 | CDK6 | 0.863 |
| SECTM1 | FAM111A | 0.863 |
| GBP1 | IRF4 | 0.863 |
| GBP5 | CDKNIC | 0.863 |
| P2RY14 | HOOK2 | 0.863 |
| VAMP5 | TIMELESS | 0.863 |
| GBP5 | CSTB | 0.863 |
| P2RY14 | SERPINF1 | 0.863 |
| GBP5 | CSTF2T | 0.863 |
| P2RY14 | GALK2 | 0.863 |
| VAMP5 | YEATS2 | 0.863 |
| GBP1 | DPF2 | 0.863 |
| P2RY14 | IMMT | 0.863 |
| GBP5 | SOS1 | 0.863 |
| GBP1 | IARS2 | 0.863 |
| DAPP1 | XPO1 | 0.863 |
| P2RY14 | STX11 | 0.863 |
| P2RY14 | E2F5 | 0.863 |
| GBP1 | PON2 | 0.863 |
| POLB | TOE1 | 0.863 |
| UBE216 | PSTPIP2 | 0.863 |
| POLB | CHRNB1 | 0.863 |
| PSME1 | MRPL49 | 0.863 |
| SECTM1 | PES1 | 0.863 |
| UBE2L6 | PML | 0.863 |
| P2RY14 | PSD4 | 0.863 |
| SUCLG1 | GBP1 | 0.863 |
| VAMP5 | ABCF2 | 0.863 |
| P2RY14 | TBRG4 | 0.863 |
| IRF1 | GNG11 | 0.863 |
| APOL2 | OGDH | 0.863 |
| UBE2L6 | ZNF76 | 0.863 |
| GBP1 | CLDND1 | 0.863 |
| POLB | RGS12 | 0.863 |
| CHMP2A | GBP1 | 0.863 |
| PSME1 | PRRG4 | 0.863 |
| P2RY14 | SCAP | 0.863 |
| GBP1 | ETF1 | 0.863 |
| P2RY14 | TMBIM4 | 0.863 |
| P2RY14 | BCCIP | 0.863 |
| ATG3 | GPBP1L1 | 0.863 |
| GBP1 | PSMD11 | 0.863 |
| GBP1 | BBS7 | 0.863 |
| P2RY14 | MED9 | 0.863 |
| SECTM1 | RRS1 | 0.863 |
| P2RY14 | TNK2 | 0.863 |
| LHFPL2 | MCM6 | 0.863 |
| APOL1 | BCR | 0.863 |
| GBP5 | SLC12A2 | 0.863 |
| POLB | GSPT2 | 0.863 |
| PSME1 | APOL1 | 0.863 |
| POLB | ZFR | 0.863 |
| POLB | UBE21 | 0.863 |
| POLB | SERGEF | 0.863 |
| GTF2B | GBP1 | 0.863 |
| SECTM1 | DENND1A | 0.863 |
| VAMP5 | UPB1 | 0.863 |
| IL15RA | POLR3B | 0.863 |
| POLB | ITGA5 | 0.863 |
| LHFPL2 | NOLC1 | 0.863 |
| GBP1 | LAX1 | 0.863 |
| GBP5 | DRAP1 | 0.863 |
| GBP1 | DNM1L | 0.863 |
| GBP1 | YWHAQ | 0.863 |
| PSME1 | ODF2 | 0.863 |
| UBE2L6 | TAF6 | 0.863 |
| POLB | RAD1 | 0.863 |
| GBP1 | STARD8 | 0.862 |
| P2RY14 | PEX7 | 0.862 |
| UBE2L6 | DPH2 | 0.862 |
| GBP5 | NR1H2 | 0.862 |
| GBP1 | YWHAZ | 0.862 |
| GBP1 | CONK | 0.862 |
| UBE2L6 | PAXIP1 | 0.862 |
| GBP1 | NUP50 | 0.862 |
| GBP1 | PMM2 | 0.862 |
| GBP1 | MIS12 | 0.862 |
| GBP1 | SLC25A12 | 0.862 |
| GBP1 | TAF4 | 0.862 |
| AIM2 | ANKS1A | 0.862 |
| GBP1 | AP3B1 | 0.862 |
| UBE2L6 | SON | 0.862 |
| C1QA | AKAP1 | 0.862 |
| GBP1 | BECN1 | 0.862 |
| P2RY14 | ARL1 | 0.862 |
| SECTM1 | AP1G1 | 0.862 |
| APOL1 | DGKD | 0.862 |
| P2RY14 | PANK2 | 0.862 |
| UBE216 | CALU | 0.862 |
| GBP5 | USP25 | 0.862 |
| POLB | PCBP4 | 0.862 |
| SECTM1 | WDR25 | 0.862 |
| GBP5 | TRIO | 0.862 |
| SECTM1 | GMIP | 0.862 |
| SECTM1 | FANCE | 0.862 |
| GBP1 | POLR2D | 0.862 |
| POLB | AMPD3 | 0.862 |
| P2RY14 | FZD1 | 0.862 |
| PSME1 | LAS1L | 0.862 |
| P2RY14 | SHB | 0.862 |
| GBP5 | CSPP1 | 0.862 |
| P2RY14 | SPHK2 | 0.862 |
| UBE216 | PPM1G | 0.862 |
| UBE216 | FLII | 0.862 |
| LHFPL2 | SLTM | 0.862 |
| UBE2L6 | PPARD | 0.862 |
| P2RY14 | SCARB1 | 0.862 |
| CD59 | GBP1 | 0.862 |
| POLB | REV3L | 0.862 |
| GBP1 | KLHL7 | 0.862 |
| GBP5 | COTL1 | 0.862 |
| UBE2L6 | EIF4ENIF1 | 0.862 |
| GBP1 | MAGED1 | 0.862 |
| POLB | USP3 | 0.862 |
| P2RY14 | RAD51C | 0.862 |
| AIM2 | CPVL | 0.862 |
| GBP5 | CCDC6 | 0.862 |
| GBP5 | NCF2 | 0.862 |
| GBP5 | TMEM126B | 0.862 |
| POLB | PDK3 | 0.862 |
| UBE2L6 | GPR132 | 0.862 |
| GBP5 | UBAP1 | 0.862 |
| SECTM1 | ERF | 0.862 |
| GBP1 | DDX24 | 0.862 |
| P2RY14 | EIF2AK1 | 0.862 |
| GBP1 | TUFT1 | 0.862 |
| C1QA | DHCR7 | 0.862 |
| SECTM1 | MED9 | 0.862 |
| P2RY14 | RNF121 | 0.862 |
| GBP5 | PRPF38B | 0.862 |
| LHFPL2 | EIF1 | 0.862 |
| GBP5 | PARP8 | 0.862 |
| SECTM1 | DEF6 | 0.862 |
| P2RY14 | KIF23 | 0.862 |
| GBP1 | AASDHPPT | 0.862 |
| POLB | CLPX | 0.862 |
| P2RY14 | CDC25B | 0.862 |
| P2RY14 | CALCOCO1 | 0.862 |
| SECTM1 | PRKAR2A | 0.862 |
| UBE216 | KLF3 | 0.862 |
| P2RY14 | TSC2 | 0.862 |
| GBP1 | ATP13A1 | 0.862 |
| GBP1 | AMPD3 | 0.862 |
| P2RY14 | TM2D3 | 0.862 |
| GBP5 | AHCYL1 | 0.862 |
| PZRY14 | RPL3L | 0.862 |
| P2RY14 | RHEB | 0.862 |
| COMMD5 | GBP1 | 0.862 |
| GBP1 | SMAD4 | 0.862 |
| P2RY14 | ZNF696 | 0.862 |
| GBP1 | ELAC2 | 0.862 |
| C1QA | TRAP1 | 0.862 |
| UBE2L6 | AIM2 | 0.862 |
| POLB | ABCC10 | 0.862 |
| GBP1 | EXT1 | 0.862 |
| LHFPL2 | SLC16A6 | 0.862 |
| C1QA | TIMM44 | 0.862 |
| GBP5 | POLR2F | 0.862 |
| GBP1 | AACS | 0.862 |
| GSTO1 | APOL2 | 0.862 |
| P2RY14 | PTPN7 | 0.862 |
| GBP5 | GAA | 0.862 |
| GBP1 | PIGB | 0.862 |
| UBE2L6 | CDK5RAP1 | 0.862 |
| UBE2L6 | ST6GAL1 | 0.862 |
| UBE2L6 | ITGA7 | 0.862 |
| POLB | ANKRD27 | 0.862 |
| GBP1 | POLH | 0.862 |
| POLB | NUP98 | 0.862 |
| P2RY14 | ADCY9 | 0.862 |
| C1QA | VPS11 | 0.862 |
| P2RY14 | ER5 | 0.862 |
| GBP1 | MGAT2 | 0.862 |
| DAPP1 | OSBP | 0.862 |
| C1QA | OGDH | 0.862 |
| APOL1 | SDC1 | 0.862 |
| POLB | CAMSAP1 | 0.862 |
| C1QA | ATP13A1 | 0.862 |
| POLB | PHB | 0.862 |
| P2RY14 | PIGV | 0.862 |
| GBP5 | FUBP1 | 0.862 |
| GBP5 | HDAC4 | 0.862 |
| GBP1 | HIC2 | 0.862 |
| AIM2 | SLC16A6 | 0.862 |
| SECTM1 | ARAF | 0.862 |
| PZRY14 | NMUR1 | 0.862 |
| POLB | PPP1R8 | 0.862 |
| GBP1 | YIPF5 | 0.862 |
| GBP1 | DENND4C | 0.862 |
| GBP5 | LSP1 | 0.862 |
| C1QA | CTCF | 0.862 |
| C1QA | ELAC2 | 0.862 |
| GBP1 | SMARCE1 | 0.862 |
| C1QA | DIAPH1 | 0.862 |
| UBE2L6 | SFXN1 | 0.862 |
| IL15RA | AKAP1 | 0.862 |
| GBP5 | MTO1 | 0.862 |
| VAMP5 | MAP4 | 0.862 |
| GBP5 | RCBTB2 | 0.862 |
| C1QA | CHAF1B | 0.862 |
| SECTM1 | AAAS | 0.862 |
| VAMP5 | CTCF | 0.862 |
| GBP5 | LMAN1 | 0.862 |
| P2RY14 | ADD1 | 0.862 |
| UBE2L6 | FZD5 | 0.862 |
| UBE2L6 | MSH2 | 0.862 |
| POLB | UBIAD1 | 0.862 |
| GBP1 | CHD4 | 0.862 |
| P2RY14 | BAIAP2 | 0.862 |
| GBP1 | PDHX | 0.862 |
| GBP1 | SEH1L | 0.862 |
| GBP1 | ASB8 | 0.862 |
| GBP1 | GMEB1 | 0.862 |
| GBP1 | KRIT1 | 0.862 |
| UBE2L6 | SETD3 | 0.862 |
| POLB | PPP4R1 | 0.862 |
| UBE2L6 | DENND2D | 0.862 |
| POLB | NMT2 | 0.862 |
| C1QA | IL1B | 0.862 |
| P2RY14 | CIDEB | 0.862 |
| GBP1 | PCYOX1 | 0.862 |
| GBP1 | PRKCH | 0.862 |
| GBP1 | SIRT1 | 0.862 |
| PZRY14 | SPSB3 | 0.862 |
| P2RY14 | FKBP4 | 0.862 |
| UBE2L6 | ATP2B4 | 0.862 |
| P2RY14 | NECAP2 | 0.862 |
| C1QA | MAP4 | 0.862 |
| P2RY14 | RFX2 | 0.862 |
| VAMP5 | CSRP1 | 0.862 |
| GBP1 | ODF2 | 0.862 |
| GBP5 | PCM1 | 0.862 |
| SECTM1 | PSTPIP1 | 0.862 |
| GBP1 | ACTR8 | 0.862 |
| GBP1 | CAMSAP1 | 0.862 |
| VAMP5 | ZNF200 | 0.862 |
| P2RY14 | ASH2L | 0.862 |
| P2RY14 | BCS1L | 0.862 |
| P2RY14 | FUCA1 | 0.862 |
| POLB | PRPF8 | 0.862 |
| SECTM1 | TBCD | 0.862 |
| ACO1 | GBP1 | 0.862 |
| POLB | CLASP1 | 0.862 |
| UBE2L6 | SATB1 | 0.862 |
| UBE2L6 | LSR | 0.862 |
| PML | GLG1 | 0.862 |
| SMARCD2 | P2RY14 | 0.862 |
| UBE2L6 | DDX23 | 0.862 |
| RHOC | GBP1 | 0.862 |
| SECTM1 | ATP9B | 0.862 |
| GBP1 | SQLE | 0.862 |
| GBP1 | PML | 0.862 |
| GBP1 | FANCE | 0.862 |
| UBE2L6 | SEC23B | 0.862 |
| VAMP5 | TNK2 | 0.862 |
| PSME1 | STK38 | 0.862 |
| UBE2L6 | PSMD2 | 0.862 |
| AIM2 | HIGD2A | 0.862 |
| GBP5 | FBXW7 | 0.862 |
| UBE2L6 | CACNA1I | 0.862 |
| UBE2L6 | KCNAB2 | 0.862 |
| GBP5 | NOTCH1 | 0.862 |
| P2RY14 | OGG1 | 0.862 |
| GBP1 | SLC38A2 | 0.862 |
| SECTM1 | SLA | 0.862 |
| VAMP5 | NUMA1 | 0.862 |
| POLB | KLF11 | 0.862 |
| GBP5 | ROCK2 | 0.862 |
| AIM2 | HK2 | 0.862 |
| POLB | CCDC25 | 0.862 |
| VSIG4 | GBP1 | 0.862 |
| ISOC2 | GBP1 | 0.862 |
| APOL2 | CAMK2G | 0.862 |
| P2RY14 | DDX54 | 0.862 |
| UBE216 | ZNF318 | 0.862 |
| PHKG2 | GBP1 | 0.862 |
| P2RY14 | LZTR1 | 0.862 |
| UBE2L6 | NDUFA1 | 0.862 |
| DUSP22 | GBP1 | 0.862 |
| GBP5 | RWDD1 | 0.862 |
| UBE2L6 | ZHX2 | 0.862 |
| GBP1 | EIF5 | 0.862 |
| GBP1 | S100A8 | 0.862 |
| P2RY14 | BCL212 | 0.862 |
| P2RY14 | LY9 | 0.862 |
| SECTM1 | CBX1 | 0.862 |
| POLB | MTHFR | 0.862 |
| POLB | PREP | 0.862 |
| POLB | GLUL | 0.862 |
| POLB | STAT1 | 0.862 |
| UBE216 | NENF | 0.862 |
| GBP1 | ARMCX5 | 0.862 |
| GBP5 | ATP6V0D1 | 0.862 |
| POLB | MUS81 | 0.862 |
| IRF1 | LASP1 | 0.862 |
| UBE2L6 | DNAJA3 | 0.862 |
| UBE2L6 | LRRC8D | 0.862 |
| GBP1 | ITCH | 0.862 |
| POLB | ZNF365 | 0.862 |
| SLC25A22 | GBP1 | 0.862 |
| P2RY14 | ASRGL1 | 0.862 |
| GBP1 | LBR | 0.862 |
| UBE2L6 | SUV39H1 | 0.862 |
| VAMP5 | RNF25 | 0.862 |
| VAMP5 | POLG | 0.862 |
| PML | VAMP5 | 0.862 |
| GBP1 | ANKRD10 | 0.862 |
| MTCH2 | GBP1 | 0.862 |
| F13A1 | VAMP5 | 0.862 |
| LHFPL2 | TOPBP1 | 0.862 |
| GBP5 | EIF4G3 | 0.862 |
| APOL2 | IL16 | 0.862 |
| GBP5 | OXR1 | 0.862 |
| GBP1 | MGAT4A | 0.862 |
| GBP5 | CREB1 | 0.862 |
| GBP1 | UBIAD1 | 0.862 |
| UBE216 | MAPK13 | 0.862 |
| GBP1 | CCNG2 | 0.862 |
| P2RY14 | LRRC41 | 0.862 |
| UBE2L6 | IL16 | 0.862 |
| NPC2 | MAX | 0.862 |
| UBE2L6 | TBRG4 | 0.862 |
| APOL1 | AKAP1 | 0.862 |
| GBP1 | KLF10 | 0.862 |
| BCKDK | GBP1 | 0.862 |
| P2RY14 | RFX5 | 0.862 |
| P2RY14 | CDKAL1 | 0.862 |
| POLB | CCNG2 | 0.862 |
| ARSB | P2RY14 | 0.862 |
| UBE216 | CNOT2 | 0.862 |
| UBE216 | PSMD1 | 0.862 |
| APOL2 | SPEN | 0.862 |
| FOLR2 | AIM2 | 0.862 |
| POLB | CHD4 | 0.862 |
| GBP1 | VILL | 0.862 |
| SECTM1 | ATP13A1 | 0.862 |
| TNFRSF14 | C1QA | 0.862 |
| C1QA | GALNT2 | 0.862 |
| GBP5 | RNF14 | 0.862 |
| SECTM1 | NENF | 0.862 |
| PSME1 | AKAP1 | 0.862 |
| P2RY14 | MLH1 | 0.862 |
| AIM2 | ETS2 | 0.862 |
| GBP1 | PICALM | 0.862 |
| VAMP5 | PRMT7 | 0.862 |
| UBE2L6 | USP48 | 0.862 |
| NPC2 | FAS | 0.862 |
| P2RY14 | NEDD4L | 0.862 |
| VAMP5 | SERPINB8 | 0.862 |
| P2RY14 | P2RY13 | 0.862 |
| GBP1 | SYNGR1 | 0.862 |
| VAMP5 | STT3A | 0.862 |
| GBP5 | DDX17 | 0.862 |
| GBP5 | TBCE | 0.862 |
| P2RY14 | POLR2H | 0.862 |
| IL15RA | SMARCC1 | 0.862 |
| UBE216 | FOLR2 | 0.862 |
| GBP5 | SSNA1 | 0.862 |
| STX8 | GBP1 | 0.862 |
| POLB | TM9SF4 | 0.862 |
| SECTM1 | MCM3 | 0.862 |
| SECTM1 | CRY2 | 0.862 |
| GBP1 | RASGRP1 | 0.862 |
| GBP5 | PPID | 0.862 |
| PSME1 | ATP2A3 | 0.862 |
| GSTO1 | IL15RA | 0.862 |
| SECTM1 | MAGED1 | 0.862 |
| APOL2 | FT122 | 0.862 |
| UBE2L6 | CBX4 | 0.862 |
| GBP5 | DYRK1A | 0.862 |
| POLB | LETMD1 | 0.862 |
| FAS | PPP3CA | 0.862 |
| GBP1 | UBE2G1 | 0.862 |
| UBE2L6 | ZZEF1 | 0.862 |
| SERTAD3 | GBP1 | 0.862 |
| KDELR1 | POLB | 0.862 |
| PSME1 | TSR1 | 0.862 |
| APOLZ | ELAC2 | 0.862 |
| GBP5 | SEL1L | 0.862 |
| SECTM1 | SSBP1 | 0.862 |
| VAMP5 | BAHD1 | 0.862 |
| PSME1 | NFATC3 | 0.862 |
| GBP5 | SLC29A1 | 0.861 |
| POLB | MIS12 | 0.861 |
| VAMP5 | ST14 | 0.861 |
| VAMP5 | PPRC1 | 0.861 |
| UBE2L6 | NIPBL | 0.861 |
| CYB561 | ATG3 | 0.861 |
| P2RY14 | MAPK7 | 0.861 |
| UBE2L6 | XPO1 | 0.861 |
| GBP5 | MSRA | 0.861 |
| VAMP5 | GTPBP4 | 0.861 |
| SECTM1 | INPP5A | 0.861 |
| VAMP5 | TBRG4 | 0.861 |
| GBP1 | PAM | 0.861 |
| GBP5 | USP33 | 0.861 |
| DAPP1 | SMARCC1 | 0.861 |
| POLB | DDX24 | 0.861 |
| P2RY14 | SDCBP | 0.861 |
| APOL2 | RPAP1 | 0.861 |
| GBP5 | PNOC | 0.861 |
| UBE2L6 | POLD3 | 0.861 |
| POLB | TEX261 | 0.861 |
| DAPP1 | NIPBL | 0.861 |
| IL1B | MAK | 0.861 |
| GBP1 | RPA1 | 0.861 |
| UBE2L6 | WBP11 | 0.861 |
| GBP5 | NAB1 | 0.861 |
| GBP1 | ATP9B | 0.861 |
| UBE216 | SNW1 | 0.861 |
| F13A1 | POLB | 0.861 |
| GBP1 | PPP1CC | 0.861 |
| APOL2 | TIMM44 | 0.861 |
| LSM1 | LIMK1 | 0.861 |
| APOL2 | ABCF3 | 0.861 |
| APOL1 | CEP250 | 0.861 |
| POLB | RRAGC | 0.861 |
| GSTO1 | PRRG4 | 0.861 |
| GBP5 | QPCT | 0.861 |
| APOL2 | ARMC7 | 0.861 |
| ACP5 | STAT1 | 0.861 |
| CD46 | VAMP5 | 0.861 |
| GBP5 | FRAT2 | 0.861 |
| UBE2L6 | HERC1 | 0.861 |
| VAMP5 | EBP | 0.861 |
| GBP1 | STIM1 | 0.861 |
| SLC43A1 | GBP1 | 0.861 |
| GBP5 | OTUD4 | 0.861 |
| GBP1 | CBX4 | 0.861 |
| GBP1 | RAD23B | 0.861 |
| UBE2L6 | YWHAZ | 0.861 |
| POLB | CCDC51 | 0.861 |
| VAMP5 | VCP | 0.861 |
| GBP1 | PINK1 | 0.861 |
| P2RY14 | DAZAP1 | 0.861 |
| UBE2L6 | KLF11 | 0.861 |
| SECTM1 | ZFPL1 | 0.861 |
| GBP1 | DOCK2 | 0.861 |
| P2RY14 | CYBRD1 | 0.861 |
| GBP1 | REL | 0.861 |
| SLC25A28 | GBP1 | 0.861 |
| GBP1 | EIF4ENIF1 | 0.861 |
| IRF1 | VAMP5 | 0.861 |
| GBP5 | TTC1 | 0.861 |
| CALCOCO2 | APOL2 | 0.861 |
| GBP5 | TRIMS | 0.861 |
| VAMP5 | OSBPL9 | 0.861 |
| GBP1 | BSDC1 | 0.861 |
| GBP5 | MGAT1 | 0.861 |
| GBP5 | STOML2 | 0.861 |
| LHFPL2 | RNGTT | 0.861 |
| VAMP5 | MAP3K4 | 0.861 |
| SECTM1 | HMGA1 | 0.861 |
| GBP1 | DLG4 | 0.861 |
| P2RY14 | CHRNA10 | 0.861 |
| POLB | ASB8 | 0.861 |
| GBP5 | CTSW | 0.861 |
| GBP5 | RAB21 | 0.861 |
| NDUFB6 | LHFPL2 | 0.861 |
| NPC2 | PRRG4 | 0.861 |
| P2RY14 | MRPS30 | 0.861 |
| GBP5 | IFITM1 | 0.861 |
| PSME1 | MCM3 | 0.861 |
| GBP5 | POLA2 | 0.861 |
| LIMK1 | FAS | 0.861 |
| UBE216 | HMGA1 | 0.861 |
| POLB | OGG1 | 0.861 |
| LHFPL2 | PHF3 | 0.861 |
| AIM2 | MTMR6 | 0.861 |
| GBP1 | SNX16 | 0.861 |
| PML | CTCF | 0.861 |
| CYB5R3 | GBP1 | 0.861 |
| CYB561 | BAHD1 | 0.861 |
| GBP5 | FURIN | 0.861 |
| POLB | SRD5A1 | 0.861 |
| UBE2L6 | PRKD2 | 0.861 |
| POLB | RBM10 | 0.861 |
| AIM2 | MX2 | 0.861 |
| P2RY14 | PCCB | 0.861 |
| UBE2L6 | USP4 | 0.861 |
| APOL2 | CHD4 | 0.861 |
| GBP5 | AGPS | 0.861 |
| VAMP5 | SETD3 | 0.861 |
| GBP5 | BACH1 | 0.861 |
| AIM2 | ALDH1A1 | 0.861 |
| UBE2L6 | HTRA2 | 0.861 |
| POLB | NFKB1 | 0.861 |
| CYB561 | CEP250 | 0.861 |
| P2RY14 | MRPL9 | 0.861 |
| P2RY14 | POLE3 | 0.861 |
| JAM3 | C1QA | 0.861 |
| VAMP5 | SYNCRIP | 0.861 |
| APOL2 | AXIN1 | 0.861 |
| POLB | UPF3A | 0.861 |
| VAMP5 | RPAP1 | 0.861 |
| F13A1 | GBP1 | 0.861 |
| GBP5 | PCSK1N | 0.861 |
| POLB | ACTR1B | 0.861 |
| AIM2 | STAG1 | 0.861 |
| APOL1 | GRWD1 | 0.861 |
| GBP5 | TRAPPC6A | 0.861 |
| CYB561 | AKAP1 | 0.861 |
| POLB | VPS39 | 0.861 |
| P2RY14 | RASGRP2 | 0.861 |
| P2RY14 | AK2 | 0.861 |
| LHFPL2 | APOL3 | 0.861 |
| UBE216 | PIK3C2B | 0.861 |
| P2RY14 | DHRS3 | 0.861 |
| UBE2L6 | GIMAP6 | 0.861 |
| GBP1 | SMU1 | 0.861 |
| GBP1 | SMAD7 | 0.861 |
| APOL2 | NCOA1 | 0.861 |
| GBP1 | NGLY1 | 0.861 |
| VAMP5 | POLD1 | 0.861 |
| VAMP5 | PFAS | 0.861 |
| GBP1 | NUP54 | 0.861 |
| GSTO1 | APOL1 | 0.861 |
| CYB561 | ILF3 | 0.861 |
| GBP1 | JARID2 | 0.861 |
| VAMP5 | PARP6 | 0.861 |
| UBE216 | VPS33B | 0.861 |
| GBP1 | IL7R | 0.861 |
| GBP1 | PRKAR1A | 0.861 |
| APOL2 | ATP2A3 | 0.861 |
| APOL2 | OSBPL9 | 0.861 |
| POLB | TLE2 | 0.861 |
| P2RY14 | CHKA | 0.861 |
| POLB | GTPBP8 | 0.861 |
| APOL2 | TNK2 | 0.861 |
| APOL1 | GEMIN4 | 0.861 |
| SECTM1 | EIF1 | 0.861 |
| PSME1 | NUMA1 | 0.861 |
| P2RY14 | UBE2G2 | 0.861 |
| UBE216 | DGKA | 0.861 |
| NMI | ACP5 | 0.861 |
| POLB | KCNK3 | 0.861 |
| GBP1 | ANGEL1 | 0.861 |
| GBP1 | MAN1C1 | 0.861 |
| AKR1A1 | LHFPL2 | 0.861 |
| P2RY14 | CYBB | 0.861 |
| GBP1 | RNMT | 0.861 |
| POLB | POLRMT | 0.861 |
| GBP1 | ABCC10 | 0.861 |
| VAMP5 | RUVBL2 | 0.861 |
| GBP1 | IL16 | 0.861 |
| DAPP1 | SATB1 | 0.861 |
| UBE2L6 | DEDD | 0.861 |
| GBP1 | GPR137 | 0.861 |
| SECTM1 | PER2 | 0.861 |
| UBE2L6 | ADNP | 0.861 |
| C1QA | NUP210 | 0.861 |
| C1QA | PLCD1 | 0.861 |
| UBE2L6 | PGM1 | 0.861 |
| UBE2L6 | ALMS1 | 0.861 |
| GBP1 | DHX38 | 0.861 |
| GBP5 | MRPL28 | 0.861 |
| POLB | LCP2 | 0.861 |
| P2RY14 | TRMT1 | 0.861 |
| POLB | EXOSC8 | 0.861 |
| TMEM59 | APOL1 | 0.861 |
| UBE216 | CCND2 | 0.861 |
| GBP1 | CDCA8 | 0.861 |
| GBP5 | PPWD1 | 0.861 |
| UBE2L6 | EXT1 | 0.861 |
| POLB | WBP11 | 0.861 |
| SECTM1 | TSC1 | 0.861 |
| POLB | THAP7 | 0.861 |
| POLB | GMPR2 | 0.861 |
| GBP1 | FNBP1 | 0.861 |
| GBP1 | SNW1 | 0.861 |
| GBP5 | SLC15A3 | 0.861 |
| P2RY14 | CAB39L | 0.861 |
| GBP1 | CASP6 | 0.861 |
| GBP1 | FLNB | 0.861 |
| POLB | ILKAP | 0.861 |
| UBE2L6 | KLF13 | 0.861 |
| P2RY14 | LRFN3 | 0.861 |
| P2RY14 | PCBP4 | 0.861 |
| POLB | CYB561D2 | 0.861 |
| VAMP5 | NXT1 | 0.861 |
| GBP5 | IL32 | 0.861 |
| C1QA | BTN2A2 | 0.861 |
| APOL1 | PDHA1 | 0.861 |
| NMI | C1QA | 0.861 |
| POLB | CTDSPL | 0.861 |
| UBE216 | GPA33 | 0.861 |
| P2RY14 | ACOX3 | 0.861 |
| POLB | RNF113A | 0.861 |
| P2RY14 | CLK2 | 0.861 |
| NPC2 | IL15RA | 0.861 |
| GBP1 | USP4 | 0.861 |
| P2RY14 | FUT7 | 0.861 |
| GBP1 | MTHFD1 | 0.861 |
| POLB | WDR74 | 0.861 |
| UBE2L6 | ELF4 | 0.861 |
| GBP5 | RTP4 | 0.861 |
| POLB | HTRA2 | 0.861 |
| GBP5 | ALDOA | 0.861 |
| POLB | LCK | 0.861 |
| POLB | SLC25A28 | 0.861 |
| PSME1 | VCP | 0.861 |
| P2RY14 | CCT4 | 0.861 |
| PZRY14 | SMOX | 0.861 |
| APOL1 | LIG1 | 0.861 |
| APOL2 | MAZ | 0.861 |
| POP7 | GBP1 | 0.861 |
| GBP5 | KIAA0319L | 0.861 |
| POLB | MBD1 | 0.861 |
| C1QA | PDIA4 | 0.861 |
| APOL2 | GANAB | 0.861 |
| SECTM1 | CNOT2 | 0.861 |
| P2RY14 | AAAS | 0.861 |
| GBP1 | NARS2 | 0.861 |
| NDUFB6 | VAMP5 | 0.861 |
| GBP5 | ABI2 | 0.861 |
| SECTM1 | RBM10 | 0.861 |
| ATP7B | VAMP5 | 0.861 |
| POLB | STARD5 | 0.861 |
| UBE2L6 | GALNT2 | 0.861 |
| C1QA | SH3TC1 | 0.861 |
| UBE2L6 | ANKRD27 | 0.861 |
| GBP5 | PTPRC | 0.861 |
| POLB | KYNU | 0.861 |
| GBP1 | DPH2 | 0.861 |
| POLB | CALU | 0.861 |
| ATG3 | CTCF | 0.861 |
| UBE216 | BRD9 | 0.861 |
| PZRY14 | CLSTN1 | 0.861 |
| GBP1 | VPS54 | 0.861 |
| GBP1 | DCP2 | 0.861 |
| GBP1 | CASP1 | 0.861 |
| GBP1 | SOAT1 | 0.861 |
| SECTM1 | RIPK2 | 0.861 |
| VTI1B | GBP1 | 0.861 |
| UBE2L6 | POLD1 | 0.861 |
| APOL1 | PSTPIP2 | 0.861 |
| NPC2 | VAMP5 | 0.861 |
| UBE2L6 | VPS13D | 0.861 |
| P2RY14 | GTPBP2 | 0.861 |
| CYB561 | EPHA4 | 0.861 |
| GBP1 | PDK3 | 0.861 |
| GBP5 | TRIP11 | 0.861 |
| GBP1 | GPA33 | 0.861 |
| GBP1 | METAP1 | 0.861 |
| P2RY14 | ST3GAL4 | 0.861 |
| GBP5 | SPTLC2 | 0.861 |
| SECTM1 | MAPKAPK2 | 0.861 |
| GBP5 | ACSL4 | 0.861 |
| UBE2L6 | PUM1 | 0.861 |
| POLB | MPPE1 | 0.861 |
| UBE2L6 | CPSF1 | 0.861 |
| IGFBP2 | P2RY14 | 0.861 |
| VAMP5 | DHX9 | 0.861 |
| VAMP5 | FT122 | 0.861 |
| GBP5 | LMNB1 | 0.861 |
| C1QA | TOB2 | 0.861 |
| GBP5 | TBXAS1 | 0.861 |
| VAMP5 | BCAT2 | 0.861 |
| GBP5 | NFAT5 | 0.861 |
| GBP5 | BCL6 | 0.861 |
| UBE2L6 | PLK3 | 0.861 |
| UBE216 | GYPC | 0.861 |
| LHFPL2 | ATF7IP | 0.861 |
| VAMP5 | GTF3C5 | 0.861 |
| CYB561D2 | GBP1 | 0.861 |
| PZRY14 | C1GALT1C1 | 0.861 |
| BTN2A2 | VAMP5 | 0.861 |
| P2RY14 | ACP1 | 0.861 |
| GBP1 | PCSK6 | 0.861 |
| PSME1 | TIMM44 | 0.861 |
| SECTM1 | RRBP1 | 0.861 |
| PZRY14 | LIMK2 | 0.861 |
| POLB | ABHD6 | 0.861 |
| P2RY14 | LAMP1 | 0.861 |
| VAMP5 | RUVBL1 | 0.86 |
| GBP5 | SYNJ1 | 0.86 |
| PSME1 | PITRM1 | 0.86 |
| HMGCL | GBP1 | 0.86 |
| SECTM1 | PXMP4 | 0.86 |
| GBP1 | CLPX | 0.86 |
| GBP1 | RAD1 | 0.86 |
| LIMK1 | CTCF | 0.86 |
| C1QA | STT3A | 0.86 |
| PSTPIP2 | VAMP5 | 0.86 |
| UBE216 | ARCN1 | 0.86 |
| GBP1 | CCR4 | 0.86 |
| P2RY14 | ABHD4 | 0.86 |
| POLB | SRP54 | 0.86 |
| AKR1A1 | SECTM1 | 0.86 |
| GBP1 | NUP107 | 0.86 |
| POLB | RRN3 | 0.86 |
| GBP1 | HSPH1 | 0.86 |
| DAPP1 | USP48 | 0.86 |
| GBP5 | PHF11 | 0.86 |
| SECTM1 | ST3GAL2 | 0.86 |
| PSME1 | PPRC1 | 0.86 |
| GBP5 | CD4 | 0.86 |
| PRRG4 | IL15RA | 0.86 |
| UBE2L6 | AAAS | 0.86 |
| SECTM1 | VDR | 0.86 |
| P2RY14 | CBX7 | 0.86 |
| UBE2L6 | CLN6 | 0.86 |
| POLB | KLHL3 | 0.86 |
| LAPTM4A | SECTM1 | 0.86 |
| C1QA | MCM3 | 0.86 |
| GBP1 | INSIG2 | 0.86 |
| GBP1 | DDX23 | 0.86 |
| C1QA | CLEC4A | 0.86 |
| UBE216 | SLTM | 0.86 |
| UBE216 | SRPK2 | 0.86 |
| GSTO1 | PDE4B | 0.86 |
| LIMK1 | DHX9 | 0.86 |
| UBE216 | GNE | 0.86 |
| VAMP5 | NFATC1 | 0.86 |
| GBP1 | ATG12 | 0.86 |
| GBP5 | AKAP10 | 0.86 |
| P2RY14 | TM6SF1 | 0.86 |
| GBP1 | SETD3 | 0.86 |
| SECTM1 | RFC1 | 0.86 |
| APOL2 | EXOC7 | 0.86 |
| PSME1 | NUP210 | 0.86 |
| GBP5 | S100A11 | 0.86 |
| GBP1 | CBX1 | 0.86 |
| UBE216 | MYH9 | 0.86 |
| SECTM1 | PSMD1 | 0.86 |
| VAMP5 | RRM1 | 0.86 |
| SECTM1 | PRDM4 | 0.86 |
| GBP1 | VCP | 0.86 |
| POLB | FLII | 0.86 |
| POLB | EXOSC9 | 0.86 |
| VAMP5 | CDCA4 | 0.86 |
| SECTM1 | UQCRC1 | 0.86 |
| GBP1 | UBE21 | 0.86 |
| GBP5 | ANPEP | 0.86 |
| POLB | METTL1 | 0.86 |
| POLB | CYP20A1 | 0.86 |
| UBE216 | VPS39 | 0.86 |
| GBP5 | TAF1 | 0.86 |
| POLB | TBC1D13 | 0.86 |
| UBE2L6 | GMPR2 | 0.86 |
| GBP1 | ECHDC2 | 0.86 |
| CLEC7A | VAMP5 | 0.86 |
| POLB | MFGE8 | 0.86 |
| VAMP3 | GBP1 | 0.86 |
| POLB | GGA1 | 0.86 |
| PSTPIP2 | SLC16A6 | 0.86 |
| VAMP5 | PSMD11 | 0.86 |
| GBP5 | ATP6V0B | 0.86 |
| P2RY14 | CNDP2 | 0.86 |
| POLB | APH1A | 0.86 |
| GBP1 | ANXA7 | 0.86 |
| VAMP5 | PTGES2 | 0.86 |
| SECTM1 | FANCL | 0.86 |
| IL15RA | RPAP1 | 0.86 |
| GBP1 | ZXDC | 0.86 |
| VAMP5 | NUP210 | 0.86 |
| P2RY14 | DCTN5 | 0.86 |
| GBP1 | PTPN22 | 0.86 |
| C1QA | DDIT4 | 0.86 |
| UBE2L6 | CD59 | 0.86 |
| UBE2L6 | GFI1B | 0.86 |
| C1QA | ARID1A | 0.86 |
| C1QA | NFATC1 | 0.86 |
| P2RY14 | CANT1 | 0.86 |
| PSME1 | PARP6 | 0.86 |
| PZRY14 | DUSP12 | 0.86 |
| GBP5 | TLR4 | 0.86 |
| APOL3 | GBP1 | 0.86 |
| PSME1 | FTSJ3 | 0.86 |
| GBP5 | PRDM1 | 0.86 |
| PSME1 | MCM7 | 0.86 |
| PSTPIP2 | STAG1 | 0.86 |
| GBP1 | TSTA3 | 0.86 |
| GBP1 | PLEKHF2 | 0.86 |
| C1QA | POLG | 0.86 |
| GBP5 | PAPOLG | 0.86 |
| GBP5 | TLN1 | 0.86 |
| UBE216 | ABLIM1 | 0.86 |
| P2RY14 | GDPD5 | 0.86 |
| GBP5 | P2RY2 | 0.86 |
| GNG11 | APOL3 | 0.86 |
| CALCOCO2 | DGKD | 0.86 |
| APOL2 | SMAD3 | 0.86 |
| P2RY14 | ANGPT1 | 0.86 |
| UBE2L6 | MINK1 | 0.86 |
| GBP5 | TARDBP | 0.86 |
| SECTM1 | VPS13D | 0.86 |
| LIMK1 | TSPAN14 | 0.86 |
| POLB | TNFRSF9 | 0.86 |
| GSTO1 | LHFPL2 | 0.86 |
| APOL1 | CALCOCO2 | 0.86 |
| YIPF3 | GBP1 | 0.86 |
| KPTN | GBP1 | 0.86 |
| SECTM1 | DDX54 | 0.86 |
| UBE2L6 | LRIG1 | 0.86 |
| GBP1 | SCMH1 | 0.86 |
| GBP1 | PDCD6IP | 0.86 |
| P2RY14 | NXF1 | 0.86 |
| GBP1 | SKP2 | 0.86 |
| SECTM1 | TTF1 | 0.86 |
| GBP5 | ANKRD49 | 0.86 |
| UBE2L6 | GTF2B | 0.86 |
| GBP5 | CDKL1 | 0.86 |
| SECTM1 | TULP4 | 0.86 |
| GBP5 | PAQR6 | 0.86 |
| LHFPL2 | CD40 | 0.86 |
| C1QA | EIF1 | 0.86 |
| GBP5 | GLA | 0.86 |
| AIM2 | GBP1 | 0.86 |
| UBE216 | SMARCC2 | 0.86 |
| P2RY14 | GNPAT | 0.86 |
| UBE2L6 | PRPF4 | 0.86 |
| GNG11 | PSTPIP2 | 0.86 |
| C1QA | PFAS | 0.86 |
| C1QA | AP181 | 0.86 |
| C1QA | BAHD1 | 0.86 |
| PSME1 | DGKD | 0.86 |
| VAMP5 | MRPL9 | 0.86 |
| AIM2 | CAPN2 | 0.86 |
| SECTM1 | PUS1 | 0.86 |
| SECTM1 | VIPR1 | 0.86 |
| LHFPL2 | MSH6 | 0.86 |
| P2RY14 | SLC35C1 | 0.86 |
| GBP5 | PRPF4B | 0.86 |
| C1QA | CD46 | 0.86 |
| P2RY14 | HDAC1 | 0.86 |
| GBP1 | NUP98 | 0.86 |
| GBP1 | TAF6 | 0.86 |
| GBP1 | NOTCH2 | 0.86 |
| LIPT1 | SECTM1 | 0.86 |
| GBP1 | BAG3 | 0.86 |
| UBE216 | CHMP7 | 0.86 |
| LHFPL2 | PTP4A1 | 0.86 |
| SECTM1 | BCORL1 | 0.86 |
| GYPC | C1QA | 0.86 |
| POLB | CSNK1G3 | 0.86 |
| POLB | AVEN | 0.86 |
| GBP1 | UCHL5 | 0.86 |
| VAMP5 | MSH6 | 0.86 |
| GBP1 | PLCD1 | 0.86 |
| COX6B1 | LHFPL2 | 0.86 |
| GBP5 | SEC61G | 0.86 |
| POLB | RERE | 0.86 |
| SECTM1 | MLF2 | 0.86 |
| GBP5 | CD8A | 0.86 |
| GBP1 | PHF20 | 0.86 |
| POLB | GORASP2 | 0.86 |
| VAMP5 | CALU | 0.86 |
| VAMP5 | CTDP1 | 0.86 |
| POLB | PTPN7 | 0.86 |
| GBP1 | INPPSE | 0.86 |
| P2RY14 | MRPS31 | 0.86 |
| GBP1 | ELMO1 | 0.86 |
| POLB | ARHGEF2 | 0.86 |
| GBP1 | SOX4 | 0.86 |
| APOL2 | MECP2 | 0.86 |
| GBP5 | NTAN1 | 0.86 |
| IL15RA | TSR1 | 0.86 |
| GBP1 | DCAKD | 0.86 |
| PZRY14 | SLCO3A1 | 0.86 |
| GBP1 | RFC5 | 0.86 |
| C1QA | ABL1 | 0.86 |
| GBP5 | FBXO28 | 0.86 |
| APOL2 | MCM7 | 0.86 |
| DAPP1 | ARID1A | 0.86 |
| P2RY14 | ITGAX | 0.86 |
| POLB | PDPK1 | 0.86 |
| SECTM1 | RBBP4 | 0.86 |
| GBP5 | GMNN | 0.86 |
| GBP5 | MOSPD3 | 0.86 |
| P2RY14 | POLRMT | 0.86 |
| GBP5 | TPR | 0.86 |
| VAMP5 | RNGTT | 0.86 |
| PML | DDX27 | 0.86 |
| PXMP4 | GBP1 | 0.86 |
| POLB | MNAT1 | 0.86 |
| UBE216 | EPHA4 | 0.86 |
| APOL2 | SNX11 | 0.86 |
| CALCOCO2 | LHFPL2 | 0.86 |
| SECTM1 | SERINC3 | 0.86 |
| GBP1 | RCOR3 | 0.86 |
| C1QA | KYNU | 0.86 |
| GBP1 | RAF1 | 0.86 |
| UBE2L6 | ZFR | 0.86 |
| APOL1 | BAHD1 | 0.86 |
| GBP1 | YEATS4 | 0.86 |
| GBP1 | RBM22 | 0.86 |
| GBP5 | PYGL | 0.86 |
| GBP1 | ZNF589 | 0.86 |
| GBP5 | GSTM3 | 0.86 |
| GBP1 | TSEN2 | 0.86 |
| P2RY14 | STK24 | 0.86 |
| VAMP5 | STAG1 | 0.86 |
| GBP1 | BRF2 | 0.86 |
| GBP1 | ADAM19 | 0.86 |
| SECTM1 | PSMD4 | 0.86 |
| GBP1 | POLR2C | 0.86 |
| P2RY14 | IFITM1 | 0.86 |
| VAMP5 | SH3TC1 | 0.86 |
| P2RY14 | UXS1 | 0.86 |
| GBP1 | IL1R1 | 0.86 |
| GBP1 | CLK4 | 0.86 |
| POLB | GIMAP6 | 0.86 |
| PSME1 | NFATC1 | 0.86 |
| PRRG4 | GBP1 | 0.86 |
| DAPP1 | USP8 | 0.86 |
| POLB | ANXA11 | 0.86 |
| LSM1 | C1QA | 0.86 |
| GBP1 | SERPINB8 | 0.86 |
| GBP1 | DR1 | 0.86 |
| GBP1 | PARP2 | 0.86 |
| APOL2 | CRY2 | 0.86 |
| GNG11 | DUSP3 | 0.86 |
| ATP6V1G1 | C1QA | 0.86 |
| PSME1 | TRAP1 | 0.86 |
| UBE216 | HMGCR | 0.86 |
| VAMP5 | GOLGA3 | 0.86 |
| P2RY14 | ZCWPW1 | 0.86 |
| GBP1 | CDCA4 | 0.86 |
| DENND1A | NDRG1 | 0.86 |
| GBP5 | IDH3A | 0.86 |
| LY86 | GBP1 | 0.86 |
| GBP1 | TM7SF3 | 0.86 |
| APOL2 | ELMO1 | 0.86 |
| LHFPL2 | USP8 | 0.86 |
| P2RY14 | ZFYVE21 | 0.86 |
| C1QA | FT122 | 0.86 |
| GBP1 | CKAP2 | 0.86 |
| GBP5 | LMAN2 | 0.86 |
| GBP5 | GNPTAB | 0.86 |
| POLB | POLH | 0.86 |
| GBP1 | ITPKB | 0.86 |
| GBP1 | EXOSC9 | 0.86 |
| DAPP1 | SEC63 | 0.86 |
| TNFRSF14 | SECTM1 | 0.86 |
| GBP1 | DAAM2 | 0.86 |
| APOL2 | TCF7 | 0.86 |
| POLB | BFAR | 0.86 |
| GBP5 | CENPB | 0.86 |
| GBP5 | ZNF12 | 0.86 |
| APOL2 | STAT6 | 0.86 |
| POLB | POLDIP3 | 0.86 |
| VAMP5 | MYC | 0.86 |
| SECTM1 | RER1 | 0.86 |
| PRRG4 | APOL2 | 0.86 |
| P2RY14 | SOCS1 | 0.86 |
| P2RY14 | TNFSF10 | 0.86 |
| UBE216 | TRAF3 | 0.86 |
| UBE2L6 | ER5 | 0.86 |
| SECTM1 | ER2 | 0.86 |
| LHFPL2 | SERPINB8 | 0.86 |
| GBP1 | FAM53C | 0.86 |
| VAMP5 | HMGA1 | 0.86 |
| UBE216 | FANCE | 0.86 |
| C1QA | RRS1 | 0.86 |
| C1QA | MSN | 0.86 |
| LHFPL2 | NIPBL | 0.86 |
| APOL1 | POLG | 0.86 |
| COX7A2 | GBP1 | 0.86 |
| GBP1 | IER5 | 0.86 |
| UBE2L6 | APOL3 | 0.86 |
| POLB | MDM1 | 0.86 |
| GBP1 | CRK | 0.86 |
| SECTM1 | PSMB1 | 0.86 |
| SECTM1 | MCM2 | 0.86 |
| UBE2L6 | POLR1B | 0.86 |
| UBE216 | YTHDC1 | 0.86 |
| APOL2 | NUP210 | 0.86 |
| VAMP5 | PHB | 0.86 |
| POLB | ANP32B | 0.86 |
| GBP5 | USPL1 | 0.86 |
| GBP1 | TSPAN2 | 0.86 |
| UBE2L6 | RTN2 | 0.86 |
| UBE2L6 | BAIAP2 | 0.86 |
| P2RY14 | UBASH3A | 0.86 |
| POLB | CACNA1I | 0.86 |
| VAMP5 | TM9SF4 | 0.86 |
| GBP1 | PXN | 0.86 |
| SECTM1 | YWHAZ | 0.86 |
| UBE2L6 | TRIM68 | 0.86 |
| SECTM1 | NUP210 | 0.86 |
| APOL2 | NOLC1 | 0.86 |
| GBP1 | TUBD1 | 0.86 |
| SECTM1 | LASP1 | 0.86 |
| GBP1 | NMT1 | 0.86 |
| GBP1 | ASTE1 | 0.86 |
| POLB | PPP2RSE | 0.86 |
| GBP5 | EXOC1 | 0.86 |
| LHFPL2 | PML | 0.86 |
| GBP1 | EP400 | 0.86 |
| P2RY14 | CD83 | 0.86 |
| UBE2L6 | TUBG1 | 0.86 |
| P2RY14 | ISG20 | 0.86 |
| P2RY14 | ATP1A1 | 0.86 |
| DENND1A | VAMP5 | 0.86 |
| P2RY14 | APBB1IP | 0.86 |
| GBP1 | SLC25A17 | 0.86 |
| GBP5 | CHERP | 0.86 |
| POLB | ATP8B2 | 0.86 |
| GBP1 | AP3M2 | 0.86 |
| SECTM1 | TMEM43 | 0.86 |
| SERPINB6 | GBP1 | 0.86 |
| GBP5 | CSF1R | 0.86 |
| STK16 | LHFPL2 | 0.86 |
| GBP5 | COPS2 | 0.86 |
| LIMK1 | VILL | 0.86 |
| SECTM1 | CBX4 | 0.86 |
| UBE2L6 | RAC2 | 0.86 |
| PSMB2 | GBP1 | 0.86 |
| VAMP5 | ARID1A | 0.86 |
| PSME1 | MAGED1 | 0.86 |
| SECTM1 | DGKA | 0.86 |
| AIM2 | PPP4R1 | 0.86 |
| PSTPIP2 | NDRG1 | 0.859 |
| SECTM1 | FGR | 0.859 |
| GBP5 | ADM | 0.859 |
| P2RY14 | RNF122 | 0.859 |
| C1QA | NUMA1 | 0.859 |
| VAMP5 | PSMD2 | 0.859 |
| UBE2L6 | EIF2B5 | 0.859 |
| VAMP5 | VPS11 | 0.859 |
| GBP1 | OVGP1 | 0.859 |
| TCTA | GBP1 | 0.859 |
| GMFG | GBP1 | 0.859 |
| UBE2L6 | RPL39L | 0.859 |
| C1QA | PPP2R5D | 0.859 |
| APOL2 | PLCG1 | 0.859 |
| SECTM1 | AP2A2 | 0.859 |
| PSME1 | VPS33B | 0.859 |
| PML | OSBP | 0.859 |
| POLB | EHD4 | 0.859 |
| POLB | PDCD2 | 0.859 |
| P2RY14 | PWP1 | 0.859 |
| SECTM1 | MFAP1 | 0.859 |
| UBE2L6 | ATIC | 0.859 |
| UBE2L6 | TRAF1 | 0.859 |
| GBP1 | STAT6 | 0.859 |
| UBE2L6 | CRY2 | 0.859 |
| C1QA | NMT1 | 0.859 |
| P2RY14 | SCFD1 | 0.859 |
| AIM2 | OSBPL9 | 0.859 |
| GSTO1 | CALCOCO2 | 0.859 |
| GBP5 | SS18L2 | 0.859 |
| P2RY14 | GPR137B | 0.859 |
| UBE2L6 | SSBP1 | 0.859 |
| GBP1 | STAM | 0.859 |
| GBP1 | RPL39L | 0.859 |
| GBP5 | ALDH2 | 0.859 |
| VAMP5 | ATIC | 0.859 |
| CASP1 | VAMP5 | 0.859 |
| COPB2 | P2RY14 | 0.859 |
| AIM2 | GUK1 | 0.859 |
| POLB | SIRT1 | 0.859 |
| UBE2L6 | SNAPC3 | 0.859 |
| C1QA | DHX9 | 0.859 |
| SECTM1 | LSG1 | 0.859 |
| APOL2 | APLP2 | 0.859 |
| P2RY14 | TNFAIP2 | 0.859 |
| P2RY14 | USP21 | 0.859 |
| GBP1 | PSD4 | 0.859 |
| SECTM1 | PHF20 | 0.859 |
| POLB | NR1D2 | 0.859 |
| SECTM1 | EIF4ENIF1 | 0.859 |
| UBE2L6 | RANBP3 | 0.859 |
| GBP1 | MRPL49 | 0.859 |
| POLB | GALNT2 | 0.859 |
| P2RY14 | MAZ | 0.859 |
| ZCWPW1 | GBP1 | 0.859 |
| POLB | DUT | 0.859 |
| UBE2L6 | PXN | 0.859 |
| UBE2L6 | SLC25A22 | 0.859 |
| GBP1 | HSPA14 | 0.859 |
| DAPP1 | GBP1 | 0.859 |
| P2RY14 | MAP3K10 | 0.859 |
| GEMIN7 | GBP1 | 0.859 |
| DAPP1 | STAG2 | 0.859 |
| C1QA | NFATC3 | 0.859 |
| GBP1 | TMEM97 | 0.859 |
| LHFPL2 | BCL2L11 | 0.859 |
| P2RY14 | E4F1 | 0.859 |
| P2RY14 | GLS | 0.859 |
| GBP1 | TEX10 | 0.859 |
| P2RY14 | ANKRA2 | 0.859 |
| P2RY14 | DUT | 0.859 |
| PRCP | P2RY14 | 0.859 |
| P2RY14 | BIN3 | 0.859 |
| AGPAT3 | VAMP5 | 0.859 |
| POLB | CORO2A | 0.859 |
| GBP1 | FNBP4 | 0.859 |
| APOL1 | TCF7 | 0.859 |
| P2RY14 | FAS | 0.859 |
| PML | DGKD | 0.859 |
| P2RY14 | RBM4B | 0.859 |
| IL15RA | YEATS2 | 0.859 |
| VAMP5 | CCDC22 | 0.859 |
| APOL2 | TRIM8 | 0.859 |
| UBE2L6 | TMED3 | 0.859 |
| GLRX5 | GBP1 | 0.859 |
| UBE2L6 | DDIT4 | 0.859 |
| POLB | CCT6A | 0.859 |
| P2RY14 | HPRT1 | 0.859 |
| GBP1 | TAF1C | 0.859 |
| SECTM1 | PDIA4 | 0.859 |
| VAMP5 | PSMB5 | 0.859 |
| GBP5 | NBN | 0.859 |
| P2RY14 | UNC93B1 | 0.859 |
| LIMK1 | PDHA1 | 0.859 |
| UBE216 | ING4 | 0.859 |
| GBP5 | CCND3 | 0.859 |
| SECTM1 | ACOX3 | 0.859 |
| C1QA | IVNS1ABP | 0.859 |
| POLB | ASNS | 0.859 |
| GBP1 | LEF1 | 0.859 |
| LHFPL2 | ATG3 | 0.859 |
| UBE2L6 | ACTN4 | 0.859 |
| SECTM1 | ANKS1A | 0.859 |
| POLB | TRIB2 | 0.859 |
| UBE2L6 | CSE1L | 0.859 |
| VAMP5 | MED9 | 0.859 |
| GBP1 | PPM1B | 0.859 |
| GBP1 | HSPA5 | 0.859 |
| LHFPL2 | USP48 | 0.859 |
| POLB | PEX7 | 0.859 |
| UBE2L6 | CASP6 | 0.859 |
| SECTM1 | KYNU | 0.859 |
| SECTM1 | EBAG9 | 0.859 |
| APOL2 | TOB2 | 0.859 |
| GBP1 | NMT2 | 0.859 |
| APOL2 | ABR | 0.859 |
| POLB | ANGEL1 | 0.859 |
| GBP1 | PPP2R5C | 0.859 |
| GBP1 | LANCL1 | 0.859 |
| POLB | TXK | 0.859 |
| AIM2 | HMGCR | 0.859 |
| APOL1 | MCM3 | 0.859 |
| VAMP5 | ODF2 | 0.859 |
| AIM2 | RGS10 | 0.859 |
| P2RY14 | ENO3 | 0.859 |
| KCTD17 | GBP1 | 0.859 |
| STAT1 | ELF1 | 0.859 |
| GBP5 | CRIP2 | 0.859 |
| CYB5R1 | GBP1 | 0.859 |
| POLB | DDX28 | 0.859 |
| P2RY14 | ANAPC5 | 0.859 |
| P2RY14 | LAMAS | 0.859 |
| VAMP5 | GMEB1 | 0.859 |
| P2RY14 | ZNF200 | 0.859 |
| DAPP1 | SYNJ2 | 0.859 |
| UBE2L6 | PER2 | 0.859 |
| POLB | PLAGLZ | 0.859 |
| UBE2L6 | TTF1 | 0.859 |
| PSTPIP2 | OSBPL9 | 0.859 |
| VAMP5 | SIRT5 | 0.859 |
| GBP1 | RUNX3 | 0.859 |
| P2RY14 | PLA2G12A | 0.859 |
| P2RY14 | CTDP1 | 0.859 |
| UBE2L6 | TSPYL2 | 0.859 |
| GBP1 | ABHD6 | 0.859 |
| SECTM1 | DNAJB1 | 0.859 |
| GBP1 | TNK2 | 0.859 |
| GBP1 | MDH2 | 0.859 |
| VAMP5 | SLTM | 0.859 |
| POLB | YWHAB | 0.859 |
| P2RY14 | MUS81 | 0.859 |
| GBP1 | ACVR1 | 0.859 |
| APOL2 | PARP16 | 0.859 |
| GBP5 | UCP3 | 0.859 |
| GBP1 | ELP3 | 0.859 |
| SECTM1 | MCM7 | 0.859 |
| P2RY14 | NUP88 | 0.859 |
| SECTM1 | ERBB2 | 0.859 |
| AIM2 | IL15RA | 0.859 |
| CALCOCO2 | DDX27 | 0.859 |
| CASP7 | VAMP5 | 0.859 |
| UBE2L6 | CAMK2G | 0.859 |
| FAS | APOL2 | 0.859 |
| VAMP5 | DPF2 | 0.859 |
| P2RY14 | KDELR2 | 0.859 |
| P2RY14 | UBE2E1 | 0.859 |
| VAMP5 | TOB2 | 0.859 |
| DAPP1 | SPEN | 0.859 |
| C1QA | PPP2R5B | 0.859 |
| UBE2L6 | ESD | 0.859 |
| IRF1 | ACP5 | 0.859 |
| DAPP1 | MAK | 0.859 |
| P2RY14 | PEBP1 | 0.859 |
| P2RY14 | GAS7 | 0.859 |
| GBP1 | TRIM24 | 0.859 |
| RGS10 | VAMP5 | 0.859 |
| VAMP5 | PUS1 | 0.859 |
| UBE2L6 | SDHB | 0.859 |
| GBP5 | GRHPR | 0.859 |
| SECTM1 | PACS1 | 0.859 |
| UBE2L6 | KIF3B | 0.859 |
| PSME1 | EXOSC10 | 0.859 |
| P2RY14 | YWHAB | 0.859 |
| VAMP5 | TAF6 | 0.859 |
| FOLR2 | LHFPL2 | 0.859 |
| GBP5 | TRIM14 | 0.859 |
| GBP1 | MAF | 0.859 |
| P2RY14 | LRRC47 | 0.859 |
| P2RY14 | CSTF2 | 0.859 |
| ADI1 | GBP1 | 0.859 |
| LHFPL2 | EHD4 | 0.859 |
| POLB | SLC35A2 | 0.859 |
| POLB | TMEM50B | 0.859 |
| VAMP5 | MRPL49 | 0.859 |
| ATP1B2 | GBP1 | 0.859 |
| GBP1 | BCAT1 | 0.859 |
| P2RY14 | ILKAP | 0.859 |
| VAMP5 | GANAB | 0.859 |
| UBE2L6 | SMARCD1 | 0.859 |
| SECTM1 | SLC35A2 | 0.859 |
| POLDIP2 | GBP1 | 0.859 |
| GBP1 | BCL2 | 0.859 |
| UBE2L6 | DDX54 | 0.859 |
| SECTM1 | MCM6 | 0.859 |
| P2RY14 | LMAN2L | 0.859 |
| POLB | PCYOX1 | 0.859 |
| SECTM1 | ASB1 | 0.859 |
| GBP5 | TUBB6 | 0.859 |
| P2RY14 | BTG2 | 0.859 |
| GBP5 | IRF5 | 0.859 |
| GBP5 | MTMR1 | 0.859 |
| GBP5 | TK1 | 0.859 |
| ABHD3 | VAMP5 | 0.859 |
| DAPP1 | MTMR6 | 0.859 |
| POLB | GTF2B | 0.859 |
| GBP1 | TCFL5 | 0.859 |
| VAMP5 | HRAS | 0.859 |
| GBP5 | AKR7A2 | 0.859 |
| PSME1 | PLCG1 | 0.859 |
| VAMP5 | RBBP4 | 0.859 |
| GBP5 | MTM1 | 0.859 |
| C1QA | SDC1 | 0.859 |
| CALCOCO2 | PRDM2 | 0.859 |
| LIMK1 | AKAP1 | 0.859 |
| C1QA | BTN3A1 | 0.859 |
| APOL2 | NMT1 | 0.859 |
| CYB561 | SMAD3 | 0.859 |
| SECTM1 | BCAT2 | 0.859 |
| GBP1 | TTLL12 | 0.859 |
| GBP1 | GANAB | 0.859 |
| AIM2 | FBXW2 | 0.859 |
| GBP1 | JOSD1 | 0.859 |
| MAX | DGKD | 0.859 |
| P2RY14 | ZNF174 | 0.859 |
| UBE2L6 | ADAM19 | 0.859 |
| GBP1 | KIF3B | 0.859 |
| LHFPL2 | TOB2 | 0.859 |
| SECTM1 | SH3TC1 | 0.859 |
| POLB | AMFR | 0.859 |
| GBP1 | HSF2 | 0.859 |
| GBP5 | IGBP1 | 0.859 |
| SECTM1 | POMT1 | 0.859 |
| P2RY14 | RIPK2 | 0.859 |
| MARCO | P2RY14 | 0.859 |
| GBP1 | DHCR7 | 0.859 |
| GBP5 | ATF2 | 0.859 |
| VAMP5 | ERBB2 | 0.859 |
| PSME1 | PPARD | 0.859 |
| POLB | SFXN1 | 0.859 |
| POLB | USP8 | 0.859 |
| DAPP1 | NDRG1 | 0.859 |
| DAPP1 | PRDM2 | 0.859 |
| LHFPL2 | PFAS | 0.859 |
| UBE2L6 | POGZ | 0.859 |
| GYPC | POLB | 0.859 |
| GBP1 | TRAM1 | 0.859 |
| GBP5 | BAG1 | 0.859 |
| P2RY14 | MR1 | 0.859 |
| GBP1 | DDX50 | 0.859 |
| GBP1 | GOLT1B | 0.859 |
| GBP5 | BST2 | 0.859 |
| FAS | SLCO4C1 | 0.859 |
| GBP1 | VPS39 | 0.859 |
| LAPTM4A | VAMP5 | 0.859 |
| GBP1 | LRRN3 | 0.859 |
| PSTPIP2 | HMGCR | 0.859 |
| UBE2L6 | JAM3 | 0.859 |
| C1QA | TERF2 | 0.859 |
| GBP1 | CEP68 | 0.859 |
| C1QA | ILF3 | 0.859 |
| POLB | CDK4 | 0.859 |
| UBE216 | AKR1A1 | 0.859 |
| GBP5 | WDR44 | 0.859 |
| UBE2L6 | IMP4 | 0.859 |
| GBP5 | IMPA2 | 0.859 |
| GBP1 | MBD1 | 0.859 |
| GBP5 | CDKN2D | 0.859 |
| LHFPL2 | BARD1 | 0.859 |
| VAMP5 | PBXIP1 | 0.859 |
| GBP1 | AGL | 0.859 |
| GBP5 | ZCCHC2 | 0.859 |
| APOL2 | GMEB1 | 0.859 |
| GBP5 | COX5A | 0.859 |
| LHFPL2 | IRS2 | 0.859 |
| GBP1 | FZD5 | 0.859 |
| VAMP5 | CDK4 | 0.859 |
| UBE2L6 | AXIN1 | 0.859 |
| VAMP5 | PSMC5 | 0.859 |
| SECTM1 | TIMELESS | 0.859 |
| UBE2L6 | MTRR | 0.859 |
| APOL2 | HIC2 | 0.859 |
| LHFPL2 | SEC63 | 0.859 |
| UBE2L6 | GNB1 | 0.859 |
| UBE2L6 | RBM22 | 0.859 |
| GBP1 | LAS1L | 0.859 |
| VAMP5 | MRPS18A | 0.859 |
| GBP1 | CACNA1 | 0.859 |
| GBP1 | PRNP | 0.859 |
| P2RY14 | RBM23 | 0.859 |
| GBP1 | SRD5A1 | 0.859 |
| AGPAT3 | APOL2 | 0.859 |
| GBP1 | PIGH | 0.859 |
| SECTM1 | ACAD8 | 0.859 |
| GBP5 | IDE | 0.859 |
| GBP5 | ZC3HAV1 | 0.859 |
| GBP5 | SEMA4G | 0.859 |
| GBP5 | LYST | 0.859 |
| IRF1 | SECTM1 | 0.859 |
| PPBP | C1QA | 0.859 |
| APOL2 | ZZEF1 | 0.859 |
| C1QA | CIDEB | 0.859 |
| GBP1 | SDK2 | 0.859 |
| GBP5 | RPS6KB1 | 0.859 |
| C1QA | CTDP1 | 0.859 |
| UBE2L6 | ABCC10 | 0.859 |
| POLB | SIDT1 | 0.859 |
| SECTM1 | PIGT | 0.858 |
| LHFPL2 | SLA | 0.858 |
| GBP1 | SACS | 0.858 |
| VAMP5 | TBCD | 0.858 |
| UBE2L6 | SIDT1 | 0.858 |
| P2RY14 | DERL2 | 0.858 |
| AIM2 | EIF1 | 0.858 |
| UBE2L6 | SLC9A1 | 0.858 |
| PSME1 | RANBP3 | 0.858 |
| GBP5 | PEF1 | 0.858 |
| SECTM1 | HK2 | 0.858 |
| NUDT2 | GBP1 | 0.858 |
| P2RY14 | TOE1 | 0.858 |
| LAPTM4A | APOL2 | 0.858 |
| GBP1 | ATPAF2 | 0.858 |
| APOL2 | TULP4 | 0.858 |
| LHFPL2 | MAPK1 | 0.858 |
| GBP1 | PUS7 | 0.858 |
| GBP5 | KIAA0586 | 0.858 |
| VAMP5 | MRPS7 | 0.858 |
| P2RY14 | TERF2IP | 0.858 |
| SECTM1 | UBAP2 | 0.858 |
| POLB | CNGB1 | 0.858 |
| P2RY14 | DVL3 | 0.858 |
| UBE2L6 | CASP2 | 0.858 |
| GBP1 | PECR | 0.858 |
| GBP5 | TAF1B | 0.858 |
| P2RY14 | CPT1A | 0.858 |
| UBE2L6 | VAMP3 | 0.858 |
| PML | TSR1 | 0.858 |
| GBP1 | IL2RB | 0.858 |
| GBP1 | CASK | 0.858 |
| C1QA | APLP2 | 0.858 |
| UBE2L6 | ENSA | 0.858 |
| GBP1 | RNF122 | 0.858 |
| UBE2L6 | DLEC1 | 0.858 |
| GBP1 | CNGB1 | 0.858 |
| UBE2L6 | POR | 0.858 |
| APOL2 | MAP4 | 0.858 |
| P2RY14 | HERPUD1 | 0.858 |
| P2RY14 | EIF2B1 | 0.858 |
| ZNF394 | GBP1 | 0.858 |
| P2RY14 | METTL1 | 0.858 |
| P2RY14 | RBMX2 | 0.858 |
| SECTM1 | MPI | 0.858 |
| SECTM1 | CASP6 | 0.858 |
| POLB | SUCLG1 | 0.858 |
| UBE2L6 | NUP98 | 0.858 |
| GBP5 | TLR5 | 0.858 |
| P2RY14 | KIFAP3 | 0.858 |
| GBP1 | THAP1 | 0.858 |
| PSME1 | RBM28 | 0.858 |
| TMEM115 | GBP1 | 0.858 |
| UBE2L6 | YTHDF1 | 0.858 |
| GBP1 | ACTN4 | 0.858 |
| POLB | MDM2 | 0.858 |
| POLB | MLH1 | 0.858 |
| AIM2 | MYD88 | 0.858 |
| UBE2L6 | GUK1 | 0.858 |
| NDUFB6 | APOL2 | 0.858 |
| SECTM1 | TAZ | 0.858 |
| LHFPL2 | TIPARP | 0.858 |
| AIM2 | NENF | 0.858 |
| PML | NFATC1 | 0.858 |
| GBP5 | FEM1B | 0.858 |
| GSR | P2RY14 | 0.858 |
| UBE2L6 | MFAP1 | 0.858 |
| TNNC2 | GBP1 | 0.858 |
| UBE2L6 | VIPR1 | 0.858 |
| RPL3L | GBP1 | 0.858 |
| UBE2L6 | PDIA6 | 0.858 |
| POLB | SLCO4C1 | 0.858 |
| LHFPL2 | SSBP1 | 0.858 |
| ADAM15 | GBP1 | 0.858 |
| GBP1 | PPARD | 0.858 |
| SECTM1 | FHOD1 | 0.858 |
| LHFPL2 | OSBPL11 | 0.858 |
| POLB | BACH2 | 0.858 |
| P2RY14 | CCDC51 | 0.858 |
| GBP5 | HMOX2 | 0.858 |
| GBP1 | ZBTB25 | 0.858 |
| GAMT | GBP1 | 0.858 |
| C1QA | LSR | 0.858 |
| APOL2 | RRS1 | 0.858 |
| VAMP5 | MPI | 0.858 |
| VAMP5 | APLP2 | 0.858 |
| P2RY14 | BFAR | 0.858 |
| UBE2L6 | ANK3 | 0.858 |
| UBE2L6 | ELAVL1 | 0.858 |
| VAMP5 | PLCG1 | 0.858 |
| VAMP5 | EXOSC10 | 0.858 |
| GBP5 | FRY | 0.858 |
| C1QA | PLAGL2 | 0.858 |
| GBP1 | SPSB3 | 0.858 |
| POLB | RNF38 | 0.858 |
| GBP1 | AOC3 | 0.858 |
| GBP5 | GTF2A2 | 0.858 |
| NPC2 | ACP5 | 0.858 |
| VAMP5 | AP181 | 0.858 |
| GBP1 | AMMECR1 | 0.858 |
| UBE2L6 | CDYL | 0.858 |
| LHFPL2 | SERINC3 | 0.858 |
| POLB | CLCN6 | 0.858 |
| UBE216 | ATP2A2 | 0.858 |
| VAMP5 | RUNX2 | 0.858 |
| SECTM1 | ITPKB | 0.858 |
| P2RY14 | SLC27A5 | 0.858 |
| UBE2L6 | MED9 | 0.858 |
| GBP1 | MPI | 0.858 |
| POLB | GNB1 | 0.858 |
| P2RY14 | PSMD1 | 0.858 |
| GBP5 | LIN7A | 0.858 |
| LIMK1 | OGDH | 0.858 |
| VAMP5 | DDIT4 | 0.858 |
| UBE2L6 | ZNF200 | 0.858 |
| POLB | OSGEPL1 | 0.858 |
| GBP5 | NSD1 | 0.858 |
| GBP5 | HIVEP1 | 0.858 |
| UBE2L6 | TSC2 | 0.858 |
| IL15RA | RUNX2 | 0.858 |
| P2RY14 | OAT | 0.858 |
| GBP5 | SLC25A3 | 0.858 |
| GBP5 | THRAP3 | 0.858 |
| PFDN5 | VAMP5 | 0.858 |
| P2RY14 | SLC27A3 | 0.858 |
| SECTM1 | ABCF3 | 0.858 |
| APOL2 | SYNJ2 | 0.858 |
| P2RY14 | MKKS | 0.858 |
| UBE2L6 | NFKB1 | 0.858 |
| CASP1 | PPP2RSA | 0.858 |
| GBP5 | SNX10 | 0.858 |
| GBP5 | PDK1 | 0.858 |
| UBE2L6 | ARHGEF7 | 0.858 |
| UBE2L6 | BRF2 | 0.858 |
| GBP1 | SLC35F2 | 0.858 |
| POLB | FAM3A | 0.858 |
| UBE2L6 | CTBP2 | 0.858 |
| GBP1 | USP11 | 0.858 |
| POLB | MPHOSPH10 | 0.858 |
| P2RY14 | CLDN15 | 0.858 |
| GBP5 | OSBPL3 | 0.858 |
| GBP1 | SERINC1 | 0.858 |
| SMARCD3 | NDRG1 | 0.858 |
| GBP5 | GIMAP4 | 0.858 |
| P2RY14 | KCNAB2 | 0.858 |
| POLB | RECQLS | 0.858 |
| GBP5 | CAPNS1 | 0.858 |
| UBE2L6 | PUS1 | 0.858 |
| P2RY14 | PSME3 | 0.858 |
| GBP1 | CA5B | 0.858 |
| IL15RA | ILF3 | 0.858 |
| GBP1 | ELAVL1 | 0.858 |
| P2RY14 | CLCN7 | 0.858 |
| GBP5 | SEMA6B | 0.858 |
| C1QA | CLEC7A | 0.858 |
| SECTM1 | SLTM | 0.858 |
| GBP5 | CFLAR | 0.858 |
| UBE2L6 | IL15 | 0.858 |
| PSMB7 | GBP1 | 0.858 |
| P2RY14 | SNN | 0.858 |
| GBP1 | ARHGEF6 | 0.858 |
| GBP1 | MOSPD2 | 0.858 |
| GBP1 | HBS1L | 0.858 |
| UBE2L6 | VPS35 | 0.858 |
| GBP1 | FLI | 0.858 |
| CALCOCO2 | GLG1 | 0.858 |
| P2RY14 | ITGAE | 0.858 |
| VAMP5 | GLUL | 0.858 |
| TNFRSF14 | VAMP5 | 0.858 |
| TXNL1 | GBP1 | 0.858 |
| POLB | MTCH2 | 0.858 |
| UBE216 | UBE2G1 | 0.858 |
| COX6B1 | UBE2L6 | 0.858 |
| P2RY14 | IGFBP3 | 0.858 |
| VAMP5 | DNAJA3 | 0.858 |
| GBP5 | CKAPS | 0.858 |
| NPC2 | TRIM8 | 0.858 |
| UBE2L6 | APLP2 | 0.858 |
| ATP7B | C1QA | 0.858 |
| GBP1 | FKRP | 0.858 |
| SECTM1 | RBM22 | 0.858 |
| LIMK1 | GEMIN4 | 0.858 |
| POLB | PPP6C | 0.858 |
| GBP5 | RAB11FIP5 | 0.858 |
| SECTM1 | MAPK1 | 0.858 |
| GBP5 | ZNF146 | 0.858 |
| VAMP5 | DDB2 | 0.858 |
| GBP1 | MANSC1 | 0.858 |
| C1QA | GOLGA3 | 0.858 |
| NPC2 | PDHA1 | 0.858 |
| GBP5 | NR2C2 | 0.858 |
| GBP1 | VPS35 | 0.858 |
| PSME1 | TBRG4 | 0.858 |
| B4GALT3 | GBP1 | 0.858 |
| VAMP5 | NFATC3 | 0.858 |
| PML | NUMA1 | 0.858 |
| POLB | APLP2 | 0.858 |
| VAMP5 | PSMD4 | 0.858 |
| GBP5 | UBQLN2 | 0.858 |
| AIM2 | PPP2RSA | 0.858 |
| TNNC2 | P2RY14 | 0.858 |
| GBP1 | POLR2B | 0.858 |
| SECTM1 | RAPGEF1 | 0.858 |
| P2RY14 | CD300C | 0.858 |
| UBEZL6 | TIPARP | 0.858 |
| C1QA | MED9 | 0.858 |
| UBE2L6 | ZNF212 | 0.858 |
| GBP1 | KLHL3 | 0.858 |
| GBP1 | OSGEPL1 | 0.858 |
| GBP1 | IFRD1 | 0.858 |
| SECTM1 | PSMD5 | 0.858 |
| UBE2L6 | EXOC7 | 0.858 |
| GBP1 | ZAP70 | 0.858 |
| P2RY14 | PITPNM1 | 0.858 |
| GBP1 | USP3 | 0.858 |
| NARF | GBP1 | 0.858 |
| LIMK1 | ILF3 | 0.858 |
| UBE2L6 | ATP9B | 0.858 |
| P2RY14 | CSAD | 0.858 |
| SECTM1 | MNT | 0.858 |
| GBP1 | BRPF1 | 0.858 |
| GBP1 | UBE4B | 0.858 |
| GBP1 | STOML1 | 0.858 |
| VAMP5 | FANCE | 0.858 |
| DUSP3 | VAMP5 | 0.858 |
| GCM1 | P2RY14 | 0.858 |
| POLB | PSMB1 | 0.858 |
| GBP1 | PAOX | 0.858 |
| LHFPL2 | XPO1 | 0.858 |
| GBP1 | CASP2 | 0.858 |
| GBP1 | YTHDF1 | 0.858 |
| GBP1 | PUS7L | 0.858 |
| GBP1 | CIC | 0.858 |
| GBP1 | DUSP11 | 0.858 |
| POLB | PLA2G12A | 0.858 |
| GBP1 | SPAG9 | 0.858 |
| PSTPIP2 | LIMK1 | 0.858 |
| LHFPL2 | POLD3 | 0.858 |
| P2RY14 | BTN3A2 | 0.858 |
| P2RY14 | FXR2 | 0.858 |
| SECTM1 | RNF41 | 0.858 |
| C1QA | RUNX2 | 0.858 |
| P2RY14 | STARD8 | 0.858 |
| AIM2 | SLA | 0.858 |
| GBP1 | CPSF1 | 0.858 |
| LHFPL2 | PRKARZA | 0.858 |
| VAMP5 | GSS | 0.858 |
| P2RY14 | TSPAN31 | 0.858 |
| GBP1 | MRPL19 | 0.858 |
| GBP5 | CPD | 0.858 |
| AIM2 | IL1R1 | 0.858 |
| P2RY14 | SCRIB | 0.858 |
| VAMP5 | XRCC1 | 0.858 |
| POLB | FOLR2 | 0.858 |
| C1QA | OSBPL9 | 0.858 |
| P2RY14 | SAE1 | 0.858 |
| GBP1 | MAP3K14 | 0.858 |
| LHFPL2 | FOXJ2 | 0.858 |
| UBE2L6 | TMSB10 | 0.858 |
| POLB | PPP1R2 | 0.858 |
| POLB | NIT2 | 0.858 |
| UBE2L6 | NMI | 0.858 |
| GBP1 | ABCF2 | 0.858 |
| C1QA | PPM1G | 0.858 |
| IL15RA | DDX27 | 0.858 |
| GBP1 | GRWD1 | 0.858 |
| VAMP5 | SNX11 | 0.858 |
| C1QA | YEATS2 | 0.858 |
| CASP1 | STAG1 | 0.858 |
| SECTM1 | TSFM | 0.858 |
| RNF13 | VAMP5 | 0.858 |
| GBP5 | JMJDIC | 0.858 |
| GBP5 | ADAR | 0.858 |
| GBP1 | IL10RA | 0.858 |
| PSME1 | DUSP3 | 0.858 |
| PML | OGDH | 0.858 |
| ASCL2 | GBP1 | 0.858 |
| VAMP5 | MAGED1 | 0.858 |
| GBP5 | SETX | 0.858 |
| GBP1 | SMAP1 | 0.858 |
| GBP5 | LRP8 | 0.858 |
| PSME1 | IL16 | 0.858 |
| LHFPL2 | SYNCRIP | 0.858 |
| UBE2L6 | CIC | 0.858 |
| POLB | MTR | 0.858 |
| P2RY14 | PLA2G4C | 0.858 |
| UBE2L6 | CC2D1A | 0.858 |
| GBP5 | TRIM22 | 0.858 |
| ACP5 | PRRG4 | 0.858 |
| GBP1 | PKD2 | 0.858 |
| NPC2 | SDC1 | 0.858 |
| AIM2 | TMED3 | 0.858 |
| GBP1 | TRIM37 | 0.858 |
| PML | APOL2 | 0.858 |
| SECTM1 | SLC25A11 | 0.858 |
| CYB561 | PML | 0.858 |
| P2RY14 | TBL2 | 0.858 |
| POLB | RPUSD2 | 0.858 |
| GBP1 | GPR132 | 0.858 |
| APOL2 | SLTM | 0.858 |
| SECTM1 | AQP3 | 0.858 |
| UBE2L6 | SEC61A1 | 0.858 |
| NDUFS4 | GBP1 | 0.858 |
| P2RY14 | AVEN | 0.858 |
| UBE2L6 | CLEC7A | 0.858 |
| POLB | DAZAP1 | 0.858 |
| POLB | SERPINB8 | 0.858 |
| GBP1 | SRP54 | 0.858 |
| ATG3 | DHX9 | 0.858 |
| UBE2L6 | PSD4 | 0.858 |
| GBP1 | TFRC | 0.858 |
| P2RY14 | FLII | 0.858 |
| GBP5 | TREM1 | 0.858 |
| GBP1 | PRPF4 | 0.858 |
| APOL1 | NFATC1 | 0.858 |
| UBE2L6 | SLC1A4 | 0.858 |
| APOL2 | PUS1 | 0.858 |
| CYB561 | POLG | 0.858 |
| IL15RA | CASP1 | 0.858 |
| C1QA | TBCD | 0.858 |
| GBP1 | BTG3 | 0.858 |
| LHFPL2 | PRDM2 | 0.858 |
| TMEM59 | CYB561 | 0.858 |
| AIM2 | TIPARP | 0.858 |
| APOL2 | PHF3 | 0.858 |
| VAMP5 | SYNJ2 | 0.858 |
| C1QA | FTSJ3 | 0.858 |
| P2RY14 | ACAD10 | 0.857 |
| GBP1 | EIF2B5 | 0.857 |
| UBE2L6 | TRIB2 | 0.857 |
| GBP5 | RPS6KA5 | 0.857 |
| POLB | PDCD6IP | 0.857 |
| GBP1 | AXIN1 | 0.857 |
| GBP1 | MPPE1 | 0.857 |
| POLB | RFX5 | 0.857 |
| COASY | GBP1 | 0.857 |
| POLB | CCND2 | 0.857 |
| EIF1 | VAMP5 | 0.857 |
| UBE2L6 | HSPAS | 0.857 |
| NPC2 | SSRP1 | 0.857 |
| VAMP5 | PSMD5 | 0.857 |
| UBE2L6 | HK2 | 0.857 |
| P2RY14 | DISC1 | 0.857 |
| GBP1 | PCNA | 0.857 |
| UBE2L6 | RPA1 | 0.857 |
| GBP1 | ZNF330 | 0.857 |
| APOL2 | TMCC1 | 0.857 |
| POLB | NOC3L | 0.857 |
| SECTM1 | BTN3A1 | 0.857 |
| UBE2L6 | MFGE8 | 0.857 |
| C1QA | MSH6 | 0.857 |
| POLB | GTDC1 | 0.857 |
| P2RY14 | LDLRAP1 | 0.857 |
| GBP5 | RALGDS | 0.857 |
| GBP1 | MRPS27 | 0.857 |
| P2RY14 | TMEM14A | 0.857 |
| GBP1 | MAP4K4 | 0.857 |
| P2RY14 | PXMP2 | 0.857 |
| GBP5 | SMPDL3A | 0.857 |
| GBP1 | LPIN1 | 0.857 |
| GBP1 | PPIG | 0.857 |
| UBE2L6 | ITGAL | 0.857 |
| SECTM1 | STIM1 | 0.857 |
| GBP1 | DCK | 0.857 |
| POLB | DDIT4 | 0.857 |
| APOL2 | NFATC3 | 0.857 |
| POLB | ZNF337 | 0.857 |
| VAMP5 | FBXW2 | 0.857 |
| SECTM1 | ATG5 | 0.857 |
| POLB | ACOX3 | 0.857 |
| UBEZL6 | SEH1L | 0.857 |
| NPC2 | DPF2 | 0.857 |
| CALCOCO2 | PHF3 | 0.857 |
| VAMP5 | LSG1 | 0.857 |
| POLB | ACBD3 | 0.857 |
| POLB | METAP1 | 0.857 |
| POLB | TBC1D22A | 0.857 |
| GBP1 | TOP2B | 0.857 |
| POLB | DYNC1LI2 | 0.857 |
| UBE2L6 | UPF2 | 0.857 |
| POLB | GALNT11 | 0.857 |
| VAMP5 | ABL1 | 0.857 |
| GBP5 | SLC25A1 | 0.857 |
| POLB | SLC43A1 | 0.857 |
| P2RY14 | MGLL | 0.857 |
| P2RY14 | GNB5 | 0.857 |
| UBE2L6 | SLC38A1 | 0.857 |
| CYB561 | APOL2 | 0.857 |
| UBE2L6 | ABCF3 | 0.857 |
| RAB20 | GBP1 | 0.857 |
| ATG3 | PHF3 | 0.857 |
| LHFPL2 | SLC6A6 | 0.857 |
| GBP1 | TEX261 | 0.857 |
| UBE2L6 | PPP3CA | 0.857 |
| APOL2 | OXSR1 | 0.857 |
| CYB561 | LIG1 | 0.857 |
| GBP1 | FUCA1 | 0.857 |
| GBP5 | RCBTB1 | 0.857 |
| IL15RA | CTCF | 0.857 |
| UBE2L6 | ELP3 | 0.857 |
| SECTM1 | UBE2G1 | 0.857 |
| UBE216 | TPP2 | 0.857 |
| P2RY14 | TIPRL | 0.857 |
| UBE2L6 | MKRN2 | 0.857 |
| GBP5 | TMOD1 | 0.857 |
| GBP1 | PCSK7 | 0.857 |
| APOL2 | GPR132 | 0.857 |
| IL15RA | BCR | 0.857 |
| VAMP5 | PTPN7 | 0.857 |
| UBE2L6 | PIK3R5 | 0.857 |
| VAMP5 | PLK3 | 0.857 |
| NPC2 | LAS1L | 0.857 |
| GBP1 | SLC3A2 | 0.857 |
| P2RY14 | FAM50B | 0.857 |
| POLB | NKTR | 0.857 |
| UBE2L6 | PARP2 | 0.857 |
| POP4 | GBP1 | 0.857 |
| IL15RA | LIG1 | 0.857 |
| P2RY14 | SCRN1 | 0.857 |
| UBE2L6 | FXR2 | 0.857 |
| AIM2 | APOL3 | 0.857 |
| CALCOCO2 | PML | 0.857 |
| GBP1 | CRLF3 | 0.857 |
| POLB | RNF25 | 0.857 |
| LHFPL2 | EIF4G1 | 0.857 |
| POLB | GUK1 | 0.857 |
| AIM2 | PF4V1 | 0.857 |
| POLB | USP11 | 0.857 |
| P2RY14 | PGM1 | 0.857 |
| LHFPL2 | AP1G1 | 0.857 |
| P2RY14 | TFE3 | 0.857 |
| CALCOCO2 | ILF3 | 0.857 |
| PSTPIP2 | ELF1 | 0.857 |
| UBE2L6 | KLF2 | 0.857 |
| P2RY14 | WIPI1 | 0.857 |
| ATG3 | POLR3B | 0.857 |
| LHFPL2 | DAAM2 | 0.857 |
| LHFPL2 | SRPK2 | 0.857 |
| UBE2L6 | QPRT | 0.857 |
| P2RY14 | SPATA5L1 | 0.857 |
| SECTM1 | HTRA2 | 0.857 |
| UBE2L6 | USP5 | 0.857 |
| UBE2L6 | PITPNC1 | 0.857 |
| DAPP1 | UPF2 | 0.857 |
| POLB | SLC39A6 | 0.857 |
| SECTM1 | BRF2 | 0.857 |
| GBP1 | GALNT11 | 0.857 |
| P2RY14 | CLEC7A | 0.857 |
| GBP1 | RYBP | 0.857 |
| UBE216 | ATG5 | 0.857 |
| C1QA | TAF6 | 0.857 |
| POLB | ZNF395 | 0.857 |
| POLB | DHX15 | 0.857 |
| APOL2 | PLCD1 | 0.857 |
| GBP1 | MOAP1 | 0.857 |
| UBE2L6 | ADAM17 | 0.857 |
| VAMP5 | SMU1 | 0.857 |
| ATG3 | RUNX2 | 0.857 |
| GSTO1 | LIMK1 | 0.857 |
| GBP1 | GTDC1 | 0.857 |
| TIMM8B | GBP1 | 0.857 |
| GBP1 | NCK1 | 0.857 |
| P2RY14 | WTAP | 0.857 |
| UBE2L6 | MBD2 | 0.857 |
| VAMP5 | FLI | 0.857 |
| C1QA | PPRC1 | 0.857 |
| VAMP5 | POLRMT | 0.857 |
| GBP5 | NDUFAB1 | 0.857 |
| CPA3 | GBP1 | 0.857 |
| SECTM1 | ZNF212 | 0.857 |
| GBP5 | CDC14A | 0.857 |
| VAMP5 | CHD4 | 0.857 |
| TXNL1 | UBE2L6 | 0.857 |
| POLB | ACTL6A | 0.857 |
| SEPHS2 | UBE2L6 | 0.857 |
| LHFPL2 | ZFR | 0.857 |
| KYNU | VAMP5 | 0.857 |
| P2RY14 | RTP4 | 0.857 |
| PSTPIP2 | UPB1 | 0.857 |
| SECTM1 | MUTYH | 0.857 |
| UBE2L6 | ERCC3 | 0.857 |
| LHFPL2 | PLAGL2 | 0.857 |
| UBE216 | MYO9B | 0.857 |
| SECTM1 | RUVBL1 | 0.857 |
| IL15RA | MAP4 | 0.857 |
| C1QA | FANCE | 0.857 |
| C1QA | PLCG1 | 0.857 |
| UBE2L6 | RBBP7 | 0.857 |
| APOL1 | POLR3B | 0.857 |
| PSME1 | ARID1A | 0.857 |
| POLB | CSTF2 | 0.857 |
| CALCOCO2 | BCR | 0.857 |
| P2RY14 | PAQR4 | 0.857 |
| UBE2L6 | CTSS | 0.857 |
| UBE2L6 | CCS | 0.857 |
| GBP1 | GORASP2 | 0.857 |
| C1QA | DENND1A | 0.857 |
| DHRS7B | GBP1 | 0.857 |
| GBP5 | TLR2 | 0.857 |
| GBP5 | BRMS1 | 0.857 |
| NME4 | GBP1 | 0.857 |
| P2RY14 | GFI1 | 0.857 |
| MAX | ATG3 | 0.857 |
| LHFPL2 | MSN | 0.857 |
| UBE2L6 | SH3TC1 | 0.857 |
| GBP1 | ASB1 | 0.857 |
| POLB | POFUT2 | 0.857 |
| GBP5 | MTF2 | 0.857 |
| ZNF672 | GBP1 | 0.857 |
| CD33 | GBP1 | 0.857 |
| POLB | SOX4 | 0.857 |
| APOL1 | TTLL12 | 0.857 |
| ODC1 | P2RY14 | 0.857 |
| C1QA | PFDN5 | 0.857 |
| P2RY14 | CITED2 | 0.857 |
| P2RY14 | PHF1 | 0.857 |
| CALCOCO2 | DIAPH1 | 0.857 |
| AIM2 | TP53BP2 | 0.857 |
| POLB | ATP6V1E1 | 0.857 |
| POLB | ECD | 0.857 |
| C1QA | PSMD11 | 0.857 |
| P2RY14 | ING2 | 0.857 |
| P2RY14 | ABCF3 | 0.857 |
| GBP1 | ALG6 | 0.857 |
| C1QA | CCDC22 | 0.857 |
| UBE2L6 | VTI1B | 0.857 |
| P2RY14 | PRKAB1 | 0.857 |
| PSME1 | GSTO1 | 0.857 |
| SECTM1 | TCF7 | 0.857 |
| ACO1 | SECTM1 | 0.857 |
| GBP1 | OGFR | 0.857 |
| GBP1 | FTSJ3 | 0.857 |
| P2RY14 | SLC2A4RG | 0.857 |
| GBP1 | HPS1 | 0.857 |
| PCK2 | POLB | 0.857 |
| C1QA | RBBP4 | 0.857 |
| SECTM1 | ZCCHC14 | 0.857 |
| GBP1 | TSNAX | 0.857 |
| GBP1 | TMEM106C | 0.857 |
| GBP1 | PGRMC2 | 0.857 |
| GBP5 | NUP133 | 0.857 |
| POLB | RAD51C | 0.857 |
| ATP6V1G1 | LHFPL2 | 0.857 |
| PSME1 | OGDH | 0.857 |
| UBE2L6 | ZDHHC6 | 0.857 |
| POLB | TIAL1 | 0.857 |
| POLB | SNX16 | 0.857 |
| GBP1 | MAGEH1 | 0.857 |
| LHFPL2 | CALU | 0.857 |
| AIM2 | RBM28 | 0.857 |
| GBP5 | AIP | 0.857 |
| GBP1 | RHEB | 0.857 |
| GBP5 | LGALS3BP | 0.857 |
| P2RY14 | CEP63 | 0.857 |
| UBEZL6 | KNTC1 | 0.857 |
| P2RY14 | SHC1 | 0.857 |
| GBP5 | SPAST | 0.857 |
| VAMP5 | PSMD1 | 0.857 |
| POLB | CD59 | 0.857 |
| VAMP5 | MUTYH | 0.857 |
| C1QA | NRG1 | 0.857 |
| GBP5 | MAD1L1 | 0.857 |
| SECTM1 | LRIG1 | 0.857 |
| VAMP5 | NMT1 | 0.857 |
| GBP1 | RABSB | 0.857 |
| L15RA | CAPN2 | 0.857 |
| GBP1 | EXOC7 | 0.857 |
| PML | LIG1 | 0.857 |
| UBE2L6 | SLC25A17 | 0.857 |
| UBE2L6 | PCBP4 | 0.857 |
| UBE2L6 | SS18 | 0.857 |
| GBP1 | TBCD | 0.857 |
| UBE216 | ZNF365 | 0.857 |
| LIMK1 | ARID1A | 0.857 |
| POLB | OGFOD1 | 0.857 |
| LHFPL2 | DNAJC1 | 0.857 |
| IRF1 | LHFPL2 | 0.857 |
| POLB | CAND1 | 0.857 |
| GBP1 | RBM15 | 0.857 |
| ITGB5 | GBP1 | 0.857 |
| AIM2 | MANSC1 | 0.857 |
| AIM2 | NCOR1 | 0.857 |
| POLB | BBS7 | 0.857 |
| UBE2L6 | RIN2 | 0.857 |
| VAMP5 | AAAS | 0.857 |
| GNG11 | CASP1 | 0.857 |
| SECTM1 | ZHX2 | 0.857 |
| GBP1 | RNF170 | 0.857 |
| ERCC2 | GBP1 | 0.857 |
| PSTPIP2 | TOPBP1 | 0.857 |
| VAMP5 | GPR132 | 0.857 |
| GBP5 | ZDHHC17 | 0.857 |
| GBP1 | PTPN7 | 0.857 |
| POLB | PIGV | 0.857 |
| GBP1 | UBE3A | 0.857 |
| PSTPIP2 | CYB561 | 0.857 |
| GBP5 | GSPT1 | 0.857 |
| RERE | VAMP5 | 0.857 |
| GBP1 | ZNF274 | 0.857 |
| SECTM1 | RGS10 | 0.857 |
| SECTM1 | AKAP8L | 0.857 |
| ATG3 | OSBP | 0.857 |
| P2RY14 | RAPSN | 0.857 |
| UBE2L6 | MRPL9 | 0.857 |
| CCS | POLB | 0.857 |
| P2RY14 | PANK4 | 0.857 |
| UBE2L6 | MTHFD1 | 0.857 |
| VAMP5 | CDK5RAP1 | 0.857 |
| C1QA | RNF13 | 0.857 |
| POLB | SLAMF1 | 0.857 |
| UBE2L6 | WTAP | 0.857 |
| UBE2L6 | POLRMT | 0.857 |
| POLB | PTPN11 | 0.857 |
| NPC2 | GLG1 | 0.857 |
| GBP1 | BACH2 | 0.857 |
| GBP1 | ACTR2 | 0.857 |
| UBE2L6 | LIPT1 | 0.857 |
| UBE216 | JARID2 | 0.857 |
| POLB | TFG | 0.857 |
| DAPP1 | NCOA1 | 0.857 |
| C1QA | MYC | 0.857 |
| UBE2L6 | SAR1A | 0.857 |
| GBP1 | MCM5 | 0.857 |
| UNC119 | GBP1 | 0.857 |
| APOL2 | FLII | 0.857 |
| LHFPL2 | DNAJC4 | 0.857 |
| POLB | SNRK | 0.857 |
| POLB | KCNAB2 | 0.857 |
| LHFPL2 | ACBD3 | 0.857 |
| LIMK1 | PPRC1 | 0.857 |
| UBE2L6 | BTN3A1 | 0.857 |
| SECTM1 | POLD1 | 0.857 |
| POLB | RNF7 | 0.857 |
| GBP1 | UGP2 | 0.857 |
| POLB | ARCN1 | 0.857 |
| GBP5 | FUT8 | 0.857 |
| GBP1 | ELF4 | 0.857 |
| VAMP5 | SERGEF | 0.857 |
| LIMK1 | PBXIP1 | 0.857 |
| PIP5K1C | GBP1 | 0.857 |
| UBE2L6 | HMG20A | 0.857 |
| NPC2 | NCL | 0.857 |
| GBP1 | ACOT7 | 0.857 |
| UBE2L6 | ADD1 | 0.857 |
| UBE2L6 | POMT1 | 0.857 |
| VAMP5 | TULP4 | 0.857 |
| SECTM1 | PCSK6 | 0.857 |
| DECR1 | GBP1 | 0.857 |
| SECTM1 | FLNB | 0.857 |
| CYB561 | MAP3K4 | 0.857 |
| GBP1 | ADAT1 | 0.857 |
| GBP1 | RIOK2 | 0.857 |
| VAMP5 | NFE2L1 | 0.857 |
| POLB | CHKA | 0.857 |
| C1QA | MAP3K4 | 0.857 |
| UBE2L6 | PSMA2 | 0.857 |
| APOL2 | MAP4K4 | 0.857 |
| APOL2 | CAPN2 | 0.857 |
| GBP1 | CCT6A | 0.857 |
| GBP1 | UBAP2L | 0.857 |
| UBE216 | MTA2 | 0.857 |
| P2RY14 | WDR70 | 0.857 |
| LHFPL2 | COROZA | 0.856 |
| POLB | ING4 | 0.856 |
| IL15RA | RBM28 | 0.856 |
| GBP5 | USP12 | 0.856 |
| UBE2L6 | DNAJB1 | 0.856 |
| POLB | DTX3 | 0.856 |
| GBP1 | PHF10 | 0.856 |
| VAMP5 | PSTPIP1 | 0.856 |
| APOL2 | VPS11 | 0.856 |
| JAM3 | VAMP5 | 0.856 |
| UBE2L6 | CDC25B | 0.856 |
| P2RY14 | MTHFD1 | 0.856 |
| GBP1 | CIDEB | 0.856 |
| SECTM1 | ANXA7 | 0.856 |
| ALDH1A1 | GBP1 | 0.856 |
| GBP1 | SLC4A7 | 0.856 |
| PSME1 | AXIN1 | 0.856 |
| UBE2L6 | BCAT2 | 0.856 |
| NDUFA1 | LHFPL2 | 0.856 |
| P2RY14 | WDR61 | 0.856 |
| P2RY14 | TIMM8A | 0.856 |
| AIM2 | TMSB10 | 0.856 |
| POLB | PSD4 | 0.856 |
| P2RY14 | TRIB3 | 0.856 |
| UBE216 | AMPD3 | 0.856 |
| SECTM1 | VPS33A | 0.856 |
| P2RY14 | CDADC1 | 0.856 |
| UBE2L6 | GPD1L | 0.856 |
| C1QA | RNGTT | 0.856 |
| SECTM1 | UBIAD1 | 0.856 |
| GBP1 | CLEC4A | 0.856 |
| GBP1 | ZNF232 | 0.856 |
| APOL2 | PIK3C2B | 0.856 |
| LHFPL2 | TSR1 | 0.856 |
| POLB | POLDIP2 | 0.856 |
| VAMP5 | ZNF76 | 0.856 |
| UBE2L6 | AP3M2 | 0.856 |
| CALCOCO2 | DHX9 | 0.856 |
| P2RY14 | DDX28 | 0.856 |
| SECTM1 | SLCO4C1 | 0.856 |
| P2RY14 | NCDN | 0.856 |
| VAMP5 | CSE1L | 0.856 |
| GBP5 | SMARCA5 | 0.856 |
| GBP1 | NOL11 | 0.856 |
| GBP5 | C1GALT1 | 0.856 |
| UBE2L6 | ABCA2 | 0.856 |
| C1QA | SCRIB | 0.856 |
| GBP1 | PPP3CC | 0.856 |
| POLB | DCTN5 | 0.856 |
| C1QA | ERBB2 | 0.856 |
| GBP1 | RS2 | 0.856 |
| POLB | ZNF573 | 0.856 |
| POLB | RPS6KC1 | 0.856 |
| PSTPIP2 | ANKS1A | 0.856 |
| GBP1 | CHFR | 0.856 |
| ATG3 | SMARCC1 | 0.856 |
| UBE2L6 | AP1G1 | 0.856 |
| SECTM1 | ELL | 0.856 |
| POLB | RNF170 | 0.856 |
| VAMP5 | MSH2 | 0.856 |
| NMI | LIMK1 | 0.856 |
| UBE2L6 | MEN1 | 0.856 |
| P2RY14 | NDE1 | 0.856 |
| VAMP5 | PPP2RSA | 0.856 |
| GBP1 | TGOLN2 | 0.856 |
| GBP5 | WSB1 | 0.856 |
| SECTM1 | HCLS1 | 0.856 |
| UBE2L6 | PIK3R1 | 0.856 |
| SECTM1 | ENSA | 0.856 |
| UBE2L6 | FAF1 | 0.856 |
| GBP1 | MORF4L1 | 0.856 |
| GBP1 | ARHGAP12 | 0.856 |
| IL2RG | P2RY14 | 0.856 |
| POLB | BUB3 | 0.856 |
| UBE216 | SAE1 | 0.856 |
| APOL2 | ZXDC | 0.856 |
| GBP1 | TBX21 | 0.856 |
| GBP1 | CHIC2 | 0.856 |
| LHFPL2 | POLR3B | 0.856 |
| ATG3 | SEC63 | 0.856 |
| LHFPL2 | SLC25A22 | 0.856 |
| P2RY14 | ST7L | 0.856 |
| GBP1 | TIMM44 | 0.856 |
| C1QA | PUS1 | 0.856 |
| GBP5 | DPEP2 | 0.856 |
| SECTM1 | VIM | 0.856 |
| APOL1 | ABCF2 | 0.856 |
| C1QA | GPR132 | 0.856 |
| P2RY14 | PPP1R16B | 0.856 |
| SECTM1 | RHOA | 0.856 |
| VAMP5 | EIF2B5 | 0.856 |
| SECTM1 | EXOSC10 | 0.856 |
| SECTM1 | EIF4G1 | 0.856 |
| GBP1 | TFB2M | 0.856 |
| UBE2L6 | PDK3 | 0.856 |
| GHI1B | C1QA | 0.856 |
| GBP1 | BZW2 | 0.856 |
| GBP5 | SCYL2 | 0.856 |
| AIM2 | MCM6 | 0.856 |
| SECTM1 | LAPTM5 | 0.856 |
| P2RY14 | VDR | 0.856 |
| GBP1 | TYMS | 0.856 |
| GBP1 | STK4 | 0.856 |
| C1QA | TIMELESS | 0.856 |
| P2RY14 | MCM5 | 0.856 |
| P2RY14 | ACTR6 | 0.856 |
| UBE2L6 | CTDSPL | 0.856 |
| IL15RA | OGDH | 0.856 |
| SMARCD3 | ACP5 | 0.856 |
| MGST2 | GBP1 | 0.856 |
| VAMP5 | UBAP2 | 0.856 |
| APOL2 | RANBP3 | 0.856 |
| GBP1 | ATP6V0A1 | 0.856 |
| PCSK6 | VAMP5 | 0.856 |
| RAB3IL1 | GBP1 | 0.856 |
| C1QA | MSH2 | 0.856 |
| VAMP5 | TSFM | 0.856 |
| P2RY14 | SLC41A3 | 0.856 |
| PSME1 | PFAS | 0.856 |
| PPCDC | GBP1 | 0.856 |
| C1QA | VAMP3 | 0.856 |
| GBP5 | EEF1D | 0.856 |
| UBE216 | IRF1 | 0.856 |
| NRG1 | VAMP5 | 0.856 |
| P2RY14 | ARHGAP1 | 0.856 |
| TMED3 | SECTM1 | 0.856 |
| GBP1 | PREP | 0.856 |
| POLB | CTNNBIP1 | 0.856 |
| SECTM1 | ITIH5 | 0.856 |
| NPC2 | APOL2 | 0.856 |
| P2RY14 | CIAPIN1 | 0.856 |
| GBP1 | SDCBP | 0.856 |
| POLB | GALK2 | 0.856 |
| AIM2 | CD40 | 0.856 |
| APOL1 | PML | 0.856 |
| AIM2 | TOPBP1 | 0.856 |
| VAMP5 | CPSF1 | 0.856 |
| IRF2 | POLB | 0.856 |
| VAMP5 | OXSR1 | 0.856 |
| POLB | NISCH | 0.856 |
| GBP5 | ARID4A | 0.856 |
| P2RY14 | APLP2 | 0.856 |
| IL15 | APOL2 | 0.856 |
| C1QA | PLEK2 | 0.856 |
| UBE2L6 | BZW2 | 0.856 |
| GBP1 | LSR | 0.856 |
| P2RY14 | TRAT1 | 0.856 |
| P2RY14 | CC2D1A | 0.856 |
| POLB | TCEAL4 | 0.856 |
| PSTPIP2 | SLA | 0.856 |
| P2RY14 | DNM2 | 0.856 |
| PSME1 | TBCD | 0.856 |
| SECTM1 | DOCK2 | 0.856 |
| APOL2 | USP48 | 0.856 |
| VAMP5 | VPS33B | 0.856 |
| UBE2L6 | POLR1D | 0.856 |
| UBE2L6 | ANP32B | 0.856 |
| GBP5 | SEC24B | 0.856 |
| GBP1 | CAP1 | 0.856 |
| GBP1 | VIPR1 | 0.856 |
| UBE2L6 | TMEM43 | 0.856 |
| C1QA | PRMT7 | 0.856 |
| SECTM1 | PDE4A | 0.856 |
| GBP1 | IDS | 0.856 |
| UBE2L6 | TES | 0.856 |
| GBP5 | NSMAF | 0.856 |
| VAMP5 | XPO1 | 0.856 |
| UBE2L6 | CD244 | 0.856 |
| UBE2L6 | DOCK2 | 0.856 |
| GBP1 | APLP2 | 0.856 |
| SECTM1 | IVNS1ABP | 0.856 |
| TRAPPC3 | VAMP5 | 0.856 |
| ODC1 | GBP1 | 0.856 |
| GBP1 | VPS33A | 0.856 |
| GBP1 | NMD3 | 0.856 |
| ACP2 | GBP1 | 0.856 |
| UBE2L6 | FAM111A | 0.856 |
| P2RY14 | PCNA | 0.856 |
| PSTPIP2 | DGKD | 0.856 |
| GBP1 | DALRD3 | 0.856 |
| P2RY14 | ELL | 0.856 |
| GBP1 | RAB11FIP3 | 0.856 |
| VAMP5 | MAZ | 0.856 |
| P2RY14 | NUP214 | 0.856 |
| VAMP5 | RPL39L | 0.856 |
| GBP5 | CCNT2 | 0.856 |
| APOL1 | CYB561 | 0.856 |
| P2RY14 | ARL3 | 0.856 |
| GBP5 | ZFYVE26 | 0.856 |
| P2RY14 | NAT10 | 0.856 |
| LIPA | P2RY14 | 0.856 |
| P2RY14 | TMEM109 | 0.856 |
| P2RY14 | POLD1 | 0.856 |
| AIM2 | RRBP1 | 0.856 |
| GBP5 | MANEA | 0.856 |
| UBE2L6 | ANGEL1 | 0.856 |
| APOL3 | DDX27 | 0.856 |
| VAMP5 | SEC61A1 | 0.856 |
| APOL1 | RPAP1 | 0.856 |
| P2RY14 | PSEN1 | 0.856 |
| GBP5 | M6PR | 0.856 |
| VAMP5 | TCF7 | 0.856 |
| STAT1 | NOLC1 | 0.856 |
| VAMP5 | CUEDC2 | 0.856 |
| APOL2 | CAMK4 | 0.856 |
| UBE2L6 | NCBP2 | 0.856 |
| SECTM1 | FYCO1 | 0.856 |
| P2RY14 | RAB33A | 0.856 |
| LIMK1 | VCP | 0.856 |
| PSME1 | RPAP1 | 0.856 |
| UBE2L6 | MBTPS1 | 0.856 |
| PTPN2 | APOL2 | 0.856 |
| GBP1 | SLC35D2 | 0.856 |
| GBP1 | ZNF573 | 0.856 |
| POLB | UBP1 | 0.856 |
| SECTM1 | CDK5RAP1 | 0.856 |
| UBE2L6 | ATP1A1 | 0.856 |
| LIMK1 | ABCF2 | 0.856 |
| POLB | CNOT4 | 0.856 |
| GBP1 | RP2 | 0.856 |
| C1QA | SCMH1 | 0.856 |
| C1QA | ODF2 | 0.856 |
| POLB | GALNT1 | 0.856 |
| P2RY14 | PVR | 0.856 |
| AKR1A1 | AIM2 | 0.856 |
| GBP1 | GNA11 | 0.856 |
| GBP5 | CONT1 | 0.856 |
| GBP1 | CSTF2 | 0.856 |
| SECTM1 | MYH9 | 0.856 |
| POLB | DHCR7 | 0.856 |
| POLB | ADIPOR2 | 0.856 |
| P2RY14 | RNASEH1 | 0.856 |
| GBP1 | PSMD6 | 0.856 |
| P2RY14 | TRIM38 | 0.856 |
| POLB | ALDH9A1 | 0.856 |
| UBE2L6 | RPN1 | 0.856 |
| AIM2 | CALCOCO2 | 0.856 |
| VAMP5 | VIPR1 | 0.856 |
| P2RY14 | SUV39H1 | 0.856 |
| CASP7 | LIMK1 | 0.856 |
| GBP5 | GMPPA | 0.856 |
| P2RY14 | FAF1 | 0.856 |
| UBE216 | ALDH1A1 | 0.856 |
| GBP5 | FHL3 | 0.856 |
| GBP1 | TNFSF8 | 0.856 |
| SMARCD3 | GNG11 | 0.856 |
| POLB | ULK1 | 0.856 |
| BST2 | P2RY14 | 0.856 |
| VAMP5 | NAGLU | 0.856 |
| VAMP5 | PFKP | 0.856 |
| GBP1 | ZBTB33 | 0.856 |
| C1QB | POLB | 0.856 |
| APOL2 | CNOT2 | 0.856 |
| SECTM1 | FBXL12 | 0.856 |
| POLB | SLC39A9 | 0.856 |
| UBE216 | MRPS27 | 0.856 |
| IL15RA | TNFRSF14 | 0.856 |
| APOL2 | LSG1 | 0.856 |
| GBP1 | BTN2A2 | 0.856 |
| GBP1 | TBXA2R | 0.856 |
| SECTM1 | MRPL49 | 0.856 |
| C1QA | FLII | 0.856 |
| APOL2 | TIMELESS | 0.856 |
| UBE2L6 | SLBP | 0.856 |
| GBP1 | NAPA | 0.856 |
| GBP1 | ZNF266 | 0.856 |
| CYB561 | CASP1 | 0.856 |
| P2RY14 | CTSC | 0.856 |
| UBE2L6 | FZD1 | 0.856 |
| SECTM1 | SAR1A | 0.856 |
| C1QA | TBRG4 | 0.856 |
| SECTM1 | BCAP31 | 0.856 |
| VAMP5 | PEMT | 0.856 |
| VAMP5 | DDX54 | 0.856 |
| P2RY14 | ZNF32 | 0.856 |
| POLB | RAB14 | 0.856 |
| VAMP5 | PXMP2 | 0.856 |
| CBR1 | GBP1 | 0.856 |
| VAMP5 | PRDM2 | 0.856 |
| UBE2L6 | ZNF589 | 0.856 |
| GBP1 | CRY2 | 0.856 |
| VAMP5 | TBC1D13 | 0.856 |
| AIM2 | OXSR1 | 0.856 |
| SECTM1 | CACNA1I | 0.856 |
| UBE2L6 | DDB2 | 0.856 |
| UBE2L6 | RGS12 | 0.856 |
| POLB | ATP2B4 | 0.856 |
| UBE2L6 | SNAPC4 | 0.856 |
| POLB | RIOK2 | 0.856 |
| UBE2L6 | ANKRD11 | 0.856 |
| GBP1 | TPM1 | 0.856 |
| GBP5 | ZNF451 | 0.856 |
| CASP7 | APOL2 | 0.856 |
| GBP1 | E2F1 | 0.856 |
| SECTM1 | JOSD1 | 0.856 |
| UBE2L6 | SRRM1 | 0.856 |
| VAMP5 | LRRC8D | 0.856 |
| ECHS1 | AIM2 | 0.856 |
| P2RY14 | TNFRSF21 | 0.856 |
| APOL2 | PDIA4 | 0.856 |
| STX10 | GBP1 | 0.856 |
| P2RY14 | NAP1L1 | 0.856 |
| GBP5 | VPS13C | 0.856 |
| UBE2L6 | AP4E1 | 0.856 |
| GBP5 | SP100 | 0.856 |
| SECTM1 | PTPN18 | 0.856 |
| POLB | KPNA6 | 0.856 |
| C1QA | OXSR1 | 0.856 |
| GBP5 | BST1 | 0.856 |
| AIM2 | CD244 | 0.856 |
| APOL2 | MCM5 | 0.856 |
| GBP5 | SLC31A2 | 0.856 |
| VAMP5 | AVEN | 0.856 |
| VAMP5 | ETFB | 0.856 |
| GBP5 | ZNF467 | 0.856 |
| PML | PPP2R5D | 0.856 |
| SECTM1 | CAMK4 | 0.856 |
| VAMP5 | PPARD | 0.856 |
| GBP5 | ARHGEF10 | 0.856 |
| GUK1 | VAMP5 | 0.856 |
| P2RY14 | PLXND1 | 0.856 |
| GBP1 | NISCH | 0.856 |
| PSME1 | PDHA1 | 0.856 |
| LIMK1 | BAHD1 | 0.856 |
| VAMP5 | FADD | 0.856 |
| APOL2 | RBBP4 | 0.856 |
| GBP1 | TRIM8 | 0.856 |
| LIMK1 | TSR1 | 0.856 |
| SECTM1 | PAXIP1 | 0.856 |
| VAMP5 | PES1 | 0.856 |
| SECTM1 | RFWD3 | 0.856 |
| UBE2L6 | EHBP1 | 0.856 |
| C1QA | SEC61A1 | 0.856 |
| GBP1 | EML3 | 0.856 |
| GBP1 | MLX | 0.856 |
| UBE2L6 | RGS14 | 0.856 |
| GBP5 | TNS1 | 0.856 |
| GTF2B | SECTM1 | 0.856 |
| APOL2 | NCOR1 | 0.856 |
| POLB | BCL2L11 | 0.856 |
| GBP5 | CHMP5 | 0.856 |
| C1QA | STK38 | 0.856 |
| UBE2L6 | ZCWPW1 | 0.856 |
| GBP1 | CACNA2D2 | 0.856 |
| APOL1 | ELAC2 | 0.856 |
| GBP5 | EPB41L3 | 0.856 |
| VAMP5 | PREB | 0.856 |
| POLB | SCARB1 | 0.856 |
| UBE2L6 | ST14 | 0.856 |
| UBE2L6 | PMM2 | 0.856 |
| GBP5 | EIF4G2 | 0.856 |
| DAPP1 | SLC38A1 | 0.856 |
| GNG11 | CALCOCO2 | 0.856 |
| P2RY14 | SLC25A37 | 0.856 |
| NPC2 | PSTPIP2 | 0.856 |
| P2RY14 | RCN3 | 0.856 |
| UBE2L6 | GTF3C2 | 0.856 |
| GNG11 | ATG3 | 0.856 |
| ATG3 | PPP3CA | 0.856 |
| GBP1 | RUVBL1 | 0.856 |
| UBE2L6 | CIAPIN1 | 0.856 |
| SECTM1 | VAT1 | 0.856 |
| POLB | AP2A2 | 0.856 |
| LHFPL2 | AMPD3 | 0.856 |
| P2RY14 | GLO1 | 0.856 |
| IL15RA | PDHA1 | 0.856 |
| GBP1 | POMT1 | 0.856 |
| MAT2B | VAMP5 | 0.856 |
| UBE2L6 | UBAP2L | 0.856 |
| UBE2L6 | MTCH2 | 0.856 |
| C1QA | NET1 | 0.856 |
| UBE2L6 | THAP7 | 0.856 |
| STAT1 | VAMP5 | 0.856 |
| C1QA | IL15 | 0.856 |
| ATG3 | DGKD | 0.856 |
| P2RY14 | DPH5 | 0.856 |
| VAMP5 | SPEN | 0.856 |
| VAMP5 | GALNT2 | 0.856 |
| C1QA | DDB2 | 0.856 |
| UBE2L6 | TBC1D13 | 0.856 |
| POLB | TPM1 | 0.856 |
| LHFPL2 | CEP250 | 0.856 |
| UBE2L6 | ARAF | 0.856 |
| LIMK1 | POLR3B | 0.856 |
| GBP1 | KIF23 | 0.856 |
| P2RY14 | PSMD6 | 0.856 |
| UBE2L6 | TOE1 | 0.856 |
| P2RY14 | DDB2 | 0.856 |
| P2RY14 | QPRT | 0.856 |
| P2RY14 | SLC1A4 | 0.856 |
| UBE2L6 | ACAD8 | 0.856 |
| SECTM1 | PRCC | 0.856 |
| POLB | GSR | 0.856 |
| LHFPL2 | BCR | 0.856 |
| GBP1 | GTF3C2 | 0.856 |
| GBP5 | SELENBP1 | 0.856 |
| UBE2L6 | MLX | 0.856 |
| UBE2L6 | ZC3H7A | 0.856 |
| P2RY14 | PACS1 | 0.856 |
| GBP1 | PUM2 | 0.856 |
| UBEZL6 | MAPK1 | 0.856 |
| UBEZL6 | STAT6 | 0.856 |
| VAMP5 | SAE1 | 0.856 |
| UBE2L6 | CDKAL1 | 0.856 |
| GBP1 | MYO9B | 0.856 |
| SECTM1 | ZNF200 | 0.856 |
| ATP6V1G1 | GBP1 | 0.856 |
| UBE2L6 | CHRNB1 | 0.856 |
| GBP1 | GBE1 | 0.856 |
| VAMP5 | THAP7 | 0.856 |
| SECTM1 | PDXK | 0.856 |
| P2RY14 | BCL11A | 0.856 |
| AIM2 | DGKD | 0.856 |
| NDUFA1 | DAPP1 | 0.855 |
| GBP1 | KLF2 | 0.855 |
| P2RY14 | ATP1B1 | 0.855 |
| LAPTM4A | C1QA | 0.855 |
| APOL1 | IL15 | 0.855 |
| SECTM1 | ITGA7 | 0.855 |
| C1QA | POLR1D | 0.855 |
| COASY | POLB | 0.855 |
| UBE2L6 | CCDC22 | 0.855 |
| GBP5 | CAM1 | 0.855 |
| UBE2L6 | SAP18 | 0.855 |
| P2RY14 | LRRC20 | 0.855 |
| POLB | TSC2 | 0.855 |
| P2RY14 | MITF | 0.855 |
| GBP1 | TRAF3IP3 | 0.855 |
| APOL2 | UBE4B | 0.855 |
| P2RY14 | THOP1 | 0.855 |
| POLB | SAMHD1 | 0.855 |
| ST3GAL5 | P2RY14 | 0.855 |
| GBP1 | RNF138 | 0.855 |
| GBP1 | NDUFS1 | 0.855 |
| AIM2 | NOLC1 | 0.855 |
| C1QA | MCM7 | 0.855 |
| P2RY14 | BYSL | 0.855 |
| IL12RB1 | VAMP5 | 0.855 |
| AIM2 | FAM53C | 0.855 |
| APOL2 | ERCC3 | 0.855 |
| C1QA | PAXIP1 | 0.855 |
| POLB | PCSK7 | 0.855 |
| GBP1 | ING1 | 0.855 |
| GBP5 | RBM7 | 0.855 |
| GBP1 | RNF41 | 0.855 |
| SECTM1 | HNMT | 0.855 |
| IL15RA | MAP3K4 | 0.855 |
| P2RY14 | POMT2 | 0.855 |
| UBE216 | MAPKAPK2 | 0.855 |
| SECTM1 | ZNF76 | 0.855 |
| UBE2L6 | RFX5 | 0.855 |
| VAMP5 | SLC2A4RG | 0.855 |
| VAMP5 | STK38 | 0.855 |
| GBP5 | CENPF | 0.855 |
| GBP5 | PLP2 | 0.855 |
| PML | TTLL12 | 0.855 |
| GBP1 | ZNF614 | 0.855 |
| P2RY14 | BCL2L13 | 0.855 |
| GBP1 | GNB1 | 0.855 |
| POLB | STOML1 | 0.855 |
| UBE2L6 | BRD4 | 0.855 |
| APOL1 | GOLGA3 | 0.855 |
| ITGA7 | APOL2 | 0.855 |
| GBP1 | RAB11FIP1 | 0.855 |
| UBE2L6 | FADD | 0.855 |
| LHFPL2 | SDC1 | 0.855 |
| GBP1 | ZNF212 | 0.855 |
| PSTPIP2 | GBP1 | 0.855 |
| DENND1A | APOL2 | 0.855 |
| UBE2L6 | ATP7B | 0.855 |
| ITPA | GBP1 | 0.855 |
| C1QA | CLN6 | 0.855 |
| DAPP1 | OSBPL9 | 0.855 |
| GBP5 | ERH | 0.855 |
| PML | KLF6 | 0.855 |
| TMEM59 | CALCOCO2 | 0.855 |
| GBP1 | LCK | 0.855 |
| GBP1 | DNM2 | 0.855 |
| POLB | POLR2C | 0.855 |
| GPR65 | C1QA | 0.855 |
| P2RY14 | IVNS1ABP | 0.855 |
| STAT1 | KLF6 | 0.855 |
| P2RY14 | VPS53 | 0.855 |
| PSTPIP2 | PPP4R1 | 0.855 |
| UBE2L6 | IRF4 | 0.855 |
| P2RY14 | AP2A2 | 0.855 |
| SECTM1 | SEC61A1 | 0.855 |
| GBP1 | MTAP | 0.855 |
| POLB | CLCF1 | 0.855 |
| ATP7B | SECTM1 | 0.855 |
| GBP1 | RFC2 | 0.855 |
| UBE2L6 | R3HDM2 | 0.855 |
| LAPTM4A | LHFPL2 | 0.855 |
| P2RY14 | ABHD5 | 0.855 |
| UBE2L6 | ATP6V1E1 | 0.855 |
| GBP1 | ARNTL | 0.855 |
| GBP1 | ICOS | 0.855 |
| SECTM1 | PTGES2 | 0.855 |
| POLB | CUTC | 0.855 |
| PSTPIP2 | SLTM | 0.855 |
| C1QA | RGS10 | 0.855 |
| GBP1 | TLE2 | 0.855 |
| GBP1 | LASP1 | 0.855 |
| P2RY14 | SSR3 | 0.855 |
| P2RY14 | CUTC | 0.855 |
| P2RY14 | VRK1 | 0.855 |
| GBP5 | LPIN2 | 0.855 |
| VAMP5 | RANBP3 | 0.855 |
| VAMP5 | LSR | 0.855 |
| GBP1 | TBRG4 | 0.855 |
| VAMP5 | SCMH1 | 0.855 |
| UBEZL6 | MAPK7 | 0.855 |
| UBE2L6 | PPM1D | 0.855 |
| C1QA | VPS33B | 0.855 |
| UBE2L6 | STARD5 | 0.855 |
| GBP1 | MPHOSPH10 | 0.855 |
| VAMP5 | PGM1 | 0.855 |
| IRF1 | ARID1A | 0.855 |
| GFI1B | VAMP5 | 0.855 |
| SECTM1 | MRPL9 | 0.855 |
| GBP1 | BCL11B | 0.855 |
| UBE2L6 | UBE2L3 | 0.855 |
| GBP5 | GCC2 | 0.855 |
| SECTM1 | EHD4 | 0.855 |
| VAMP5 | RFC1 | 0.855 |
| SECTM1 | SYNCRIP | 0.855 |
| VAMP5 | RPN1 | 0.855 |
| P2RY14 | GOT1 | 0.855 |
| APOL2 | TBCD | 0.855 |
| VAMP5 | POMT1 | 0.855 |
| MS4A6A | GBP1 | 0.855 |
| C1QA | GANAB | 0.855 |
| GBP1 | CCDC25 | 0.855 |
| VAMP5 | PSMD7 | 0.855 |
| UBE2L6 | FKRP | 0.855 |
| C1QA | POR | 0.855 |
| POLB | PEX14 | 0.855 |
| DAPP1 | RNGTT | 0.855 |
| ZNHIT1 | POLB | 0.855 |
| GBP1 | STMN1 | 0.855 |
| GBP5 | NME3 | 0.855 |
| C1QA | DUSP22 | 0.855 |
| POLB | MEN1 | 0.855 |
| APOL2 | TLE4 | 0.855 |
| VAMP5 | POU2F1 | 0.855 |
| UBE2L6 | PHF1 | 0.855 |
| P2RY14 | SLC24A3 | 0.855 |
| GBP1 | PRPSAP1 | 0.855 |
| UBE2L6 | STMN1 | 0.855 |
| UBE2L6 | NPAT | 0.855 |
| C1QA | SYNCRIP | 0.855 |
| GBP1 | HNMT | 0.855 |
| C1QA | TNK2 | 0.855 |
| P2RY14 | PDE6D | 0.855 |
| LHFPL2 | ETS2 | 0.855 |
| SECTM1 | BRD9 | 0.855 |
| POLB | GGA2 | 0.855 |
| APOL2 | DGKA | 0.855 |
| P2RY14 | TBC1D15 | 0.855 |
| VAMP5 | ABCF3 | 0.855 |
| C1QA | DNAJA3 | 0.855 |
| LHFPL2 | UPF2 | 0.855 |
| CYB561 | TCF7 | 0.855 |
| POLB | MRPL44 | 0.855 |
| GBP1 | SLAMF1 | 0.855 |
| P2RY14 | KHSRP | 0.855 |
| VAMP5 | MAPK13 | 0.855 |
| P2RY14 | PHKA2 | 0.855 |
| UBE2L6 | ETF1 | 0.855 |
| P2RY14 | CLN6 | 0.855 |
| POLB | SLC5A6 | 0.855 |
| POLB | POLD1 | 0.855 |
| SMARCD3 | SLC16A5 | 0.855 |
| P2RY14 | CD1A | 0.855 |
| P2RY14 | DCTD | 0.855 |
| APOL2 | RNGTT | 0.855 |
| PSTPIP2 | HK2 | 0.855 |
| VAMP5 | RNASEH2A | 0.855 |
| GBP1 | FBXL12 | 0.855 |
| GBP1 | ISOC1 | 0.855 |
| LIMK1 | LIG1 | 0.855 |
| VAMP5 | CASP6 | 0.855 |
| GBP1 | OSBPL2 | 0.855 |
| UBE2L6 | MAT2A | 0.855 |
| VAMP5 | DENND2D | 0.855 |
| SECTM1 | ATF7IP | 0.855 |
| VAMP5 | DDX23 | 0.855 |
| POLB | KLF13 | 0.855 |
| SECTM1 | CSE1L | 0.855 |
| DAPP1 | RPS6KA3 | 0.855 |
| POLB | PSME3 | 0.855 |
| UBE2L6 | CCDC51 | 0.855 |
| STX11 | C1QA | 0.855 |
| C1QA | PSMD5 | 0.855 |
| P2RY14 | TMC01 | 0.855 |
| APOL2 | PLK3 | 0.855 |
| UBE2L6 | SUCLG1 | 0.855 |
| GBP1 | EXOSC2 | 0.855 |
| POLB | ASXL2 | 0.855 |
| SECTM1 | SUV39H1 | 0.855 |
| UBE2L6 | ARHGEF2 | 0.855 |
| GBP1 | PRF1 | 0.855 |
| POLB | RGS10 | 0.855 |
| P2RY14 | WDR73 | 0.855 |
| GBP1 | PPT1 | 0.855 |
| GBP5 | PADI4 | 0.855 |
| AIM2 | GFI1B | 0.855 |
| GBP1 | MCFD2 | 0.855 |
| GBP1 | AHR | 0.855 |
| C1QA | CALU | 0.855 |
| CALCOCO2 | XPC | 0.855 |
| GBP5 | PLD3 | 0.855 |
| UBE2L6 | CUL4A | 0.855 |
| LHFPL2 | PSMD5 | 0.855 |
| P2RY14 | ATP13A1 | 0.855 |
| GBP1 | H6PD | 0.855 |
| VAMP5 | FYCO1 | 0.855 |
| GBP5 | APAF1 | 0.855 |
| PSME1 | BAHD1 | 0.855 |
| SECTM1 | CSK | 0.855 |
| SECTM1 | PCBP4 | 0.855 |
| UBE216 | CLEC4A | 0.855 |
| VAMP5 | PCNA | 0.855 |
| UBE2L6 | MAN1C1 | 0.855 |
| P2RY14 | CPT2 | 0.855 |
| POLB | GNA11 | 0.855 |
| PYCARD | GBP1 | 0.855 |
| TJP2 | POLB | 0.855 |
| NDUFB6 | LIMK1 | 0.855 |
| P2RY14 | ATG4A | 0.855 |
| P2RY14 | BCAT2 | 0.855 |
| POLB | CCDC22 | 0.855 |
| GBP1 | PIGA | 0.855 |
| GBP1 | VPS33B | 0.855 |
| POLB | MOAP1 | 0.855 |
| MYL9 | POLB | 0.855 |
| GBP1 | TTC19 | 0.855 |
| UBE2E1 | SECTM1 | 0.855 |
| SECTM1 | MYO1F | 0.855 |
| SECTM1 | TGOLN2 | 0.855 |
| DAPP1 | YEATS2 | 0.855 |
| GBP5 | CHI312 | 0.855 |
| SECTM1 | CAP1 | 0.855 |
| APOL2 | SETD3 | 0.855 |
| UBE2L6 | PHF20 | 0.855 |
| GBP1 | CCNB1 | 0.855 |
| AIM2 | SYNJ2 | 0.855 |
| UBE2L6 | MAP3K14 | 0.855 |
| PSME1 | PARP16 | 0.855 |
| P2RY14 | RABIF | 0.855 |
| VAMP5 | ITGA7 | 0.855 |
| PSME1 | PML | 0.855 |
| P2RY14 | ARHGEF2 | 0.855 |
| GBP1 | STARD5 | 0.855 |
| GBP1 | PRMT7 | 0.855 |
| DAPP1 | DCTN4 | 0.855 |
| GBP1 | PSMD1 | 0.855 |
| POLB | NMI | 0.855 |
| P2RY14 | NIT2 | 0.855 |
| UBE2L6 | ZXDC | 0.855 |
| GBP1 | PPP2CB | 0.855 |
| C1QA | POMT1 | 0.855 |
| LIMK1 | MYC | 0.855 |
| POLB | GNRH1 | 0.855 |
| SECTM1 | SMAD3 | 0.855 |
| GBP5 | ORMDL2 | 0.855 |
| P2RY14 | CDC42EP2 | 0.855 |
| TSPAN32 | GBP1 | 0.855 |
| LIMK1 | ELAC2 | 0.855 |
| APOL1 | PIK3C2B | 0.855 |
| UBE2L6 | ECHDC2 | 0.855 |
| ACO1 | VAMP5 | 0.855 |
| P2RY14 | CXCR3 | 0.855 |
| P2RY14 | HMGN4 | 0.855 |
| UBE2L6 | ZNF394 | 0.855 |
| SECTM1 | TBL2 | 0.855 |
| UBE2L6 | ATP6AP1 | 0.855 |
| C1QA | KLF6 | 0.855 |
| PML | GEMIN4 | 0.855 |
| POLB | ITPR3 | 0.855 |
| VAMP5 | CNOT2 | 0.855 |
| UBE2L6 | PSMD7 | 0.855 |
| GBP5 | EVI2A | 0.855 |
| GBP5 | KIF1B | 0.855 |
| GBP5 | TTC13 | 0.855 |
| GBP1 | GSPT2 | 0.855 |
| DUSP22 | VAMP5 | 0.855 |
| POLB | MCCC1 | 0.855 |
| SECTM1 | SCARB1 | 0.855 |
| LHFPL2 | BTN2A2 | 0.855 |
| C1QA | AACS | 0.855 |
| UBE2L6 | SLC16A6 | 0.855 |
| APOL2 | TRAP1 | 0.855 |
| GBP1 | ITGA5 | 0.855 |
| P2RY14 | SETD4 | 0.855 |
| PSME1 | NDUFB6 | 0.855 |
| POLB | MKKS | 0.855 |
| P2RY14 | RAB27A | 0.855 |
| GBP1 | PITPNC1 | 0.855 |
| C1QA | CDCA4 | 0.855 |
| POLB | ARHGAP1 | 0.855 |
| GBP5 | INHBB | 0.855 |
| P2RY14 | EAF2 | 0.855 |
| C1QA | GUK1 | 0.855 |
| UBE2L6 | CD33 | 0.855 |
| GBP1 | CHRNB1 | 0.855 |
| GBP1 | PBXIP1 | 0.855 |
| UBE2L6 | BAG3 | 0.855 |
| P2RY14 | MRPS28 | 0.855 |
| APOL1 | DHX9 | 0.855 |
| GBP1 | NPEPPS | 0.855 |
| APOL2 | MED9 | 0.855 |
| GBP1 | SETBP1 | 0.855 |
| GMPR2 | GBP1 | 0.855 |
| AIM2 | ADAM17 | 0.855 |
| UBE2L6 | SLC35A2 | 0.855 |
| CASP1 | CAPN2 | 0.855 |
| POLB | RIN3 | 0.855 |
| UBE2L6 | PES1 | 0.855 |
| PSME1 | PPM1G | 0.855 |
| POLB | PUS7 | 0.855 |
| VAMP5 | PLAA | 0.855 |
| GBP1 | VAC14 | 0.855 |
| GBP1 | SH3TC1 | 0.855 |
| STK16 | SECTM1 | 0.855 |
| UBE2L6 | TRIM28 | 0.855 |
| SECTM1 | NSFL1C | 0.855 |
| GBP5 | CHD1 | 0.855 |
| POLB | MAP3K10 | 0.855 |
| C1QA | PBXIP1 | 0.855 |
| POLB | RIPK2 | 0.855 |
| UBE2L6 | MDK | 0.855 |
| UBE2L6 | LETM1 | 0.855 |
| ELP4 | P2RY14 | 0.855 |
| UBE2L6 | PDE4A | 0.855 |
| LIMK1 | NCL | 0.855 |
| C1QA | GTPBP4 | 0.855 |
| GBP1 | DGKA | 0.855 |
| GBP1 | IFT122 | 0.855 |
| UBE2L6 | CLSTN1 | 0.855 |
| LIMK1 | CYB561 | 0.855 |
| UBE2L6 | RCL1 | 0.855 |
| GSTO1 | VAMP5 | 0.855 |
| AIM2 | SMARCC1 | 0.855 |
| SECTM1 | ING1 | 0.855 |
| C1QA | DPF2 | 0.855 |
| AIM2 | PLAGL2 | 0.855 |
| UBE2L6 | CNIH4 | 0.855 |
| CALCOCO2 | OXSR1 | 0.855 |
| GBP1 | SLCO3A1 | 0.855 |
| LHFPL2 | AKAP1 | 0.855 |
| DAPP1 | CAMK4 | 0.855 |
| SECTM1 | POGZ | 0.855 |
| IL15RA | POLG | 0.855 |
| SECTM1 | THAP7 | 0.855 |
| UBE2L6 | ASB1 | 0.855 |
| PSTPIP2 | XPO1 | 0.855 |
| GBP1 | CDC25B | 0.855 |
| C1QA | PARP6 | 0.855 |
| MRPL44 | GBP1 | 0.855 |
| GBP1 | PRPSAP2 | 0.855 |
| P2RY14 | CCNK | 0.855 |
| VAMP5 | TP53BP2 | 0.855 |
| POLB | FNBP1 | 0.855 |
| GBP5 | DNAJB14 | 0.855 |
| UBE2L6 | BCOR | 0.855 |
| UBE2L6 | EXOSC9 | 0.855 |
| UBE2L6 | SCAMP2 | 0.855 |
| POLB | FAF1 | 0.855 |
| SECTM1 | SLC25A17 | 0.855 |
| ACP5 | LIMK1 | 0.855 |
| GBP1 | TBC1D1 | 0.855 |
| PML | DHX9 | 0.855 |
| GBP5 | COPE | 0.855 |
| GBP5 | GADD45A | 0.855 |
| UBE2L6 | RIPK2 | 0.855 |
| VAMP5 | EXOC7 | 0.855 |
| UBE2L6 | NR3C2 | 0.855 |
| POLB | PPBP | 0.855 |
| VAMP5 | ACOX3 | 0.855 |
| SECTM1 | SOX4 | 0.855 |
| GBP1 | CHST2 | 0.855 |
| GBP1 | ING3 | 0.854 |
| POLB | STX11 | 0.854 |
| GBP1 | PBX1 | 0.854 |
| GBP1 | PUS1 | 0.854 |
| APOL2 | KLF2 | 0.854 |
| CPVL | LHFPL2 | 0.854 |
| UBE2L6 | CLASP1 | 0.854 |
| GBP1 | PTS | 0.854 |
| C1QA | CTDSPL | 0.854 |
| LIMK1 | STAT1 | 0.854 |
| PSTPIP2 | TP53BP2 | 0.854 |
| GBP1 | RIN3 | 0.854 |
| GBP5 | GNA12 | 0.854 |
| GNAZ | P2RY14 | 0.854 |
| POLB | TICAM1 | 0.854 |
| SECTM1 | ABCC10 | 0.854 |
| UBE2L6 | ANXA7 | 0.854 |
| SECTM1 | ALOX5 | 0.854 |
| UBE2L6 | ZFPL1 | 0.854 |
| PML | RPAP1 | 0.854 |
| POLB | ARHGEF6 | 0.854 |
| POLB | ING3 | 0.854 |
| LRP1 | GBP1 | 0.854 |
| COX6B1 | SECTM1 | 0.854 |
| PML | ILF3 | 0.854 |
| APOL2 | GPBP1L1 | 0.854 |
| SECTM1 | DVL3 | 0.854 |
| CALCOCO2 | TRAPPC3 | 0.854 |
| DAPP1 | PML | 0.854 |
| LHFPL2 | MAP4 | 0.854 |
| GBP5 | UXT | 0.854 |
| UBE2L6 | PREB | 0.854 |
| GBP1 | CCNG1 | 0.854 |
| P2RY14 | FAM111A | 0.854 |
| DAPP1 | ADNP | 0.854 |
| P2RY14 | UBE2B | 0.854 |
| POLB | RNMT | 0.854 |
| PSTPIP2 | OXSR1 | 0.854 |
| NDUFB6 | PML | 0.854 |
| P2RY14 | ENY2 | 0.854 |
| P2RY14 | RBKS | 0.854 |
| IL15RA | ABL1 | 0.854 |
| GNG11 | CYB561 | 0.854 |
| APOL1 | LRIG1 | 0.854 |
| AIM2 | TLE4 | 0.854 |
| GBP1 | ECD | 0.854 |
| UBE2L6 | MTSS1 | 0.854 |
| GBP1 | GTPBP8 | 0.854 |
| GBP1 | HSDL2 | 0.854 |
| GBP1 | ZNF235 | 0.854 |
| C1QA | PLAA | 0.854 |
| UBE2L6 | CSRP1 | 0.854 |
| GBP1 | FLAD1 | 0.854 |
| C1QA | EXOSC10 | 0.854 |
| SECTM1 | PITRM1 | 0.854 |
| POLB | FZD2 | 0.854 |
| LHFPL2 | MDM2 | 0.854 |
| VAMP5 | SEC63 | 0.854 |
| POLB | PRPF4 | 0.854 |
| GBP5 | MPST | 0.854 |
| GBP1 | PPP1R8 | 0.854 |
| GBP1 | ZNF426 | 0.854 |
| LHFPL2 | ENSA | 0.854 |
| UBE2L6 | MRPL44 | 0.854 |
| VAMP5 | SMAD3 | 0.854 |
| C1QA | RFWD3 | 0.854 |
| VAMP5 | POLR2H | 0.854 |
| P2RY14 | TBC1D13 | 0.854 |
| POLB | PREB | 0.854 |
| C1QA | ETS1 | 0.854 |
| P2RY14 | RUFY3 | 0.854 |
| GBP1 | THOC1 | 0.854 |
| P2RY14 | SIGLEC7 | 0.854 |
| C1QA | CSRP1 | 0.854 |
| VAMP5 | HIC2 | 0.854 |
| GBP5 | RENBP | 0.854 |
| CASP7 | C1QA | 0.854 |
| C1QA | STAG1 | 0.854 |
| C1QA | UBAP2 | 0.854 |
| P2RY14 | PSMD7 | 0.854 |
| UBE2L6 | MUTYH | 0.854 |
| LHFPL2 | LIG1 | 0.854 |
| PSME1 | DPF2 | 0.854 |
| GBP1 | NUDT21 | 0.854 |
| AIM2 | ATF7IP | 0.854 |
| GBP1 | KCNMA1 | 0.854 |
| UBE2L6 | SLCO3A1 | 0.854 |
| GBP1 | MAPRE1 | 0.854 |
| UBE2L6 | PPAT | 0.854 |
| POLB | TMEM106C | 0.854 |
| GBP5 | NSUN3 | 0.854 |
| UBE2L6 | SLC39A6 | 0.854 |
| VAMP5 | CLN6 | 0.854 |
| UBEZL6 | CETN2 | 0.854 |
| GBP1 | WBP4 | 0.854 |
| STX11 | VAMP5 | 0.854 |
| SEPHS2 | GBP1 | 0.854 |
| VAMP5 | VPS72 | 0.854 |
| P2RY14 | PAFAH1B2 | 0.854 |
| SECTM1 | RABEPK | 0.854 |
| VAMP5 | TICAM1 | 0.854 |
| LIMK1 | RBM28 | 0.854 |
| GBP5 | NCOA3 | 0.854 |
| LHFPL2 | DHX9 | 0.854 |
| P2RY14 | TRIM8 | 0.854 |
| HEBP1 | GBP1 | 0.854 |
| DAPP1 | NOLC1 | 0.854 |
| C1QA | PRPF8 | 0.854 |
| POLB | WRN | 0.854 |
| UBE2L6 | TEX10 | 0.854 |
| GBP1 | IMMT | 0.854 |
| SECTM1 | FKRP | 0.854 |
| GBP1 | ETFDH | 0.854 |
| GBP1 | ARHGEF2 | 0.854 |
| APOL1 | ILF3 | 0.854 |
| UBE2L6 | BCL2L2 | 0.854 |
| SECTM1 | TUFT1 | 0.854 |
| GBP1 | ITGAL | 0.854 |
| GBP1 | TAPBPL | 0.854 |
| UBE2L6 | HERC2 | 0.854 |
| GBP5 | TALDO1 | 0.854 |
| GBP1 | CSDE1 | 0.854 |
| GBP5 | TGFBI | 0.854 |
| C1QA | RUVBL1 | 0.854 |
| P2RY14 | SLC35A2 | 0.854 |
| UBE2L6 | POLR2D | 0.854 |
| GBP1 | MNAT1 | 0.854 |
| UBE2L6 | NONO | 0.854 |
| UBE2L6 | MYO9A | 0.854 |
| PSME1 | RRS1 | 0.854 |
| SLC22A18 | GBP1 | 0.854 |
| UBE2L6 | ZCCHC14 | 0.854 |
| LIMK1 | NFATC1 | 0.854 |
| POLB | H6PD | 0.854 |
| APOL2 | FANCE | 0.854 |
| GBP1 | NONO | 0.854 |
| GBP1 | RNF130 | 0.854 |
| GBP1 | ACTL6A | 0.854 |
| SECTM1 | IL1R1 | 0.854 |
| SMARCD3 | CYB561 | 0.854 |
| PML | NFATC3 | 0.854 |
| SECTM1 | MBD2 | 0.854 |
| APOL1 | CASP1 | 0.854 |
| VAMP5 | SMARCD1 | 0.854 |
| C1QA | BCAT2 | 0.854 |
| VAMP5 | KLF2 | 0.854 |
| GBP1 | PITPNM1 | 0.854 |
| PML | YEATS2 | 0.854 |
| GBP5 | PRKAB2 | 0.854 |
| P2RY14 | EEF1E1 | 0.854 |
| C1QA | TRAPPC3 | 0.854 |
| UBE2L6 | CBX1 | 0.854 |
| IRF1 | GBP1 | 0.854 |
| SECTM1 | PRPF8 | 0.854 |
| LHFPL2 | LRRC8D | 0.854 |
| GBP1 | MBNL2 | 0.854 |
| POLB | BCOR | 0.854 |
| CD53 | P2RY14 | 0.854 |
| POLB | ADAM17 | 0.854 |
| GBP5 | RABSA | 0.854 |
| UBE2L6 | RPS6KA3 | 0.854 |
| ACP1 | GBP1 | 0.854 |
| P2RY14 | PEX14 | 0.854 |
| VAMP5 | PCBP4 | 0.854 |
| LHFPL2 | PLAA | 0.854 |
| GBP5 | PANK3 | 0.854 |
| LHFPL2 | MAX | 0.854 |
| UBE2L6 | TACC1 | 0.854 |
| SECTM1 | MFGE8 | 0.854 |
| UBE2L6 | POLR2C | 0.854 |
| UBE2L6 | DHX38 | 0.854 |
| UBE2L6 | PREP | 0.854 |
| SECTM1 | BAP1 | 0.854 |
| GBP1 | EXOSC8 | 0.854 |
| APOL2 | POU2F1 | 0.854 |
| PLEK2 | VAMP5 | 0.854 |
| STAT1 | CAPN2 | 0.854 |
| SLC7A7 | GBP1 | 0.854 |
| AIM2 | ATP2B4 | 0.854 |
| POLB | WWC3 | 0.854 |
| CYB561 | SMARCC1 | 0.854 |
| GBP1 | SNX11 | 0.854 |
| GBP1 | RELA | 0.854 |
| VAMP3 | VAMP5 | 0.854 |
| GBP1 | GLMN | 0.854 |
| GBP1 | PLCL2 | 0.854 |
| PSME1 | PRDM2 | 0.854 |
| P2RY14 | RDH11 | 0.854 |
| GBP1 | TMC6 | 0.854 |
| UBE2L6 | RASGRP2 | 0.854 |
| UBE2L6 | AQR | 0.854 |
| APOL2 | TBRG4 | 0.854 |
| VAMP5 | POR | 0.854 |
| P2RY14 | GNL2 | 0.854 |
| P2RY14 | WWOX | 0.854 |
| GBP5 | UFC1 | 0.854 |
| SECTM1 | ZC3H7A | 0.854 |
| VAMP5 | IL15 | 0.854 |
| UBE2L6 | DENND4A | 0.854 |
| GBP1 | PGM1 | 0.854 |
| VAMP5 | NET1 | 0.854 |
| GBP1 | SPG21 | 0.854 |
| APOL2 | ITPKB | 0.854 |
| VAMP5 | ADRM1 | 0.854 |
| APOL1 | TSR1 | 0.854 |
| GBP1 | BRD9 | 0.854 |
| UBE2L6 | CDCA8 | 0.854 |
| GBP1 | MAP2K4 | 0.854 |
| GBP1 | ATP2A3 | 0.854 |
| GBP1 | TRIM46 | 0.854 |
| L15RA | PRDM2 | 0.854 |
| P2RY14 | ASGR1 | 0.854 |
| UBE2L6 | MDM2 | 0.854 |
| GBP1 | PDK2 | 0.854 |
| GBP1 | ZNF211 | 0.854 |
| IL15RA | GLG1 | 0.854 |
| POLB | RCE1 | 0.854 |
| PSME1 | RUNX2 | 0.854 |
| GBP1 | MCM7 | 0.854 |
| SECTM1 | AMPD3 | 0.854 |
| PSME1 | RASGRP2 | 0.854 |
| LHFPL2 | IL1R1 | 0.854 |
| UBE2L6 | OVGP1 | 0.854 |
| UBE2L6 | SLC39A8 | 0.854 |
| IL15RA | NFATC1 | 0.854 |
| GBP1 | IL1B | 0.854 |
| SECTM1 | CDCA4 | 0.854 |
| CNIH4 | GBP1 | 0.854 |
| CASP1 | PPP3CA | 0.854 |
| C1QA | HIC2 | 0.854 |
| C1QA | RFC1 | 0.854 |
| UBE2L6 | SNIP1 | 0.854 |
| UBE2L6 | ITIH5 | 0.854 |
| C1QA | TSFM | 0.854 |
| UBE2L6 | TMEM87A | 0.854 |
| SECTM1 | TBRG4 | 0.854 |
| PSME1 | PSTPIP2 | 0.854 |
| UBE2L6 | VPS37B | 0.854 |
| SECTM1 | UBTF | 0.854 |
| UBE2L6 | EXOSC2 | 0.854 |
| UBE2L6 | RUFY3 | 0.854 |
| APOL2 | MYH9 | 0.854 |
| VAMP5 | CAMK4 | 0.854 |
| AIM2 | RUNX2 | 0.854 |
| POLB | CBX7 | 0.854 |
| P2RY14 | SENP3 | 0.854 |
| C1QA | PSMA2 | 0.854 |
| APOL2 | POGZ | 0.854 |
| VAMP5 | ABR | 0.854 |
| SECTM1 | ALMS1 | 0.854 |
| GBP1 | R3HDM2 | 0.854 |
| POLB | ZNF614 | 0.854 |
| VAMP5 | DGKA | 0.854 |
| SECTM1 | ZNF589 | 0.854 |
| UBE2L6 | BTN2A1 | 0.854 |
| LHFPL2 | CPNE3 | 0.854 |
| UBE2L6 | PPP4R1 | 0.854 |
| GBP1 | PRKACA | 0.854 |
| PSME1 | BRD9 | 0.854 |
| UBE2L6 | TPM1 | 0.854 |
| APOL2 | APBA2 | 0.854 |
| GBP1 | GGA1 | 0.854 |
| GBP1 | DDX1 | 0.854 |
| APOL2 | VIPR1 | 0.854 |
| RAB32 | GBP1 | 0.854 |
| UBE2L6 | ULK1 | 0.854 |
| GBP1 | BCCIP | 0.854 |
| P2RY14 | GOSR2 | 0.854 |
| GBP1 | MINK1 | 0.854 |
| GBP5 | SOD1 | 0.854 |
| APOL2 | STT3A | 0.854 |
| AIM2 | SLTM | 0.854 |
| IL15RA | DHX9 | 0.854 |
| APOL2 | DLEC1 | 0.854 |
| POLB | PFDN5 | 0.854 |
| SMARCD3 | APOL2 | 0.854 |
| C1QA | PSMC5 | 0.854 |
| SECTM1 | TOLLIP | 0.854 |
| SECTM1 | PPP4R1 | 0.854 |
| CYB561 | OSBP | 0.854 |
| AIM2 | TRAPPC3 | 0.854 |
| C1QA | SEC23B | 0.854 |
| GBP1 | PSMD2 | 0.854 |
| RHOC | POLB | 0.854 |
| GBP1 | SHC1 | 0.854 |
| GBP1 | PLK3 | 0.854 |
| SECTM1 | TMSB10 | 0.854 |
| GBP1 | TNFRSF9 | 0.854 |
| GBP1 | GMDS | 0.854 |
| APOL2 | MYC | 0.854 |
| UBE2L6 | GGA1 | 0.854 |
| UBE2L6 | DECR1 | 0.854 |
| UBE2L6 | LCK | 0.854 |
| APOL1 | RRS1 | 0.854 |
| ABHD3 | C1QA | 0.854 |
| VAMP5 | TRIM28 | 0.854 |
| P2RY14 | NAT1 | 0.854 |
| GBP1 | CLSTN1 | 0.854 |
| VAMP5 | ITPKC | 0.854 |
| GBP5 | SEC23IP | 0.854 |
| C1QA | GPBP1L1 | 0.854 |
| UBE2L6 | SHC1 | 0.854 |
| PSME1 | DGKA | 0.854 |
| P2RY14 | LRRC42 | 0.854 |
| VAMP5 | USP48 | 0.854 |
| SECTM1 | GPR65 | 0.854 |
| LHFPL2 | POU2F1 | 0.854 |
| APOL1 | NMT1 | 0.854 |
| POLB | ICMT | 0.854 |
| GSTO1 | FAS | 0.854 |
| SECTM1 | PEX14 | 0.854 |
| PSME1 | SMARCD1 | 0.854 |
| GBP5 | COPZ1 | 0.854 |
| INPPL1 | GBP1 | 0.854 |
| TMEM59 | NDRG1 | 0.854 |
| VAMP5 | RFWD3 | 0.854 |
| SECTM1 | SCAP | 0.854 |
| VAMP5 | TMEM97 | 0.854 |
| UBE2L6 | SLC6A6 | 0.854 |
| P2RY14 | KMO | 0.854 |
| GBP1 | CMT | 0.854 |
| DAPP1 | CTCF | 0.854 |
| GBP1 | UFM1 | 0.854 |
| PSMA2 | GBP1 | 0.854 |
| C1QA | IMP4 | 0.854 |
| PHC2 | P2RY14 | 0.854 |
| P2RY14 | MUC1 | 0.854 |
| VAMP5 | INPP1 | 0.854 |
| P2RY14 | GATA1 | 0.854 |
| STK16 | DAPP1 | 0.854 |
| GBP1 | RASSF1 | 0.854 |
| MTHFD2 | PZRY14 | 0.854 |
| GBP1 | ABCC5 | 0.854 |
| UBE2L6 | GORASP2 | 0.854 |
| GBP1 | CASP9 | 0.854 |
| AIM2 | PPP2R5B | 0.854 |
| PKIG | GBP1 | 0.854 |
| P2RY14 | MAP3K8 | 0.854 |
| POLB | BAG3 | 0.854 |
| GBP5 | CDKN1A | 0.854 |
| GBP1 | CYP20A1 | 0.854 |
| P2RY14 | BRD4 | 0.854 |
| GBP1 | KLHL2 | 0.854 |
| POLB | RCL1 | 0.854 |
| POLB | RAB11FIP3 | 0.854 |
| GBP1 | TOE1 | 0.854 |
| GBP1 | MAZ | 0.854 |
| POLB | ACO1 | 0.854 |
| UBE2L6 | POLH | 0.854 |
| UBE2L6 | RNASEH2A | 0.854 |
| GBP1 | CTDP1 | 0.854 |
| P2RY14 | ABCG1 | 0.854 |
| IRF1 | DPF2 | 0.854 |
| SECTM1 | ATP2B4 | 0.854 |
| UBE2L6 | SLC2A9 | 0.854 |
| PSME1 | ERCC3 | 0.854 |
| APOL2 | DNAJA3 | 0.854 |
| GBP1 | SAMSN1 | 0.854 |
| AIM2 | CTCF | 0.854 |
| LHFPL2 | CAMK2G | 0.854 |
| LHFPL2 | ARFGEF1 | 0.854 |
| APOL1 | VILL | 0.854 |
| C1QA | RERE | 0.854 |
| APOL2 | SNAPC4 | 0.854 |
| C1QA | SLTM | 0.854 |
| UBE2L6 | TSC1 | 0.854 |
| POLB | ABHD11 | 0.854 |
| POLB | THOP1 | 0.854 |
| APOL1 | APOL2 | 0.854 |
| POLB | SUV39H1 | 0.854 |
| AIM2 | ACO1 | 0.854 |
| VAMP5 | CCDC51 | 0.854 |
| GBP1 | HMGN4 | 0.854 |
| P2RY14 | NRIP3 | 0.854 |
| P2RY14 | MLYCD | 0.854 |
| SLC22A1 | GBP1 | 0.854 |
| GBP1 | RCN3 | 0.854 |
| GBP1 | BANK1 | 0.854 |
| POLB | CHMP6 | 0.854 |
| C1QA | PSMD2 | 0.854 |
| P2RY14 | KCTD5 | 0.854 |
| GBP1 | HCFC2 | 0.854 |
| DAPP1 | SLCO4C1 | 0.854 |
| VAMP5 | ZNF589 | 0.854 |
| SECTM1 | GTF3C5 | 0.854 |
| PSME1 | MED9 | 0.854 |
| CASP1 | SLTM | 0.854 |
| LHFPL2 | VDAC3 | 0.854 |
| GFI1B | SECTM1 | 0.854 |
| C1QA | PLK3 | 0.854 |
| GBP5 | MYH3 | 0.854 |
| APOL2 | CPSF1 | 0.854 |
| NMI | VAMP5 | 0.854 |
| GBP1 | RB1CC1 | 0.854 |
| C1QA | SIRTS | 0.854 |
| GBP5 | MGMT | 0.854 |
| UBE2L6 | PSME3 | 0.854 |
| APOL2 | PFAS | 0.854 |
| GBP5 | MRPL40 | 0.854 |
| ITIH5 | GBP1 | 0.854 |
| POLB | PSMD2 | 0.854 |
| UBE2L6 | DDX46 | 0.854 |
| AIM2 | DNPEP | 0.854 |
| GBP1 | ECE1 | 0.854 |
| GBP1 | SLC9A1 | 0.854 |
| VAMP5 | PARP2 | 0.854 |
| PTPN2 | VAMP5 | 0.854 |
| C1QA | EHD4 | 0.854 |
| UBE2L6 | SKP2 | 0.854 |
| CALCOCO2 | RUNX2 | 0.854 |
| GBP1 | RANBP3 | 0.853 |
| P2RY14 | PSMD9 | 0.853 |
| P2RY14 | HIP1 | 0.853 |
| VAMP5 | CHMP7 | 0.853 |
| POLB | BECN1 | 0.853 |
| P2RY14 | AGPAT4 | 0.853 |
| GBP1 | AGTPBP1 | 0.853 |
| UBE2L6 | PDE4B | 0.853 |
| STAT1 | PFDN5 | 0.853 |
| VAMP5 | BCCIP | 0.853 |
| POLB | PTGES2 | 0.853 |
| P2RY14 | CDS2 | 0.853 |
| LHFPL2 | CD244 | 0.853 |
| SECTM1 | ETF1 | 0.853 |
| DAPP1 | NCOR1 | 0.853 |
| DAPP1 | NPAT | 0.853 |
| GBP1 | BFAR | 0.853 |
| POLB | PAPOLA | 0.853 |
| TMEM59 | AGPAT3 | 0.853 |
| VAMP5 | SUV39H1 | 0.853 |
| GBP1 | CPT2 | 0.853 |
| TRAPPC3 | PML | 0.853 |
| P2RY14 | PRSS23 | 0.853 |
| P2RY14 | WDR74 | 0.853 |
| P2RY14 | STYXL1 | 0.853 |
| UBE2L6 | PTGES2 | 0.853 |
| LHFPL2 | RGS10 | 0.853 |
| VAMP5 | CC2D1A | 0.853 |
| ATG3 | PPP2RSA | 0.853 |
| UBE216 | ASB8 | 0.853 |
| POLB | SSH3 | 0.853 |
| P2RY14 | FBN2 | 0.853 |
| POLB | CSDE1 | 0.853 |
| P2RY14 | EPHB6 | 0.853 |
| VAMP5 | HAX1 | 0.853 |
| IL15RA | ARID1A | 0.853 |
| C1QA | ELOVL5 | 0.853 |
| UBE2L6 | MALT1 | 0.853 |
| CDK5RAP3 | GBP1 | 0.853 |
| GBP1 | GOLGA7 | 0.853 |
| APOL2 | DNM2 | 0.853 |
| P2RY14 | SLC25A20 | 0.853 |
| APOL2 | PGM1 | 0.853 |
| UBE2L6 | USP47 | 0.853 |
| NPC2 | PF4V1 | 0.853 |
| PRRG4 | TMSB10 | 0.853 |
| LHFPL2 | RFC1 | 0.853 |
| GBP5 | PTGS2 | 0.853 |
| GBP1 | PTPNZ | 0.853 |
| GBP1 | HSD17B12 | 0.853 |
| P2RY14 | SIRPB1 | 0.853 |
| SECTM1 | CALU | 0.853 |
| GBP5 | RB1 | 0.853 |
| UBE2L6 | ACTR8 | 0.853 |
| P2RY14 | ACTN4 | 0.853 |
| MAX | PML | 0.853 |
| LSM1 | VAMP5 | 0.853 |
| NENF | APOL2 | 0.853 |
| AIM2 | PPP3CA | 0.853 |
| UBE2L6 | CAND1 | 0.853 |
| GBP1 | DEDD | 0.853 |
| GBP1 | WDR26 | 0.853 |
| UBE2L6 | BSDC1 | 0.853 |
| SECTM1 | SRF | 0.853 |
| GBP1 | GFI1 | 0.853 |
| GBP5 | FES | 0.853 |
| VAMP5 | RASGRP2 | 0.853 |
| PML | TOB2 | 0.853 |
| CALCOCO2 | SPEN | 0.853 |
| GBP5 | SNCA | 0.853 |
| VAMP5 | AQP3 | 0.853 |
| UBE2L6 | WDR12 | 0.853 |
| GYPC | SECTM1 | 0.853 |
| GBP5 | UBE20 | 0.853 |
| APOL2 | VPS33B | 0.853 |
| VAMP5 | VPS4A | 0.853 |
| GBP1 | MED9 | 0.853 |
| POLB | KCTD17 | 0.853 |
| GBP5 | EPB42 | 0.853 |
| C1QA | FBXW2 | 0.853 |
| POLB | PGRMC2 | 0.853 |
| VAMP5 | BARD1 | 0.853 |
| SECTM1 | LRRC8D | 0.853 |
| AIM2 | TSPAN14 | 0.853 |
| PSME1 | FAS | 0.853 |
| PPBP | VAMP5 | 0.853 |
| GBP1 | TNFAIP8 | 0.853 |
| P2RY14 | TRAF3IP2 | 0.853 |
| SECTM1 | IL21R | 0.853 |
| POLB | CPSF4 | 0.853 |
| VAMP5 | NCOR1 | 0.853 |
| POLB | PDLIM5 | 0.853 |
| POLB | DNAJB6 | 0.853 |
| VAMP5 | IL1B | 0.853 |
| P2RY14 | TRPC4AP | 0.853 |
| UBE216 | RNF38 | 0.853 |
| GBP5 | OGFRL1 | 0.853 |
| C1QA | MECP2 | 0.853 |
| ATP6V1H | GBP1 | 0.853 |
| CASP1 | APOL2 | 0.853 |
| AIM2 | XPO1 | 0.853 |
| C1QA | RIPK2 | 0.853 |
| GBP5 | IMPA1 | 0.853 |
| SECTM1 | VAMP3 | 0.853 |
| DRG1 | GBP1 | 0.853 |
| GBP1 | ADIPOR2 | 0.853 |
| C1QA | TP53BP2 | 0.853 |
| SECTM1 | CHMP7 | 0.853 |
| VAMP5 | FOXJ2 | 0.853 |
| VWF | GBP1 | 0.853 |
| POLB | FAM508 | 0.853 |
| POLB | MCM2 | 0.853 |
| POLB | BCL2 | 0.853 |
| RIPK2 | VAMP5 | 0.853 |
| CALCOCO2 | ATG3 | 0.853 |
| VAMP5 | AACS | 0.853 |
| P2RY14 | INVS | 0.853 |
| APOL1 | GPR132 | 0.853 |
| VAMP5 | ATP2A3 | 0.853 |
| UBE2L6 | CENPJ | 0.853 |
| C1QA | PRDM4 | 0.853 |
| SECTM1 | ELAVL1 | 0.853 |
| P2RY14 | AKAP8L | 0.853 |
| APOL1 | STAT1 | 0.853 |
| CALCOCO2 | CEP250 | 0.853 |
| C1QA | PTGES2 | 0.853 |
| POLB | ABT1 | 0.853 |
| POLB | ANKRD10 | 0.853 |
| SECTM1 | CASP2 | 0.853 |
| P2RY14 | STX1A | 0.853 |
| LHFPL2 | PPM1D | 0.853 |
| PSME1 | TOB2 | 0.853 |
| IL15RA | DENND1A | 0.853 |
| GBP5 | CDK5RAP2 | 0.853 |
| VAMP5 | BCS1L | 0.853 |
| GBP1 | PSMD10 | 0.853 |
| VAMP5 | OGFOD1 | 0.853 |
| WSB2 | P2RY14 | 0.853 |
| POLB | BARD1 | 0.853 |
| C1QA | TULP4 | 0.853 |
| GBP5 | PFKL | 0.853 |
| C1QA | PCSK6 | 0.853 |
| GBP1 | EIFSA2 | 0.853 |
| UBE2L6 | TGFBRAP1 | 0.853 |
| ECHS1 | LHFPL2 | 0.853 |
| LHFPL2 | HERC1 | 0.853 |
| AIM2 | PPBP | 0.853 |
| P2RY14 | ZFPL1 | 0.853 |
| P2RY14 | GSN | 0.853 |
| APOL2 | MUTYH | 0.853 |
| APOL2 | PSMD2 | 0.853 |
| ACP5 | CALCOCO2 | 0.853 |
| APOL2 | ALMS1 | 0.853 |
| POLB | RHEB | 0.853 |
| POLB | COMMD4 | 0.853 |
| C1QA | TNFAIP6 | 0.853 |
| GBP1 | RPGR | 0.853 |
| PSME1 | CASP1 | 0.853 |
| UBE2L6 | CBLB | 0.853 |
| UBE2L6 | PXMP4 | 0.853 |
| SLC2A9 | GBP1 | 0.853 |
| GBP1 | KCNAB2 | 0.853 |
| VAMP5 | BZW2 | 0.853 |
| POLB | PTGIR | 0.853 |
| SECTM1 | SH3GL1 | 0.853 |
| SECTM1 | KATNB1 | 0.853 |
| POLB | CBR3 | 0.853 |
| PSTPIP2 | CAPN2 | 0.853 |
| VAMP5 | RNF122 | 0.853 |
| C1QA | MAGED1 | 0.853 |
| GBP1 | TAOK2 | 0.853 |
| POLB | SAP18 | 0.853 |
| UBE2L6 | PIK3CB | 0.853 |
| UBE2L6 | KHDRBS1 | 0.853 |
| LHFPL2 | ING4 | 0.853 |
| P2RY14 | SOCS3 | 0.853 |
| GBP1 | SPATA2 | 0.853 |
| GBP1 | DNAJB9 | 0.853 |
| L15RA | PPP2RSD | 0.853 |
| UBE2L6 | SCRIB | 0.853 |
| VAMP5 | UBIAD1 | 0.853 |
| LHFPL2 | DNPEP | 0.853 |
| SECTM1 | SRRM1 | 0.853 |
| CRYM | P2RY14 | 0.853 |
| UBE2L6 | WWC3 | 0.853 |
| SECTM1 | ELF4 | 0.853 |
| UBE2L6 | TBC1D1 | 0.853 |
| POLB | SORT1 | 0.853 |
| UBE2L6 | NXT1 | 0.853 |
| APOL2 | VPS39 | 0.853 |
| APOL1 | CTCF | 0.853 |
| DAPP1 | MSH2 | 0.853 |
| POLB | DHX29 | 0.853 |
| SMARCD3 | PF4V1 | 0.853 |
| VAMP5 | SNW1 | 0.853 |
| GBP1 | DCTN5 | 0.853 |
| POLB | XRCC5 | 0.853 |
| UBEZL6 | GYS1 | 0.853 |
| POLB | PDE6B | 0.853 |
| MAT2B | C1QA | 0.853 |
| GBP1 | GNRH1 | 0.853 |
| POLB | ZNF672 | 0.853 |
| UBE216 | TCF12 | 0.853 |
| P2RY14 | ERF | 0.853 |
| GBP5 | CD38 | 0.853 |
| VAMP5 | MLX | 0.853 |
| APOL2 | ANKRD11 | 0.853 |
| POLB | ASB7 | 0.853 |
| PSTPIP2 | NCOA1 | 0.853 |
| SECTM1 | ARRB2 | 0.853 |
| UBEZL6 | ARHGAP1 | 0.853 |
| GBP1 | FAM49B | 0.853 |
| POLB | ZNF200 | 0.853 |
| AIM2 | DAAM2 | 0.853 |
| CYP11A1 | P2RY14 | 0.853 |
| SECTM1 | USP47 | 0.853 |
| SECTM1 | NUP98 | 0.853 |
| FAS | STAG1 | 0.853 |
| C1QA | SPEN | 0.853 |
| P2RY14 | PPT1 | 0.853 |
| GBP5 | MTMR4 | 0.853 |
| GBP5 | GTF2A1 | 0.853 |
| UBE2L6 | POLD4 | 0.853 |
| P2RY14 | PLEK | 0.853 |
| UBE2L6 | MBD1 | 0.853 |
| PSME1 | CDK5RAP1 | 0.853 |
| IL15RA | NOLC1 | 0.853 |
| UBE2L6 | METTL1 | 0.853 |
| VAMP5 | ASB1 | 0.853 |
| POLB | ISOC2 | 0.853 |
| POLB | SMAD4 | 0.853 |
| VAMP5 | PMM1 | 0.853 |
| UBE216 | OGG1 | 0.853 |
| VAMP5 | CD320 | 0.853 |
| GBP1 | DNAJB6 | 0.853 |
| GBP1 | SPATASL1 | 0.853 |
| IL15RA | STAG1 | 0.853 |
| SECTM1 | JARID2 | 0.853 |
| GBP5 | BAZ1B | 0.853 |
| VAMP5 | TUBG1 | 0.853 |
| AIM2 | MAPK13 | 0.853 |
| APOL2 | PSMD11 | 0.853 |
| POLB | DSTN | 0.853 |
| POLB | DOCK2 | 0.853 |
| POLB | HERC2 | 0.853 |
| UBE2L6 | CDK4 | 0.853 |
| SECTM1 | CTNNBIP1 | 0.853 |
| LHFPL2 | DYNC1LI2 | 0.853 |
| VAMP5 | SEC23B | 0.853 |
| P2RY14 | TBL1X | 0.853 |
| APOL3 | POLG | 0.853 |
| PF4V1 | APOL2 | 0.853 |
| GBP5 | BCKDHB | 0.853 |
| PSTPIP2 | MAK | 0.853 |
| CASP7 | YEATS2 | 0.853 |
| APOL2 | RBM22 | 0.853 |
| UBE2L6 | BYSL | 0.853 |
| UBE2L6 | ALAS1 | 0.853 |
| SECTM1 | SLC25A22 | 0.853 |
| GBP1 | TAL1 | 0.853 |
| C1QA | HMGA1 | 0.853 |
| NPC2 | UQCRC1 | 0.853 |
| P2RY14 | COG7 | 0.853 |
| APOL2 | DNAJB1 | 0.853 |
| CYB561D2 | SECTM1 | 0.853 |
| PSME1 | PSMD11 | 0.853 |
| UBE2L6 | TSEN2 | 0.853 |
| IL15RA | DIAPH1 | 0.853 |
| GBP1 | ADD1 | 0.853 |
| UBE2L6 | SLC2A4RG | 0.853 |
| GBP1 | SLC5A6 | 0.853 |
| APOL2 | CDK5RAP1 | 0.853 |
| UBE2L6 | FAM3A | 0.853 |
| UBE216 | MAP4K4 | 0.853 |
| SECTM1 | PTP4A3 | 0.853 |
| POLB | CLN6 | 0.853 |
| VAMP5 | GPBP1L1 | 0.853 |
| APOL2 | SATB1 | 0.853 |
| CD1A | GBP1 | 0.853 |
| P2RY14 | CNOT4 | 0.853 |
| C1QA | SMAD3 | 0.853 |
| GBP1 | METAP2 | 0.853 |
| APOL2 | NFE2L1 | 0.853 |
| IL15RA | PFAS | 0.853 |
| AIM2 | PTP4A1 | 0.853 |
| POP4 | POLB | 0.853 |
| SMARCD3 | VAMP5 | 0.853 |
| GBP1 | BCORL1 | 0.853 |
| CREB3 | GBP1 | 0.853 |
| UBE2L6 | FXR1 | 0.853 |
| P2RY14 | CCT2 | 0.853 |
| SECTM1 | CTNNB1 | 0.853 |
| GBP1 | UBE2A | 0.853 |
| SECTM1 | MRPS18A | 0.853 |
| C1QA | ABR | 0.853 |
| SECTM1 | EXT1 | 0.853 |
| P2RY14 | COL9A2 | 0.853 |
| SECTM1 | MFN1 | 0.853 |
| C1QA | TM9SF4 | 0.853 |
| P2RY14 | UBE2Z | 0.853 |
| AIM2 | PLEK2 | 0.853 |
| CYB561 | PIK3C2B | 0.853 |
| PFDN5 | APOL2 | 0.853 |
| VAMP5 | RNF113A | 0.853 |
| APOL2 | ANXA11 | 0.853 |
| GBP1 | BNIP1 | 0.853 |
| GBP5 | GNLY | 0.853 |
| APOL2 | AACS | 0.853 |
| P2RY14 | DUSP13 | 0.853 |
| IL15RA | GEMIN4 | 0.853 |
| APOL1 | VCP | 0.853 |
| UBE2L6 | PIGT | 0.853 |
| SECTM1 | GTPBP4 | 0.853 |
| F13A1 | APOL1 | 0.853 |
| GBP1 | FADD | 0.853 |
| SECTM1 | MINK1 | 0.853 |
| P2RY14 | SGSH | 0.853 |
| P2RY14 | SRPRB | 0.853 |
| UBE2L6 | ATP6V1H | 0.853 |
| GBP1 | ZNF419 | 0.853 |
| GBP1 | GLUL | 0.853 |
| C1QA | EIF4G1 | 0.853 |
| C1QB | GBP1 | 0.853 |
| UBE2L6 | GNPDA1 | 0.853 |
| POLB | IARS2 | 0.853 |
| APOL1 | CRY2 | 0.853 |
| SMARCD3 | C1QA | 0.853 |
| PML | VPS11 | 0.853 |
| AIM2 | NIPBL | 0.853 |
| UBE2L6 | KPTN | 0.853 |
| P2RY14 | RELA | 0.853 |
| SECTM1 | FZD1 | 0.853 |
| AKR1A1 | DAPP1 | 0.853 |
| UBE2L6 | GTF3C5 | 0.853 |
| PSME1 | MPI | 0.853 |
| P2RY14 | HDAC6 | 0.853 |
| VAMP5 | ZNF212 | 0.853 |
| VAMP5 | TMEM106C | 0.853 |
| GBP5 | PA2G4 | 0.853 |
| GBP1 | RECQLS | 0.853 |
| AIM2 | SLC6A6 | 0.853 |
| UBE2L6 | KEAP1 | 0.853 |
| VAMP5 | KEAP1 | 0.853 |
| GBP1 | KLF9 | 0.853 |
| GBP1 | CBLL1 | 0.853 |
| SECTM1 | CDYL | 0.853 |
| TMSB10 | PSTPIP2 | 0.853 |
| SECTM1 | NET1 | 0.853 |
| PSTPIP2 | PFDN5 | 0.853 |
| DUSP3 | APOL2 | 0.853 |
| SECTM1 | ADAM17 | 0.853 |
| GBP1 | ULK1 | 0.853 |
| GBP1 | XYLT2 | 0.853 |
| SECTM1 | YTHDF1 | 0.853 |
| AIM2 | RIN2 | 0.853 |
| SECTM1 | PPM1D | 0.853 |
| SECTM1 | MSH2 | 0.853 |
| GBP1 | NKRF | 0.853 |
| P2RY14 | CUL1 | 0.853 |
| UBE2L6 | TOR1AIP1 | 0.853 |
| ATG3 | ARID1A | 0.853 |
| LHFPL2 | MAML1 | 0.853 |
| VAMP5 | METTL1 | 0.853 |
| APOL2 | POMT1 | 0.853 |
| APOL2 | FXR2 | 0.853 |
| ZNHIT1 | LHFPL2 | 0.853 |
| LHFPL2 | ZC3H7A | 0.853 |
| UBE2L6 | TSFM | 0.853 |
| P2RY14 | POLR2K | 0.853 |
| UBE2L6 | BRD3 | 0.853 |
| APOL1 | DENND1A | 0.853 |
| UBE2L6 | ARHGDIA | 0.853 |
| UBE2L6 | TBL2 | 0.853 |
| AIM2 | BARD1 | 0.853 |
| GBP5 | NVL | 0.853 |
| LHFPL2 | NFATC3 | 0.853 |
| GBP1 | ICAM2 | 0.853 |
| GBP5 | PLVAP | 0.853 |
| GSTO1 | PML | 0.853 |
| C1QA | CSE1L | 0.853 |
| SECTM1 | TRAF1 | 0.853 |
| GBP1 | DDB2 | 0.853 |
| SDC1 | LIMK1 | 0.853 |
| VAMP5 | NCBP2 | 0.853 |
| P2RY14 | EIF4EBP2 | 0.853 |
| UBE2L6 | IARS2 | 0.853 |
| GBP5 | ACADM | 0.853 |
| UBE2L6 | MAPKAPK5 | 0.853 |
| P2RY14 | SLC25A4 | 0.853 |
| SECTM1 | ARCN1 | 0.853 |
| GBP1 | CDC42 | 0.853 |
| POLB | IL1B | 0.853 |
| POLB | HMGA1 | 0.853 |
| SECTM1 | KLF13 | 0.853 |
| C1QA | PPARD | 0.853 |
| GBP1 | TBL2 | 0.853 |
| UBE2L6 | ACOX3 | 0.853 |
| IL15RA | PSTPIP2 | 0.853 |
| VAMP5 | ATP6AP1 | 0.853 |
| VAMP5 | IL21R | 0.853 |
| GBP5 | SLC20A1 | 0.853 |
| GBP1 | ZNF7 | 0.853 |
| DNAJC1 | VAMP5 | 0.853 |
| GBP1 | ASB6 | 0.853 |
| VAMP5 | AXIN1 | 0.853 |
| UBE2L6 | OXCT1 | 0.853 |
| C1QA | CAMK4 | 0.853 |
| GBP1 | SLC1A4 | 0.853 |
| GBP1 | FZD1 | 0.853 |
| POLB | PIP5K1C | 0.853 |
| POLB | TBC1D2B | 0.853 |
| VAMP5 | ATF7IP | 0.853 |
| GBP1 | DDB1 | 0.853 |
| PSME1 | ZNF76 | 0.853 |
| POLB | WDR13 | 0.853 |
| POLB | DDB2 | 0.852 |
| GBP1 | XBP1 | 0.852 |
| P2RY14 | NXT1 | 0.852 |
| POLB | TBC1D1 | 0.852 |
| APOL1 | TULP4 | 0.852 |
| POLB | POLR2B | 0.852 |
| C1QA | ACAD8 | 0.852 |
| PYGB | P2RY14 | 0.852 |
| LIMK1 | MCM3 | 0.852 |
| UBE2L6 | CAMSAP1 | 0.852 |
| GBP5 | CHD2 | 0.852 |
| UBE2L6 | NR1D2 | 0.852 |
| P2RY14 | STAT4 | 0.852 |
| APOL2 | MAGED1 | 0.852 |
| SECTM1 | DDX28 | 0.852 |
| ELMO3 | GBP1 | 0.852 |
| SMARCD3 | SECTM1 | 0.852 |
| POLB | IMP4 | 0.852 |
| C1QA | SYNJ2 | 0.852 |
| GBP1 | ATIC | 0.852 |
| GBP1 | PKNOX1 | 0.852 |
| LIMK1 | PLCG1 | 0.852 |
| PCBD1 | DAPP1 | 0.852 |
| P2RY14 | SLC39A9 | 0.852 |
| C1QA | RUVBL2 | 0.852 |
| MRPL44 | NDRG1 | 0.852 |
| LHFPL2 | ATP2B4 | 0.852 |
| VAMP5 | PAFAH1B3 | 0.852 |
| POLB | SPATA2 | 0.852 |
| GBP1 | PDCD2 | 0.852 |
| PCK2 | GBP1 | 0.852 |
| UBE2L6 | XPNPEP1 | 0.852 |
| UBE2L6 | PLEK2 | 0.852 |
| IGFBP2 | GBP1 | 0.852 |
| MDK | SECTM1 | 0.852 |
| AIM2 | RNGTT | 0.852 |
| P2RY14 | CXCR4 | 0.852 |
| GBP1 | CDK10 | 0.852 |
| VAMP5 | PITRM1 | 0.852 |
| DAPP1 | CAPN2 | 0.852 |
| VAMP5 | ACAD8 | 0.852 |
| LHFPL2 | OGDH | 0.852 |
| VAMP5 | FHOD1 | 0.852 |
| DAPP1 | KLF3 | 0.852 |
| LHFPL2 | TFAM | 0.852 |
| C1QA | ITGA5 | 0.852 |
| UBE2L6 | COG2 | 0.852 |
| TMEM59 | AIM2 | 0.852 |
| APOL1 | CTDP1 | 0.852 |
| VAMP5 | BRD9 | 0.852 |
| IL15RA | EIF1 | 0.852 |
| C1QA | XRCC1 | 0.852 |
| KCNJ2 | C1QA | 0.852 |
| GBP5 | GDF11 | 0.852 |
| LHFPL2 | SIRT5 | 0.852 |
| P2RY14 | TIAL1 | 0.852 |
| SECTM1 | EIF2B5 | 0.852 |
| P2RY14 | THADA | 0.852 |
| POLB | MED8 | 0.852 |
| PRDX4 | GBP1 | 0.852 |
| GBP5 | P2RX7 | 0.852 |
| MAN2C1 | P2RY14 | 0.852 |
| GBP5 | LSM3 | 0.852 |
| P2RY14 | MON1B | 0.852 |
| UBE2L6 | BRPF1 | 0.852 |
| VAMP5 | TRAF3IP3 | 0.852 |
| GBP1 | RCL1 | 0.852 |
| POLB | TM7SF3 | 0.852 |
| C1QA | NCOR1 | 0.852 |
| GNG11 | PRRG4 | 0.852 |
| POLB | POLE3 | 0.852 |
| GBP5 | PIK3CA | 0.852 |
| POLB | SLC3A2 | 0.852 |
| SECTM1 | KIF3B | 0.852 |
| C1QA | VIPR1 | 0.852 |
| POLB | PXMP2 | 0.852 |
| GBP1 | SMARCD1 | 0.852 |
| P2RY14 | CCNH | 0.852 |
| UBE2L6 | ABHD6 | 0.852 |
| POLB | PBX1 | 0.852 |
| PSME1 | RBBP4 | 0.852 |
| POLB | ARPC2 | 0.852 |
| VAMP5 | SRPRB | 0.852 |
| GBP5 | RGS2 | 0.852 |
| VAMP5 | MRPS27 | 0.852 |
| GBP1 | PTPRN2 | 0.852 |
| GBP1 | PTGS1 | 0.852 |
| LIMK1 | GANAB | 0.852 |
| C1QA | HRAS | 0.852 |
| UBE2L6 | MAML1 | 0.852 |
| POLB | TIPARP | 0.852 |
| POLB | GAS7 | 0.852 |
| P2RY14 | SLC16A5 | 0.852 |
| C1QA | BTN2A1 | 0.852 |
| LHFPL2 | MAP3K4 | 0.852 |
| SECTM1 | DCUN1D2 | 0.852 |
| FZD1 | VAMP5 | 0.852 |
| GBP5 | CD48 | 0.852 |
| GBP5 | CDIPT | 0.852 |
| SECTM1 | APBA2 | 0.852 |
| GBP5 | MBNL1 | 0.852 |
| LIMK1 | HIC2 | 0.852 |
| P2RY14 | ZNF22 | 0.852 |
| LHFPL2 | DHCR7 | 0.852 |
| ALG8 | P2RY14 | 0.852 |
| LHFPL2 | STAG2 | 0.852 |
| CYB561 | TRAF1 | 0.852 |
| C1QA | MAZ | 0.852 |
| LHFPL2 | GOLGA3 | 0.852 |
| C1QA | ABCF3 | 0.852 |
| GBP1 | GPR137B | 0.852 |
| UBE2L6 | DNPEP | 0.852 |
| POLB | OSBPL11 | 0.852 |
| POLB | POLR2D | 0.852 |
| WWC3 | VAMP5 | 0.852 |
| LHFPL2 | PER2 | 0.852 |
| P2RY14 | SCYL3 | 0.852 |
| UBE2L6 | DDB1 | 0.852 |
| GBP1 | YPEL1 | 0.852 |
| GBP5 | SELPLG | 0.852 |
| UBE2L6 | GSS | 0.852 |
| UBE2L6 | FBXO21 | 0.852 |
| POLB | RAB11FIP1 | 0.852 |
| PSME1 | RUVBL1 | 0.852 |
| GBP1 | YWHAB | 0.852 |
| P2RY14 | ATP6AP2 | 0.852 |
| PSTPIP2 | MTMR6 | 0.852 |
| UBE2L6 | CSTF2 | 0.852 |
| UBE2L6 | ITPR3 | 0.852 |
| POLB | NOL11 | 0.852 |
| APOL1 | PLK3 | 0.852 |
| P2RY14 | CLNS1A | 0.852 |
| UBE2L6 | STOML1 | 0.852 |
| VAMP5 | NDUFAF1 | 0.852 |
| CYB561 | GEMIN4 | 0.852 |
| SECTM1 | SNIP1 | 0.852 |
| IL15RA | OSBPL9 | 0.852 |
| POLB | INVS | 0.852 |
| POLB | EIF2B1 | 0.852 |
| ASRGL1 | GBP1 | 0.852 |
| AIM2 | YEATS2 | 0.852 |
| UBE2L6 | ZNF232 | 0.852 |
| C1QA | SETD3 | 0.852 |
| P2RY14 | FYN | 0.852 |
| UBE216 | TMEM39B | 0.852 |
| POLB | ASCL2 | 0.852 |
| C1QA | PMVK | 0.852 |
| C1QA | SNW1 | 0.852 |
| C1QA | ASB1 | 0.852 |
| GBP1 | MUS81 | 0.852 |
| P2RY14 | HMGN3 | 0.852 |
| VAMP5 | TRAF1 | 0.852 |
| GBP1 | INPP5A | 0.852 |
| CPA3 | AIM2 | 0.852 |
| GBP1 | POLD1 | 0.852 |
| TMEM59 | ACP5 | 0.852 |
| P2RY14 | RAB3D | 0.852 |
| GBP5 | SENP7 | 0.852 |
| APOL3 | NOLC1 | 0.852 |
| ECE2 | GBP1 | 0.852 |
| BTN2A1 | VAMP5 | 0.852 |
| AIM2 | EXT1 | 0.852 |
| VAMP5 | STMN1 | 0.852 |
| UBE2L6 | UBE4B | 0.852 |
| VAMP5 | DPH2 | 0.852 |
| IL15RA | SCMH1 | 0.852 |
| SECTM1 | AQR | 0.852 |
| GBP1 | MTMR9 | 0.852 |
| P2RY14 | CBR3 | 0.852 |
| GBP1 | KDELR2 | 0.852 |
| UBE216 | CDC23 | 0.852 |
| AIM2 | NCOA1 | 0.852 |
| UBE2L6 | WDR4 | 0.852 |
| VAMP5 | ELMO1 | 0.852 |
| AIM2 | PECR | 0.852 |
| GBP1 | WIP12 | 0.852 |
| NPC2 | MYC | 0.852 |
| POLB | CBLB | 0.852 |
| VAMP5 | HMGCR | 0.852 |
| AIM2 | MSH6 | 0.852 |
| GBP1 | ZNF562 | 0.852 |
| UBE2L6 | ZNF160 | 0.852 |
| CALCOCO2 | POLR3B | 0.852 |
| P2RY14 | SSH3 | 0.852 |
| C1QA | SEC63 | 0.852 |
| LHFPL2 | PUM1 | 0.852 |
| C1QA | ATP9B | 0.852 |
| P2RY14 | PSMA1 | 0.852 |
| SECTM1 | YWHAB | 0.852 |
| GBP1 | MRPL9 | 0.852 |
| UBE2L6 | ECHDC1 | 0.852 |
| GBP1 | HADHA | 0.852 |
| POLB | CNIH4 | 0.852 |
| GBP1 | SLC25A20 | 0.852 |
| GSTO1 | AIM2 | 0.852 |
| LHFPL2 | ABR | 0.852 |
| P2RY14 | PRMT5 | 0.852 |
| GBP1 | AVEN | 0.852 |
| APOL2 | BRD9 | 0.852 |
| GBP1 | GTPBP3 | 0.852 |
| POLB | SLC16A5 | 0.852 |
| GBP1 | COG2 | 0.852 |
| POLB | TBXA2R | 0.852 |
| P2RY14 | ZDHHC6 | 0.852 |
| C1QA | CDK5RAP1 | 0.852 |
| SECTM1 | PSD4 | 0.852 |
| UBE2L6 | TLE2 | 0.852 |
| AIM2 | PSMB1 | 0.852 |
| P2RY14 | RPS6KC1 | 0.852 |
| SECTM1 | TMEM38B | 0.852 |
| VAMP5 | SLC27A5 | 0.852 |
| P2RY14 | HDDC2 | 0.852 |
| COPS7A | GBP1 | 0.852 |
| GBP1 | ABCC3 | 0.852 |
| P2RY14 | ICOSLG | 0.852 |
| P2RY14 | CDCA8 | 0.852 |
| SECTM1 | SATB1 | 0.852 |
| POLB | CERK | 0.852 |
| C1QA | FYCO1 | 0.852 |
| LHFPL2 | SAP18 | 0.852 |
| GBP1 | CCDC51 | 0.852 |
| GBP1 | UBE2D4 | 0.852 |
| GBP5 | GPRCSC | 0.852 |
| STK25 | P2RY14 | 0.852 |
| C1QA | TMBIM4 | 0.852 |
| C1QA | NAGLU | 0.852 |
| C1QA | GMEB1 | 0.852 |
| VAMP5 | STAT6 | 0.852 |
| LHFPL2 | MYH9 | 0.852 |
| POLB | NARS2 | 0.852 |
| P2RY14 | P2RX5 | 0.852 |
| UBE2L6 | BCL7B | 0.852 |
| C1QA | PCBP4 | 0.852 |
| P2RY14 | HMGA1 | 0.852 |
| C1QA | RRM1 | 0.852 |
| PSTPIP2 | APOL2 | 0.852 |
| POLB | ZNF236 | 0.852 |
| VAMP5 | CD40 | 0.852 |
| UBE2L6 | INPPSE | 0.852 |
| P2RY14 | TNFAIP6 | 0.852 |
| POLB | PARVB | 0.852 |
| VAMP5 | IL16 | 0.852 |
| GBP1 | AP1B1 | 0.852 |
| SECTM1 | CWF19L1 | 0.852 |
| P2RY14 | PDLIM5 | 0.852 |
| LHFPL2 | GALNT2 | 0.852 |
| SECTM1 | PRKCSH | 0.852 |
| AIM2 | DIAPH1 | 0.852 |
| VAMP5 | BYSL | 0.852 |
| UBE2L6 | RUNX3 | 0.852 |
| IL15RA | TRAPPC3 | 0.852 |
| GBP1 | ANKRD12 | 0.852 |
| TMEM59 | OSBPL9 | 0.852 |
| CCT5 | GBP1 | 0.852 |
| POLB | SCAP | 0.852 |
| POLB | PACS1 | 0.852 |
| POLB | MAML1 | 0.852 |
| POLB | DNM2 | 0.852 |
| GBP1 | CLMN | 0.852 |
| SECTM1 | BSDC1 | 0.852 |
| APOL2 | ADAM19 | 0.852 |
| P2RY14 | ME2 | 0.852 |
| VAMP5 | ELF1 | 0.852 |
| CLCN6 | P2RY14 | 0.852 |
| LHFPL2 | ZMPSTE24 | 0.852 |
| VAMP5 | SNAPC4 | 0.852 |
| POLB | RBKS | 0.852 |
| UBE2L6 | CDC42 | 0.852 |
| AIM2 | LHFPL2 | 0.852 |
| C1QA | ATP2A3 | 0.852 |
| POLB | DERA | 0.852 |
| P2RY14 | YY1AP1 | 0.852 |
| GBP5 | JTB | 0.852 |
| SCCPDH | GBP1 | 0.852 |
| PSME1 | SLC3A2 | 0.852 |
| UBE2L6 | CORO1C | 0.852 |
| UBE2L6 | HDAC1 | 0.852 |
| VAMP5 | ELOVLS | 0.852 |
| GBP1 | TSFM | 0.852 |
| GBP1 | BLMH | 0.852 |
| AIM2 | COROZA | 0.852 |
| SMUG1 | GBP1 | 0.852 |
| SECTM1 | XYLT2 | 0.852 |
| UBE2L6 | NUP50 | 0.852 |
| VAMP5 | TAZ | 0.852 |
| UBE2L6 | RBM15 | 0.852 |
| ATG3 | PRDM2 | 0.852 |
| SECTM1 | DDIT4 | 0.852 |
| UBE2L6 | CSAD | 0.852 |
| SECTM1 | SLC2A4RG | 0.852 |
| VAMP5 | RBKS | 0.852 |
| UBE216 | SCYL3 | 0.852 |
| UBE216 | RERE | 0.852 |
| AIM2 | DNAJC4 | 0.852 |
| GBP1 | MBIP | 0.852 |
| POLB | CDC25B | 0.852 |
| POLB | PLEKHB2 | 0.852 |
| P2RY14 | TBC1D2B | 0.852 |
| APOL3 | GLG1 | 0.852 |
| POLB | SLC35F2 | 0.852 |
| UBE2L6 | GPR65 | 0.852 |
| C1QA | DNAJC1 | 0.852 |
| UBE2L6 | UQCRC1 | 0.852 |
| UBE2L6 | CCT5 | 0.852 |
| P2RY14 | MYB | 0.852 |
| UBE2L6 | FBXL12 | 0.852 |
| TMBIM4 | GBP1 | 0.852 |
| APOL2 | ZNF212 | 0.852 |
| VAMP5 | TFAM | 0.852 |
| C1QA | UPB1 | 0.852 |
| UBE2L6 | CNGB1 | 0.852 |
| VAMP5 | XPC | 0.852 |
| GBP1 | POLDIP3 | 0.852 |
| PSME1 | DDX54 | 0.852 |
| UBE2L6 | CCDC47 | 0.852 |
| GBP1 | RNF146 | 0.852 |
| GBP1 | ATP1A1 | 0.852 |
| VAMP5 | MRPS2 | 0.852 |
| GBP1 | XRCC5 | 0.852 |
| NDUFA1 | POLB | 0.852 |
| SECTM1 | MAP3K10 | 0.852 |
| UBE2L6 | S100A9 | 0.852 |
| APOL2 | ZNF76 | 0.852 |
| VAMP5 | DNAJB1 | 0.852 |
| VAMP5 | CORO2A | 0.852 |
| C1QA | TTF1 | 0.852 |
| P2RY14 | RPGRIP1 | 0.852 |
| C1QA | PES1 | 0.852 |
| UBE2L6 | PEMT | 0.852 |
| CALCOCO2 | NCOA1 | 0.852 |
| LHFPL2 | CANX | 0.852 |
| P2RY14 | CCDC28B | 0.852 |
| UBE2L6 | EFHC1 | 0.852 |
| GBP1 | LDLRAP1 | 0.852 |
| POLB | HEXIM1 | 0.852 |
| APOL2 | FYCO1 | 0.852 |
| APOL1 | ARID1A | 0.852 |
| STAT5A | POLB | 0.852 |
| UBE2L6 | MAP3K10 | 0.852 |
| P2RY14 | DAZAP2 | 0.852 |
| C1QA | RAPGEF1 | 0.852 |
| GBP1 | TGFBR3 | 0.852 |
| UBE2L6 | SLC4A2 | 0.852 |
| P2RY14 | GP1BA | 0.852 |
| LHFPL2 | CTNNB1 | 0.852 |
| SECTM1 | RPS6KC1 | 0.852 |
| P2RY14 | AMD1 | 0.852 |
| P2RY14 | HRAS | 0.852 |
| AIM2 | PML | 0.852 |
| GBP1 | TSPYL2 | 0.852 |
| MRPL16 | GBP1 | 0.852 |
| P2RY14 | PSMD14 | 0.852 |
| UBE2L6 | HS1BP3 | 0.852 |
| GBP1 | WDR4 | 0.852 |
| P2RY14 | SLC9A1 | 0.852 |
| LHFPL2 | SON | 0.852 |
| GBP5 | OAZ1 | 0.852 |
| APOL2 | ITGA5 | 0.852 |
| IRF1 | IL15RA | 0.852 |
| C1QA | POLD1 | 0.852 |
| APOL2 | PPM1G | 0.852 |
| GBP1 | MFGE8 | 0.852 |
| GBP1 | SLC25A4 | 0.852 |
| GBP5 | SERPINB9 | 0.852 |
| SECTM1 | ERCC3 | 0.852 |
| UBE2L6 | GALK2 | 0.852 |
| VAMP5 | SNRPA | 0.852 |
| POLR1D | VAMP5 | 0.852 |
| UBE2L6 | PAK1IP1 | 0.852 |
| IL15RA | PSMD5 | 0.852 |
| UBE2L6 | MRPS18A | 0.852 |
| NPC2 | XRCC1 | 0.852 |
| GBP1 | NEDD4L | 0.852 |
| P2RY14 | DEDD | 0.852 |
| VAMP5 | SLC25A17 | 0.852 |
| POLB | CEP68 | 0.852 |
| LIMK1 | IL16 | 0.852 |
| P2RY14 | SF1 | 0.852 |
| SECTM1 | PITPNC1 | 0.852 |
| C1QA | MPI | 0.852 |
| C1QA | GSS | 0.852 |
| CALCOCO2 | POLG | 0.852 |
| P2RY14 | MEFV | 0.852 |
| CYB561 | DDX27 | 0.852 |
| POLB | ANKS1A | 0.852 |
| C1QA | IL21R | 0.852 |
| C1QA | WWC3 | 0.852 |
| LHFPL2 | RAB11B | 0.852 |
| CALCOCO2 | MAP3K4 | 0.852 |
| P2RY14 | PRMT2 | 0.852 |
| P2RY14 | COQ6 | 0.852 |
| POLB | ETFDH | 0.852 |
| P2RY14 | LRRFIP2 | 0.852 |
| VAMP5 | ETS1 | 0.852 |
| GBP5 | AKAP11 | 0.852 |
| GBP1 | DNAJB1 | 0.852 |
| SECTM1 | ASB8 | 0.852 |
| LHFPL2 | GLG1 | 0.852 |
| AIM2 | MAPK1 | 0.852 |
| KIFC3 | GBP1 | 0.852 |
| NPC2 | VILL | 0.852 |
| POLB | ZAP70 | 0.851 |
| ATG3 | MSH2 | 0.851 |
| UBE2L6 | RFC5 | 0.851 |
| P2RY14 | BIRC5 | 0.851 |
| LHFPL2 | CTBP2 | 0.851 |
| SECTM1 | ATP1B1 | 0.851 |
| LIMK1 | RPAP1 | 0.851 |
| POLB | PTK2 | 0.851 |
| VAMP5 | HDAC1 | 0.851 |
| C1QA | NENF | 0.851 |
| APOL2 | RNF122 | 0.851 |
| LHFPL2 | TNPO1 | 0.851 |
| LIPT1 | P2RY14 | 0.851 |
| POLB | CASP9 | 0.851 |
| POLB | CIDEB | 0.851 |
| P2RY14 | POFUT2 | 0.851 |
| UBE2L6 | ARHGEF3 | 0.851 |
| DAD1 | LHFPL2 | 0.851 |
| GNG11 | ACP5 | 0.851 |
| UBE2L6 | STIP1 | 0.851 |
| SECTM1 | CHST2 | 0.851 |
| PSTPIP2 | NENF | 0.851 |
| GBP5 | ALDH3B1 | 0.851 |
| UBE2L6 | ERF | 0.851 |
| POLB | UBE2Q1 | 0.851 |
| P2RY14 | GLRX5 | 0.851 |
| GBP5 | HK3 | 0.851 |
| VAMP5 | PAXIP1 | 0.851 |
| P2RY14 | CDK4 | 0.851 |
| POLB | JAM3 | 0.851 |
| AIM2 | DPM2 | 0.851 |
| POLB | LDLRAP1 | 0.851 |
| AIM2 | USP48 | 0.851 |
| GBP1 | MRPS30 | 0.851 |
| APOL2 | UBAP2 | 0.851 |
| FAS | SRPK2 | 0.851 |
| GBP1 | DTX3 | 0.851 |
| UBE2L6 | RNMT | 0.851 |
| POLB | CACNB3 | 0.851 |
| AIM2 | SYNCRIP | 0.851 |
| APOL1 | PPARD | 0.851 |
| APOL2 | MCM6 | 0.851 |
| PSME1 | ABCF3 | 0.851 |
| P2RY14 | DUSP2 | 0.851 |
| GTF2B | VAMP5 | 0.851 |
| UBE2L6 | SLC35C1 | 0.851 |
| VAMP5 | EIF4G1 | 0.851 |
| AIM2 | MAX | 0.851 |
| CTDSPL | VAMP5 | 0.851 |
| GBP5 | DPEP3 | 0.851 |
| GBP1 | BRD4 | 0.851 |
| PSMA4 | SECTM1 | 0.851 |
| APOL2 | EXOSC10 | 0.851 |
| C1QA | TCF7 | 0.851 |
| UBE2L6 | SLC25A28 | 0.851 |
| UBE2L6 | LRFN3 | 0.851 |
| LHFPL2 | UBE2L3 | 0.851 |
| DAD1 | UBE2L6 | 0.851 |
| SECTM1 | GALK2 | 0.851 |
| VAMP5 | PEX14 | 0.851 |
| CALCOCO2 | STAG1 | 0.851 |
| UBE2L6 | GFOD1 | 0.851 |
| LHFPL2 | ILF3 | 0.851 |
| RFXANK | GBP1 | 0.851 |
| APOL3 | PLCG1 | 0.851 |
| C1QA | BRD9 | 0.851 |
| WWC3 | GBP1 | 0.851 |
| AIM2 | UPF2 | 0.851 |
| AIM2 | BCL2L11 | 0.851 |
| POLB | ITGA2B | 0.851 |
| GBP1 | CTSC | 0.851 |
| UBE2L6 | AKAP8L | 0.851 |
| P2RY14 | ATF6 | 0.851 |
| APOL2 | RPN1 | 0.851 |
| CYB561 | PPRC1 | 0.851 |
| AIM2 | CLEC10A | 0.851 |
| GBP1 | DFFA | 0.851 |
| APOL3 | SSRP1 | 0.851 |
| C1QA | FLNB | 0.851 |
| APOL2 | RGS14 | 0.851 |
| IL15RA | RFC1 | 0.851 |
| TJP2 | P2RY14 | 0.851 |
| C1QA | ALMS1 | 0.851 |
| APOL2 | SIRT5 | 0.851 |
| VTI1B | VAMP5 | 0.851 |
| P2RY14 | THAP7 | 0.851 |
| P2RY14 | CD36 | 0.851 |
| P2RY14 | MAPK14 | 0.851 |
| POLB | PEBP1 | 0.851 |
| APOL1 | PRMT7 | 0.851 |
| SECTM1 | PARP2 | 0.851 |
| P2RY14 | SLC37A1 | 0.851 |
| VAMP5 | PRDM4 | 0.851 |
| UBE216 | SLC5A6 | 0.851 |
| VAMP5 | LRIG1 | 0.851 |
| NPC2 | ELAC2 | 0.851 |
| C1QA | PLXND1 | 0.851 |
| POLB | CC2D1A | 0.851 |
| LIMK1 | TCF7 | 0.851 |
| APOL2 | PPP2R5B | 0.851 |
| LHFPL2 | NFE2L1 | 0.851 |
| UBE2L6 | SESN1 | 0.851 |
| GBP5 | EIFSB | 0.851 |
| VAMP5 | PRKCQ | 0.851 |
| SECTM1 | UBE2Q1 | 0.851 |
| AIM2 | SPEN | 0.851 |
| P2RY14 | UTP14A | 0.851 |
| UBE2L6 | SMARCE1 | 0.851 |
| LIMK1 | LAS1L | 0.851 |
| C1QA | POLRMT | 0.851 |
| SMARCD3 | LHFPL2 | 0.851 |
| POLB | ALAS1 | 0.851 |
| VAMP5 | SAR1A | 0.851 |
| GBP1 | USP16 | 0.851 |
| TARBP2 | GBP1 | 0.851 |
| GBP5 | AKT3 | 0.851 |
| UBE2L6 | XYLT2 | 0.851 |
| GBP5 | GNA13 | 0.851 |
| VAMP5 | VPS33A | 0.851 |
| P2RY14 | HCCS | 0.851 |
| POLB | UXS1 | 0.851 |
| C1QA | PDIA6 | 0.851 |
| POLB | SLC6A6 | 0.851 |
| CYB561 | RBM28 | 0.851 |
| GBP5 | BASP1 | 0.851 |
| LHFPL2 | ZNF318 | 0.851 |
| GBP5 | PKN2 | 0.851 |
| COX6B1 | PSTPIP2 | 0.851 |
| P2RY14 | CHST7 | 0.851 |
| UBE2L6 | TAX1BP1 | 0.851 |
| P2RY14 | XRCC1 | 0.851 |
| P2RY14 | ASB7 | 0.851 |
| VAMP5 | ITPKB | 0.851 |
| UBE2L6 | THOP1 | 0.851 |
| POLB | SLC16A6 | 0.851 |
| P2RY14 | ACAT2 | 0.851 |
| C1QA | MFGE8 | 0.851 |
| SECTM1 | SLC27A5 | 0.851 |
| C1QA | MRPL49 | 0.851 |
| UBE2L6 | RASSF1 | 0.851 |
| GRAP2 | GBP1 | 0.851 |
| UBE2L6 | PXMP2 | 0.851 |
| C1QA | LRRC8D | 0.851 |
| IL15RA | VILL | 0.851 |
| GBP5 | DMTF1 | 0.851 |
| SECTM1 | USP11 | 0.851 |
| P2RY14 | MPL | 0.851 |
| P2RY14 | PTDSS1 | 0.851 |
| SECTM1 | TRIP6 | 0.851 |
| SECTM1 | EFHD2 | 0.851 |
| GBP1 | EIF2AK1 | 0.851 |
| UBE2L6 | MUS81 | 0.851 |
| UBE2L6 | TIAL1 | 0.851 |
| P2RY14 | CDK9 | 0.851 |
| GBP1 | MED8 | 0.851 |
| DAPP1 | CYB561 | 0.851 |
| APOL1 | RUNX2 | 0.851 |
| P2RY14 | LCP1 | 0.851 |
| UBE2L6 | CDADC1 | 0.851 |
| UBE2L6 | TAF1C | 0.851 |
| UBE2L6 | GPKOW | 0.851 |
| FAS | RUNX2 | 0.851 |
| P2RY14 | CASZ1 | 0.851 |
| APOL2 | SRPK2 | 0.851 |
| VAMP5 | GIMAP6 | 0.851 |
| GBP5 | MACF1 | 0.851 |
| C1QA | DDX54 | 0.851 |
| GBP1 | RSAD1 | 0.851 |
| LHFPL2 | KPNA6 | 0.851 |
| SECTM1 | EPHA4 | 0.851 |
| NPC2 | TTLL12 | 0.851 |
| UBE2L6 | TMEM127 | 0.851 |
| C1QA | CC2D1A | 0.851 |
| GBP5 | CHM | 0.851 |
| C1QA | EIF2B5 | 0.851 |
| POLB | RPGR | 0.851 |
| P2RY14 | PREB | 0.851 |
| GBP1 | MAPK7 | 0.851 |
| POLB | HK2 | 0.851 |
| CD2BP2 | GBP1 | 0.851 |
| UBE2L6 | DERA | 0.851 |
| PSTPIP2 | ZBTB16 | 0.851 |
| LIMK1 | CRY2 | 0.851 |
| IL12RB1 | C1QA | 0.851 |
| CASP1 | ELF1 | 0.851 |
| GBP1 | HSD17B4 | 0.851 |
| P2RY14 | SNX11 | 0.851 |
| RERE | GBP1 | 0.851 |
| C1QA | LSG1 | 0.851 |
| UBE2L6 | SLC35F2 | 0.851 |
| UBE2L6 | SMAP1 | 0.851 |
| POLB | ZFPL1 | 0.851 |
| UBE2L6 | BCS1L | 0.851 |
| UBE2L6 | DCUN1D2 | 0.851 |
| GBP1 | ELL | 0.851 |
| PML | NOLC1 | 0.851 |
| GBP1 | GPR27 | 0.851 |
| LHFPL2 | SATB1 | 0.851 |
| SECTM1 | SEH1L | 0.851 |
| POLB | IVNS1ABP | 0.851 |
| UBE2L6 | SERTAD3 | 0.851 |
| UBE2L6 | TMEM106C | 0.851 |
| UBE2L6 | ZNF189 | 0.851 |
| PSME1 | KLF2 | 0.851 |
| UBE2L6 | ARIH2 | 0.851 |
| GBP1 | PILRA | 0.851 |
| LHFPL2 | VPS13D | 0.851 |
| UBE2L6 | ADIPOR2 | 0.851 |
| GBP1 | SYT11 | 0.851 |
| C1QA | TBC1D13 | 0.851 |
| AIM2 | ZFR | 0.851 |
| ZNHIT1 | UBE2L6 | 0.851 |
| CLTCL1 | P2RY14 | 0.851 |
| P2RY14 | TGFBR3 | 0.851 |
| SECTM1 | GTF3C2 | 0.851 |
| UBE2L6 | GTPBP3 | 0.851 |
| VAMP5 | ALMS1 | 0.851 |
| SECTM1 | RAF1 | 0.851 |
| SECTM1 | ING4 | 0.851 |
| GBP1 | PDE4A | 0.851 |
| SECTM1 | ZNF318 | 0.851 |
| UBE2L6 | ING1 | 0.851 |
| GBP1 | CCT4 | 0.851 |
| VAMP5 | MECP2 | 0.851 |
| ACP5 | PML | 0.851 |
| C1QA | GTF3C5 | 0.851 |
| VAMP5 | SFXN1 | 0.851 |
| GBP1 | HARS2 | 0.851 |
| C1QA | ATP6AP1 | 0.851 |
| P2RY14 | SYT11 | 0.851 |
| DNAJC17 | GBP1 | 0.851 |
| UBE2L6 | GOLPH3 | 0.851 |
| AIM2 | BCAT1 | 0.851 |
| VAMP5 | TRIM8 | 0.851 |
| LHFPL2 | ESD | 0.851 |
| SECTM1 | ECE1 | 0.851 |
| SECTM1 | POLRMT | 0.851 |
| IL15RA | TOB2 | 0.851 |
| UBE2L6 | H6PD | 0.851 |
| VAMP5 | KCNAB2 | 0.851 |
| AIM2 | PHF3 | 0.851 |
| SECTM1 | PLA2G12A | 0.851 |
| DAPP1 | ARFGEF1 | 0.851 |
| VAMP5 | DUS1L | 0.851 |
| P2RY14 | DCTN6 | 0.851 |
| VAMP5 | RGS14 | 0.851 |
| GTF2B | APOL2 | 0.851 |
| LHFPL2 | ETF1 | 0.851 |
| DAPP1 | DNAJC4 | 0.851 |
| P2RY14 | TKTL1 | 0.851 |
| P2RY14 | FANCL | 0.851 |
| APOL2 | ERBB2 | 0.851 |
| AIM2 | CYP1B1 | 0.851 |
| POLB | GCDH | 0.851 |
| VAMP5 | CTNNBL1 | 0.851 |
| GBP1 | CDC7 | 0.851 |
| UBE2L6 | BBS7 | 0.851 |
| PRRG4 | SRPK2 | 0.851 |
| UBE2L6 | ATP13A2 | 0.851 |
| PML | AKAP1 | 0.851 |
| PML | POLR3B | 0.851 |
| GBP1 | MDFIC | 0.851 |
| GATA1 | GBP1 | 0.851 |
| UBEZL6 | PON2 | 0.851 |
| CALCOCO2 | FOXJ2 | 0.851 |
| P2RY14 | SLC35E1 | 0.851 |
| POLB | PHF10 | 0.851 |
| POLB | PTGS1 | 0.851 |
| ATG3 | YEATS2 | 0.851 |
| VAMP5 | XYLT2 | 0.851 |
| GBP1 | SLC25A36 | 0.851 |
| GBP1 | KLHL22 | 0.851 |
| GBP5 | MYBPC3 | 0.851 |
| POLB | PXN | 0.851 |
| UBE2L6 | DAAM2 | 0.851 |
| APOL2 | RBM10 | 0.851 |
| VAMP5 | JOSD1 | 0.851 |
| VAMP5 | THOP1 | 0.851 |
| LHFPL2 | CCDC47 | 0.851 |
| P2RY14 | SH3TC1 | 0.851 |
| C1QA | SMU1 | 0.851 |
| C1QA | UBIAD1 | 0.851 |
| POLB | SSBP1 | 0.851 |
| GBP1 | CDKAL1 | 0.851 |
| VAMP5 | MRPS11 | 0.851 |
| KCNMB1 | GBP1 | 0.851 |
| SECTM1 | CLASP1 | 0.851 |
| LHFPL2 | TMEM39B | 0.851 |
| AIM2 | SLC25A22 | 0.851 |
| GBP1 | SLC25A37 | 0.851 |
| POLB | RUFY3 | 0.851 |
| APOL3 | ILF3 | 0.851 |
| UBE2L6 | NDUFS4 | 0.851 |
| UBE2L6 | MAGEH1 | 0.851 |
| NMI | SECTM1 | 0.851 |
| UBE2L6 | RNF41 | 0.851 |
| CYB561 | NDRG1 | 0.851 |
| LHFPL2 | CUL4A | 0.851 |
| DUSP3 | ZBTB16 | 0.851 |
| GBP1 | IL15 | 0.851 |
| SECTM1 | ABHD5 | 0.851 |
| GBP1 | MLH1 | 0.851 |
| P2RY14 | ZDHHC7 | 0.851 |
| AIM2 | ZBTB16 | 0.851 |
| GBP1 | CRNKL1 | 0.851 |
| ACP5 | STX11 | 0.851 |
| PSTPIP2 | MAPK1 | 0.851 |
| GBP1 | SLC35E3 | 0.851 |
| VAMP5 | PSMC3 | 0.851 |
| GBP1 | DDX54 | 0.851 |
| POLB | TBX19 | 0.851 |
| PSME1 | GTF3C2 | 0.851 |
| STEAP3 | GBP1 | 0.851 |
| VAMP5 | SLC39A8 | 0.851 |
| C1QA | SATB1 | 0.851 |
| APOL2 | GNB1 | 0.851 |
| P2RY14 | KLHL22 | 0.851 |
| P2RY14 | MAP1LC3B | 0.851 |
| GBP1 | SNAPC4 | 0.851 |
| P2RY14 | DERL1 | 0.851 |
| RUFY3 | VAMP5 | 0.851 |
| PARP3 | GBP1 | 0.851 |
| C1QA | CDK4 | 0.851 |
| APOL2 | HMGCR | 0.851 |
| BLCAP | GBP1 | 0.851 |
| PEA15 | GBP1 | 0.851 |
| DOLPP1 | GBP1 | 0.851 |
| DAPP1 | CCNG2 | 0.851 |
| GBP1 | OSBPL10 | 0.851 |
| VAMP5 | SEH1L | 0.851 |
| AGPAT3 | LIMK1 | 0.851 |
| VAMP5 | IDH2 | 0.851 |
| GBP1 | CFDP1 | 0.851 |
| POLB | TFB2M | 0.851 |
| SECTM1 | ACTR8 | 0.851 |
| P2RY14 | EBAG9 | 0.851 |
| POLB | PSMD9 | 0.851 |
| COX6B1 | AIM2 | 0.851 |
| GBP1 | OPN3 | 0.851 |
| UBE2L6 | ASB6 | 0.851 |
| UBE2L6 | SOX4 | 0.851 |
| SMARCD3 | POR | 0.851 |
| PSME1 | TRIM28 | 0.851 |
| GYPC | LHFPL2 | 0.851 |
| VAMP5 | TTF1 | 0.851 |
| APOL1 | LSG1 | 0.851 |
| GBP1 | TM9SF2 | 0.851 |
| C1QA | RNASEL | 0.851 |
| APOL1 | PUS1 | 0.851 |
| LHFPL2 | AGPAT3 | 0.851 |
| C1QA | RNH1 | 0.851 |
| POLB | EHD3 | 0.851 |
| PSME1 | MAP4 | 0.851 |
| CALCOCO2 | TOB2 | 0.851 |
| P2RY14 | CHCHD7 | 0.851 |
| AIM2 | PRKAR2A | 0.851 |
| PML | PPRC1 | 0.851 |
| VAMP5 | POLD3 | 0.851 |
| P2RY14 | FXR1 | 0.851 |
| C1QA | USP48 | 0.851 |
| ATP1B3 | GBP1 | 0.851 |
| APOL2 | R3HDM2 | 0.851 |
| SECTM1 | GPKOW | 0.851 |
| POLB | DHX38 | 0.851 |
| STATSA | GBP1 | 0.851 |
| UROS | POLB | 0.851 |
| UBE2L6 | RACGAP1 | 0.851 |
| C1QA | EPHA4 | 0.851 |
| CYB561 | PLCG1 | 0.851 |
| GBP5 | POLR2L | 0.851 |
| MYLK | P2RY14 | 0.851 |
| LHFPL2 | AP4E1 | 0.851 |
| P2RY14 | DVL2 | 0.851 |
| C1QA | GNE | 0.851 |
| P2RY14 | RGS14 | 0.851 |
| GBP5 | SEC23A | 0.851 |
| GBP5 | CD86 | 0.851 |
| VAMP5 | PRPF8 | 0.851 |
| VAMP5 | SS18 | 0.851 |
| C1QA | MRPL9 | 0.851 |
| P2RY14 | P2RY6 | 0.851 |
| YWHAH | P2RY14 | 0.851 |
| C1QA | SEH1L | 0.851 |
| UBE2L6 | DTX3 | 0.851 |
| VAMP5 | VAT1 | 0.851 |
| ZNF646 | P2RY14 | 0.851 |
| POLB | CRK | 0.851 |
| P2RY14 | RAB3IL1 | 0.851 |
| UBE2L6 | YWHAB | 0.851 |
| LHFPL2 | STK38 | 0.851 |
| LIMK1 | GPR132 | 0.851 |
| C1QA | PHB | 0.851 |
| UBE2L6 | PRPSAP1 | 0.851 |
| APOL2 | PXN | 0.851 |
| C1QA | CASP6 | 0.851 |
| AIM2 | AMPD3 | 0.851 |
| C1QA | ZNF76 | 0.851 |
| GBP1 | KATNA1 | 0.851 |
| CASP5 | C1QA | 0.851 |
| UBE2L6 | ACP1 | 0.851 |
| POLB | HBS1L | 0.851 |
| VAMP5 | WDR74 | 0.851 |
| POLB | DCTN4 | 0.851 |
| VAMP5 | YWHAZ | 0.851 |
| AIM2 | BTN2A2 | 0.851 |
| UBE216 | MAP2K1 | 0.851 |
| GBP5 | RASSF7 | 0.851 |
| GBP1 | CDV3 | 0.851 |
| GBP1 | RPA2 | 0.851 |
| SECTM1 | GSS | 0.851 |
| UBE2L6 | CEP63 | 0.851 |
| C1QA | SNAPC4 | 0.851 |
| POLB | GTF3C5 | 0.851 |
| GBP1 | COPS6 | 0.851 |
| SECTM1 | ETS1 | 0.851 |
| GBP1 | FBN2 | 0.851 |
| LHFPL2 | RFWD3 | 0.851 |
| GBP1 | MNT | 0.851 |
| LHFPL2 | CASP1 | 0.851 |
| POLB | WDR25 | 0.851 |
| P2RY14 | PMP22 | 0.851 |
| CYB561 | DGKD | 0.851 |
| APOL2 | TSC1 | 0.851 |
| LHFPL2 | RER1 | 0.851 |
| POLB | TM7SF2 | 0.851 |
| POLB | TRIM37 | 0.851 |
| UBE2L6 | SLC35D2 | 0.851 |
| POLB | ATP13A1 | 0.851 |
| LHFPL2 | ZNF189 | 0.851 |
| P2RY14 | GGCX | 0.851 |
| UBE2L6 | GLUL | 0.851 |
| GSTO1 | PDHA1 | 0.851 |
| DYNLT1 | GBP1 | 0.851 |
| AIM2 | KLHL2 | 0.851 |
| LHFPL2 | FYCO1 | 0.851 |
| LHFPL2 | MSH2 | 0.851 |
| UBE2L6 | PTPN11 | 0.851 |
| PSME1 | FXR2 | 0.851 |
| PSME1 | PUS1 | 0.851 |
| UBE2L6 | ZNF236 | 0.851 |
| GBP1 | B4GALT1 | 0.851 |
| UBEZL6 | MS4A6A | 0.851 |
| VAMP5 | CXXC1 | 0.851 |
| VAMP5 | GPS1 | 0.851 |
| P2RY14 | KLHL26 | 0.851 |
| GBP1 | LETM1 | 0.851 |
| UBE216 | DHRS3 | 0.851 |
| C1QA | KLF13 | 0.851 |
| LHFPL2 | PAPOLA | 0.851 |
| C1QA | SP110 | 0.851 |
| AFF1 | VAMP5 | 0.851 |
| DAPP1 | ACBD3 | 0.851 |
| VAMP5 | LCK | 0.851 |
| P2RY14 | PTP4A3 | 0.851 |
| VAMP5 | RBBP7 | 0.85 |
| VAMP5 | GPA33 | 0.85 |
| VAMP5 | CWF19L1 | 0.85 |
| STK16 | VAMP5 | 0.85 |
| GBP5 | SRPK1 | 0.85 |
| CALCOCO2 | KLF6 | 0.85 |
| APOL2 | FOXJ2 | 0.85 |
| POLB | MRFAP1L1 | 0.85 |
| VAMP5 | SATB1 | 0.85 |
| CASP7 | GBP1 | 0.85 |
| SECTM1 | SDHB | 0.85 |
| C1QA | TMEM97 | 0.85 |
| SECTM1 | EHD1 | 0.85 |
| C1QA | PRDM2 | 0.85 |
| UBE2L6 | PSMA4 | 0.85 |
| SECTM1 | DNPEP | 0.85 |
| UBE2L6 | NISCH | 0.85 |
| VAMP5 | CCT7 | 0.85 |
| PML | CAPN2 | 0.85 |
| P2RY14 | MNT | 0.85 |
| IL15RA | SYNJ2 | 0.85 |
| VAMP5 | WDR25 | 0.85 |
| PTGIR | GBP1 | 0.85 |
| VAMP5 | ECHDC2 | 0.85 |
| UBE2L6 | NAGLU | 0.85 |
| APOL1 | KLF6 | 0.85 |
| VAMP5 | ANAPC5 | 0.85 |
| VAMP5 | PHF3 | 0.85 |
| APOL2 | SEC63 | 0.85 |
| VAMP5 | PSMD8 | 0.85 |
| AIM2 | EHD4 | 0.85 |
| UBE2L6 | BTG1 | 0.85 |
| GBP1 | PHKA2 | 0.85 |
| GBP1 | DUSP2 | 0.85 |
| VAMP5 | CIAPIN1 | 0.85 |
| SECTM1 | PSME3 | 0.85 |
| PML | POLG | 0.85 |
| UBE2L6 | GOT1 | 0.85 |
| CYB561 | RUNX2 | 0.85 |
| APOL2 | OSBPL2 | 0.85 |
| CASP1 | SLCO4C1 | 0.85 |
| SECTM1 | KEAP1 | 0.85 |
| GBP1 | GADD45B | 0.85 |
| C1QA | MUTYH | 0.85 |
| P2RY14 | PLEKHB2 | 0.85 |
| GBP1 | CYP1B1 | 0.85 |
| C1QA | PARP2 | 0.85 |
| VAMP5 | DBR1 | 0.85 |
| PSTPIP2 | ARID1A | 0.85 |
| AIM2 | IRS2 | 0.85 |
| SECTM1 | HERC1 | 0.85 |
| UBE2L6 | TXK | 0.85 |
| C1QA | NFE2L1 | 0.85 |
| LHFPL2 | TTLL12 | 0.85 |
| GAS7 | GBP1 | 0.85 |
| PSTPIP2 | PPP3CA | 0.85 |
| C1QA | DGKA | 0.85 |
| GBP5 | ACTR3 | 0.85 |
| POLB | PDK2 | 0.85 |
| GBP5 | FAM50A | 0.85 |
| VAMP5 | FXR2 | 0.85 |
| C1QA | SLC2A4RG | 0.85 |
| P2RY14 | NCSTN | 0.85 |
| UBE2L6 | STAM2 | 0.85 |
| UBE2L6 | EPHB6 | 0.85 |
| APOL1 | PPRC1 | 0.85 |
| SECTM1 | OGG1 | 0.85 |
| VAMP5 | YTHDF1 | 0.85 |
| C1QA | JOSD1 | 0.85 |
| GBP5 | CEACAM1 | 0.85 |
| P2RY14 | CACYBP | 0.85 |
| UBE2L6 | APEX2 | 0.85 |
| LHFPL2 | ABL1 | 0.85 |
| VAMP5 | EIF4ENIF1 | 0.85 |
| VAMP5 | SLC16A5 | 0.85 |
| YIPF1 | GBP1 | 0.85 |
| DAPP1 | SON | 0.85 |
| GBP5 | CNNM4 | 0.85 |
| POLB | SERTAD3 | 0.85 |
| LHFPL2 | APH1A | 0.85 |
| LHFPL2 | DDX27 | 0.85 |
| VAMP5 | ERCC3 | 0.85 |
| GBP1 | PSMD7 | 0.85 |
| LHFPL2 | RRM1 | 0.85 |
| AIM2 | KNTC1 | 0.85 |
| NFYC | GBP1 | 0.85 |
| GBP1 | WIP11 | 0.85 |
| P2RY14 | CPSF4 | 0.85 |
| ATP1B2 | SECTM1 | 0.85 |
| LHFPL2 | ATP2A2 | 0.85 |
| BTN3A1 | VAMP5 | 0.85 |
| IRF1 | BCR | 0.85 |
| GBP5 | SUPT16H | 0.85 |
| UBE2L6 | GTF3C3 | 0.85 |
| AIM2 | SRPK2 | 0.85 |
| UBE2L6 | MTR | 0.85 |
| VAMP5 | CRY2 | 0.85 |
| VAMP5 | MFGE8 | 0.85 |
| GNAZ | GBP1 | 0.85 |
| POLB | CORO1B | 0.85 |
| KDELR1 | UBE216 | 0.85 |
| C1QA | RNF122 | 0.85 |
| GBP1 | CTSS | 0.85 |
| POLB | SFXN3 | 0.85 |
| C1QA | LRIG1 | 0.85 |
| PSME1 | SMAD3 | 0.85 |
| GBP5 | HRK | 0.85 |
| SECTM1 | ST14 | 0.85 |
| LHFPL2 | NET1 | 0.85 |
| UBE2L6 | PALLD | 0.85 |
| LHFPL2 | DAPP1 | 0.85 |
| P2RY14 | ZMYM3 | 0.85 |
| LIMK1 | STT3A | 0.85 |
| ACO1 | LHFPL2 | 0.85 |
| C1QA | ITPKC | 0.85 |
| AIM2 | PIK3CB | 0.85 |
| C1QA | PPP2R5A | 0.85 |
| P2RY14 | BTN2A1 | 0.85 |
| GMPR2 | C1QA | 0.85 |
| PRRG4 | RUNX2 | 0.85 |
| IRF1 | XPC | 0.85 |
| POLB | RPL39L | 0.85 |
| POLB | THUMPD1 | 0.85 |
| UBE2L6 | ZAP70 | 0.85 |
| UBE2L6 | MAPRE2 | 0.85 |
| GBP1 | FGR | 0.85 |
| STX11 | KLF6 | 0.85 |
| APTX | GBP1 | 0.85 |
| UBE2L6 | C1GALT1C1 | 0.85 |
| POLB | TGOLN2 | 0.85 |
| PSME1 | AAAS | 0.85 |
| POLB | TOR1AIP1 | 0.85 |
| TBX19 | GBP1 | 0.85 |
| AAMP | GBP1 | 0.85 |
| SECTM1 | PIK3C2B | 0.85 |
| SECTM1 | SERGEF | 0.85 |
| APOL2 | MPI | 0.85 |
| LHFPL2 | BTN3A1 | 0.85 |
| PSME1 | SDC1 | 0.85 |
| UBE2L6 | SLC27A5 | 0.85 |
| UBE2L6 | REEP4 | 0.85 |
| C1QA | AAAS | 0.85 |
| GBP1 | PSEN2 | 0.85 |
| VAMP5 | MPDU1 | 0.85 |
| GBP5 | IL1R2 | 0.85 |
| LHFPL2 | TXN2 | 0.85 |
| VAMP5 | DDX24 | 0.85 |
| ALOX12 | GBP1 | 0.85 |
| APOL2 | TRAF1 | 0.85 |
| UBEZL6 | TMC6 | 0.85 |
| DNAJC1 | APOL2 | 0.85 |
| KCNK3 | GBP1 | 0.85 |
| ACAD10 | GBP1 | 0.85 |
| P2RY14 | SERGEF | 0.85 |
| SECTM1 | GUSB | 0.85 |
| C1QA | IL16 | 0.85 |
| UBE2L6 | SPIN1 | 0.85 |
| POLB | C1GALT1C1 | 0.85 |
| POLB | UPB1 | 0.85 |
| CASP5 | GBP1 | 0.85 |
| DAPP1 | ETS1 | 0.85 |
| UBE2L6 | RPUSD2 | 0.85 |
| GBP1 | RBM3 | 0.85 |
| UBEZL6 | PITPNM1 | 0.85 |
| APOL1 | LSR | 0.85 |
| STAT1 | XPO1 | 0.85 |
| PLAGL1 | VAMP5 | 0.85 |
| UBE216 | GMIP | 0.85 |
| GBP1 | CLCF1 | 0.85 |
| UBE2L6 | PCBD1 | 0.85 |
| SECTM1 | PSMD3 | 0.85 |
| UBE2L6 | WDR74 | 0.85 |
| POLB | PPP3CC | 0.85 |
| POLB | ATP2A2 | 0.85 |
| AIM2 | SDC1 | 0.85 |
| POLB | TBL2 | 0.85 |
| GBP1 | ACTR1B | 0.85 |
| SECTM1 | DPH2 | 0.85 |
| UBE2L6 | PUS3 | 0.85 |
| GBP1 | TICAM1 | 0.85 |
| CYB561 | SATB1 | 0.85 |
| SECTM1 | IRS2 | 0.85 |
| UBE2L6 | ASTE1 | 0.85 |
| P2RY14 | DIABLO | 0.85 |
| SECTM1 | ASB6 | 0.85 |
| SECTM1 | MAP3K14 | 0.85 |
| VAMP5 | GPD1L | 0.85 |
| GBP1 | ALDH9A1 | 0.85 |
| LHFPL2 | TULP4 | 0.85 |
| MYL9 | C1QA | 0.85 |
| VAMP5 | ELP3 | 0.85 |
| VAMP5 | PEPD | 0.85 |
| POLB | MYH9 | 0.85 |
| UBE2L6 | GCDH | 0.85 |
| UBE216 | E2F1 | 0.85 |
| UBE2L6 | AP2A2 | 0.85 |
| P2RY14 | BAG5 | 0.85 |
| UBE2L6 | TBX21 | 0.85 |
| C1QA | FAM111A | 0.85 |
| P2RY14 | ELF4 | 0.85 |
| LHFPL2 | ZNF394 | 0.85 |
| IL15RA | BAHD1 | 0.85 |
| VAMP5 | ZNF232 | 0.85 |
| UBE2L6 | CCNE1 | 0.85 |
| VAMP5 | EXOSC5 | 0.85 |
| GBP1 | LDHA | 0.85 |
| VAMP5 | MAT2A | 0.85 |
| SECTM1 | PREP | 0.85 |
| NPC2 | ATP6AP1 | 0.85 |
| LHFPL2 | CHAF1B | 0.85 |
| DAPP1 | PUM1 | 0.85 |
| APOL2 | MRPL9 | 0.85 |
| HOOK2 | GBP1 | 0.85 |
| P2RY14 | SSB | 0.85 |
| UBE2L6 | RHEB | 0.85 |
| PSME1 | TCF7 | 0.85 |
| APOL2 | YTHDF1 | 0.85 |
| NPC2 | GRWD1 | 0.85 |
| C1QA | PSMB1 | 0.85 |
| UBE2L6 | SMAD7 | 0.85 |
| VAMP5 | ITGA5 | 0.85 |
| GBP5 | ZBP1 | 0.85 |
| SECTM1 | FZD5 | 0.85 |
| VAMP5 | TRIP6 | 0.85 |
| LIMK1 | HMGA1 | 0.85 |
| SECTM1 | SMARCD1 | 0.85 |
| VAMP5 | DLEC1 | 0.85 |
| P2RY14 | SMYD3 | 0.85 |
| GBP1 | PSME3 | 0.85 |
| P2RY14 | SCNN1D | 0.85 |
| AIM2 | ACBD3 | 0.85 |
| DAPP1 | OXSR1 | 0.85 |
| APOL2 | HMGA1 | 0.85 |
| P2RY14 | ABT1 | 0.85 |
| P2RY14 | MTA2 | 0.85 |
| FAM111A | VAMP5 | 0.85 |
| APOL2 | TRAF3IP3 | 0.85 |
| P2RY14 | RABEPK | 0.85 |
| POLB | MR1 | 0.85 |
| SECTM1 | SS18 | 0.85 |
| P2RY14 | VBP1 | 0.85 |
| VAMP5 | CUTA | 0.85 |
| UBE2L6 | IDH2 | 0.85 |

TABLE 6

| Gene 1 | Gene 2 | AUROC |
|---|---|---|
| GBP5 | ACP5 | 0.923 |
| GBP5 | NDRG1 | 0.916 |
| GBP5 | POLB | 0.913 |
| P2RY14 | NPC2 | 0.913 |
| GBP5 | C1QA | 0.912 |
| POLB | NDRG1 | 0.912 |
| GBP5 | ETS1 | 0.912 |
| GBP5 | SMARCD3 | 0.912 |
| P2RY14 | C1QA | 0.911 |
| GBP5 | RRM1 | 0.910 |
| P2RY14 | POLB | 0.91 |
| GBP5 | NPC2 | 0.909 |
| GBP5 | POLR3B | 0.908 |
| GBP5 | MAK | 0.908 |
| GBP5 | PSME1 | 0.907 |
| GBP5 | TOB1 | 0.906 |
| GBP5 | EPHA4 | 0.906 |
| GBP5 | SIDT1 | 0.906 |
| P2RY14 | PSME1 | 0.906 |
| P2RY14 | ACP5 | 0.906 |
| GBP5 | P2RY14 | 0.905 |
| P2RY14 | VAMP5 | 0.905 |
| GBP5 | EHD4 | 0.904 |
| GBP5 | KLF13 | 0.904 |
| GBP5 | GFOD1 | 0.904 |
| P2RY14 | ETS1 | 0.903 |
| APOL2 | NDRG1 | 0.903 |
| P2RY14 | APOL2 | 0.903 |
| POLB | POLR3B | 0.903 |
| P2RY14 | UBE2L6 | 0.903 |
| GBP5 | CD74 | 0.903 |
| GBP5 | ZCCHC14 | 0.903 |
| GBP5 | CD40 | 0.903 |
| P2RY14 | APOL1 | 0.903 |
| P2RY14 | SMARCD3 | 0.903 |
| GBP5 | APOL1 | 0.902 |
| GBP5 | CALCOCO2 | 0.902 |
| P2RY14 | MAK | 0.902 |
| GBP5 | APOL2 | 0.902 |
| GBP5 | VAMP5 | 0.902 |
| GBP5 | LAX1 | 0.902 |
| POLB | C1QA | 0.901 |
| GBP5 | SESN1 | 0.901 |
| GBP5 | PLCD1 | 0.901 |
| GBP5 | DUSP3 | 0.901 |
| GBP5 | UPB1 | 0.9 |
| GBP5 | DENND1A | 0.9 |
| GBP5 | CLDND1 | 0.9 |
| GBP5 | ACTN4 | 0.9 |
| GBP5 | CYB561 | 0.9 |
| GBP5 | CASB | 0.898 |
| UBE2L6 | NDRG1 | 0.898 |
| P2RY14 | SESN1 | 0.898 |
| POLB | ACP5 | 0.898 |
| P2RY14 | EPHA4 | 0.897 |
| UBE2L6 | POLB | 0.897 |
| GBP5 | SMAD7 | 0.897 |
| GBP5 | UBE2L6 | 0.897 |
| CALCOCO2 | POLB | 0.897 |
| GBP5 | GPR132 | 0.897 |
| GBP5 | JOSD1 | 0.896 |
| GBP5 | KLF2 | 0.896 |
| GBP5 | NMT2 | 0.896 |
| GBP5 | LHFPL2 | 0.896 |
| GBP1 | ACP5 | 0.896 |
| GBP5 | MYL9 | 0.896 |
| GBP5 | PRPF4 | 0.896 |
| GBP5 | MCM5 | 0.895 |
| GBP5 | MSRB2 | 0.895 |
| GBP5 | PRRG4 | 0.895 |
| GBP5 | CXCR3 | 0.895 |
| GBP5 | TCFL5 | 0.895 |
| POLB | PSME1 | 0.894 |
| GBP5 | GFI1 | 0.894 |
| GBP5 | CASP1 | 0.893 |
| POLB | DENND1A | 0.893 |
| P2RY14 | CD74 | 0.893 |
| POLB | LHFPL2 | 0.893 |
| P2RY14 | DUSP3 | 0.893 |
| GBP5 | CLCF1 | 0.893 |
| GBP5 | TSPAN2 | 0.893 |
| P2RY14 | KLF13 | 0.892 |
| GBP5 | ATP1B2 | 0.892 |
| GBP5 | SLC25A4 | 0.892 |
| POLB | APOL2 | 0.892 |
| P2RY14 | ZCCHC14 | 0.892 |
| P2RY14 | NDRG1 | 0.892 |
| GBP5 | WWC3 | 0.892 |
| GBP5 | RAB20 | 0.891 |
| P2RY14 | POLR3B | 0.891 |
| GBP5 | TAPBPL | 0.891 |
| APOL1 | NDRG1 | 0.891 |
| VAMP5 | POLB | 0.891 |
| GBP5 | TRIM8 | 0.89 |
| P2RY14 | TOB1 | 0.89 |
| GBP5 | VRK1 | 0.89 |
| P2RY14 | MSRB2 | 0.889 |
| P2RY14 | CYB561 | 0.889 |
| P2RY14 | SIDT1 | 0.889 |
| PSME1 | NDRG1 | 0.889 |
| GBP5 | RASGRP2 | 0.889 |
| GBP1 | C1QA | 0.889 |
| GBP5 | PSTPIP2 | 0.889 |
| P2RY14 | CALCOCO2 | 0.888 |
| P2RY14 | CASP1 | 0.888 |
| GBP5 | CD83 | 0.888 |
| GBP5 | DHRS3 | 0.888 |
| P2RY14 | DENND1A | 0.888 |
| P2RY14 | RRM1 | 0.888 |
| GBP5 | PPP1R2 | 0.887 |
| GBP5 | NXT1 | 0.887 |
| GBP5 | SAMHD1 | 0.887 |
| GBP5 | SORT1 | 0.887 |
| GBP5 | ZNF395 | 0.887 |
| P2RY14 | LHFPL2 | 0.886 |
| POLB | CYB561 | 0.886 |
| P2RY14 | GBP1 | 0.886 |
| GBP5 | AIM2 | 0.886 |
| GBP5 | IRF1 | 0.886 |
| GBP5 | IL15 | 0.885 |
| GBP5 | ALDH1A1 | 0.885 |
| GBP5 | OSBPL10 | 0.885 |
| GBP5 | CHST7 | 0.885 |
| P2RY14 | TSPAN2 | 0.885 |
| GBP1 | POLB | 0.885 |
| UBE2L6 | C1QA | 0.885 |
| GBP5 | CDK9 | 0.885 |
| LHFPL2 | ACP5 | 0.885 |
| GBP5 | VPS37B | 0.884 |
| NPC2 | NDRG1 | 0.884 |
| POLB | APOL1 | 0.884 |
| P2RY14 | IRF1 | 0.884 |
| GBP5 | POR | 0.884 |
| GBP5 | LDHA | 0.883 |
| P2RY14 | CASB | 0.883 |
| GBP5 | FAS | 0.883 |
| ACP5 | CASP1 | 0.883 |
| GBP5 | P2RY10 | 0.883 |
| POLB | SESN1 | 0.883 |
| GBP5 | SLC6A12 | 0.883 |
| GBP5 | PSMA4 | 0.882 |
| P2RY14 | TAPBPL | 0.882 |
| POLB | ZCCHC14 | 0.882 |
| P2RY14 | AIM2 | 0.882 |
| CALCOCO2 | NDRG1 | 0.882 |
| POLB | DUSP3 | 0.882 |
| GBP5 | GBP1 | 0.882 |
| UBE2L6 | EHD4 | 0.881 |
| P2RY14 | GFOD1 | 0.881 |
| UBE2L6 | CYB561 | 0.881 |
| GBP5 | BTN3A1 | 0.881 |
| GBP1 | ETS1 | 0.881 |
| UBE2L6 | ACP5 | 0.881 |
| P2RY14 | MYL9 | 0.881 |
| P2RY14 | LAX1 | 0.881 |
| GBP1 | NDRG1 | 0.881 |
| P2RY14 | CD40 | 0.88 |
| GBP1 | SMARCD3 | 0.88 |
| C1QA | NDRG1 | 0.88 |
| GBP1 | NPC2 | 0.88 |
| POLB | PLCD1 | 0.88 |
| GBP5 | FAM111A | 0.88 |
| GBP5 | TNFAIP2 | 0.88 |
| POLB | SMARCD3 | 0.88 |
| GBP5 | NELL2 | 0.88 |
| GBP5 | ZBTB32 | 0.88 |
| P2RY14 | RAB20 | 0.88 |
| P2RY14 | NMT2 | 0.879 |
| P2RY14 | CLDND1 | 0.879 |
| POLB | NPC2 | 0.879 |
| APOL2 | C1QA | 0.879 |
| C1QA | PRRG4 | 0.879 |
| GBP5 | COL9A2 | 0.879 |
| UBE2L6 | CALCOCO2 | 0.879 |
| UBE2L6 | APOL2 | 0.879 |
| POLB | PRRG4 | 0.879 |
| P2RY14 | PRRG4 | 0.878 |
| GBP1 | APOL1 | 0.878 |
| APOL2 | ACP5 | 0.878 |
| VAMP5 | NDRG1 | 0.878 |
| APOL2 | LHFPL2 | 0.878 |
| GBP5 | CNDP2 | 0.878 |
| POLB | CD40 | 0.878 |
| GBP1 | MAK | 0.878 |
| GBP5 | CCR7 | 0.878 |
| UBE2L6 | APOL1 | 0.878 |
| PSME1 | LHFPL2 | 0.878 |
| VAMP5 | GBP1 | 0.877 |
| GBP5 | GPR65 | 0.877 |
| GBP5 | STX11 | 0.877 |
| POLB | RASGRP2 | 0.877 |
| GBP1 | TOB1 | 0.877 |
| GBP5 | C1QB | 0.877 |
| UBE2L6 | NPC2 | 0.877 |
| P2RY14 | TCFL5 | 0.876 |
| P2RY14 | ZNF395 | 0.876 |
| LHFPL2 | APOL1 | 0.876 |
| P2RY14 | PSTPIP2 | 0.876 |
| GBP5 | VAT1 | 0.876 |
| P2RY14 | ATP1B2 | 0.876 |
| CYB561 | LHFPL2 | 0.876 |
| GBP5 | CD46 | 0.876 |
| GBP1 | RRM1 | 0.876 |
| POLB | AIM2 | 0.875 |
| GBP5 | BTN3A2 | 0.875 |
| P2RY14 | C1QB | 0.875 |
| VAMP5 | PRRG4 | 0.875 |
| AIM2 | CD74 | 0.875 |
| GBP5 | CASP5 | 0.875 |
| POLB | EPHA4 | 0.875 |
| PSTPIP2 | ACP5 | 0.874 |
| VAMP5 | UBE2L6 | 0.874 |
| P2RY14 | SLC6A12 | 0.874 |
| P2RY14 | PLCD1 | 0.874 |
| GBP5 | SP110 | 0.874 |
| UBE2L6 | SMARCD3 | 0.874 |
| POLB | PSTPIP2 | 0.874 |
| LHFPL2 | C1QA | 0.874 |
| VAMP5 | CALCOCO2 | 0.874 |
| VAMP5 | ACP5 | 0.874 |
| GBP5 | MTHFD2 | 0.873 |
| LHFPL2 | NPC2 | 0.873 |
| DUSP3 | NDRG1 | 0.873 |
| GBP1 | PSME1 | 0.873 |
| AIM2 | ACP5 | 0.873 |
| GBP1 | CYB561 | 0.873 |
| GBP1 | APOL2 | 0.873 |
| UBE2L6 | LHFPL2 | 0.873 |
| GBP1 | EHD4 | 0.873 |
| POLB | CA5B | 0.873 |
| AIM2 | UPB1 | 0.873 |
| P2RY14 | UPB1 | 0.873 |
| GBP5 | IL23A | 0.873 |
| CALCOCO2 | C1QA | 0.872 |
| DUSP3 | ACP5 | 0.872 |
| LHFPL2 | NDRG1 | 0.872 |
| STAT1 | P2RY14 | 0.872 |
| PSME1 | POLR3B | 0.872 |
| AIM2 | C1QA | 0.872 |
| ACP5 | C1QA | 0.872 |
| P2RY14 | BTN3A1 | 0.872 |
| UBE2L6 | PLCD1 | 0.872 |
| APOL2 | POLR3B | 0.871 |
| POLB | GPR132 | 0.871 |
| VAMP5 | APOL2 | 0.871 |
| GBP5 | IGF2BP3 | 0.871 |
| GBP5 | GPX7 | 0.871 |
| P2RY14 | PPP1R2 | 0.871 |
| POLB | CASP1 | 0.871 |
| PSME1 | C1QA | 0.871 |
| UBE216 | DUSP3 | 0.871 |
| IRF1 | NDRG1 | 0.871 |
| GBP5 | TRIM21 | 0.871 |
| P2RY14 | SMAD7 | 0.871 |
| POLB | IL15 | 0.871 |
| GBP1 | POLR3B | 0.871 |
| VAMP5 | LHFPL2 | 0.871 |
| GBP5 | AFF1 | 0.871 |
| STAT1 | GBP5 | 0.871 |
| GBP5 | SOCS1 | 0.871 |
| P2RY14 | CLCF1 | 0.87 |
| UBE2L6 | PRRG4 | 0.87 |
| GBP1 | EPHA4 | 0.87 |
| POLB | NXT1 | 0.87 |
| POLB | KLF2 | 0.87 |
| APOL1 | ACP5 | 0.87 |
| GBP5 | XAB2 | 0.87 |
| VAMP5 | MCM5 | 0.87 |
| LHFPL2 | MAK | 0.87 |
| UBE2L6 | PSME1 | 0.87 |
| GBP1 | KLF13 | 0.87 |
| FAS | C1QA | 0.87 |
| CYB561 | C1QA | 0.87 |
| AIM2 | APOL1 | 0.869 |
| UBE2L6 | MAK | 0.869 |
| P2RY14 | TRIM21 | 0.869 |
| UBE2L6 | POLR3B | 0.869 |
| GBP5 | MARCKSL1 | 0.869 |
| POLB | RRM1 | 0.869 |
| POLB | TCFLS | 0.869 |
| P2RY14 | JOSD1 | 0.869 |
| VAMP5 | APOL1 | 0.869 |
| GBP1 | CD74 | 0.869 |
| GBP5 | ITM2C | 0.869 |
| GBP5 | TNFSF10 | 0.869 |
| LHFPL2 | UPB1 | 0.869 |
| POLB | ETS1 | 0.869 |
| P2RY14 | PSMA4 | 0.869 |
| GBP5 | CTRL | 0.868 |
| P2RY14 | P2RY10 | 0.868 |
| GBP1 | SIDT1 | 0.868 |
| P2RY14 | SAMHD1 | 0.868 |
| P2RY14 | PRPF4 | 0.868 |
| POLB | MAK | 0.868 |
| GBP1 | UPB1 | 0.868 |
| VAMP5 | FAS | 0.868 |
| P2RY14 | WWC3 | 0.868 |
| STAT1 | C1QA | 0.868 |
| P2RY14 | GPR132 | 0.868 |
| VAMP5 | CYB561 | 0.868 |
| IRF1 | C1QA | 0.868 |
| APOL1 | C1QA | 0.868 |
| CASP1 | NDRG1 | 0.868 |
| C1QA | MCM5 | 0.868 |
| UBE2L6 | CASP1 | 0.867 |
| UBE2L6 | RRM1 | 0.867 |
| PSTPIP2 | C1QA | 0.867 |
| UBE2L6 | TOB1 | 0.867 |
| AIM2 | CYB561 | 0.867 |
| GBP1 | UBE2L6 | 0.867 |
| AIM2 | MAK | 0.867 |
| GBP1 | DUSP3 | 0.867 |
| GBP1 | CD40 | 0.867 |
| GBP1 | LHFPL2 | 0.867 |
| UBE2L6 | MCM5 | 0.867 |
| VAMP5 | C1QA | 0.867 |
| POLB | FAS | 0.867 |
| AIM2 | NPC2 | 0.866 |
| GBP5 | ISG20 | 0.866 |
| P2RY14 | NELL2 | 0.866 |
| P2RY14 | EHD4 | 0.866 |
| GBP1 | CALCOCO2 | 0.866 |
| GBP1 | SESN1 | 0.866 |
| CASP1 | C1QA | 0.866 |
| P2RY14 | IL15 | 0.866 |
| VAMP5 | PLCD1 | 0.866 |
| VAMP5 | POLR3B | 0.866 |
| GBP1 | DENND1A | 0.866 |
| GBP5 | GCH1 | 0.866 |
| POLB | TRIM8 | 0.866 |
| APOL2 | RASGRP2 | 0.865 |
| VAMP5 | PSME1 | 0.865 |
| POLB | ATP1B2 | 0.865 |
| UBE2L6 | UPB1 | 0.865 |
| CD74 | LHFPL2 | 0.865 |
| POLB | JOSD1 | 0.865 |
| POLB | RAB20 | 0.865 |
| APOL1 | FAS | 0.865 |
| P2RY14 | SORT1 | 0.865 |
| P2RY14 | OSBPL10 | 0.865 |
| P2RY14 | SP110 | 0.865 |
| PSME1 | PLCD1 | 0.865 |
| DUSP3 | C1QA | 0.865 |
| GBP1 | MSRB2 | 0.865 |
| CALCOCO2 | PSME1 | 0.865 |
| APOL1 | PRRG4 | 0.865 |
| UBE2L6 | FAS | 0.864 |
| UBE2L6 | DENND1A | 0.864 |
| GBP1 | MYL9 | 0.864 |
| PSME1 | APOL2 | 0.864 |
| UBE2L6 | CD40 | 0.864 |
| P2RY14 | KLF2 | 0.864 |
| POLB | IRF1 | 0.864 |
| P2RY14 | ALDH1A1 | 0.864 |
| C1QA | POLR3B | 0.864 |
| VAMP5 | AIM2 | 0.864 |
| GBP5 | MEF2D | 0.864 |
| PSME1 | MCM5 | 0.864 |
| GBP1 | ZCCHC14 | 0.864 |
| UBE2L6 | TRIM8 | 0.864 |
| NPC2 | C1QA | 0.864 |
| P2RY14 | CASP5 | 0.864 |
| GBP1 | GFOD1 | 0.864 |
| UBE2L6 | JOSD1 | 0.864 |
| AIM2 | NDRG1 | 0.863 |
| VAMP5 | EHD4 | 0.863 |
| PSME1 | AIM2 | 0.863 |
| AIM2 | APOL2 | 0.863 |
| GBP5 | FAM8A1 | 0.863 |
| POLB | DHRS3 | 0.863 |
| POLB | MCM5 | 0.863 |
| POLB | TOB1 | 0.863 |
| UBE2L6 | ETS1 | 0.863 |
| ACP5 | FAS | 0.863 |
| GBP5 | CDKN1C | 0.863 |
| P2RY14 | STX11 | 0.863 |
| UBE2L6 | PSTPIP2 | 0.863 |
| GBP1 | CLDND1 | 0.863 |
| PSME1 | PRRG4 | 0.863 |
| PSME1 | APOL1 | 0.863 |
| VAMP5 | UPB1 | 0.863 |
| GBP1 | LAX1 | 0.863 |
| UBE2L6 | GPR132 | 0.862 |
| UBEZL6 | AIM2 | 0.862 |
| POLB | NMT2 | 0.862 |
| STAT1 | POLB | 0.862 |
| FAS | NPC2 | 0.862 |
| STAT1 | ACP5 | 0.861 |
| VAMP5 | CD46 | 0.861 |
| VAMP5 | IRF1 | 0.861 |
| CALCOCO2 | APOL2 | 0.861 |
| NPC2 | PRRG4 | 0.861 |
| ALDH1A1 | AIM2 | 0.861 |
| P2RY14 | RASGRP2 | 0.861 |
| P2RY14 | DHRS3 | 0.861 |
| GBP1 | SMAD7 | 0.861 |
| C1QA | PLCD1 | 0.861 |
| UBE2L6 | KLF13 | 0.861 |
| VAMP5 | NXT1 | 0.861 |
| GBP5 | RTP4 | 0.861 |
| GBP1 | CASP1 | 0.861 |
| PSTPIP2 | APOL1 | 0.861 |
| VAMP5 | NPC2 | 0.861 |
| CYB561 | EPHA4 | 0.861 |
| VAMP5 | PSTPIP2 | 0.86 |
| VAMP5 | RRM1 | 0.86 |
| P2RY14 | CNDP2 | 0.86 |
| CALCOCO2 | APOL1 | 0.86 |
| LHFPL2 | CD40 | 0.86 |
| GBP1 | AIM2 | 0.86 |
| UBE2L6 | PRPF4 | 0.86 |
| CD46 | C1QA | 0.86 |
| GBP1 | PLCD1 | 0.86 |
| UBE216 | EPHA4 | 0.86 |
| CALCOCO2 | LHFPL2 | 0.86 |
| GBP1 | PRRG4 | 0.86 |
| DENND1A | NDRG1 | 0.86 |
| APOL2 | PRRG4 | 0.86 |
| P2RY14 | SOCS1 | 0.86 |
| P2RY14 | TNFSF10 | 0.86 |
| GBP1 | TSPAN2 | 0.86 |
| P2RY14 | CD83 | 0.86 |
| P2RY14 | ISG20 | 0.86 |
| VAMP5 | DENND1A | 0.86 |
| PSTPIP2 | NDRG1 | 0.859 |
| POLB | EHD4 | 0.859 |
| VAMP5 | CASP1 | 0.859 |
| P2RY14 | TNFAIP2 | 0.859 |
| P2RY14 | FAS | 0.859 |
| APOL2 | TRIM8 | 0.859 |
| UBE2L6 | ACTN4 | 0.859 |
| GBP1 | NMT2 | 0.859 |
| APOL2 | FAS | 0.859 |
| IRF1 | ACP5 | 0.859 |
| LHFPL2 | EHD4 | 0.859 |
| GBP1 | ATP182 | 0.859 |
| GBP1 | TCFLS | 0.859 |
| BTN3A1 | C1QA | 0.859 |
| GBP1 | JOSD1 | 0.859 |
| GBP5 | BST2 | 0.859 |
| POLB | SIDT1 | 0.859 |
| UBE2L6 | SIDT1 | 0.858 |
| UBE2L6 | POR | 0.858 |
| GBP1 | ACTN4 | 0.858 |
| ACP5 | NPC2 | 0.858 |
| GBP5 | SNX10 | 0.858 |
| SMARCD3 | NDRG1 | 0.858 |
| GBP1 | CA5B | 0.858 |
| UBE2L6 | IL15 | 0.858 |
| NPC2 | TRIM8 | 0.858 |
| VAMP5 | DUSP3 | 0.858 |
| P2RY14 | BTN3A2 | 0.858 |
| POLB | PPP1R2 | 0.858 |
| PSME1 | DUSP3 | 0.858 |
| GBP5 | TRIM22 | 0.858 |
| ACP5 | PRRG4 | 0.858 |
| GBP1 | GPR132 | 0.858 |
| GBP1 | PRPF4 | 0.858 |
| CYB561 | APOL2 | 0.857 |
| GBP1 | RAB20 | 0.857 |
| APOL2 | GPR132 | 0.857 |
| UBE2L6 | KLF2 | 0.857 |
| POLB | ZNF395 | 0.857 |
| APOL2 | PLCD1 | 0.857 |
| P2RY14 | RTP4 | 0.857 |
| PSTPIP2 | UPB1 | 0.857 |
| APOL1 | POLR3B | 0.857 |
| DENND1A | CIQA | 0.857 |
| P2RY14 | GFI1 | 0.857 |
| GBP5 | LGALS3BP | 0.857 |
| LHFPL2 | IRF1 | 0.857 |
| VAMP5 | GPR132 | 0.857 |
| CYB561 | PSTPIP2 | 0.857 |
| GBP1 | MCM5 | 0.857 |
| BTN3A1 | UBE2L6 | 0.857 |
| GBP1 | ALDH1A1 | 0.856 |
| LHFPL2 | POLR3B | 0.856 |
| C1QA | GPR132 | 0.856 |
| P2RY14 | MCM5 | 0.856 |
| SMARCD3 | ACP5 | 0.856 |
| UBE2L6 | IRF1 | 0.856 |
| APOL2 | NPC2 | 0.856 |
| AIM2 | CD40 | 0.856 |
| APOL2 | IL15 | 0.856 |
| UBE216 | FAM111A | 0.856 |
| CYB561 | APOL1 | 0.856 |
| CALCOCO2 | AIM2 | 0.856 |
| UBE2L6 | ALDH1A1 | 0.856 |
| P2RY14 | BST2 | 0.856 |
| POLB | C1QB | 0.856 |
| CYB561 | CASP1 | 0.856 |
| APOL2 | MCM5 | 0.856 |
| GBP1 | TRIM8 | 0.856 |
| PSTPIP2 | NPC2 | 0.856 |
| STAT1 | VAMP5 | 0.856 |
| IL15 | C1QA | 0.856 |
| GBP1 | KLF2 | 0.855 |
| APOL1 | IL15 | 0.855 |
| POLB | SAMHD1 | 0.855 |
| GBP1 | PSTPIP2 | 0.855 |
| APOL2 | DENND1A | 0.855 |
| C1QA | GPR65 | 0.855 |
| POLB | CLCF1 | 0.855 |
| P2RY14 | VRK1 | 0.855 |
| C1QA | POR | 0.855 |
| POLB | KLF13 | 0.855 |
| STX11 | C1QA | 0.855 |
| POLB | MYL9 | 0.855 |
| P2RY14 | CXCR3 | 0.855 |
| POLB | STX11 | 0.854 |
| APOL2 | KLF2 | 0.854 |
| P2RY14 | FAM111A | 0.854 |
| POLB | PRPF4 | 0.854 |
| C1QA | ETS1 | 0.854 |
| VAMP5 | STX11 | 0.854 |
| P2RY14 | TRIM8 | 0.854 |
| GBP1 | TAPBPL | 0.854 |
| UBE2L6 | ZCCHC14 | 0.854 |
| CYB561 | SMARCD3 | 0.854 |
| APOL1 | CASP1 | 0.854 |
| VAMP5 | KLF2 | 0.854 |
| GBP1 | IRF1 | 0.854 |
| POLB | WWC3 | 0.854 |
| UBE2L6 | RASGRP2 | 0.854 |
| VAMP5 | POR | 0.854 |
| VAMP5 | IL15 | 0.854 |
| PSME1 | RASGRP2 | 0.854 |
| PSME1 | PSTPIP2 | 0.854 |
| UBE2L6 | VPS37B | 0.854 |
| SMARCD3 | APOL2 | 0.854 |
| P2RY14 | MTHFD2 | 0.854 |
| APOL2 | APOL1 | 0.854 |
| C1QA | EHD4 | 0.854 |
| P2RY14 | ACTN4 | 0.853 |
| GBP1 | GFI1 | 0.853 |
| VAMP5 | RASGRP2 | 0.853 |
| PSME1 | FAS | 0.853 |
| APOL2 | CASP1 | 0.853 |
| APOL1 | GPR132 | 0.853 |
| STAT1 | APOL1 | 0.853 |
| CALCOCO2 | ACP5 | 0.853 |
| PSME1 | CASP1 | 0.853 |
| UBE2L6 | WWC3 | 0.853 |
| POLB | SORT1 | 0.853 |
| UBE2L6 | NXT1 | 0.853 |
| PSTPIP2 | MAK | 0.853 |
| VAMP5 | SMARCD3 | 0.853 |
| P2RY14 | COL9A2 | 0.853 |
| GBP1 | C1QB | 0.853 |
| SMARCD3 | CIQA | 0.853 |
| DUSP3 | APOL2 | 0.853 |
| APOL1 | DENND1A | 0.853 |
| P2RY14 | SLC25A4 | 0.853 |
| P2RY14 | NXT1 | 0.852 |
| VAMP5 | WWC3 | 0.852 |
| CALCOCO2 | POLR3B | 0.852 |
| C1QA | RRM1 | 0.852 |
| PSTPIP2 | APOL2 | 0.852 |
| VAMP5 | CD40 | 0.852 |
| AIM2 | LHFPL2 | 0.852 |
| UBE2L6 | GPR65 | 0.852 |
| C1QA | UPB1 | 0.852 |
| GBP1 | SLC25A4 | 0.852 |
| WWC3 | C1QA | 0.852 |
| VAMP5 | ETS1 | 0.852 |
| UBE2L6 | GFOD1 | 0.851 |
| GBP1 | WWC3 | 0.851 |
| UBE2L6 | SESN1 | 0.851 |
| SMARCD3 | LHFPL2 | 0.851 |
| P2RY14 | CHST7 | 0.851 |
| P2RY14 | CDK9 | 0.851 |
| PSME1 | KLF2 | 0.851 |
| VAMP5 | TRIM8 | 0.851 |
| VAMP5 | JOSD1 | 0.851 |
| CYB561 | NDRG1 | 0.851 |
| GBP1 | IL15 | 0.851 |
| ACP5 | STX11 | 0.851 |
| GBP1 | OSBPL10 | 0.851 |
| SMARCD3 | POR | 0.851 |
| C1QA | EPHA4 | 0.851 |
| VAMP5 | VAT1 | 0.851 |
| C1QA | CASP5 | 0.851 |
| LHFPL2 | CASP1 | 0.851 |
| UBE2L6 | DHRS3 | 0.851 |
| C1QA | KLF13 | 0.851 |
| SP110 | C1QA | 0.851 |
| VAMP5 | AFF1 | 0.851 |
| UBE2L6 | PSMA4 | 0.85 |
| AIM2 | EHD4 | 0.85 |
| C1QA | JOSD1 | 0.85 |
| GBP5 | CEACAM1 | 0.85 |
| LHFPL2 | RRM1 | 0.85 |
| BTN3A1 | VAMP5 | 0.85 |
| GBP5 | HRK | 0.85 |
| BTN3A1 | LHFPL2 | 0.85 |
| POLB | UPB1 | 0.85 |
| GBP1 | CASP5 | 0.85 |
| GBP1 | CLCF1 | 0.85 |
| C1QA | MYL9 | 0.85 |
| FAM111A | C1QA | 0.85 |
| GBP1 | LDHA | 0.85 |
| UBEZL6 | SMAD7 | 0.85 |
| GBP5 | ZBP1 | 0.85 |
| VAMP5 | FAM111A | 0.85 |
| UBE2L6 | ZNF395 | 0.85 |
| UBE2L6 | CA5B | 0.85 |
| POLB | CDK9 | 0.85 |
| AFF1 | C1QA | 0.85 |
| C1QA | KLF2 | 0.85 |
| PSME1 | CYB561 | 0.85 |
| PSME1 | NPC2 | 0.85 |
| C1QA | SESN1 | 0.85 |
| GBP1 | PPP1R2 | 0.85 |
| GBP1 | ZNF395 | 0.85 |
| GBP1 | SORT1 | 0.85 |
| STAT1 | ETS1 | 0.85 |
| P2RY14 | GPR65 | 0.85 |
| PSME1 | IL15 | 0.85 |
| DUSP3 | APOL1 | 0.85 |
| C1QA | ZCCHC14 | 0.849 |
| GBP1 | FAS | 0.849 |
| UBE2L6 | ATP1B2 | 0.849 |
| BTN3A1 | AIM2 | 0.849 |
| VAMP5 | EPHA4 | 0.849 |
| UBE2L6 | RAB20 | 0.849 |
| POLB | SP110 | 0.849 |
| AIM2 | SMARCD3 | 0.849 |
| AIM2 | IRF1 | 0.849 |
| GBP1 | VRK1 | 0.849 |
| GBP1 | PSMA4 | 0.849 |
| GBP1 | SLC6A12 | 0.849 |
| CD40 | C1QA | 0.849 |
| AIM2 | DENND1A | 0.849 |
| GBP1 | RASGRP2 | 0.849 |
| GBP1 | CXCR3 | 0.849 |
| PRRG4 | MAK | 0.849 |
| APOL2 | JOSD1 | 0.849 |
| VAMP5 | SESN1 | 0.849 |
| APOL2 | VPS37B | 0.848 |
| PSME1 | ACP5 | 0.848 |
| CALCOCO2 | NPC2 | 0.848 |
| LHFPL2 | TOB1 | 0.848 |
| BTN3A1 | GBP1 | 0.848 |
| P2RY14 | CCR7 | 0.848 |
| LHFPL2 | KLF13 | 0.848 |
| PSTPIP2 | CD74 | 0.848 |
| UBE2L6 | CXCR3 | 0.848 |
| UBE2L6 | MYL9 | 0.848 |
| UBE2L6 | NMT2 | 0.848 |
| VAMP5 | GPR65 | 0.848 |
| CYB561 | DUSP3 | 0.848 |
| AIM2 | TOB1 | 0.848 |
| PSME1 | DENND1A | 0.848 |
| AIM2 | RRM1 | 0.848 |
| GBP1 | NELL2 | 0.848 |
| GBP5 | LGALS1 | 0.848 |
| C1QA | VAT1 | 0.848 |
| APOL1 | NPC2 | 0.847 |
| PSME1 | SMARCD3 | 0.847 |
| APOL2 | ETS1 | 0.847 |
| UBE2L6 | MSRB2 | 0.847 |
| LHFPL2 | ETS1 | 0.847 |
| CYB561 | NPC2 | 0.847 |
| PRRG4 | NDRG1 | 0.847 |
| APOL2 | EPHA4 | 0.847 |
| NPC2 | POLR3B | 0.847 |
| SMARCD3 | APOL1 | 0.847 |
| POLB | VPS37B | 0.847 |
| ATP1B2 | C1QA | 0.847 |
| C1QA | NXT1 | 0.847 |
| VAMP5 | DHRS3 | 0.847 |
| CALCOCO2 | SMARCD3 | 0.847 |
| CYB561 | PLCD1 | 0.847 |
| CASP1 | MAK | 0.847 |
| GBP1 | SOCS1 | 0.847 |
| PSME1 | JOSD1 | 0.846 |
| POLB | CD74 | 0.846 |
| STAT1 | NPC2 | 0.846 |
| STAT1 | SMARCD3 | 0.846 |
| VAMP5 | ATP1B2 | 0.846 |
| GBP1 | DHRS3 | 0.846 |
| VAMP5 | VPS37B | 0.846 |
| VAMP5 | KLF13 | 0.846 |
| VAMP5 | ZCCHC14 | 0.846 |
| GBP1 | P2RY10 | 0.846 |
| TNFSF10 | C1QA | 0.846 |
| AIM2 | TSPAN2 | 0.846 |
| POLB | TNFSF10 | 0.846 |
| LHFPL2 | DENND1A | 0.846 |
| DUSP3 | LHFPL2 | 0.846 |
| VAMP5 | TOB1 | 0.846 |
| GBP1 | CD83 | 0.846 |
| GBP1 | SAMHD1 | 0.846 |
| UBE2L6 | VAT1 | 0.846 |
| C1QA | TRIM8 | 0.846 |
| UBE2L6 | CD74 | 0.846 |
| GBP1 | NXT1 | 0.846 |
| GBP1 | CHST7 | 0.845 |
| VAMP5 | MAK | 0.845 |
| DUSP3 | TRIM8 | 0.845 |
| C1QA | TOB1 | 0.845 |
| VAMP5 | CXCR3 | 0.845 |
| P2RY14 | ZBP1 | 0.845 |
| BTN3A1 | POLB | 0.845 |
| P2RY14 | TRIM22 | 0.845 |
| SMARCD3 | UPB1 | 0.845 |
| C1QA | MAK | 0.845 |
| UBE2L6 | CASP5 | 0.845 |
| P2RY14 | VPS37B | 0.845 |
| C1QA | RASGRP2 | 0.845 |
| CALCOCO2 | CYB561 | 0.845 |
| GBP1 | POR | 0.845 |
| UBE2L6 | CD46 | 0.845 |
| STAT1 | NDRG1 | 0.845 |
| UBE2L6 | TNFAIP2 | 0.845 |
| AIM2 | POLR3B | 0.845 |
| PSTPIP2 | CD40 | 0.845 |
| LHFPL2 | TSPAN2 | 0.845 |
| ACP5 | TNFSF10 | 0.845 |
| TRIM22 | C1QA | 0.844 |
| APOL2 | ZCCHC14 | 0.844 |
| CALCOCO2 | CASP1 | 0.844 |
| P2RY14 | GCH1 | 0.844 |
| GBP5 | CD79B | 0.844 |
| APOL2 | MAK | 0.844 |
| POLB | CLDND1 | 0.844 |
| POLB | SOCS1 | 0.844 |
| APOL1 | PLCD1 | 0.844 |
| C1QA | FAM8A1 | 0.844 |
| PSMA4 | CIQA | 0.844 |
| LHFPL2 | PRRG4 | 0.844 |
| STAT1 | UBE2L6 | 0.844 |
| AIM2 | DUSP3 | 0.844 |
| P2RY14 | GPX7 | 0.844 |
| UBE2L6 | C1QB | 0.844 |
| GBP1 | VPS37B | 0.844 |
| POLB | GFOD1 | 0.844 |
| ALDH1A1 | LHFPL2 | 0.844 |
| LHFPL2 | GPR132 | 0.844 |
| CYB561 | PRRG4 | 0.844 |
| APOL1 | ETS1 | 0.844 |
| UBE216 | CLCF1 | 0.844 |
| AIM2 | PRRG4 | 0.843 |
| GBP1 | FAM111A | 0.843 |
| RAB20 | NDRG1 | 0.843 |
| CYB561 | POLR3B | 0.843 |
| UBE2L6 | STX11 | 0.843 |
| LHFPL2 | SESN1 | 0.843 |
| GBP1 | CNDP2 | 0.843 |
| STX11 | MAK | 0.843 |
| GBP1 | CCR7 | 0.843 |
| AIM2 | ETS1 | 0.843 |
| SMARCD3 | PRRG4 | 0.843 |
| UBE2L6 | TAPBPL | 0.843 |
| POLB | MSRB2 | 0.843 |
| C1QA | CXCR3 | 0.843 |
| PSME1 | GPR132 | 0.843 |
| VAMP5 | SP110 | 0.843 |
| STAT1 | LHFPL2 | 0.843 |
| VAMP5 | LGALS1 | 0.843 |
| GBP1 | TNFAIP2 | 0.843 |
| VAMP5 | GFOD1 | 0.843 |
| CASP1 | POLR3B | 0.843 |
| POLB | MTHFD2 | 0.843 |
| APOL1 | DHRS3 | 0.843 |
| POLB | TAPBPL | 0.842 |
| PSTPIP2 | LHFPL2 | 0.842 |
| POLB | CASP5 | 0.842 |
| LHFPL2 | EPHA4 | 0.842 |
| VAMP5 | FAM8A1 | 0.842 |
| UBE2L6 | LDHA | 0.842 |
| P2RY14 | IL23A | 0.842 |
| SOCS1 | C1QA | 0.842 |
| APOL2 | CD40 | 0.842 |
| APOL1 | STX11 | 0.842 |
| P2RY14 | POR | 0.842 |
| P2RY14 | AFF1 | 0.842 |
| P2RY14 | LGALS3BP | 0.842 |
| AIM2 | CASP1 | 0.842 |
| AIM2 | TAPBPL | 0.842 |
| C1QA | VPS37B | 0.842 |
| VAMP5 | MYL9 | 0.842 |
| VAMP5 | CDK9 | 0.842 |
| POLB | OSBPL10 | 0.842 |
| LHFPL2 | PLCD1 | 0.842 |
| P2RY14 | CTRL | 0.842 |
| APOL1 | TNFSF10 | 0.842 |
| PSME1 | ETS1 | 0.842 |
| UBE216 | CDK9 | 0.841 |
| POLB | SLC6A12 | 0.841 |
| APOL2 | DHRS3 | 0.841 |
| C1QA | GFOD1 | 0.841 |
| APOL1 | ZCCHC14 | 0.841 |
| UBE2L6 | TSPAN2 | 0.841 |
| POLB | CD83 | 0.841 |
| VAMP5 | CASP5 | 0.841 |
| POLB | FAM111A | 0.841 |
| PSTPIP2 | POLR3B | 0.841 |
| C1QB | APOL1 | 0.841 |
| VAMP5 | CA5B | 0.841 |
| GBP1 | BTN3A2 | 0.841 |
| PSTPIP2 | ETS1 | 0.841 |
| LHFPL2 | GFOD1 | 0.841 |
| AIM2 | PSTPIP2 | 0.841 |
| C1QA | DHRS3 | 0.841 |
| CALCOCO2 | DUSP3 | 0.841 |
| VAMP5 | PRPF4 | 0.841 |
| AIM2 | TRIM8 | 0.841 |
| LHFPL2 | ZCCHC14 | 0.841 |
| ACP5 | IL15 | 0.841 |
| APOL1 | MCM5 | 0.841 |
| VAMP5 | ZNF395 | 0.84 |
| P2RY14 | IGF2BP3 | 0.84 |
| VAMP5 | RAB20 | 0.84 |
| VAMP5 | ACTN4 | 0.84 |
| POLB | AFF1 | 0.84 |
| GBP1 | STX11 | 0.84 |
| VAMP5 | SIDT1 | 0.84 |
| AIM2 | LDHA | 0.84 |
| AIM2 | POR | 0.84 |
| VAMP5 | SAMHD1 | 0.84 |
| UBE216 | TNFSF10 | 0.84 |
| ATP1B2 | NPC2 | 0.84 |
| VAMP5 | TNFSF10 | 0.84 |
| C1QB | APOL2 | 0.84 |
| POLB | PSMA4 | 0.84 |
| CYB561 | ETS1 | 0.84 |
| UBE216 | LAX1 | 0.84 |
| AIM2 | MYL9 | 0.84 |
| FAS | MAK | 0.84 |
| UBE2L6 | SORT1 | 0.84 |
| RAB20 | C1QA | 0.84 |
| PRRG4 | ETS1 | 0.84 |
| APOL1 | EPHA4 | 0.84 |
| STAT1 | APOL2 | 0.84 |
| AIM2 | GPR132 | 0.84 |
| ALDH1A1 | C1QA | 0.84 |
| GBP1 | ZBTB32 | 0.84 |
| UBE2L6 | SAMHD1 | 0.839 |
| NPC2 | GPR132 | 0.839 |
| APOL2 | EHD4 | 0.839 |
| VAMP5 | LDHA | 0.839 |
| VAMP5 | TCFL5 | 0.839 |
| UBE2L6 | SLC6A12 | 0.839 |
| C1QA | SIDT1 | 0.839 |
| IRF1 | APOL1 | 0.839 |
| NPC2 | CASP1 | 0.839 |
| GBP1 | GPR65 | 0.839 |
| SMARCD3 | FAS | 0.839 |
| POLB | VRK1 | 0.839 |
| P2RY14 | VAT1 | 0.839 |
| CD40 | CASP1 | 0.839 |
| TAPBPL | LHFPL2 | 0.839 |
| LHFPL2 | FAS | 0.839 |
| RTP4 | C1QA | 0.839 |
| NPC2 | KLF2 | 0.839 |
| GBP1 | COL9A2 | 0.839 |
| P2RY14 | LDHA | 0.839 |
| UBE2L6 | SLC25A4 | 0.839 |
| IL15 | NPC2 | 0.839 |
| CYB561 | ACP5 | 0.839 |
| C1QA | PRPF4 | 0.839 |
| ACP5 | CASP5 | 0.839 |
| CIQA | CLCF1 | 0.838 |
| P2RY14 | MEF2D | 0.838 |
| UBE2L6 | CLDND1 | 0.838 |
| CALCOCO2 | PSTPIP2 | 0.838 |
| C1QA | CA5B | 0.838 |
| TNFAIP2 | C1QA | 0.838 |
| AIM2 | ACTN4 | 0.838 |
| AIM2 | EPHA4 | 0.838 |
| UBE2L6 | TCFL5 | 0.838 |
| POLB | RTP4 | 0.838 |
| GBP1 | CDK9 | 0.838 |
| SOCS1 | AIM2 | 0.838 |
| AIM2 | SESN1 | 0.838 |
| VAMP5 | TRIM22 | 0.838 |
| DUSP3 | CD40 | 0.838 |
| STAT1 | GBP1 | 0.838 |
| IGF2BP3 | C1QA | 0.838 |
| UBE2L6 | AFF1 | 0.838 |
| APOL1 | CD46 | 0.838 |
| VAMP5 | NMT2 | 0.838 |
| ACP5 | PLCD1 | 0.838 |
| STAT1 | AIM2 | 0.838 |
| VAMP5 | PSMA4 | 0.838 |
| VAMP5 | CD74 | 0.838 |
| UBE2L6 | COL9A2 | 0.837 |
| APOL2 | SIDT1 | 0.837 |
| DUSP3 | MAK | 0.837 |
| LHFPL2 | TRIM8 | 0.837 |
| P2RY14 | CEACAM1 | 0.837 |
| UBE2L6 | MTHFD2 | 0.837 |
| AIM2 | KLF13 | 0.837 |
| APOL2 | TOB1 | 0.837 |
| CD40 | PRRG4 | 0.837 |
| GBP1 | ISG20 | 0.837 |
| DENND1A | CASP1 | 0.837 |
| C1QA | NMT2 | 0.837 |
| ACP5 | ETS1 | 0.837 |
| DENND1A | NPC2 | 0.837 |
| GBP1 | RTP4 | 0.837 |
| P2RY14 | CD46 | 0.836 |
| APOL2 | ATP1B2 | 0.836 |
| P2RY14 | MARCKSL1 | 0.836 |
| LHFPL2 | ACTN4 | 0.836 |
| VAMP5 | COL9A2 | 0.836 |
| AIM2 | ATP1B2 | 0.836 |
| DUSP3 | FAS | 0.836 |
| CASP5 | MAK | 0.836 |
| UBE2L6 | SP110 | 0.836 |
| GBP1 | SP110 | 0.836 |
| CD74 | PRRG4 | 0.836 |
| LHFPL2 | IL15 | 0.836 |
| CD74 | C1QA | 0.836 |
| APOL2 | KLF13 | 0.836 |
| CASP1 | PRRG4 | 0.836 |
| APOL1 | MYL9 | 0.836 |
| AIM2 | RAB20 | 0.836 |
| FAM111A | AIM2 | 0.836 |
| GBP1 | VAT1 | 0.836 |
| APOL2 | SESN1 | 0.836 |
| POLB | COL9A2 | 0.836 |
| MSRB2 | C1QA | 0.836 |
| UBE216 | OSBPL10 | 0.836 |
| SMARCD3 | PSTPIP2 | 0.836 |
| APOL1 | KLF2 | 0.836 |
| LHFPL2 | PSMA4 | 0.836 |
| UBE2L6 | CHST7 | 0.836 |
| PSME1 | EPHA4 | 0.836 |
| AIM2 | PLCD1 | 0.836 |
| C1QA | CDK9 | 0.836 |
| POLB | GCH1 | 0.836 |
| C1QB | LHFPL2 | 0.836 |
| C1QA | ZNF395 | 0.836 |
| UBE2L6 | CD83 | 0.835 |
| STX11 | NDRG1 | 0.835 |
| LHFPL2 | MSRB2 | 0.835 |
| CASP1 | TRIM8 | 0.835 |
| DUSP3 | GPR132 | 0.835 |
| AIM2 | IL15 | 0.835 |
| APOL1 | RASGRP2 | 0.835 |
| ATP1B2 | APOL1 | 0.835 |
| APOL2 | UPB1 | 0.835 |
| STAT1 | PSME1 | 0.835 |
| APOL2 | CA5B | 0.835 |
| VAMP5 | ALDH1A1 | 0.835 |
| GBP1 | CD46 | 0.835 |
| AIM2 | FAS | 0.835 |
| GBP1 | GPX7 | 0.835 |
| C1QB | AIM2 | 0.835 |
| UBE2L6 | SOCS1 | 0.835 |
| PSTPIP2 | PRRG4 | 0.835 |
| VAMP5 | CLCF1 | 0.835 |
| APOL2 | ZNF395 | 0.835 |
| LHFPL2 | MCM5 | 0.835 |
| LHFPL2 | POR | 0.835 |
| C1QB | ACP5 | 0.835 |
| VAMP5 | C1QB | 0.835 |
| CNDP2 | AIM2 | 0.835 |
| APOL2 | RAB20 | 0.835 |
| CASP1 | UPB1 | 0.835 |
| GBP1 | TRIM21 | 0.835 |
| POLB | TSPAN2 | 0.834 |
| PSME1 | NXT1 | 0.834 |
| APOL1 | NXT1 | 0.834 |
| VAMP5 | SOCS1 | 0.834 |
| GBP1 | ITM2C | 0.834 |
| IRF1 | CASP1 | 0.834 |
| GBP1 | TNFSF10 | 0.834 |
| CYB561 | DENND1A | 0.834 |
| PSTPIP2 | TOB1 | 0.834 |
| SMARCD3 | IRF1 | 0.834 |
| GBP1 | CTRL | 0.834 |
| GBP1 | MTHFD2 | 0.834 |
| C1QA | TRIM21 | 0.834 |
| GBP1 | MARCKSL1 | 0.834 |
| P2RY14 | FAM8A1 | 0.834 |
| STAT1 | CYB561 | 0.834 |
| APOL2 | ACTN4 | 0.834 |
| UBE2L6 | CCR7 | 0.834 |
| VAMP5 | SORT1 | 0.834 |
| C1QA | SNX10 | 0.834 |
| STAT1 | POLR3B | 0.834 |
| AIM2 | TNFAIP2 | 0.834 |
| GBP1 | AFF1 | 0.834 |
| C1QB | NDRG1 | 0.834 |
| NPC2 | NXT1 | 0.834 |
| CALCOCO2 | ETS1 | 0.834 |
| VAMP5 | TAPBPL | 0.834 |
| FAS | ETS1 | 0.834 |
| APOL2 | IRF1 | 0.834 |
| APOL2 | POR | 0.834 |
| AIM2 | BTN3A2 | 0.834 |
| IRF1 | TRIM8 | 0.834 |
| AIM2 | JOSD1 | 0.834 |
| STAT1 | DUSP3 | 0.834 |
| AIM2 | MSRB2 | 0.834 |
| VAMP5 | CCR7 | 0.834 |
| NPC2 | MCM5 | 0.834 |
| PSME1 | IRF1 | 0.833 |
| AIM2 | TCFL5 | 0.833 |
| POLB | CXCR3 | 0.833 |
| SMARCD3 | CD40 | 0.833 |
| POLB | IL23A | 0.833 |
| PSTPIP2 | SESN1 | 0.833 |
| APOL2 | RRM1 | 0.833 |
| VAMP5 | XAB2 | 0.833 |
| LHFPL2 | ATP1B2 | 0.833 |
| VAMP5 | TNFAIP2 | 0.833 |
| C1QA | ACTN4 | 0.833 |
| MTHFD2 | C1QA | 0.833 |
| STAT1 | TOB1 | 0.833 |
| PSTPIP2 | DENND1A | 0.833 |
| APOL2 | STX11 | 0.833 |
| DUSP3 | POLR3B | 0.833 |
| GBP5 | IFIH1 | 0.833 |
| SORT1 | C1QA | 0.833 |
| AIM2 | GFOD1 | 0.833 |
| PSME1 | PRPF4 | 0.833 |
| LHFPL2 | JOSD1 | 0.833 |
| AIM2 | ZCCHC14 | 0.833 |
| PSTPIP2 | DUSP3 | 0.833 |
| APOL2 | NXT1 | 0.833 |
| C1QB | CYB561 | 0.833 |
| NDRG1 | ETS1 | 0.833 |
| STAT1 | MAK | 0.833 |
| SLC6A12 | AIM2 | 0.833 |
| LHFPL2 | RAB20 | 0.833 |
| UBE2L6 | GCH1 | 0.832 |
| IRF1 | KLF2 | 0.832 |
| GBP1 | IL23A | 0.832 |
| CALCOCO2 | DENND1A | 0.832 |
| CALCOCO2 | PRRG4 | 0.832 |
| NPC2 | ZCCHC14 | 0.832 |
| CYB561 | ZNF395 | 0.832 |
| ACP5 | NDRG1 | 0.832 |
| CD40 | APOL1 | 0.832 |
| P2RY14 | ZBTB32 | 0.832 |
| APOL2 | PRPF4 | 0.832 |
| APOL1 | CA5B | 0.832 |
| CD74 | FAS | 0.832 |
| APOL1 | ZNF395 | 0.832 |
| FAM111A | LHFPL2 | 0.832 |
| UBE2L6 | CNDP2 | 0.832 |
| C1QB | PRRG4 | 0.832 |
| VAMP5 | RTP4 | 0.832 |
| DUSP3 | NPC2 | 0.832 |
| UBE2L6 | PPP1R2 | 0.832 |
| SMARCD3 | TRIM8 | 0.832 |
| APOL1 | RAB20 | 0.832 |
| VAMP5 | VRK1 | 0.832 |
| UBE2L6 | ZBTB32 | 0.832 |
| C1QA | TCFL5 | 0.832 |
| STAT1 | EPHA4 | 0.832 |
| PSTPIP2 | RRM1 | 0.832 |
| PSME1 | DHRS3 | 0.831 |
| APOL2 | MYL9 | 0.831 |
| VAMP5 | MTHFD2 | 0.831 |
| ACP5 | RAB20 | 0.831 |
| ACP5 | MSRB2 | 0.831 |
| C1QB | PSME1 | 0.831 |
| AIM2 | SIDT1 | 0.831 |
| APOL1 | SORT1 | 0.831 |
| NPC2 | DHRS3 | 0.831 |
| APOL1 | GPR65 | 0.831 |
| VAMP5 | MSRB2 | 0.831 |
| PSTPIP2 | IRF1 | 0.831 |
| AIM2 | WWC3 | 0.831 |
| C1QA | SAMHD1 | 0.831 |
| GBP1 | IGF2BP3 | 0.831 |
| APOL2 | PSMA4 | 0.831 |
| LHFPL2 | MYL9 | 0.831 |
| LHFPL2 | SIDT1 | 0.831 |
| SLC6A12 | C1QA | 0.831 |
| VAMP5 | SLC25A4 | 0.831 |
| ACP5 | DENND1A | 0.831 |
| C1QA | BTN3A2 | 0.831 |
| LHFPL2 | TCFL5 | 0.831 |
| VAMP5 | CLDND1 | 0.831 |
| LHFPL2 | PRPF4 | 0.831 |
| STAT1 | MYL9 | 0.831 |
| C1QB | IRF1 | 0.831 |
| STAT1 | RRM1 | 0.831 |
| PSME1 | TRIM8 | 0.831 |
| TAPBPL | C1QA | 0.83 |
| POLB | TRIM22 | 0.83 |
| PSME1 | SESN1 | 0.83 |
| PSTPIP2 | KLF13 | 0.83 |
| DUSP3 | PRRG4 | 0.83 |
| UBE216 | MARCKSL1 | 0.83 |
| C1QB | NPC2 | 0.83 |
| VAMP5 | GCH1 | 0.83 |
| CD74 | CASP1 | 0.83 |
| POLB | CCR7 | 0.83 |
| CTRL | C1QA | 0.83 |
| UBE216 | RTP4 | 0.83 |
| RTP4 | APOL1 | 0.83 |
| APOL1 | SP110 | 0.83 |
| PSME1 | ZCCHC14 | 0.83 |
| CASP1 | SESN1 | 0.83 |
| GBP5 | TIMP1 | 0.83 |
| LHFPL2 | CASP5 | 0.83 |
| DUSP3 | UPB1 | 0.83 |
| AIM2 | PSMA4 | 0.83 |
| TRIM22 | ACP5 | 0.83 |
| UBE2L6 | VRK1 | 0.83 |
| DUSP3 | CASP1 | 0.83 |
| PSTPIP2 | CASP1 | 0.83 |
| POLB | LAX1 | 0.83 |
| CYB561 | SESN1 | 0.83 |
| LHFPL2 | GPR65 | 0.83 |
| IRF1 | IL15 | 0.83 |
| C1QA | LDHA | 0.83 |
| POLB | GPR65 | 0.83 |
| SOCS1 | LHFPL2 | 0.83 |
| APOL2 | CDK9 | 0.83 |
| C1QA | COL9A2 | 0.83 |
| CYB561 | CA5B | 0.83 |
| LHFPL2 | CA5B | 0.83 |
| IRF1 | ETS1 | 0.83 |
| C1QA | SMAD7 | 0.83 |
| GBP5 | C4BPA | 0.829 |
| NPC2 | PLCD1 | 0.829 |
| AIM2 | CA5B | 0.829 |
| GCH1 | C1QA | 0.829 |
| C1QB | PSTPIP2 | 0.829 |
| VAMP5 | IGF2BP3 | 0.829 |
| C1QB | C1QA | 0.829 |
| UBE2L6 | FAM8A1 | 0.829 |
| RTP4 | LHFPL2 | 0.829 |
| NDRG1 | POLR3B | 0.829 |
| PSTPIP2 | TRIM8 | 0.829 |
| P2RY14 | ITM2C | 0.829 |
| NPC2 | STX11 | 0.829 |
| AIM2 | SAMHD1 | 0.829 |
| PRRG4 | EPHA4 | 0.829 |
| AIM2 | LAX1 | 0.829 |
| IRF1 | MAK | 0.829 |
| POLB | SMAD7 | 0.829 |
| PSME1 | STX11 | 0.829 |
| SMARCD3 | GPR132 | 0.829 |
| AIM2 | CLDND1 | 0.829 |
| IRF1 | POLR3B | 0.829 |
| C1QB | UPB1 | 0.829 |
| FAS | SESN1 | 0.829 |
| AIM2 | MCM5 | 0.829 |
| APOL1 | CASP5 | 0.829 |
| NDRG1 | SESN1 | 0.829 |
| IL15 | ETS1 | 0.829 |
| STAT1 | C1QB | 0.829 |
| AIM2 | VPS37B | 0.829 |
| NPC2 | RASGRP2 | 0.829 |
| STAT1 | CASP1 | 0.829 |
| VAMP5 | SNX10 | 0.829 |
| ATP1B2 | CASP1 | 0.829 |
| C1QA | VRK1 | 0.829 |
| PSME1 | SIDT1 | 0.829 |
| TRIM22 | APOL1 | 0.829 |
| C1QA | CCR7 | 0.829 |
| BTN3A1 | ETS1 | 0.828 |
| VAMP5 | SMAD7 | 0.828 |
| POLB | NELL2 | 0.828 |
| MSRB2 | NDRG1 | 0.828 |
| CALCOCO2 | C1QB | 0.828 |
| VAMP5 | LAX1 | 0.828 |
| DUSP3 | EHD4 | 0.828 |
| APOL2 | MTHFD2 | 0.828 |
| CYB561 | RAB20 | 0.828 |
| POLB | POR | 0.828 |
| GBP1 | CDKN1C | 0.828 |
| CALCOCO2 | CD40 | 0.828 |
| C1QB | FAS | 0.828 |
| IRF1 | NPC2 | 0.828 |
| VAMP5 | CD83 | 0.828 |
| C1QA | SLC25A4 | 0.828 |
| CD40 | ETS1 | 0.828 |
| SMARCD3 | IL15 | 0.828 |
| C1QB | CASP1 | 0.828 |
| ACP5 | EPHA4 | 0.828 |
| CALCOCO2 | IL15 | 0.828 |
| PSTPIP2 | EPHA4 | 0.828 |
| UBE2L6 | BTN3A2 | 0.828 |
| SMARCD3 | POLR3B | 0.828 |
| APOL1 | MTHFD2 | 0.828 |
| CYB561 | FAS | 0.828 |
| PSTPIP2 | POR | 0.828 |
| C1QA | XAB2 | 0.828 |
| PSTPIP2 | TSPAN2 | 0.828 |
| RTP4 | ACP5 | 0.828 |
| RTP4 | AIM2 | 0.828 |
| APOL2 | TNFSF10 | 0.828 |
| GBP1 | GCH1 | 0.828 |
| RTP4 | APOL2 | 0.828 |
| VAMP5 | SLC6A12 | 0.828 |
| AIM2 | KLF2 | 0.828 |
| PSME1 | KLF13 | 0.827 |
| POLB | ACTN4 | 0.827 |
| DUSP3 | IRF1 | 0.827 |
| PSME1 | CD40 | 0.827 |
| C1QA | CD83 | 0.827 |
| AIM2 | RASGRP2 | 0.827 |
| IRF1 | PRRG4 | 0.827 |
| VAMP5 | ZBTB32 | 0.827 |
| CASP1 | ETS1 | 0.827 |
| VAMP5 | MARCKSL1 | 0.827 |
| IRF1 | GPR132 | 0.827 |
| AIM2 | STX11 | 0.827 |
| UBE2L6 | P2RY10 | 0.827 |
| UBE2L6 | CTRL | 0.827 |
| CASP5 | NDRG1 | 0.827 |
| UBE2L6 | NELL2 | 0.827 |
| VAMP5 | IL23A | 0.827 |
| PSTPIP2 | GFOD1 | 0.827 |
| LHFPL2 | STX11 | 0.827 |
| APOL1 | SESN1 | 0.827 |
| PSME1 | RRM1 | 0.827 |
| POLB | IGF2BP3 | 0.827 |
| CYB561 | ZCCHC14 | 0.827 |
| NPC2 | ETS1 | 0.826 |
| APOL2 | CLCF1 | 0.826 |
| UBE2L6 | XAB2 | 0.826 |
| STAT1 | SIDT1 | 0.826 |
| LHFPL2 | KLF2 | 0.826 |
| C1QA | ZBTB32 | 0.826 |
| STAT1 | CD40 | 0.826 |
| APOL1 | TRIM8 | 0.826 |
| APOL1 | JOSD1 | 0.826 |
| UPB1 | ETS1 | 0.826 |
| POLB | CNDP2 | 0.826 |
| LHFPL2 | VPS37B | 0.826 |
| APOL1 | PSMA4 | 0.826 |
| POLB | TRIM21 | 0.826 |
| GBP1 | LGALS3BP | 0.826 |
| LHFPL2 | SP110 | 0.826 |
| CASP1 | EHD4 | 0.826 |
| STAT1 | CALCOCO2 | 0.826 |
| APOL1 | POR | 0.826 |
| IRF1 | FAS | 0.826 |
| C1QA | LAX1 | 0.826 |
| C1QA | ISG20 | 0.826 |
| NPC2 | TNFSF10 | 0.826 |
| CD40 | NPC2 | 0.826 |
| LHFPL2 | NMT2 | 0.826 |
| PSTPIP2 | GPR132 | 0.826 |
| DUSP3 | DENND1A | 0.826 |
| LHFPL2 | BTN3A2 | 0.826 |
| APOL2 | NMT2 | 0.826 |
| CD40 | NDRG1 | 0.826 |
| SMARCD3 | CASP1 | 0.826 |
| LHFPL2 | SAMHD1 | 0.826 |
| UBE2L6 | IGF2BP3 | 0.825 |
| AIM2 | PRPF4 | 0.825 |
| APOL2 | GPR65 | 0.825 |
| APOL1 | CCR7 | 0.825 |
| CYB561 | IL15 | 0.825 |
| GBP5 | IFIT2 | 0.825 |
| PSTPIP2 | ZCCHC14 | 0.825 |
| CALCOCO2 | FAS | 0.825 |
| RTP4 | PSTPIP2 | 0.825 |
| AIM2 | CASP5 | 0.825 |
| NDRG1 | EPHA4 | 0.825 |
| LHFPL2 | WWC3 | 0.825 |
| VAMP5 | OSBPL10 | 0.825 |
| PSME1 | CA5B | 0.825 |
| CALCOCO2 | IRF1 | 0.825 |
| DUSP3 | POR | 0.825 |
| VAMP5 | TSPAN2 | 0.825 |
| AIM2 | NMT2 | 0.825 |
| IL15 | NDRG1 | 0.825 |
| C1QA | CLDND1 | 0.825 |
| NPC2 | MYL9 | 0.825 |
| AIM2 | OSBPL10 | 0.825 |
| STAT1 | MSRB2 | 0.825 |
| DENND1A | FAS | 0.825 |
| PSTPIP2 | ATP1B2 | 0.825 |
| STAT1 | DENND1A | 0.825 |
| DENND1A | PRRG4 | 0.824 |
| ALDH1A1 | C1QB | 0.824 |
| DENND1A | POLR3B | 0.824 |
| CYB561 | IRF1 | 0.824 |
| CASP1 | MYL9 | 0.824 |
| APOL2 | CASP5 | 0.824 |
| PSTPIP2 | EHD4 | 0.824 |
| AIM2 | SMAD7 | 0.824 |
| AIM2 | GPR65 | 0.824 |
| C1QA | ZBP1 | 0.824 |
| PSTPIP2 | IL15 | 0.824 |
| C1QA | TSPAN2 | 0.824 |
| GBP1 | XAB2 | 0.824 |
| UBE2L6 | TRIM22 | 0.824 |
| STAT1 | PSTPIP2 | 0.824 |
| PRRG4 | POLR3B | 0.824 |
| C1QB | DUSP3 | 0.824 |
| BTN3A1 | SMARCD3 | 0.824 |
| SMARCD3 | NPC2 | 0.824 |
| LHFPL2 | VAT1 | 0.824 |
| POLB | TNFAIP2 | 0.824 |
| PSTPIP2 | MYL9 | 0.824 |
| GBP1 | BST2 | 0.824 |
| FAM111A | ETS1 | 0.824 |
| APOL2 | SAMHD1 | 0.824 |
| SMARCD3 | MAK | 0.824 |
| PSME1 | RAB20 | 0.824 |
| VAMP5 | PPP1R2 | 0.824 |
| CALCOCO2 | RTP4 | 0.824 |
| VAMP5 | TRIM21 | 0.824 |
| LHFPL2 | ZNF395 | 0.824 |
| AIM2 | VAT1 | 0.824 |
| AIM2 | SORT1 | 0.824 |
| AIM2 | VRK1 | 0.824 |
| POLB | CHST7 | 0.823 |
| CALCOCO2 | EPHA4 | 0.823 |
| IRF1 | PSMA4 | 0.823 |
| SLC6A12 | LHFPL2 | 0.823 |
| CD40 | IRF1 | 0.823 |
| SOCS1 | NPC2 | 0.823 |
| AIM2 | GCH1 | 0.823 |
| UBE216 | TRIM21 | 0.823 |
| CALCOCO2 | PLCD1 | 0.823 |
| POLB | VAT1 | 0.823 |
| SMARCD3 | DENND1A | 0.823 |
| STAT1 | SESN1 | 0.823 |
| C1QA | LGALS1 | 0.823 |
| FAM111A | NPC2 | 0.823 |
| CYB561 | SIDT1 | 0.823 |
| AIM2 | CLCF1 | 0.823 |
| POLB | SLC25A4 | 0.823 |
| APOL2 | SORT1 | 0.823 |
| RTP4 | IRF1 | 0.823 |
| STAT1 | KLF13 | 0.823 |
| CNDP2 | LHFPL2 | 0.823 |
| ACP5 | MYL9 | 0.823 |
| STAT1 | PRRG4 | 0.823 |
| RTP4 | NPC2 | 0.823 |
| BTN3A1 | PSTPIP2 | 0.823 |
| PSME1 | VPS37B | 0.823 |
| GBP1 | FAM8A1 | 0.823 |
| PSME1 | CASP5 | 0.822 |
| ACP5 | GPR65 | 0.822 |
| PSME1 | TOB1 | 0.822 |
| FAS | MYL9 | 0.822 |
| SMARCD3 | ATP1B2 | 0.822 |
| C1QB | MAK | 0.822 |
| DUSP3 | IL15 | 0.822 |
| PSME1 | AFF1 | 0.822 |
| STAT1 | GFOD1 | 0.822 |
| LHFPL2 | RASGRP2 | 0.822 |
| GBP5 | CD79A | 0.822 |
| PSTPIP2 | TCFL5 | 0.822 |
| C1QB | ETS1 | 0.822 |
| PRRG4 | TOB1 | 0.822 |
| DUSP3 | ETS1 | 0.822 |
| C1QB | CD40 | 0.822 |
| GBP1 | TRIM22 | 0.822 |
| GBP1 | CEACAM1 | 0.822 |
| POLB | CD46 | 0.822 |
| PSTPIP2 | RAB20 | 0.822 |
| AIM2 | ZNF395 | 0.822 |
| C1QB | SMARCD3 | 0.822 |
| NPC2 | ZNF395 | 0.822 |
| PSTPIP2 | PLCD1 | 0.822 |
| IL15 | MYL9 | 0.822 |
| APOL2 | GFOD1 | 0.822 |
| RTP4 | DUSP3 | 0.822 |
| PSME1 | ZNF395 | 0.822 |
| APOL1 | GCH1 | 0.821 |
| C1QA | OSBPL10 | 0.821 |
| CALCOCO2 | ZCCHC14 | 0.821 |
| C1QA | PPP1R2 | 0.821 |
| STAT1 | EHD4 | 0.821 |
| CYB561 | TNFSF10 | 0.821 |
| VAMP5 | GPX7 | 0.821 |
| UBE216 | SNX10 | 0.821 |
| GBP5 | OAS1 | 0.821 |
| SORT1 | NDRG1 | 0.821 |
| CASP1 | RRM1 | 0.821 |
| LHFPL2 | TNFAIP2 | 0.821 |
| APOL1 | CXCR3 | 0.821 |
| GPR65 | ETS1 | 0.821 |
| UBE2L6 | GFI1 | 0.821 |
| AIM2 | TNFSF10 | 0.821 |
| PSME1 | EHD4 | 0.821 |
| CASP1 | ZCCHC14 | 0.821 |
| SOCS1 | APOL1 | 0.821 |
| SMARCD3 | CD74 | 0.821 |
| APOL1 | RRM1 | 0.821 |
| VAMP5 | BTN3A2 | 0.821 |
| LHFPL2 | CLCF1 | 0.821 |
| AIM2 | SP110 | 0.821 |
| CEACAM1 | C1QA | 0.821 |
| NPC2 | PSMA4 | 0.821 |
| APOL2 | MSRB2 | 0.821 |
| AIM2 | ZBTB32 | 0.821 |
| APOL1 | UPB1 | 0.821 |
| CD40 | FAS | 0.821 |
| MYL9 | PRRG4 | 0.821 |
| AIM2 | SLC25A4 | 0.821 |
| NPC2 | EPHA4 | 0.821 |
| STAT1 | ZCCHC14 | 0.821 |
| LHFPL2 | TNFSF10 | 0.821 |
| ACP5 | SORT1 | 0.821 |
| PSME1 | ATP1B2 | 0.821 |
| UBE216 | GPX7 | 0.82 |
| GBP1 | MEF2D | 0.82 |
| PSME1 | CDK9 | 0.82 |
| BTN3A1 | C1QB | 0.82 |
| LHFPL2 | ISG20 | 0.82 |
| APOL1 | KLF13 | 0.82 |
| C1QA | NELL2 | 0.82 |
| AIM2 | CD46 | 0.82 |
| RTP4 | PRRG4 | 0.82 |
| FAS | NDRG1 | 0.82 |
| TNFSF10 | MAK | 0.82 |
| APOL1 | MAK | 0.82 |
| VAMP5 | ISG20 | 0.82 |
| VAMP5 | CTRL | 0.82 |
| SOCS1 | CASP1 | 0.82 |
| PSTPIP2 | JOSD1 | 0.82 |
| UBE2L6 | IL23A | 0.82 |
| DUSP3 | CD74 | 0.82 |
| NPC2 | SESN1 | 0.82 |
| CD40 | DENND1A | 0.82 |
| POLB | ALDH1A1 | 0.82 |
| IRF1 | TOB1 | 0.82 |
| BTN3A1 | ACP5 | 0.82 |
| VAMP5 | CNDP2 | 0.82 |
| AIM2 | NXT1 | 0.82 |
| PSME1 | TNFSF10 | 0.82 |
| DENND1A | IL15 | 0.82 |
| IRF1 | DENND1A | 0.82 |
| LHFPL2 | CLDND1 | 0.82 |
| SMARCD3 | DUSP3 | 0.819 |
| DUSP3 | ATP1B2 | 0.819 |
| STAT1 | RAB20 | 0.819 |
| APOL1 | TOB1 | 0.819 |
| AIM2 | TRIM21 | 0.819 |
| C1QB | CD46 | 0.819 |
| SLC6A12 | ACP5 | 0.819 |
| PSTPIP2 | SIDT1 | 0.819 |
| AIM2 | PPP1R2 | 0.819 |
| LHFPL2 | LAX1 | 0.819 |
| C1QB | MYL9 | 0.819 |
| TRIM22 | NPC2 | 0.819 |
| APOL1 | WWC3 | 0.819 |
| AIM2 | TRIM22 | 0.819 |
| FAM111A | APOL1 | 0.819 |
| APOL1 | PRPF4 | 0.819 |
| C1QB | MCM5 | 0.819 |
| CD46 | NPC2 | 0.819 |
| NPC2 | EHD4 | 0.819 |
| PSTPIP2 | MCM5 | 0.819 |
| LHFPL2 | LDHA | 0.819 |
| AIM2 | ISG20 | 0.819 |
| PSTPIP2 | FAS | 0.819 |
| PSME1 | MYL9 | 0.819 |
| LHFPL2 | TRIM21 | 0.819 |
| CYB561 | STX11 | 0.819 |
| FAS | EPHA4 | 0.819 |
| C1QA | IL23A | 0.819 |
| APOL2 | SP110 | 0.819 |
| DUSP3 | ZCCHC14 | 0.819 |
| C1QB | IL15 | 0.819 |
| MAK | ETS1 | 0.819 |
| PSME1 | PSMA4 | 0.819 |
| VAMP5 | CHST7 | 0.819 |
| CASP1 | EPHA4 | 0.819 |
| C1QB | PLCD1 | 0.818 |
| UBE2L6 | ISG20 | 0.818 |
| NPC2 | CASP5 | 0.818 |
| DUSP3 | PSMA4 | 0.818 |
| CYB561 | CASP5 | 0.818 |
| PRRG4 | KLF13 | 0.818 |
| SOCS1 | PSTPIP2 | 0.818 |
| C1QB | POLR3B | 0.818 |
| VAMP5 | NELL2 | 0.818 |
| STAT1 | CASP5 | 0.818 |
| CYB561 | MYL9 | 0.818 |
| IL15 | SESN1 | 0.818 |
| PSTPIP2 | MSRB2 | 0.818 |
| SP110 | NPC2 | 0.818 |
| PSME1 | NMT2 | 0.818 |
| C1QA | MARCKSL1 | 0.818 |
| GBP1 | ZBP1 | 0.818 |
| C1QB | CD74 | 0.818 |
| PSTPIP2 | CASP5 | 0.818 |
| PSTPIP2 | KLF2 | 0.818 |
| IL15 | POLR3B | 0.818 |
| ACP5 | POLR3B | 0.818 |
| APOL1 | FAM8A1 | 0.818 |
| GBP1 | SNX10 | 0.818 |
| APOL1 | EHD4 | 0.818 |
| APOL2 | TCFL5 | 0.818 |
| RTP4 | PSME1 | 0.818 |
| ALDH1A1 | PSTPIP2 | 0.818 |
| STAT1 | CD74 | 0.818 |
| IRF1 | EPHA4 | 0.818 |
| BTN3A1 | NPC2 | 0.818 |
| APOL1 | SIDT1 | 0.818 |
| AFF1 | NPC2 | 0.818 |
| PSMA4 | PRRG4 | 0.818 |
| PSTPIP2 | PSMA4 | 0.818 |
| P2RY14 | XAB2 | 0.818 |
| C1QB | EHD4 | 0.818 |
| APOL2 | VAT1 | 0.818 |
| SLC6A12 | APOL1 | 0.818 |
| AIM2 | CXCR3 | 0.817 |
| DUSP3 | EPHA4 | 0.817 |
| CD74 | CASP5 | 0.817 |
| C1QB | STX11 | 0.817 |
| UBE216 | ITM2C | 0.817 |
| NPC2 | CA5B | 0.817 |
| FAM111A | C1QB | 0.817 |
| SP110 | ACP5 | 0.817 |
| CASP1 | KLF2 | 0.817 |
| P2RY14 | IFIH1 | 0.817 |
| FAS | CASP1 | 0.817 |
| RTP4 | MYL9 | 0.817 |
| RTP4 | SMARCD3 | 0.817 |
| IRF1 | PLCD1 | 0.817 |
| APOL1 | VPS37B | 0.817 |
| CALCOCO2 | RAB20 | 0.817 |
| APOL2 | GCH1 | 0.817 |
| FAS | POLR3B | 0.817 |
| CNDP2 | C1QA | 0.817 |
| CYB561 | KLF13 | 0.817 |
| SMARCD3 | VAT1 | 0.817 |
| IRF1 | MCM5 | 0.817 |
| SAMHD1 | NDRG1 | 0.817 |
| C1QB | CASP5 | 0.817 |
| ACP5 | MCM5 | 0.817 |
| LHFPL2 | SMAD7 | 0.817 |
| ACP5 | PSMA4 | 0.817 |
| SMARCD3 | GPR65 | 0.817 |
| APOL1 | CLCF1 | 0.817 |
| AIM2 | CTRL | 0.817 |
| C1QB | DENND1A | 0.817 |
| IFIH1 | CIQA | 0.817 |
| LHFPL2 | TRIM22 | 0.817 |
| SOCS1 | SMARCD3 | 0.817 |
| PRRG4 | ZCCHC14 | 0.817 |
| SP110 | NDRG1 | 0.817 |
| RTP4 | CYB561 | 0.817 |
| FAM111A | SMARCD3 | 0.817 |
| PSME1 | GPR65 | 0.817 |
| PSMA4 | ETS1 | 0.816 |
| RAB20 | PRRG4 | 0.816 |
| IFIT2 | C1QA | 0.816 |
| FAS | TSPAN2 | 0.816 |
| P2RY14 | SNX10 | 0.816 |
| CYB561 | DHRS3 | 0.816 |
| POLB | CTRL | 0.816 |
| C1QB | RRM1 | 0.816 |
| PSME1 | MTHFD2 | 0.816 |
| LHFPL2 | GCH1 | 0.816 |
| C1QA | ITM2C | 0.816 |
| ACP5 | CASB | 0.816 |
| CIQB | TOB1 | 0.816 |
| CYB561 | SORT1 | 0.816 |
| SMARCD3 | PSMA4 | 0.816 |
| PRRG4 | SESN1 | 0.816 |
| AIM2 | AFF1 | 0.816 |
| NPC2 | GPR65 | 0.816 |
| RTP4 | NDRG1 | 0.816 |
| MSRB2 | PRRG4 | 0.816 |
| SOCS1 | APOL2 | 0.816 |
| APOL1 | MSRB2 | 0.816 |
| SMARCD3 | MYL9 | 0.816 |
| NPC2 | MAK | 0.816 |
| NPC2 | CCR7 | 0.816 |
| AIM2 | DHRS3 | 0.816 |
| CALCOCO2 | SESN1 | 0.815 |
| AIM2 | MTHFD2 | 0.815 |
| CASP1 | TCFL5 | 0.815 |
| IRF1 | ATP1B2 | 0.815 |
| SMARCD3 | STX11 | 0.815 |
| IRF1 | MYL9 | 0.815 |
| LHFPL2 | DHRS3 | 0.815 |
| CASP1 | GPR132 | 0.815 |
| CASP1 | PLCD1 | 0.815 |
| CD40 | POLR3B | 0.815 |
| NPC2 | FAM8A1 | 0.815 |
| FAS | TOB1 | 0.815 |
| C1QB | EPHA4 | 0.815 |
| P2RY14 | HRK | 0.815 |
| CASP1 | TOB1 | 0.815 |
| STAT1 | IRF1 | 0.815 |
| STAT1 | PLCD1 | 0.815 |
| BTN3A1 | DUSP3 | 0.815 |
| PSTPIP2 | CA5B | 0.815 |
| GPR65 | MYL9 | 0.815 |
| ACP5 | DHRS3 | 0.815 |
| NPC2 | RAB20 | 0.815 |
| C1QB | GPR132 | 0.815 |
| CALCOCO2 | TOB1 | 0.815 |
| STAT1 | ATP182 | 0.815 |
| APOL1 | IL23A | 0.815 |
| AIM2 | CDK9 | 0.814 |
| LHFPL2 | COL9A2 | 0.814 |
| C1QB | GPR65 | 0.814 |
| AIM2 | CHST7 | 0.814 |
| APOL1 | VAT1 | 0.814 |
| CALCOCO2 | JOSD1 | 0.814 |
| LHFPL2 | MTHFD2 | 0.814 |
| CD40 | EPHA4 | 0.814 |
| APOL2 | CCR7 | 0.814 |
| APOL1 | CD83 | 0.814 |
| BTN3A1 | APOL1 | 0.814 |
| CALCOCO2 | GPR132 | 0.814 |
| CYB561 | NMT2 | 0.814 |
| LHFPL2 | CD46 | 0.814 |
| PSTPIP2 | ACTN4 | 0.814 |
| APOL1 | OSBPL10 | 0.814 |
| POLB | FAM8A1 | 0.814 |
| VAMP5 | ZBP1 | 0.814 |
| VAMP5 | CEACAM1 | 0.814 |
| RTP4 | DENND1A | 0.814 |
| SMARCD3 | PLCD1 | 0.814 |
| CD74 | MCM5 | 0.814 |
| PSTPIP2 | RASGRP2 | 0.814 |
| CASP1 | RAB20 | 0.814 |
| CYB561 | CLCF1 | 0.814 |
| PSMA4 | MYL9 | 0.814 |
| IL15 | EPHA4 | 0.814 |
| FAM111A | APOL2 | 0.814 |
| IL15 | PRRG4 | 0.814 |
| CASP1 | POR | 0.814 |
| C1QB | POR | 0.814 |
| LHFPL2 | PPP1R2 | 0.814 |
| CALCOCO2 | KLF13 | 0.814 |
| AIM2 | COL9A2 | 0.814 |
| CALCOCO2 | MAK | 0.814 |
| VAMP5 | ITM2C | 0.814 |
| IL15 | CASP1 | 0.814 |
| RTP4 | CASP1 | 0.814 |
| ACP5 | ZNF395 | 0.814 |
| CASP1 | MCM5 | 0.814 |
| ALDH1A1 | MYL9 | 0.814 |
| BTN3A1 | CASP1 | 0.814 |
| LHFPL2 | CDK9 | 0.814 |
| RTP4 | ETS1 | 0.814 |
| CD40 | CASP5 | 0.814 |
| C1QB | TRIM8 | 0.813 |
| C1QB | SOCS1 | 0.813 |
| TNFSF10 | NDRG1 | 0.813 |
| SMARCD3 | ETS1 | 0.813 |
| DENND1A | PSMA4 | 0.813 |
| LHFPL2 | NXT1 | 0.813 |
| IRF1 | ZCCHC14 | 0.813 |
| CYB561 | MSRB2 | 0.813 |
| IRF1 | ACTN4 | 0.813 |
| GCH1 | ETS1 | 0.813 |
| FAS | RAB20 | 0.813 |
| ACP5 | GCH1 | 0.813 |
| STX11 | TOB1 | 0.813 |
| STAT1 | NMT2 | 0.813 |
| DENND1A | PLCD1 | 0.813 |
| C1QA | CHST7 | 0.813 |
| STAT1 | UPB1 | 0.813 |
| AIM2 | GF1 | 0.813 |
| APOL1 | SNX10 | 0.813 |
| NPC2 | POR | 0.813 |
| FAS | ZCCHC14 | 0.813 |
| CD40 | STX11 | 0.813 |
| SLC6A12 | NDRG1 | 0.813 |
| PSME1 | SORT1 | 0.813 |
| APOL2 | CXCR3 | 0.813 |
| IRF1 | KLF13 | 0.812 |
| SMARCD3 | RASGRP2 | 0.812 |
| SMARCD3 | MCM5 | 0.812 |
| PSTPIP2 | TAPBPL | 0.812 |
| DUSP3 | MYL9 | 0.812 |
| PSME1 | SP110 | 0.812 |
| LHFPL2 | CXCR3 | 0.812 |
| C1QB | ATP1B2 | 0.812 |
| PSTPIP2 | VPS37B | 0.812 |
| C1QB | KLF13 | 0.812 |
| PSME1 | SAMHD1 | 0.812 |
| BTN3A1 | APOL2 | 0.812 |
| SOCS1 | DUSP3 | 0.812 |
| MYL9 | NDRG1 | 0.812 |
| DUSP3 | TOB1 | 0.812 |
| LHFPL2 | OSBPL10 | 0.812 |
| STAT1 | CA5B | 0.812 |
| P2RY14 | IFIT2 | 0.812 |
| SMARCD3 | KLF2 | 0.812 |
| AIM2 | LGALS3BP | 0.812 |
| APOL1 | SAMHD1 | 0.812 |
| RTP4 | C1QB | 0.812 |
| CD40 | MYL9 | 0.812 |
| BTN3A1 | EPHA4 | 0.812 |
| APOL1 | NMT2 | 0.812 |
| APOL2 | AFF1 | 0.812 |
| ACP5 | SIDT1 | 0.812 |
| PSTPIP2 | NMT2 | 0.812 |
| LHFPL2 | SORT1 | 0.812 |
| FAS | MSRB2 | 0.812 |
| DUSP3 | KLF13 | 0.812 |
| NPC2 | VAT1 | 0.812 |
| DUSP3 | PLCD1 | 0.812 |
| PSTPIP2 | SAMHD1 | 0.812 |
| C1QB | GFOD1 | 0.811 |
| C1QB | PSMA4 | 0.811 |
| MTHFD2 | NPC2 | 0.811 |
| NPC2 | GCH1 | 0.811 |
| CASP1 | KLF13 | 0.811 |
| TRIM22 | ETS1 | 0.811 |
| PSME1 | MSRB2 | 0.811 |
| SLC6A12 | APOL2 | 0.811 |
| POLB | MARCKSL1 | 0.811 |
| AIM2 | IGF2BP3 | 0.811 |
| POR | ETS1 | 0.811 |
| NDRG1 | ZCCHC14 | 0.811 |
| CALCOCO2 | MYL9 | 0.811 |
| AIM2 | BST2 | 0.811 |
| C1QB | SESN1 | 0.811 |
| CD46 | ETS1 | 0.811 |
| STAT1 | IL15 | 0.811 |
| APOL2 | CD46 | 0.811 |
| PSME1 | CLCF1 | 0.811 |
| STAT1 | FAS | 0.811 |
| STAT1 | MCM5 | 0.811 |
| BTN3A1 | NDRG1 | 0.811 |
| UBE2L6 | MEF2D | 0.811 |
| TRIM8 | ETS1 | 0.811 |
| AIM2 | P2RY10 | 0.811 |
| AIM2 | CD83 | 0.811 |
| APOL2 | TRIM22 | 0.811 |
| SMARCD3 | TRIM22 | 0.811 |
| PSME1 | MAK | 0.811 |
| POLB | ZBP1 | 0.811 |
| RTP4 | POLR3B | 0.811 |
| POLB | CEACAM1 | 0.811 |
| STX11 | ETS1 | 0.811 |
| C1QB | TNFSF10 | 0.811 |
| CD74 | ETS1 | 0.811 |
| C1QB | WWC3 | 0.811 |
| CYB561 | TOB1 | 0.811 |
| AIM2 | NELL2 | 0.811 |
| RTP4 | STX11 | 0.811 |
| FAM111A | ACP5 | 0.811 |
| IRF1 | RASGRP2 | 0.811 |
| LHFPL2 | VRK1 | 0.81 |
| FAM111A | PSME1 | 0.81 |
| IRF1 | CASP5 | 0.81 |
| STAT1 | RTP4 | 0.81 |
| ACP5 | FAM8A1 | 0.81 |
| C1QB | TRIM22 | 0.81 |
| C1QB | ACTN4 | 0.81 |
| SLC6A12 | PSTPIP2 | 0.81 |
| C1QB | KLF2 | 0.81 |
| AFF1 | LHFPL2 | 0.81 |
| PSTPIP2 | CLDND1 | 0.81 |
| NPC2 | KLF13 | 0.81 |
| APOL2 | CD83 | 0.81 |
| GBP1 | HRK | 0.81 |
| VAMP5 | IFIT2 | 0.81 |
| NPC2 | RRM1 | 0.81 |
| CYB561 | ATP1B2 | 0.81 |
| CALCOCO2 | CASP5 | 0.81 |
| STAT1 | PSMA4 | 0.81 |
| APOL1 | NELL2 | 0.81 |
| SMARCD3 | CASP5 | 0.81 |
| UBE2L6 | ZBP1 | 0.81 |
| APOL2 | OSBPL10 | 0.81 |
| SMARCD3 | SESN1 | 0.81 |
| SOCS1 | ETS1 | 0.81 |
| IL15 | ZCCHC14 | 0.81 |
| CD40 | ACP5 | 0.81 |
| SMARCD3 | VPS37B | 0.81 |
| PSTPIP2 | LDHA | 0.81 |
| IGF2BP3 | APOL1 | 0.81 |
| NPC2 | JOSD1 | 0.81 |
| PSTPIP2 | SP110 | 0.81 |
| APOL2 | CD74 | 0.81 |
| STAT1 | KLF2 | 0.81 |
| VAMP5 | GF1 | 0.81 |
| DUSP3 | ZNF395 | 0.81 |
| C1QB | RAB20 | 0.809 |
| UBE2L6 | CEACAM1 | 0.809 |
| ACP5 | JOSD1 | 0.809 |
| C1QA | GPX7 | 0.809 |
| AIM2 | CCR7 | 0.809 |
| PSTPIP2 | STX11 | 0.809 |
| VAMP5 | IFIH1 | 0.809 |
| CALCOCO2 | PSMA4 | 0.809 |
| C1QB | JOSD1 | 0.809 |
| IRF1 | JOSD1 | 0.809 |
| DUSP3 | SESN1 | 0.809 |
| CASP1 | JOSD1 | 0.809 |
| ALDH1A1 | APOL2 | 0.809 |
| C1QB | SP110 | 0.809 |
| C1QB | ZCCHC14 | 0.809 |
| C1QB | VAT1 | 0.809 |
| IRF1 | RAB20 | 0.809 |
| ACP5 | SESN1 | 0.809 |
| IRF1 | DHRS3 | 0.809 |
| C1QB | MSRB2 | 0.809 |
| GBP1 | LGALS1 | 0.809 |
| ALDH1A1 | APOL1 | 0.809 |
| STAT1 | LAX1 | 0.809 |
| RTP4 | RRM1 | 0.809 |
| SMARCD3 | ZCCHC14 | 0.809 |
| CD74 | TNFSF10 | 0.809 |
| LHFPL2 | CTRL | 0.809 |
| WWC3 | NPC2 | 0.809 |
| IGF2BP3 | NPC2 | 0.809 |
| CASP1 | RASGRP2 | 0.809 |
| ATP1B2 | PRRG4 | 0.809 |
| CYB561 | MCM5 | 0.809 |
| POLB | BTN3A2 | 0.808 |
| STAT1 | JOSD1 | 0.808 |
| CALCOCO2 | TNFSF10 | 0.808 |
| IGF2BP3 | APOL2 | 0.808 |
| NPC2 | VPS37B | 0.808 |
| DUSP3 | KLF2 | 0.808 |
| SMARCD3 | SP110 | 0.808 |
| STX11 | CASP1 | 0.808 |
| C1QB | AFF1 | 0.808 |
| DUSP3 | TNFSF10 | 0.808 |
| DENND1A | ETS1 | 0.808 |
| CASP1 | CA5B | 0.808 |
| C1QB | CXCR3 | 0.808 |
| PRRG4 | RRM1 | 0.808 |
| LHFPL2 | SLC25A4 | 0.808 |
| APOL2 | SMAD7 | 0.808 |
| APOL2 | COL9A2 | 0.808 |
| APOL1 | CDK9 | 0.808 |
| PLCD1 | ETS1 | 0.808 |
| PSTPIP2 | VAT1 | 0.808 |
| SOCS1 | ACP5 | 0.808 |
| PSTPIP2 | PRPF4 | 0.808 |
| STAT1 | CLDND1 | 0.808 |
| CYB561 | PSMA4 | 0.808 |
| APOL1 | TCFL5 | 0.808 |
| NPC2 | PRPF4 | 0.808 |
| C1QB | FAM8A1 | 0.808 |
| CD40 | TNFSF10 | 0.808 |
| ALDH1A1 | ETS1 | 0.808 |
| BTN3A1 | PSME1 | 0.808 |
| TRIM21 | NDRG1 | 0.807 |
| APOL2 | WWC3 | 0.807 |
| CALCOCO2 | SIDT1 | 0.807 |
| IRF1 | EHD4 | 0.807 |
| TNFSF10 | ETS1 | 0.807 |
| TNFAIP2 | ACP5 | 0.807 |
| STAT1 | GPR132 | 0.807 |
| RTP4 | CASP5 | 0.807 |
| IL15 | RRM1 | 0.807 |
| SOCS1 | FAS | 0.807 |
| IRF1 | SIDT1 | 0.807 |
| GBP1 | CD79B | 0.807 |
| APOL1 | GFOD1 | 0.807 |
| PRRG4 | SIDT1 | 0.807 |
| PSME1 | WWC3 | 0.807 |
| LHFPL2 | NELL2 | 0.807 |
| PSME1 | CXCR3 | 0.807 |
| SMARCD3 | EHD4 | 0.807 |
| APOL2 | TAPBPL | 0.807 |
| RTP4 | CD74 | 0.807 |
| NPC2 | CDK9 | 0.807 |
| C1QB | LDHA | 0.807 |
| ACP5 | MTHFD2 | 0.807 |
| SOCS1 | CASP5 | 0.807 |
| SMARCD3 | TNFSF10 | 0.807 |
| P2RY14 | CDKN1C | 0.807 |
| DENND1A | EPHA4 | 0.807 |
| CYB561 | GPR132 | 0.807 |
| CALCOCO2 | KLF2 | 0.807 |
| CALCOCO2 | TRIM8 | 0.807 |
| RTP4 | UPB1 | 0.807 |
| IRF1 | TNFSF10 | 0.806 |
| C1QA | GF1 | 0.806 |
| ACP5 | RRM1 | 0.806 |
| C1QB | TNFAIP2 | 0.806 |
| SLC6A12 | C1QB | 0.806 |
| RAB20 | POR | 0.806 |
| FAM111A | DUSP3 | 0.806 |
| DUSP3 | TRIM22 | 0.806 |
| AIM2 | FAM8A1 | 0.806 |
| PSTPIP2 | TNFSF10 | 0.806 |
| CYB561 | CD40 | 0.806 |
| POLB | ISG20 | 0.806 |
| SMARCD3 | EPHA4 | 0.806 |
| MSRB2 | IL15 | 0.806 |
| CYB561 | JOSD1 | 0.806 |
| STAT1 | PRPF4 | 0.806 |
| SORT1 | MAK | 0.806 |
| STAT1 | SMAD7 | 0.806 |
| RTP4 | RAB20 | 0.806 |
| RTP4 | EPHA4 | 0.806 |
| CALCOCO2 | MSRB2 | 0.806 |
| PSTPIP2 | BTN3A2 | 0.806 |
| AIM2 | ZBP1 | 0.806 |
| CASP5 | UPB1 | 0.806 |
| DUSP3 | RAB20 | 0.806 |
| CALCOCO2 | MCM5 | 0.806 |
| PSTPIP2 | ZNF395 | 0.806 |
| PSTPIP2 | LAX1 | 0.806 |
| DUSP3 | RRM1 | 0.806 |
| CYB561 | SP110 | 0.806 |
| ACP5 | CXCR3 | 0.806 |
| CALCOCO2 | CA5B | 0.806 |
| RTP4 | FAS | 0.806 |
| GPR65 | SESN1 | 0.806 |
| PSTPIP2 | DHRS3 | 0.805 |
| SMARCD3 | GCH1 | 0.805 |
| LHFPL2 | CHST7 | 0.805 |
| SMARCD3 | SAMHD1 | 0.805 |
| STAT1 | SORT1 | 0.805 |
| CASP5 | MYL9 | 0.805 |
| GPR132 | ETS1 | 0.805 |
| TRIM22 | MYL9 | 0.805 |
| CASP5 | PRRG4 | 0.805 |
| IRF1 | VPS37B | 0.805 |
| SMARCD3 | CD46 | 0.805 |
| MYL9 | MAK | 0.805 |
| BTN3A1 | POLR3B | 0.805 |
| IRF1 | SESN1 | 0.805 |
| DENND1A | GPR132 | 0.805 |
| NPC2 | SORT1 | 0.805 |
| C1QB | TAPBPL | 0.805 |
| FAS | KLF13 | 0.805 |
| CALCOCO2 | PRPF4 | 0.805 |
| CD74 | IL15 | 0.805 |
| LHFPL2 | ZBP1 | 0.805 |
| DUSP3 | VAT1 | 0.805 |
| PRRG4 | PLCD1 | 0.805 |
| PSTPIP2 | WWC3 | 0.805 |
| PRRG4 | TSPAN2 | 0.805 |
| DUSP3 | STX11 | 0.805 |
| C1QB | SIDT1 | 0.805 |
| CD40 | MCM5 | 0.805 |
| DUSP3 | MCM5 | 0.805 |
| STAT1 | ZNF395 | 0.805 |
| CD40 | RAB20 | 0.805 |
| RTP4 | CD40 | 0.805 |
| CD40 | PLCD1 | 0.805 |
| C1QB | DHRS3 | 0.805 |
| NDRG1 | NMT2 | 0.805 |
| RAB20 | ETS1 | 0.805 |
| PSTPIP2 | ISG20 | 0.805 |
| ATP1B2 | ETS1 | 0.805 |
| DENND1A | CASP5 | 0.804 |
| PSME1 | GCH1 | 0.804 |
| APOL1 | ZBP1 | 0.804 |
| STAT1 | ACTN4 | 0.804 |
| PSMA4 | NDRG1 | 0.804 |
| CASP5 | ETS1 | 0.804 |
| STAT1 | SLC6A12 | 0.804 |
| AFF1 | APOL1 | 0.804 |
| C1QB | RASGRP2 | 0.804 |
| SLC6A12 | PRRG4 | 0.804 |
| TNFAIP2 | APOL1 | 0.804 |
| CASP1 | CLDND1 | 0.804 |
| APOL2 | NELL2 | 0.804 |
| ACP5 | RASGRP2 | 0.804 |
| GBP5 | RSAD2 | 0.804 |
| DUSP3 | CASP5 | 0.804 |
| C1QB | NXT1 | 0.804 |
| STAT1 | NELL2 | 0.804 |
| DUSP3 | JOSD1 | 0.804 |
| PSTPIP2 | CLCF1 | 0.804 |
| UBEZL6 | CDKN1C | 0.804 |
| PSME1 | VAT1 | 0.804 |
| CALCOCO2 | ATP1B2 | 0.804 |
| NDRG1 | PLCD1 | 0.804 |
| CASP1 | CASP5 | 0.804 |
| SOCS1 | NDRG1 | 0.804 |
| DUSP3 | GFOD1 | 0.804 |
| POLB | GPX7 | 0.804 |
| CYB561 | RRM1 | 0.804 |
| P2RY14 | OAS1 | 0.804 |
| AIM2 | ITM2C | 0.804 |
| BTN3A1 | MYL9 | 0.804 |
| POLB | SNX10 | 0.804 |
| RTP4 | WWC3 | 0.804 |
| CD74 | STX11 | 0.804 |
| SP110 | CASP1 | 0.804 |
| SMARCD3 | DHRS3 | 0.803 |
| ETS1 | COL9A2 | 0.803 |
| SOCS1 | PRRG4 | 0.803 |
| SMARCD3 | NXT1 | 0.803 |
| CALCOCO2 | STX11 | 0.803 |
| C1QB | GCH1 | 0.803 |
| STAT1 | TRIM8 | 0.803 |
| PSME1 | GFOD1 | 0.803 |
| TNFSF10 | POLR3B | 0.803 |
| PRRG4 | CA5B | 0.803 |
| SLC6A12 | NPC2 | 0.803 |
| CALCOCO2 | RRM1 | 0.803 |
| APOL1 | PPP1R2 | 0.803 |
| CYB561 | MTHFD2 | 0.803 |
| C1QA | P2RY10 | 0.803 |
| BTN3A1 | RAB20 | 0.803 |
| FAM111A | PSTPIP2 | 0.803 |
| SAMHD1 | ETS1 | 0.803 |
| C1QB | IGF2BP3 | 0.803 |
| IL15 | CASP5 | 0.803 |
| MSRB2 | CASP1 | 0.803 |
| C1QA | CDKN1C | 0.803 |
| FAS | PRRG4 | 0.803 |
| NPC2 | CLCF1 | 0.803 |
| APOL2 | CLDND1 | 0.803 |
| CD40 | IL15 | 0.803 |
| MTHFD2 | ETS1 | 0.803 |
| SIDT1 | NDRG1 | 0.803 |
| AFF1 | ETS1 | 0.803 |
| APOL1 | TRIM21 | 0.803 |
| NDRG1 | ZNF395 | 0.803 |
| CASP1 | GFOD1 | 0.803 |
| CASP1 | SAMHD1 | 0.803 |
| RTP4 | SESN1 | 0.803 |
| SMARCD3 | WWC3 | 0.803 |
| CASP1 | PSMA4 | 0.803 |
| CD74 | APOL1 | 0.802 |
| IGF2BP3 | ACP5 | 0.802 |
| C1QB | CA5B | 0.802 |
| STAT1 | SOCS1 | 0.802 |
| CEACAM1 | ACP5 | 0.802 |
| CYB561 | RASGRP2 | 0.802 |
| PSTPIP2 | PPP1R2 | 0.802 |
| C1QB | VPS37B | 0.802 |
| APOL1 | COL9A2 | 0.802 |
| BTN3A1 | SIDT1 | 0.802 |
| ATP1B2 | CASP5 | 0.802 |
| DUSP3 | GPR65 | 0.802 |
| LHFPL2 | CCR7 | 0.802 |
| DUSP3 | SP110 | 0.802 |
| LHFPL2 | BST2 | 0.802 |
| DENND1A | MCM5 | 0.802 |
| PSTPIP2 | SMAD7 | 0.802 |
| RAB20 | POLR3B | 0.802 |
| LHFPL2 | CD83 | 0.802 |
| APOL2 | TSPAN2 | 0.802 |
| DUSP3 | RASGRP2 | 0.802 |
| CD40 | SESN1 | 0.802 |
| IL15 | RAB20 | 0.802 |
| NPC2 | CXCR3 | 0.802 |
| ACP5 | TRIM21 | 0.802 |
| ACP5 | ZCCHC14 | 0.802 |
| GBP5 | IF144 | 0.802 |
| VAMP5 | P2RY10 | 0.802 |
| C1QB | CCR7 | 0.802 |
| CALCOCO2 | ZNF395 | 0.802 |
| STAT1 | STX11 | 0.802 |
| IRF1 | ZNF395 | 0.802 |
| NPC2 | SAMHD1 | 0.802 |
| CASP1 | ZNF395 | 0.802 |
| IRF1 | MTHFD2 | 0.802 |
| C1QB | BTN3A2 | 0.802 |
| RAB20 | UPB1 | 0.802 |
| PSTPIP2 | SORT1 | 0.802 |
| PSME1 | CCR7 | 0.802 |
| TAPBPL | PRRG4 | 0.802 |
| ACP5 | TOB1 | 0.802 |
| AIM2 | GPX7 | 0.802 |
| NDRG1 | CA5B | 0.802 |
| CASP5 | TRIM8 | 0.801 |
| CALCOCO2 | SP110 | 0.801 |
| AIM2 | IL23A | 0.801 |
| DENND1A | MAK | 0.801 |
| SP110 | ETS1 | 0.801 |
| STAT1 | TCFL5 | 0.801 |
| SMARCD3 | JOSD1 | 0.801 |
| P2RY14 | CD79B | 0.801 |
| STAT1 | RASGRP2 | 0.801 |
| DENND1A | GPR65 | 0.801 |
| LHFPL2 | P2RY10 | 0.801 |
| CASP1 | SIDT1 | 0.801 |
| RTP4 | ALDH1A1 | 0.801 |
| RTP4 | MAK | 0.801 |
| C1QB | SAMHD1 | 0.801 |
| C1QB | TSPAN2 | 0.801 |
| C1QB | MTHFD2 | 0.801 |
| NPC2 | TOB1 | 0.801 |
| C1QB | ZNF395 | 0.801 |
| C1QB | ISG20 | 0.801 |
| STX11 | MYL9 | 0.801 |
| C1QB | NMT2 | 0.801 |
| C1QB | SORT1 | 0.801 |
| CNDP2 | PSTPIP2 | 0.801 |
| ATP1B2 | NDRG1 | 0.801 |
| TNFSF10 | UPB1 | 0.801 |
| ATP1B2 | FAS | 0.801 |
| GBP1 | IFIH1 | 0.801 |
| MCM5 | ETS1 | 0.801 |
| TAPBPL | CASP1 | 0.801 |
| PSME1 | TRIM22 | 0.801 |
| APOL2 | PPP1R2 | 0.801 |
| SLC6A12 | FAS | 0.801 |
| AFF1 | ACP5 | 0.801 |
| FAS | CLDND1 | 0.801 |
| C1QB | PRPF4 | 0.801 |
| DUSP3 | MSRB2 | 0.801 |
| TNFSF10 | MYL9 | 0.801 |
| SOCS1 | PSME1 | 0.801 |
| ALDH1A1 | CASP5 | 0.801 |
| STX11 | KLF13 | 0.801 |
| DUSP3 | GCH1 | 0.801 |
| STAT1 | TAPBPL | 0.8 |
| C1QB | SMAD7 | 0.8 |
| RAB20 | MAK | 0.8 |
| APOL2 | FAM8A1 | 0.8 |
| PRRG4 | GFOD1 | 0.8 |
| TNFSF10 | SESN1 | 0.8 |
| TRIM22 | MAK | 0.8 |
| PSTPIP2 | NELL2 | 0.8 |
| IRF1 | GFOD1 | 0.8 |
| APOL2 | LAX1 | 0.8 |
| DENND1A | TNFSF10 | 0.8 |
| NPC2 | GFOD1 | 0.8 |
| C1QB | CLCF1 | 0.8 |
| PSMA4 | CASP5 | 0.8 |
| IGF2BP3 | LHFPL2 | 0.8 |
| SMARCD3 | RAB20 | 0.8 |
| RTP4 | ATP1B2 | 0.8 |
| IRF1 | CA5B | 0.8 |
| STAT1 | ALDH1A1 | 0.8 |
| LHFPL2 | ZBTB32 | 0.8 |
| CASP5 | POLR3B | 0.8 |
| BST2 | C1QA | 0.8 |
| SMARCD3 | AFF1 | 0.8 |
| PSTPIP2 | GPR65 | 0.8 |
| FAM111A | MYL9 | 0.8 |
| PSME1 | IGF2BP3 | 0.8 |
| LHFPL2 | GFI1 | 0.8 |
| DUSP3 | VPS37B | 0.8 |

TABLE 7

| Gene 1 | Gene 2 | AUROC |
|---|---|---|
| GBP5 | EPHA4 | 0.906 |
| PLAAT4 | GBP5 | 0.903 |
| GBP5 | LAX1 | 0.901 |
| CYB561 | GBP5 | 0.899 |
| GBP5 | CA5B | 0.898 |
| GBP5 | SMAD7 | 0.896 |
| GBP5 | CD83 | 0.887 |
| GBP5 | CDKN1C | 0.862 |
| CYB561 | EPHA4 | 0.860 |
| PLAAT4 | CA5B | 0.843 |
| PLAAT4 | EPHA4 | 0.836 |
| CYB561 | CA5B | 0.829 |
| PLAAT4 | CYB561 | 0.820 |
| PLAAT4 | CD83 | 0.811 |
| PLAAT4 | LAX1 | 0.806 |
| PLAAT4 | SMAD7 | 0.790 |
| CYB561 | SMAD7 | 0.783 |
| CYB561 | LAX1 | 0.778 |
| CYB561 | CD83 | 0.771 |
| CA5B | EPHA4 | 0.756 |
| PLAAT4 | CDKN1C | 0.747 |
| LAX1 | EPHA4 | 0.732 |
| SMAD7 | EPHA4 | 0.730 |
| EPHA4 | CD83 | 0.723 |
| LAX1 | CA5B | 0.706 |
| SMAD7 | CA5B | 0.702 |
| CYB561 | CDKN1C | 0.695 |
| CDKN1C | EPHA4 | 0.694 |
| CA5B | CD83 | 0.684 |
| LAX1 | CD83 | 0.677 |
| SMAD7 | LAX1 | 0.661 |
| SMAD7 | CD83 | 0.660 |
| CDKN1C | CA5B | 0.656 |
| LAX1 | CDKN1C | 0.654 |
| SMAD7 | CDKN1C | 0.628 |
| CDKN1C | CD83 | 0.618 |

What is claimed is:

1. A method of analyzing a sample, the method comprising:
(a) obtaining a sample of RNA from a subject; and
(b) measuring the amount of RNA transcripts encoded by PLAAT4, CYB561, GBP5, SMAD7, LAX1, CDKN1C, CA5B, EPHA4 and CD83 in the sample, to produce gene expression data.

2. The method of claim 1, wherein the subject has latent tuberculosis or symptoms of active tuberculosis.

3. The method of any prior claim, wherein the sample comprises RNA isolated from whole blood, white blood cells, neutrophils, peripheral blood mononuclear cells (PBMCs), or buffy coat.

4. The method of any prior claim, further comprising:
(c) based on the gene expression data, generating a report indicating whether the subject has active tuberculosis or is progressing to active tuberculosis, wherein:
(i) increased PLAAT4, CYB561, and GBP5 expression indicates that the subject has active tuberculosis or is progressing to active tuberculosis; and
(ii) increased SMAD7, LAX1, CDKN1C, CA5B, EPHA4, and CD83 indicates that the subject does not have active tuberculosis and is not progressing to active tuberculosis.

5. The method of any prior claim, further comprising calculating a tuberculosis score based on the levels of expression of the RNA transcripts in the subject, wherein a higher tuberculosis score for the subject indicates that the subject is more likely to have active tuberculosis or progression to active tuberculosis.

6. The method of any prior claim further comprising diagnosing the patent as having active tuberculosis or progression to active tuberculosis based on the gene expression data, wherein:
(i) increased PLAAT4, CYB561 and GBP5 expression indicates that the subject has active tuberculosis or is progressing to active tuberculosis; and
(ii) increased SMAD7, LAX1, CDKN1C, CA5B, EPHA4 and CD83 indicates that the subject does not have active tuberculosis and is not progressing to active tuberculosis.

7. The method of any prior claim, further comprising identifying the subject as having active tuberculosis or progression to active tuberculosis based on the gene expression data, and administering antibiotics to the patient.

8. The method of claim 7, wherein the antibiotics comprise one or more of isoniazid, rifampin, pyrazinamide, and ethambutol.

9. The method of claim any prior claim, wherein the measuring step is done by RT-PCR.

10. The method of any of claims 1-8, wherein the measuring step is done using a quantitative isothermal amplification method.

11. The method of any of claims 1-8, wherein the measuring step is done by sequencing.

12. The method of any of claims 1-8, wherein the measuring step is done by labeling the RNA or cDNA made from the same and hybridizing the labeled RNA or cDNA to a support.

13. A method for treating a subject, comprising:
(a) identifying a patient as having active tuberculosis or progression to active tuberculosis based on the amount of RNA transcripts encoded by PLAAT4, CYB561, GBP5, SMAD7, LAX1, CDKN1C, CA5B, EPHA4 and CD83 in a sample from the subject; and
(b) treating the subject with antibiotics.

14. The method of claim 13, wherein the antibiotics comprise one or more of isoniazid, rifampin, pyrazinamide, and ethambutol.

15. The method of any of claims 13-14, wherein the identifying is done by reviewing a report indicating whether the subject has active tuberculosis or is progressing to active tuberculosis.

16. The method of claim 15, wherein the report comprises a tuberculosis score, wherein a higher tuberculosis score for the subject indicates that the subject is more likely to have active tuberculosis or progression to active tuberculosis.

17. The method of any of claims 13-16, further comprising monitoring the subject's response to antibiotics.

18. The method of claim 17, further comprising calculating a tuberculosis score before and after treatment, wherein an increasing tuberculosis score indicates that the tuberculosis infection is worsening and a decreasing tuberculosis score indicates tat the subject is recovering.

19. A kit for diagnosing active tuberculosis or progression to active tuberculosis, comprising reagents for measuring the amount of RNA transcripts encoded by PLAAT4, CYB561, GBP5, SMAD7, LAX1, CDKN1C, CA5B, EPHA4 and CD83.

20. The kit of claim 19, wherein the reagents comprise, for each RNA transcript, a sequence-specific oligonucleotide that hybridizes to the transcript.

21. The kit of claim 20, wherein sequence-specific oligonucleotide is biotinylated and/or labeled with an optically-detectable moiety.

22. The kit of any of claims 19-21, wherein the reagents comprises, for each RNA transcript, at least a pair of PCR primers that amplify a sequence from the RNA transcript, or cDNA made from the same.

23. The kit of claim 19, wherein the reagents comprise multiple reaction vessels, each comprising at least one sequence-specific isothermal amplification primer that hybridizes to the transcript, or cDNA made from the same.
